(19) United States
(12) Patent Application Publication
(10) Pub. No.: US 2024/0132901 A1
(43) Pub. Date: Apr. 25, 2024

(54) METHODS AND COMPOSITIONS FOR REGULATING ALKALOIDS IN TOBACCO

(71) Applicant: ALTRIA CLIENT SERVICES LLC, Richmond, VA (US)

(72) Inventors: Chengalrayan KUDITHIPUDI, Midlothian, VA (US); Rajanikanth GOVINDARAJULU, Henrico, VA (US); Raja S. PAYYAVULA, Henrico, VA (US)

(73) Assignee: ALTRIA CLIENT SERVICES LLC, Richmond, VA (US)

(21) Appl. No.: 18/365,270

(22) Filed: Aug. 3, 2023

Related U.S. Application Data
(60) Provisional application No. 63/395,459, filed on Aug. 5, 2022.

(51) Int. Cl. C12N 15/82; A24B 13/00; A24B 15/10; C07K 14/415

(52) U.S. Cl. CPC C12N 15/8243; A24B 13/00; A24B 15/10; C07K 14/415

(57) ABSTRACT

The present disclosure relates to tobacco plants, plant parts, seeds, compositions, and methods related to modulating the expression of novel genes in tobacco to regulate alkaloid levels.

### CROSS-REFERENCE TO RELATED APPLICATION

This application claims the benefit of U.S. Provisional Application No. 63/395,459, filed on Aug. 5, 2022, the contents of which are incorporated herein by reference in its entirety.

### FIELD

The present disclosure relates to tobacco plants, plant parts, seeds, compositions, and methods related to modulating the expression of novel genes in tobacco to regulate alkaloid levels.

### INCORPORATION OF SEQUENCE LISTING

A sequence listing conforming to the rules of WIPO Standard ST.26 is hereby incorporated by reference in its entirety. Said sequence listing has been filed as an electronic document via PatentCenter encoded as XML in UTF-8 text. The electronic document is named “P34753US01_SL.xml”, and is 341,150 bytes in size (measured in MS-Windows®) and created on Jul. 26, 2023.

A listing of nucleic acid sequences and amino acid sequences is provided in Table 1.

TABLE 1
Nucleic acid sequences and amino acid sequences

| SEQ ID NO | Sequence Description | Sequence Type |
|---|---|---|
| 1 | g12941 | Nucleic Acid |
| 2 | g14663 | Nucleic Acid |
| 3 | g20986 | Nucleic Acid |
| 4 | g20989a | Nucleic Acid |
| 5 | g20989b | Nucleic Acid |
| 6 | g22648 | Nucleic Acid |
| 7 | g23499 | Nucleic Acid |
| 8 | g26597 | Nucleic Acid |
| 9 | g31004 | Nucleic Acid |
| 10 | g31698 | Nucleic Acid |
| 11 | g31724 | Nucleic Acid |
| 12 | g44455 | Nucleic Acid |
| 13 | g44587 | Nucleic Acid |
| 14 | g47147 | Nucleic Acid |
| 15 | g49713 | Nucleic Acid |
| 16 | g52130 | Nucleic Acid |
| 17 | g57292 | Nucleic Acid |
| 18 | g59464 | Nucleic Acid |
| 19 | g59465 | Nucleic Acid |
| 20 | g60786 | Nucleic Acid |
| 21 | g62380 | Nucleic Acid |
| 22 | g62763 | Nucleic Acid |
| 23 | g66085 | Nucleic Acid |
| 24 | g66301 | Nucleic Acid |
| 25 | g68242 | Nucleic Acid |
| 26 | g69182 | Nucleic Acid |
| 27 | g69391 | Nucleic Acid |
| 28 | g71598 | Nucleic Acid |
| 29 | g74738 | Nucleic Acid |
| 30 | g74782 | Nucleic Acid |
| 31 | g74912 | Nucleic Acid |
| 32 | g75157 | Nucleic Acid |
| 33 | g75446 | Nucleic Acid |
| 34 | g75500 | Nucleic Acid |
| 35 | g76096 | Nucleic Acid |
| 36 | g77018 | Nucleic Acid |
| 37 | g77780 | Nucleic Acid |
| 38 | g78265 | Nucleic Acid |
| 39 | g78408 | Nucleic Acid |
| 40 | g79607 | Nucleic Acid |
| 41 | g79774 | Nucleic Acid |
| 42 | g82124 | Nucleic Acid |
| 43 | g82252 | Nucleic Acid |
| 44 | g82744 | Nucleic Acid |
| 45 | g83936 | Nucleic Acid |
| 46 | g86315 | Nucleic Acid |
| 47 | g86407 | Nucleic Acid |
| 48 | g86563 | Nucleic Acid |
| 49 | g89013 | Nucleic Acid |
| 50 | g91123 | Nucleic Acid |
| 51 | g91334 | Nucleic Acid |
| 52 | g91336 | Nucleic Acid |
| 53 | g91411 | Nucleic Acid |
| 54 | g92376 | Nucleic Acid |
| 55 | g92662 | Nucleic Acid |
| 56 | g93191 | Nucleic Acid |
| 57 | g95317 | Nucleic Acid |
| 58 | g95400 | Nucleic Acid |
| 59 | g12941 | Nucleic Acid |
| 60 | g14663 | Nucleic Acid |
| 61 | g20986 | Nucleic Acid |
| 62 | g20989a | Nucleic Acid |
| 63 | g20989b | Nucleic Acid |
| 64 | g22648 | Nucleic Acid |
| 65 | g23499 | Nucleic Acid |
| 66 | g26597 | Nucleic Acid |
| 67 | g31004 | Nucleic Acid |
| 68 | g31698 | Nucleic Acid |
| 69 | g31724 | Nucleic Acid |
| 70 | g44455 | Nucleic Acid |
| 71 | g44587 | Nucleic Acid |
| 72 | g47147 | Nucleic Acid |
| 73 | g49713 | Nucleic Acid |
| 74 | g52130 | Nucleic Acid |
| 75 | g57292 | Nucleic Acid |
| 76 | g59464 | Nucleic Acid |
| 77 | g59465 | Nucleic Acid |
| 78 | g60786 | Nucleic Acid |
| 79 | g62380 | Nucleic Acid |
| 80 | g62763 | Nucleic Acid |
| 81 | g66085 | Nucleic Acid |
| 82 | g66301 | Nucleic Acid |
| 83 | g68242 | Nucleic Acid |
| 84 | g69182 | Nucleic Acid |
| 85 | g69391 | Nucleic Acid |
| 86 | g71598 | Nucleic Acid |
| 87 | g74738 | Nucleic Acid |
| 88 | g74782 | Nucleic Acid |
| 89 | g74912 | Nucleic Acid |
| 90 | g75157 | Nucleic Acid |
| 91 | g75446 | Nucleic Acid |
| 92 | g75500 | Nucleic Acid |
| 93 | g76096 | Nucleic Acid |
| 94 | g77018 | Nucleic Acid |
| 95 | g77780 | Nucleic Acid |
| 96 | g78265 | Nucleic Acid |
| 97 | g78408 | Nucleic Acid |
| 98 | g79607 | Nucleic Acid |
| 99 | g79774 | Nucleic Acid |
| 100 | g82124 | Nucleic Acid |
| 101 | g82252 | Nucleic Acid |
| 102 | g82744 | Nucleic Acid |
| 103 | g83936 | Nucleic Acid |
| 104 | g86315 | Nucleic Acid |
| 105 | g86407 | Nucleic Acid |
| 106 | g86563 | Nucleic Acid |
| 107 | g89013 | Nucleic Acid |
| 108 | g91123 | Nucleic Acid |
| 109 | g91334 | Nucleic Acid |
| 110 | g91336 | Nucleic Acid |
| 111 | g91411 | Nucleic Acid |
| 112 | g92376 | Nucleic Acid |
| 113 | g92662 | Nucleic Acid |
| 114 | g93191 | Nucleic Acid |
| 115 | g95317 | Nucleic Acid |
| 116 | g95400 | Nucleic Acid |
| 117 | g12941 | Amino Acid |
| 118 | g14663 | Amino Acid |
| 119 | g20986 | Amino Acid |
| 120 | g20989a | Amino Acid |
| 121 | g20989b | Amino Acid |
| 122 | g22648 | Amino Acid |
| 123 | g23499 | Amino Acid |
| 124 | g26597 | Amino Acid |
| 125 | g31004 | Amino Acid |
| 126 | g31698 | Amino Acid |
| 127 | g31724 | Amino Acid |
| 128 | g44455 | Amino Acid |
| 129 | g44587 | Amino Acid |
| 130 | g47147 | Amino Acid |
| 131 | g49713 | Amino Acid |
| 132 | g52130 | Amino Acid |
| 133 | g57292 | Amino Acid |
| 134 | g59464 | Amino Acid |
| 135 | g59465 | Amino Acid |
| 136 | g60786 | Amino Acid |
| 137 | g62380 | Amino Acid |
| 138 | g62763 | Amino Acid |
| 139 | g66085 | Amino Acid |
| 140 | g66301 | Amino Acid |
| 141 | g68242 | Amino Acid |
| 142 | g69182 | Amino Acid |
| 143 | g69391 | Amino Acid |
| 144 | g71598 | Amino Acid |
| 145 | g74738 | Amino Acid |
| 146 | g74782 | Amino Acid |
| 147 | g74912 | Amino Acid |
| 148 | g75157 | Amino Acid |
| 149 | g75446 | Amino Acid |
| 150 | g75500 | Amino Acid |
| 151 | g76096 | Amino Acid |
| 152 | g77018 | Amino Acid |
| 153 | g77780 | Amino Acid |
| 154 | g78265 | Amino Acid |
| 155 | g78408 | Amino Acid |
| 156 | g79607 | Amino Acid |
| 157 | g79774 | Amino Acid |
| 158 | g82124 | Amino Acid |
| 159 | g82252 | Amino Acid |
| 160 | g82744 | Amino Acid |
| 161 | g83936 | Amino Acid |
| 162 | g86315 | Amino Acid |
| 163 | g86407 | Amino Acid |
| 164 | g86563 | Amino Acid |
| 165 | g89013 | Amino Acid |
| 166 | g91123 | Amino Acid |
| 167 | g91334 | Amino Acid |
| 168 | g91336 | Amino Acid |
| 169 | g91411 | Amino Acid |
| 170 | g92376 | Amino Acid |
| 171 | g92662 | Amino Acid |
| 172 | g93191 | Amino Acid |
| 173 | g95317 | Amino Acid |
| 174 | g95400 | Amino Acid |
| 175 | Expression | Nucleic acid |
| | construct | |
| 176 | Tobacco | Nucleic acid |
| | eukaryotic | |
| | translation | |
| | elongation | |
| | factor 1 alpha | |
| | (EF1α) | |
| 177 | Tobacco | Nucleic acid |
| | eukaryotic | |
| | translation | |
| | elongation | |
| | factor 1 alpha | |
| | (EF1α) | |
| 178 | Tobacco | Nucleic acid |
| | eukaryotic | |
| | translation | |
| | elongation | |
| | factor 1 alpha | |
| | (EF1α) | |

### BACKGROUND

Four major alkaloids are found in tobacco: nicotine, nornicotine, anabasine, and anatabine. Nicotine is the predominant alkaloid, usually accounting for more than 90% of the total alkaloids in commercial tobacco cultivars. Nicotine biosynthesis occurs predominantly in tobacco roots. Tobacco plants then transport nicotine through the vascular bundle to leaves where nicotine is then stored in the vacuoles. Cumulative levels of nicotine and related alkaloids in tobacco leaves depend on biosynthesis and transport, both of which can be influenced by environmental conditions, phytohormones, developmental status, and gene regulatory factors.

Methods and compositions for modulating novel tobacco genes to modulate alkaloid levels in tobacco are provided herein.

### SUMMARY

In one aspect, this disclosure provides a modified tobacco plant, or part thereof, comprising at least one non-natural mutation in an endogenous nucleic acid sequence, where the endogenous nucleic acid sequence encodes a polypeptide comprising an amino acid sequence at least 80% identical or similar to an amino acid sequence selected from the group consisting of SEQ ID NOs: 117-174.

In one aspect, this disclosure provides a modified tobacco plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 80% identical or similar to an amino acid sequence selected from the group consisting of SEQ ID NOs: 117-174.

In one aspect, this disclosure provides a modified tobacco plant, or part thereof, comprising a recombinant DNA construct comprising a heterologous promoter operably linked to a nucleic acid sequence encoding a polypeptide comprising an amino acid sequence at least 80% identical or similar to an amino acid sequence selected from the group consisting of SEQ ID NOs: 117-174.

In one aspect, this disclosure provides a method of producing a modified tobacco plant, the method comprising: (a) inducing a non-natural mutation in at least one tobacco cell in an endogenous nucleic acid sequence encoding a polypeptide comprising an amino acid sequence at least 80% identical or similar to an amino acid sequence selected from the group consisting of SEQ ID NOs: 117-174; (b) selecting at least one tobacco cell comprising the non-natural mutation from step (a); and (c) regenerating at least one modified tobacco plant from the at least one tobacco cell selected in step (b).

In one aspect, this disclosure provides a method of producing a modified tobacco plant, the method comprising: (a) introducing a recombinant DNA construct to at least one tobacco cell, where the recombinant DNA construct comprises a heterologous promoter operably linked to a nucleic acid encoding at least one small RNA molecule capable of binding to and reducing the expression of an endogenous nucleic acid sequence encoding a polypeptide at least 80% identical or similar to an amino acid sequence selected from the group consisting of SEQ ID NOs: 117-174; (b) selecting at least one tobacco cell comprising the recombinant DNA construct; and; (c) regenerating at least one modified tobacco plant from the at least one tobacco cell selected in step (b).

In one aspect, this disclosure provides a method of producing a modified tobacco plant, the method comprising: (a) introducing a recombinant DNA construct to at least one tobacco cell, where the recombinant DNA construct comprises a heterologous promoter operably linked to a nucleic acid sequence encoding a polypeptide comprising an amino acid sequence at least 80% identical or similar to an amino acid sequence selected from the group consisting of SEQ ID NOs: 117-174; (b) selecting at least one tobacco cell comprising the recombinant DNA construct; and (c) regenerating at least one modified tobacco plant from the at least one tobacco cell selected in step (b).

In one aspect, this disclosure provides a method comprising preparing a tobacco product using cured tobacco material from a modified tobacco plant, where the modified tobacco plant comprises a non-natural mutation in an endogenous nucleic acid sequence, where the endogenous nucleic acid sequence encodes a polypeptide comprising an amino acid sequence at least 80% identical or similar to an amino acid sequence selected from the group consisting of SEQ ID NOs: 117-174.

In one aspect, this disclosure provides a method comprising preparing a tobacco product using cured tobacco material from a modified tobacco plant, where the modified tobacco plant comprises a recombinant DNA construct, and where the recombinant DNA construct comprises a heterologous promoter operably linked to a nucleic acid sequence encoding at least one small RNA molecule capable of binding to and reducing the expression of an endogenous nucleic acid sequence encoding a polypeptide at least 80% identical or similar to an amino acid sequence selected from the group consisting of SEQ ID NOs: 117-174.

In one aspect, this disclosure provides a method comprising preparing a tobacco product using cured tobacco material from a modified tobacco plant, where the modified tobacco plant comprises a recombinant DNA construct, and where the recombinant DNA construct comprises a heterologous promoter operably linked to a nucleic acid sequence encoding a polypeptide comprising an amino acid sequence at least 80% identical or similar to an amino acid sequence selected from the group consisting of SEQ ID NOs: 117-174.

In one aspect, this disclosure provides a method comprising transforming a tobacco cell with a recombinant DNA construct, where the recombinant DNA construct comprises a heterologous promoter operably linked to a nucleic acid sequence encoding at least one small RNA molecule capable of binding to and reducing the expression of an endogenous nucleic acid sequence encoding a polypeptide at least 80% identical or similar to an amino acid sequence selected from the group consisting of SEQ ID NOs: 117-174.

In one aspect, this disclosure provides a method comprising transforming a tobacco cell with a recombinant DNA construct, where the recombinant DNA construct comprises a heterologous promoter operably linked to a nucleic acid sequence encoding a polypeptide comprising an amino acid sequence at least 80% identical or similar to an amino acid sequence selected from the group consisting of SEQ ID NOs: 117-174.

In one aspect, this disclosure provides a method for producing a modified tobacco plant, the method comprising: (a) crossing at least one tobacco plant of a first tobacco variety with at least one tobacco plant of a second tobacco variety to produce at least one progeny tobacco seed, where the at least one tobacco plant of the first tobacco variety comprises a non-natural mutation in an endogenous nucleic acid sequence, where the endogenous nucleic acid sequence encodes a polypeptide comprising an amino acid sequence at least 80% identical or similar to an amino acid sequence selected from the group consisting of SEQ ID NOs: 117-174, where the non-natural mutation is not present in the endogenous nucleic acid sequence in a control tobacco plant of the first tobacco variety; and; (b) selecting for at least one progeny tobacco seed, or a plant germinated therefrom, where the at least one tobacco seed or plant germinated therefrom comprises the non-natural mutation.

In one aspect, this disclosure provides a method for producing a modified tobacco plant, the method comprising: (a) crossing at least one tobacco plant of a first tobacco variety with at least one tobacco plant of a second tobacco variety to produce at least one progeny tobacco seed, where the at least one tobacco plant of the first tobacco variety comprises a recombinant DNA construct, where the recombinant DNA construct comprises a heterologous promoter operably linked to a nucleic acid sequence encoding at least one small RNA molecule capable of binding to and reducing the expression of an endogenous nucleic acid sequence encoding a polypeptide at least 80% identical or similar to an amino acid sequence selected from the group consisting of SEQ ID NOs: 117-174, where the recombinant DNA construct is not present in the endogenous nucleic acid sequence in a control tobacco plant of the same variety; and; (b) selecting for at least one progeny tobacco seed, or a plant germinated therefrom, where the at least one tobacco seed or plant germinated therefrom comprises the recombinant DNA construct.

In one aspect, this disclosure provides a method for producing a modified tobacco plant, the method comprising: (a) crossing at least one tobacco plant of a first tobacco variety with at least one tobacco plant of a second tobacco variety to produce at least one progeny tobacco seed, where the at least one tobacco plant of the first tobacco variety comprises a recombinant DNA construct, where the recombinant DNA construct comprises a heterologous promoter operably linked to a nucleic acid sequence encoding a polypeptide comprising an amino acid sequence at least 80% identical or similar to an amino acid sequence selected from the group consisting of SEQ ID NOs: 117-174, where the recombinant DNA construct is not present in the nucleic acid sequence in a control tobacco plant of the first tobacco variety; and (b) selecting for at least one progeny tobacco seed, or a plant germinated therefrom, where the at least one tobacco seed or plant germinated therefrom comprises the recombinant DNA construct.

In one aspect, this disclosure provides a modified tobacco plant, or part thereof, comprising at least one non-natural mutation in an endogenous nucleic acid sequence that modulates the expression or functional activity of a gene, where the gene encodes a polypeptide comprising an amino acid sequence at least 80% identical or similar to an amino acid sequence selected from the group consisting of SEQ ID NOs: 117-174.

### BRIEF DESCRIPTION OF THE DRAWINGS

FIG. 1 depicts clustering of genes based on dissimilarity of topological overlap measurement. The bars at the bottom of the figure indicate different network modules.

FIG. 2 depicts subnetworks extracted from wgcna and visualized in Cytoscape. Genes (nodes) labelled in the center of each subnetwork are known nicotine biosynthetic genes. Abbreviations: AO3 (L-ascorbate oxidase 3); AO-2a (L-ascorbate oxidase 2a); BBL-3 (berberine bridge enzyme-like 3); ADC-1b (arginine decarboxylase 1b); BBL-4 (berberine bridge enzyme-like 4); QS1b (quinolinate synthase 1b); QS1a (quinolinate synthase 1a); ADC-1a (arginine decarboxylase 1a); PMT1b (putrescine methyltransferase 1b); BBL-1 (berberine bridge enzyme-like 1); PMT3 (putrescine methyltransferase 3); AO7 (L-ascorbate oxidase 7); ODC-1a (ornithine decarboxylase 1a); AO11 (L-ascorbate oxidase 11); ODC-1b (ornithine decarboxylase 1b); PMT1a (putrescine methyltransferase 1a); AIC-1b (agmatine deiminase 1b); A622-1 (isoflavone reductase homolog A622 1); PMT4 (putrescine methyltransferase 4); MPO1a (N-methylputrescine oxidase 1a); AO12 (L-ascorbate oxidase 12); ADC-1c (arginine decarboxylase 1c); LDC (lysine decarboxylase); AO4 (L-ascorbate oxidase 4); AO17 (L-ascorbate oxidase 17); A622-2 (isoflavone reductase homolog A622 2); A622-3 (isoflavone reductase homolog A622 3); BBL-2 (berberine bridge enzyme-like 2); and A622-4 (isoflavone reductase homolog A622 4).

FIG. 3 depicts a heat map showing the expression in terms of log 2 fold-change between before topping (BT), three-days post-topping (3 D), and two-weeks post-topping (2 W) in TN90 tobacco root samples.

FIGS. 4 to 16 depict the relative expression (as compared to tobacco eukaryotic translation elongation factor 1 alpha (EF1α)) of fourteen novel tobacco genes in root tissue of the tobacco lines TN90 and CS118. Expression is examined before topping (BT), three days after topping (3 d) and one week after topping (1 w). Error bars, if present, represent one standard deviation. FIG. 4 depicts relative expression of g95400 (SEQ ID NO: 58). FIG. 5 depicts relative expression of g82124 (SEQ ID NO: 42). FIG. 6 depicts relative expression of g23499 (SEQ ID NO: 7). FIG. 7 depicts relative expression of g69182 (SEQ ID NO: 26). FIG. 8 depicts relative expression of g14663 (SEQ ID NO: 2). FIG. 9 depicts relative expression of g71598 (SEQ ID NO: 28). FIG. 10 depicts relative expression of g74782 (SEQ ID NO: 30).

FIG. 11 depicts relative expression of g92662 (SEQ ID NO: 55). FIG. 12 depicts relative expression of g91334 (SEQ ID NO: 51). FIG. 13 depicts relative expression of g93191 (SEQ ID NO: 56). FIG. 14 depicts relative expression of g74738 (SEQ ID NO: 29). FIG. 15 depicts relative expression of g66301 (SEQ ID NO: 24). FIG. 16 depicts relative expression of g86407 (SEQ ID NO: 47).

FIG. 17 depicts relative expression (as compared to EF1α) of g22648 in over-expression (OX) and knockdown (RNAi) lines as compared to three vector control lines (VC-1, VC-2, and VC-3).

FIG. 18 depicts the relative expression (as compared to EF1α) of putrescine N-methyltransferase (PMT) and quinolinate phosphoribosyltransferase (QPT) gene families in g22648 overexpression (OX) and knockdown (RNAi) tobacco lines, as compared to three vector control lines (VC-1, VC-2, and VC-3).

FIG. 19 depicts nicotine levels in g22648 overexpression (OX) and knockdown (RNAi) tobacco lines, as compared to three vector control lines (VC).

FIG. 20 depicts nornicotine, anatabine, and anabasine levels in g22648 overexpression (OX) and knockdown (RNAi) tobacco lines, as compared to three vector control lines (VC).

FIG. 21 depicts relative expression (as compared to EF1α) of g95400 in an RNAi knockdown tobacco line as compared to a vector control line (VC).

FIG. 22 depicts relative expression (as compared to EF1α) of PMT and QPT gene families in a g95400 RNAi knockdown tobacco line as compared to a vector control line (VC).

FIG. 23 depicts nicotine levels in a g95400 RNAi knockdown line as compared to a vector control line (VC).

FIG. 24 depicts nornicotine, anatabine, and anabasine levels in a g95400 RNAi knockdown tobacco line as compared to a vector control line (VC).

FIG. 25 depicts relative expression (as compared to EF1α) of g74912 in RNAi knockdown tobacco line as compared to three vector control lines (VC).

FIG. 26 depicts relative expression (as compared to EF1α) of PMT and QPT gene families in g74912 RNAi knockdown tobacco lines as compared to two vector control lines (VC).

FIG. 27 depicts nicotine levels in g74912 RNAi knockdown tobacco lines as compared to two vector control lines (VC).

FIG. 28 depicts nornicotine, anatabine, and anabasine levels in g74912 RNAi knockdown tobacco lines as compared to two vector control lines (VC).

FIG. 29 depicts relative expression (as compared to EF1α) of g12941 in over-expression (OX) and knockdown (RNAi) lines as compared to three vector control lines (VC-1, VC-2, and VC-3).

FIG. 30 depicts relative expression (as compared to EF1α) of putrescine N-methyltransferase (PMT) and quinolinate phosphoribosyltransferase (QPT) in g12941 overexpression (OX) and knockdown (RNAi) tobacco lines, as compared to three vector control lines (VC-1, VC-2, and VC-3).

FIG. 31 depicts nicotine levels in g12941 overexpression (OX) and knockdown (RNAi) tobacco lines, as compared to three vector control lines (VC-1, VC-2, and VC-3).

FIG. 32 depicts nornicotine, anatabine, and anabasine levels in g12941 overexpression (OX) and knockdown (RNAi) tobacco lines, as compared to three vector control lines (VC-1, VC-2, and VC-3).

FIG. 33 depicts relative expression (as compared to EF1α) of g75446 in RNAi knockdown tobacco lines as compared to two vector control lines (VC).

FIG. 34 depicts relative expression (as compared to EF1α) of PMT and QPT gene families gene families in g75446 RNAi knockdown tobacco lines as compared to two vector control lines (VC).

FIG. 35 depicts nicotine levels in g75446 RNAi knockdown tobacco lines as compared to two vector control lines (VC).

FIG. 36 depicts nornicotine, anatabine, and anabasine levels in g75446 RNAi knockdown tobacco lines as compared to two vector control lines (VC).

FIG. 37 depicts relative expression (as compared to EF1α) of g31724 in two RNAi knockdown tobacco lines as compared to a vector control line (VC).

FIG. 38 depicts relative expression (as compared to EF1α) of PMT and QPT gene families gene families in two g31724 RNAi knockdown tobacco lines as compared to a vector control line (VC).

FIG. 39 depicts nicotine levels in two g31724 RNAi knockdown tobacco lines as compared to a vector control line (VC).

FIG. 40 depicts nornicotine, anatabine, and anabasine levels in two g31724 RNAi knockdown tobacco lines as compared to a vector control line (VC).

FIG. 41 depicts relative expression (as compared to EF1α) of g14663 overexpression (OX) in two tobacco lines as compared to a vector control line (VC).

FIG. 42 depicts relative expression (as compared to EF1α) of PMT and QPT gene families in two g14663 overexpression (OX) tobacco lines as compared to a vector control line (VC).

FIG. 43 depicts nicotine levels in two g14663 overexpression (OX) tobacco lines as compared to a vector control line (VC).

FIG. 44 depicts nornicotine, anatabine, and anabasine levels in two g14663 overexpression (OX) tobacco lines as compared to a vector control line (VC).

FIG. 45 depicts relative expression (as compared to EF1α) of g71598 overexpression (OX) in two tobacco lines as compared to a vector control line (VC).

FIG. 46 depicts relative expression (as compared to EF1α) of PMT and QPT gene families in two g71598 overexpression (OX) tobacco lines as compared to a vector control line (VC).

FIG. 47 depicts nicotine levels in two g71598 overexpression (OX) tobacco lines as compared to a vector control line (VC).

FIG. 48 depicts nornicotine, anatabine, and anabasine levels in two g71598 overexpression (OX) tobacco lines as compared to a vector control line (VC).

FIG. 49 depicts relative expression (as compared to EF1α) of g82744 in two RNAi knockdown tobacco lines as compared to two vector control lines (VC).

FIG. 50 depicts relative expression (as compared to EF1α) of PMT and QPT gene families in two g82744 RNAi knockdown tobacco lines as compared to two vector control lines (VC).

FIG. 51 depicts nicotine levels in two g82744 RNAi knockdown tobacco lines as compared to two vector control lines (VC).

FIG. 52 depicts nornicotine, anatabine, and anabasine levels in two g82744 RNAi knockdown tobacco lines as compared to two vector control lines (VC).

FIG. 53 depicts relative expression (as compared to EF1α) of g79774 in three RNAi knockdown tobacco lines as compared to two vector control lines (VC).

FIG. 54 depicts relative expression (as compared to EF1α) of PMT and QPT gene families in three g79774 RNAi knockdown tobacco lines as compared to two vector control lines (VC).

FIG. 55 depicts nicotine levels in three g79774 RNAi knockdown tobacco lines as compared to two vector control lines (VC).

FIG. 56 depicts nornicotine, anatabine, and anabasine levels in three g79774 RNAi knockdown tobacco lines as compared to two vector control lines (VC).

FIG. 57 depicts relative expression (as compared to EF1α) of g82124 in three RNAi knockdown tobacco lines as compared to three vector control lines (VC).

FIG. 58 depicts relative expression (as compared to EF1α) of PMT and QPT gene families in three g82124 RNAi knockdown tobacco lines as compared to three vector control lines (VC).

FIG. 59 depicts nicotine levels in three g82124 RNAi knockdown tobacco lines as compared to a vector control line (VC).

FIG. 60 depicts nornicotine, anatabine, and anabasine levels in three g82124 RNAi knockdown tobacco lines as compared to a vector control line (VC).

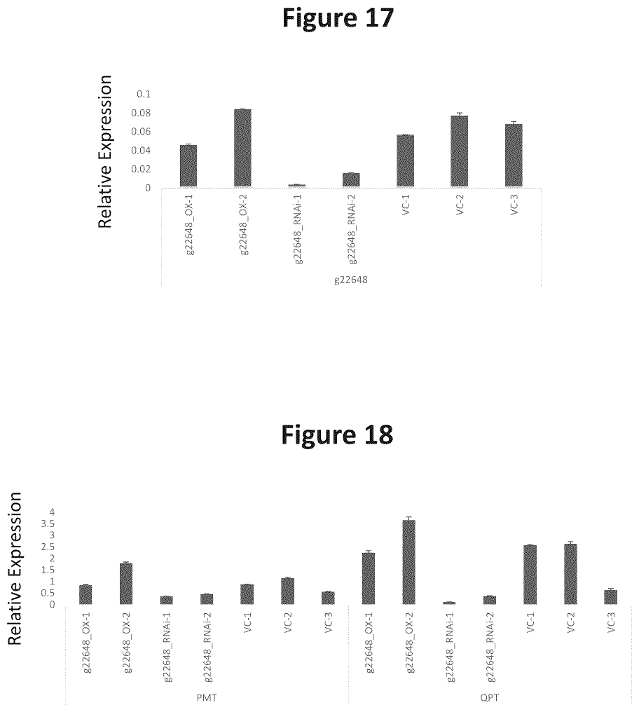

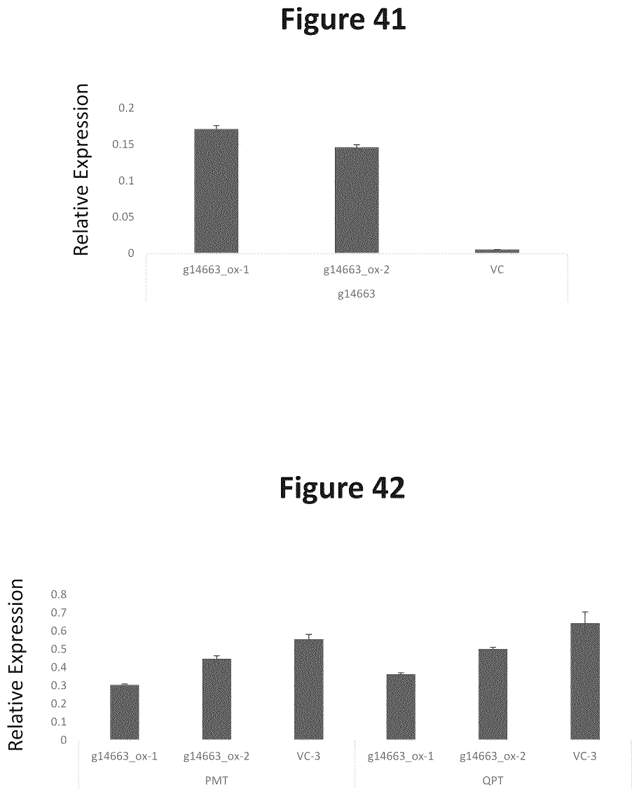

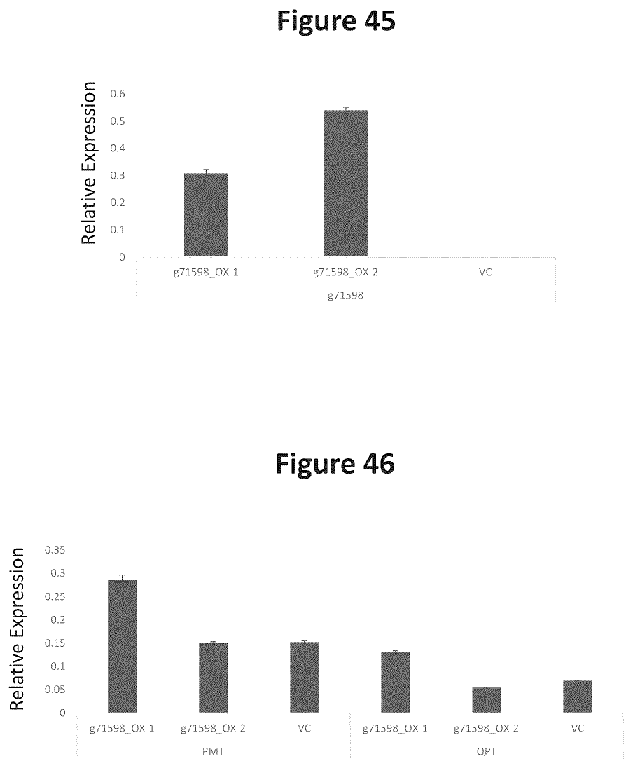

For FIGS. 17, 18, 22, 25, 26, 29, 30, 33, 34, 37, 38, 41, 42, 45, 46, 49, 50, 53, 54, 57, and 58, error bars represent the range of observed data.

### DETAILED DESCRIPTION

Unless defined otherwise, all technical and scientific terms used have the same meaning as commonly understood by one of ordinary skill in the art to which this disclosure belongs. Where a term is provided in the singular, the inventors also contemplate aspects of the disclosure described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6th edition, 2002, McGraw-Hill, New York), or the “Oxford Dictionary of Biology” (6th edition, 2008, Oxford University Press, Oxford and New York).

Any references cited herein, including, e.g., all patents, published patent applications, and non-patent publications, are incorporated herein by reference in their entirety.

When a grouping of alternatives is presented, any and all combinations of the members that make up that grouping of alternatives is specifically envisioned. For example, if an item is selected from a group consisting of A, B, C, and D, the inventors specifically envision each alternative individually (e.g., A alone, B alone, etc.), as well as combinations such as A, B, and D; A and C; B and C; etc. The term “and/or” when used in a list of two or more items means any one of the listed items by itself or in combination with any one or more of the other listed items. For example, the expression “A and/or B” is intended to mean either or both of A and B—i.e., A alone, B alone, or A and B in combination. The expression “A, B and/or C” is intended to mean A alone, B alone, C alone, A and B in combination, A and C in combination, B and C in combination, or A, B, and C in combination.

When a range of numbers is provided herein, the range is understood to inclusive of the edges of the range as well as any number between the defined edges of the range. For example, “between 1 and 10” includes any number between 1 and 10, as well as the number 1 and the number 10.

When the term “about” is used in reference to a number, it is understood to mean plus or minus 10%. For example, “about 100” would include from 90 to 110.

As used herein, the singular form “a,” “an,” and “the” include plural references unless the context clearly dictates otherwise. For example, the term “a compound” or “at least one compound” may include a plurality of compounds, including mixtures thereof.

Any tobacco plant, or part thereof, provided herein is specifically envisioned for use with any method provided herein. Similarly, any modified tobacco plant, or part thereof, is specifically envisioned for use with any method provided herein. Any nucleic acid sequence, amino acid sequence, or other composition provided herein is specifically envisioned for use with any method provided herein.

Tobacco plants typically produce alkaloids at levels between 2% and 4% of their total dry weight. Nicotine is a major alkaloid compound in tobacco plants, and it often accounts for approximately 95% of the total alkaloid content of the plants. The remaining pool of alkaloids primarily comprises other structurally-related alkaloids such as anabasine, anatabine, and nornicotine.

Nicotine is synthesized in roots via one of two primary metabolic pathways: the pyridine nicotine biosynthesis pathway and the pyrrolidine nicotine biosynthesis pathway.

In the pyridine nicotine biosynthesis pathway, quinolinate synthetase (QS) produces quinolinic acid, and the conversion of quinolinic acid to nicotinic acid mono-nucleotide (NAMN) is catalyzed by quinolinate phosphoribosyl-transferase (QPT). NAMN can be converted into nicotinic acid either directly by a NAMN glycohydrolase, or through a multi-step process involving the synthesis and degradation of nicotine adenine dinucleotide (NAD). Further reduction of nicotinic acid subsequently forms 3,6-dihydronicotinic acid.

In the pyrrolidine nicotine biosynthesis pathway, the decarboxylation of amino acids (e.g., arginine) is required to form putrescine, and methylation of putrescine to N-methylputrescine is catalyzed by N-methyltransferase (PMT). N-methylputrescine is then oxidized by a diamine oxidase and cyclized spontaneously to the N-methyl-Δ¹-pyrrolinium cation. This N-methyl-Δ¹-pyrrolinium cation is then coupled with 3,6-dihydronicotinic acid or another nicotinic acid intermediate to form nicotine. However, the exact substrate and enzymes involved in these reactions remains unknown.

Here, novel genes involved in the biosynthesis of nicotine or other alkaloids in tobacco are provided.

In an aspect, this disclosure provides a modified tobacco plant, or part thereof, comprising at least one non-natural mutation in an endogenous nucleic acid sequence, where the endogenous nucleic acid sequence encodes a polypeptide comprising an amino acid sequence at least 80% identical or similar to an amino acid sequence selected from the group consisting of SEQ ID NOs: 117-174. In another aspect, this disclosure provides a modified tobacco plant, or part thereof, comprising at least one non-natural mutation in an endogenous nucleic acid sequence at least 80% identical to a nucleotide sequence selected from the group consisting of SEQ ID NOs: 1-58. In another aspect, this disclosure provides a modified tobacco plant, or part thereof, comprising at least one non-natural mutation in an endogenous nucleic acid sequence at least 80% identical to a nucleotide sequence selected from the group consisting of SEQ ID NOs: 59-116. In another aspect, this disclosure provides a modified tobacco plant, or part thereof, comprising at least one non-natural mutation in an endogenous nucleic acid sequence selected from the group consisting of SEQ ID NOs: 1-116.

In an aspect, this disclosure provides a modified tobacco plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 80% identical or similar to an amino acid sequence selected from the group consisting of SEQ ID NOs: 117-174. In another aspect, this disclosure provides a modified tobacco plant, or part thereof, comprising a recombinant DNA construct comprising a heterologous promoter operably linked to a nucleic acid encoding at least one small RNA molecule capable of binding to and reducing the expression of an endogenous nucleic acid sequence at least 80% identical to a nucleic acid sequence selected from the group consisting of SEQ ID NOs: 1-58. In yet another aspect, this disclosure provides a modified tobacco plant, or part thereof, comprising a recombinant DNA construct comprising a heterologous promoter operably linked to a nucleic acid encoding at least one small RNA molecule capable of binding to and reducing the expression of an endogenous nucleic acid sequence at least 80% identical to a nucleic acid sequence selected from the group consisting of SEQ ID NOs: 59-116.

In an aspect, this disclosure provides a modified tobacco plant, or part thereof, comprising a recombinant DNA construct comprising a heterologous promoter operably linked to a nucleic acid sequence encoding a polypeptide comprising an amino acid sequence at least 80% identical or similar to an amino acid sequence selected from the group consisting of SEQ ID NOs: 117-174. In another aspect, this disclosure provides a modified tobacco plant, or part thereof, comprising a recombinant DNA construct comprising a heterologous promoter operably linked to a nucleic acid sequence at least 80% identical to a nucleic acid sequence selected from the group consisting of SEQ ID NOs: 1-58. In another aspect, this disclosure provides a modified tobacco plant, or part thereof, comprising a recombinant DNA construct comprising a heterologous promoter operably linked to a nucleic acid sequence at least 80% identical to a nucleic acid sequence selected from the group consisting of SEQ ID NOs: 59-116.

In an aspect, this disclosure provides a modified tobacco plant, or part thereof, comprising at least one non-natural mutation in an endogenous nucleic acid sequence that modulates the expression or activity of a gene, where the gene encodes a polypeptide comprising an amino acid sequence at least 80% identical or similar to an amino acid sequence selected from the group consisting of SEQ ID NOs: 117-174. In another aspect, this disclosure provides a modified tobacco plant, or part thereof, comprising at least one non-natural mutation in an endogenous nucleic acid sequence that modulates the expression or activity of a gene, where the gene encodes an RNA sequence at least 80% identical or complementary to a nucleic acid sequence selected from the group consisting of SEQ ID NOs: 59-116. In an aspect, an endogenous nucleic acid sequence encodes a transcription factor that is capable of biding to a nucleic acid sequence encoding the gene. As used herein, a “transcription factor” refers to a protein that controls the rate of transcription of a gene from DNA to messenger RNA by binding to a specific DNA sequence of the gene. Without being limited by any scientific theory, if a transcription factor is bound to a gene, it turns on, or enhances, transcription of the gene as compared to transcription in the absence of the transcription factor. In another aspect, an endogenous nucleic acid sequence encodes a repressor that is capable of binding to a nucleic acid sequence encoding the gene. As used herein, a “repressor” refers to a protein that inhibits the transcription of a gene from DNA to messenger RNA by binding to a specific DNA sequence of the gene. Without being limited by any scientific theory, if a repressor is bound to a gene, it turns off, or reduces, transcription of the gene as compared to transcription in the absence of the repressor. In a further aspect, an endogenous nucleic acid sequence encodes a protein that generates a precursor required for the polypeptide encoded by the gene to function. As a non-limiting example of a precursor, quinolate synthase produces quinolinic acid, which is a precursor required for quinolinate phosphoribosyltransferase to function. In an aspect, a gene comprises a nucleic acid sequence at least 80% identical or similar to SEQ ID NOs: 1-58.

In an aspect, this disclosure provides a modified tobacco plant, or part thereof, comprising (a) a genetic modification in a gene; or (b) a genetic modification targeting the gene; where the genetic modification downregulates the expression or activity of the gene, where the gene encodes an amino acid sequence having at least 80% identity or similarity to an amino acid sequence selected from the group consisting of SEQ ID NOs: 117-174. In another aspect, this disclosure provides a modified tobacco plant, or part thereof, comprising (a) a genetic modification in a gene; or (b) a genetic modification targeting the gene; where the genetic modification downregulates the expression or activity of the gene, where the gene encodes a nucleic acid sequence having at least 80% identity to a polynucleotide sequence selected from the group consisting of SEQ ID NOs: 1-58. In another aspect, this disclosure provides a modified tobacco plant, or part thereof, comprising (a) a genetic modification in a gene; or (b) a genetic modification targeting the gene; where the genetic modification downregulates the expression or activity of the gene, where the gene encodes a nucleic acid sequence having at least 80% identity to a polynucleotide sequence selected from the group consisting of SEQ ID NOs: 59-116. In another aspect, this disclosure provides a modified tobacco plant, or part thereof, comprising (a) a genetic modification in a gene; or (b) a genetic modification targeting the gene; where the genetic modification downregulates the expression or activity of the gene, where the gene encodes a nucleic acid sequence having at least 80% identity to a polynucleotide sequence selected from the group consisting of SEQ ID NOs: 1-116.

As used herein, a “genetic modification” refers to a change in the genetic makeup of a plant or plant genome. A genetic modification can be introduced by methods including, but not limited to, mutagenesis, genome editing, genetic transformation, or a combination thereof. A genetic modification includes, for example, a mutation (e.g., a non-natural mutation) in a gene or a transgene targeting a gene (e.g., an arginine decarboxylase (ADC) transgene targets an ADC gene). As used here, “targeting” refers to either directly upregulating or directly downregulating the expression or activity of a gene. As used here, “directly”, in the context of a transgene impacting the expression or activity of a gene, refers to the impact being exerted over the gene via a physical contact or chemical interaction between the gene (e.g., a promoter region or a UTR region) or a product encoded therein (e.g., a mRNA molecule or a polypeptide) and a product encoded by the transgene (e.g., a small RNA molecule or a protein such as a transcription factor or a dominant negative polypeptide variant). In an aspect, a transgene impacts the expression or activity of a target gene without involving a transcription factor (e.g., the transgene does not encode a transcription factor and/or does not suppress the expression or activity of a transcription factor that in turn regulates the target gene).

### Mutations

As used herein, “modified,” in the context of a plant, refers to a plant comprising a genetic alteration introduced for certain purposes and beyond natural polymorphisms. Without being limiting, a modified plant can comprise a non-natural mutation or a recombinant DNA construct. In an aspect, a modified tobacco plant comprises a non-natural mutation. In another aspect, a modified tobacco plant comprises a recombinant DNA construct. In another aspect, a modified tobacco plant comprises a genetic modification.

As used herein, a “mutation” refers to an inheritable genetic modification introduced into a gene to alter the expression or activity of a product encoded by a reference sequence of the gene. A mutation in a certain gene, such as, for example, an arginine decarboxylase (ADC) is referred to as an ADC mutant. Such a modification can be in any sequence region of a gene, for example, in a promoter, 5′untranslated region (UTR), exon, intron, 3′-UTR, or terminator region. In an aspect, a mutation reduces, inhibits, or eliminates the expression or activity of a gene product. In another aspect, a mutation increases, elevates, strengthens, or augments the expression or activity of a gene product.

In an aspect, mutations are not natural polymorphisms that exist in a particular tobacco variety or cultivar. In an aspect, a mutation is a “non-natural” or “non-naturally occurring” mutation. As used herein, a “non-natural” or “non-naturally occurring” mutation refers to a non-spontaneous mutation generated via human intervention, and does not correspond to a spontaneous mutation generated without human intervention. Non-limiting examples of human intervention include mutagenesis (e.g., chemical mutagenesis, ionizing radiation mutagenesis) and targeted genetic modifications (e.g., CRISPR-based methods, TALEN-based methods, zinc finger-based methods). Non-natural mutations and non-naturally occurring mutations do not include spontaneous mutations that arise naturally (e.g., via aberrant DNA replication in a germ line of a plant).

In an aspect, mutations are not natural polymorphisms that exist in a particular tobacco variety or cultivar. It will be appreciated that, when identifying a mutation, the reference DNA sequence should be from the same variety of tobacco. For example, if a modified tobacco plant comprising a mutation is from the variety TN90, then the endogenous reference sequence must be the endogenous TN90 sequence, not a homologous sequence from a different tobacco variety (e.g., K326). Similarly, if a modified tobacco cell comprising a mutation is a TN90 cell, then the endogenous reference sequence must be the endogenous TN90 sequence, not a homologous sequence from a tobacco cell from a different tobacco variety (e.g., K326).

In an aspect, a tobacco plant, or part thereof, is homozygous for at least one non-natural mutation. In another aspect, a tobacco plant, or part thereof, is heterozygous for at least one non-natural mutation. In another aspect, a tobacco plant, or part thereof, is homozygous for an introduced recombinant DNA construct. In another aspect, a tobacco plant, or part thereof, is hemizygous for an introduced recombinant DNA construction. In a further aspect, a tobacco plant, or part thereof, is heterozygous for an introduced recombinant DNA construct.

In an aspect, a mutation provided herein creates a dominant allele of the mutated locus. Dominant alleles are alleles that mask the contribution of a second allele at the same locus. A dominant allele can be a “dominant negative allele” or a “dominant positive allele.” Dominant negative alleles, or antimorphs, are alleles that act in opposition to normal allelic function. A dominant negative allele typically does not function normally and either directly inhibits the activity of a wild-type protein (e.g., through dimerization) or inhibits the activity of a second protein that is required for the normal function of the wild-type protein (e.g., an activator or a downstream component of a pathway). For example, a dominant negative allele abrogates or reduces the normal function of an allele in a heterozygous or homozygous state. Dominant positive alleles can increase normal gene function (e.g., a hypermorph) or provide new functions for a gene (e.g., a neomorph). A semi-dominant allele occurs when penetrance of a linked phenotype in individuals heterozygous for the allele is less than that which is observed in individuals homozygous for the allele.

In an aspect, a mutation provided herein creates a dominant negative allele of the mutated locus. In another aspect, a mutation provided herein creates a dominant positive allele of a mutated locus.

As used herein, “inducing” a mutation refers to generating a mutation in a polynucleotide sequence via human intervention. Many suitable methods for inducing mutations in tobacco are known in the art. Non-limiting examples of such methods include use of chemical mutagens, use of irradiation, use of nucleases, use of transposons, and use of Agrobacterium. In an aspect, inducing a mutation comprises the use of an agent selected from the group consisting of a chemical mutagen, irradiation, a transposon, Agrobacterium, and a nuclease.

In an aspect, inducing a mutation comprises the use of a chemical mutagen. In an aspect, a chemical mutagen comprises ethyl methanesulfonate (EMS).

In another aspect, inducing a mutation comprises the use of irradiation. In an aspect, irradiation comprises gamma rays, X-rays, ionizing radiation, or fast neutrons.

In an aspect, inducing a mutation comprises the use of a transposon. In another aspect, inducing a mutation comprises the use of Agrobacterium.

In a further aspect, inducing a mutation comprises the use of a nuclease. In an aspect, a nuclease is selected from the group consisting of a meganuclease, a zinc-finger nuclease, a transcription activator-like effector nuclease, a CRISPR/Cas9 nuclease, a CRISPR/Cpf1 nuclease, a CRISPR/CasX nuclease, a CRISPR/CasY nuclease, and a Csm1 nuclease. In an aspect, inducing a mutation comprises the use of a CRISPR/Cas9 nuclease. In an aspect, inducing a mutation comprises the use of a CRISPR/Cpf1 nuclease. In an aspect, inducing a mutation comprises the use of a CRISPR/CasX nuclease. In an aspect, inducing a mutation comprises the use of a CRISPR/CasY nuclease. In an aspect, inducing a mutation comprises the use of a Csm1 nuclease.

Several types of mutations are known in the art. In an aspect, a mutation comprises an insertion. An “insertion” refers to the addition of one or more nucleotides or amino acids to a given polynucleotide or amino acid sequence, respectively, as compared to an endogenous reference polynucleotide or amino acid sequence. In another aspect, a mutation comprises a deletion. A “deletion” refers to the removal of one or more nucleotides or amino acids to a given polynucleotide or amino acid sequence, respectively, as compared to an endogenous reference polynucleotide or amino acid sequence. In another aspect, a mutation comprises a substitution. A “substitution” refers to the replacement of one or more nucleotides or amino acids to a given polynucleotide or amino acid sequence, respectively, as compared to an endogenous reference polynucleotide or amino acid sequence. In another aspect, a mutation comprises an inversion. An “inversion” refers to when a segment of a polynucleotide or amino acid sequence is reversed end-to-end. A “duplication” refers to when a segment of a polynucleotide or amino acid sequence is repeated. The repeated segment can immediately follow the original segment, or it can be separated from the original segment by one or more nucleotides or amino acids. In an aspect, a mutation provided herein comprises a mutation selected from the group consisting of an insertion, a deletion, a substitution, a duplication, and an inversion.

In an aspect, a non-natural mutation comprises a mutation selected from the group consisting of a substitution, a deletion, an insertion, a duplication, and an inversion of one or more nucleotides relative to an endogenous nucleic acid sequence selected from the group consisting of SEQ ID NOs: 1-116.

In an aspect, a non-natural mutation comprises a mutation selected from the group consisting of a substitution, a deletion, an insertion, a duplication, and an inversion of one or more nucleotides relative to an endogenous nucleic acid sequence encoding an amino acid sequence selected from the group consisting of SEQ ID NOs: 117-174.

In an aspect, a non-natural mutation comprises one or more mutation types selected from the group consisting of a nonsense mutation, a missense mutation, a frameshift mutation, a splice-site mutation, and any combinations thereof. As used herein, a “nonsense mutation” refers to a mutation to a nucleic acid sequence that introduces a premature stop codon to an amino acid sequence by the nucleic acid sequence. As used herein, a “missense mutation” refers to a mutation to a nucleic acid sequence that causes a substitution within the amino acid sequence encoded by the nucleic acid sequence. As used herein, a “frameshift mutation” refers to an insertion or deletion to a nucleic acid sequence that shifts the frame for translating the nucleic acid sequence to an amino acid sequence. A “splice-site mutation” refers to a mutation in a nucleic acid sequence that causes an intron to be retained for protein translation, or, alternatively, for an exon to be excluded from protein translation. Splice-site mutations can cause nonsense, missense, or frameshift mutations.

Mutations in coding regions of genes (e.g., exonic mutations) can result in a truncated protein or polypeptide when a mutated messenger RNA (mRNA) is translated into a protein or polypeptide. In an aspect, this disclosure provides a mutation that results in the truncation of a protein or polypeptide. As used herein, a “truncated” protein or polypeptide comprises at least one fewer amino acid as compared to an endogenous control protein or polypeptide. For example, if endogenous Protein A comprises 100 amino acids, a truncated version of Protein A can comprise between 1 and 99 amino acids. In an aspect, a non-natural mutation results in a truncation of a polypeptide.

Without being limited by any scientific theory, one way to cause a protein or polypeptide truncation is by the introduction of a premature stop codon in an mRNA transcript of an endogenous gene. In an aspect, this disclosure provides a mutation that results in a premature stop codon in an mRNA transcript of an endogenous gene. As used herein, a “stop codon” refers to a nucleotide triplet within an mRNA transcript that signals a termination of protein translation. A “premature stop codon” refers to a stop codon positioned earlier (e.g., on the 5′-side) than the normal stop codon position in an endogenous mRNA transcript. Without being limiting, several stop codons are known in the art, including “UAG,” “UAA,” “UGA,” “TAG,” “TAA,” and “TGA.”

In an aspect, a mutation provided herein comprises a null mutation. As used herein, a “null mutation” refers to a mutation that confers a complete loss-of-function for a protein encoded by a gene comprising the mutation, or, alternatively, a mutation that confers a complete loss-of-function for a small RNA encoded by a genomic locus. A null mutation can cause lack of mRNA transcript production, a lack of small RNA transcript production, a lack of protein function, or a combination thereof.

A mutation provided herein can be positioned in any part of an endogenous gene. In an aspect, a mutation provided herein is positioned within an exon of an endogenous gene. In another aspect, a mutation provided herein is positioned within an intron of an endogenous gene. In a further aspect, a mutation provided herein is positioned within a 5′-UTR of an endogenous gene. In still another aspect, a mutation provided herein is positioned within a 3′-UTR of an endogenous gene. In yet another aspect, a mutation provided herein is positioned within a promoter of an endogenous gene. In yet another aspect, a mutation provided herein is positioned within a terminator of an endogenous gene. In an aspect, a non-natural mutation provided herein comprises a mutation in a sequence region selected from the group consisting of a promoter, a 5′-UTR, a 3′-UTR, an exon, an intron, and a terminator.

The screening and selection of mutagenized tobacco plants can be through any methodologies known to those having ordinary skill in the art. Examples of screening and selection methodologies include, but are not limited to, Southern analysis, PCR amplification for detection of a polynucleotide, Northern blots, RNase protection, primer-extension, RT-PCR amplification for detecting RNA transcripts, Sanger sequencing, Next Generation sequencing technologies (e.g., Illumina, PacBio, Ion Torrent, 454) enzymatic assays for detecting enzyme or ribozyme activity of polypeptides and polynucleotides, and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known.

### Nucleic Acids and Amino Acids

As used herein, an “endogenous” nucleic acid sequence refers to a nucleic acid sequence that occurs naturally in the genome of an organism. Endogenous nucleic acid sequences do not include heterologous sequences inserted into a genome via deliberate human intervention. Similarly, endogenous amino acid sequences are sequences that exist naturally via translation of an endogenous nucleic acid molecule. In an aspect, a nucleic acid sequence provided herein is an endogenous nucleic acid sequence.

As used herein, “heterologous” refers to a sequence (nucleic acid or amino acid) that originates from a foreign species, or, if from the same species, is substantially modified from its native form in composition and/or genomic locus by deliberate human intervention. The term also is applicable to nucleic acid constructs, also referred to herein as “polynucleotide constructs” or “nucleotide constructs.” In this manner, a “heterologous” nucleic acid construct is intended to mean a construct that originates from a foreign species, or, if from the same species, is substantially modified from its native form in composition and/or genomic locus by deliberate human intervention. Heterologous nucleic acid constructs include, but are not limited to, recombinant nucleotide constructs that have been introduced into a plant or plant part thereof, for example, via transformation methods or subsequent breeding of a transgenic plant with another plant of interest.

As used herein, a “gene” refers to a polynucleotide that can produce a functional unit (e.g., without being limiting, for example, a protein, or a small RNA molecule). A gene can comprise a promoter, an enhancer sequence, a leader sequence, a transcriptional start site, a transcriptional stop site, a polyadenylation site, one or more exons, one or more introns, a 5′-UTR, a 3′-UTR, or any combination thereof. A “gene sequence” can comprise a polynucleotide sequence encoding a promoter, an enhancer sequence, a leader sequence, a transcriptional start site, a transcriptional stop site, a polyadenylation site, one or more exons, one or more introns, a 5′-UTR, a 3′-UTR, or any combination thereof. In one aspect, a gene encodes a small RNA molecule or a precursor thereof. In another aspect, a gene encodes a protein.

The terms “percent identity” or “percent identical” as used herein in reference to two or more nucleotide or amino acid sequences is calculated by (i) comparing two optimally aligned sequences (nucleotide or amino acid) over a window of comparison (the “alignable” region or regions), (ii) determining the number of positions at which the identical nucleic acid base (for nucleotide sequences) or amino acid residue (for proteins and polypeptides) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present application, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%.

When percentage of sequence identity is used in reference to amino acids it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity can be adjusted upwards to correct for the conservative nature of the substitution. Sequences that differ by such conservative substitutions are said to have “sequence similarity” or “similarity.”

For optimal alignment of sequences to calculate their percent identity, various pair-wise or multiple sequence alignment algorithms and programs are known in the art, such as ClustalW or Basic Local Alignment Search Tool® (BLAST™), etc., that can be used to compare the sequence identity or similarity between two or more nucleotide or amino acid sequences. Although other alignment and comparison methods are known in the art, the alignment and percent identity between two sequences (including the percent identity ranges described above) can be as determined by the ClustalW algorithm, see, e.g., Chenna et al., “Multiple sequence alignment with the Clustal series of programs,” Nucleic Acids Research 31: 3497-3500 (2003); Thompson et al., “Clustal W: Improving the sensitivity of progressive multiple sequence alignment through sequence weighting, position-specific gap penalties and weight matrix choice,” Nucleic Acids Research 22: 4673-4680 (1994); Larkin M A et al., “Clustal W and Clustal X version 2.0,” Bioinformatics 23: 2947-48 (2007); and Altschul et al. “Basic local alignment search tool.” J. Mol. Biol. 215:403-410 (1990), the entire contents and disclosures of which are incorporated herein by reference.

The terms “percent complementarity” or “percent complementary” as used herein in reference to two nucleotide sequences is similar to the concept of percent identity but refers to the percentage of nucleotides of a query sequence that optimally base-pair or hybridize to nucleotides a subject sequence when the query and subject sequences are linearly arranged and optimally base paired without secondary folding structures, such as loops, stems or hairpins. Such a percent complementarity can be between two DNA strands, two RNA strands, or a DNA strand and a RNA strand. The “percent complementarity” can be calculated by (i) optimally base-pairing or hybridizing the two nucleotide sequences in a linear and fully extended arrangement (i.e., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences can be determined based on the known pairings of nucleotide bases, such as G-C, A-T, and A-U, through hydrogen binding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present application, when two sequences (query and subject) are optimally base-paired (with allowance for mismatches or non-base-paired nucleotides), the “percent complementarity” for the query sequence is equal to the number of base-paired positions between the two sequences divided by the total number of positions in the query sequence over its length, which is then multiplied by 100%.

The use of the term “polynucleotide” or “nucleic acid molecule” is not intended to limit the present disclosure to polynucleotides comprising deoxyribonucleic acid (DNA). For example, ribonucleic acid (RNA) molecules are also envisioned. Those of ordinary skill in the art will recognize that polynucleotides and nucleic acid molecules can comprise ribonucleotides and combinations of ribonucleotides and deoxyribonucleotides. Such deoxyribonucleotides and ribonucleotides include both naturally occurring molecules and synthetic analogues. The polynucleotides of the present disclosure also encompass all forms of sequences including, but not limited to, single-stranded forms, double-stranded forms, hairpins, stem-and-loop structures, and the like. In an aspect, a nucleic acid molecule provided herein is a DNA molecule. In another aspect, a nucleic acid molecule provided herein is an RNA molecule. In an aspect, a nucleic acid molecule provided herein is single-stranded. In another aspect, a nucleic acid molecule provided herein is double-stranded. A nucleic acid molecule can encode a polypeptide or a small RNA.

As used herein, a “recombinant nucleic acid” refers to a nucleic acid molecule formed by laboratory methods of genetic recombination, such as, without being limiting, molecular cloning. Similarly, a “recombinant DNA construct” refers to a DNA molecule formed by laboratory methods of genetic recombination.

Nucleic acids can be isolated using techniques routine in the art. For example, nucleic acids can be isolated using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides. Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

In one aspect, this disclosure provides methods of detecting recombinant nucleic acids and polypeptides in plant cells. Without being limiting, nucleic acids also can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, NY).

In an aspect, a nucleic acid sequence provided herein is at least 70% identical or complementary to a sequence selected from the group consisting of SEQ ID NOs: 1-116. In another aspect, a nucleic acid sequence provided herein is at least 75% identical or complementary to a sequence selected from the group consisting of SEQ ID NOs: 1-116. In another aspect, a nucleic acid sequence provided herein is at least 80% identical or complementary to a sequence selected from the group consisting of SEQ ID NOs: 1-116. In another aspect, a nucleic acid sequence provided herein is at least 85% identical or complementary to a sequence selected from the group consisting of SEQ ID NOs: 1-116. In another aspect, a nucleic acid sequence provided herein is at least 88% identical or complementary to a sequence selected from the group consisting of SEQ ID NOs: 1-116. In another aspect, a nucleic acid sequence provided herein is at least 90% identical or complementary to a sequence selected from the group consisting of SEQ ID NOs: 1-116. In another aspect, a nucleic acid sequence provided herein is at least 91% identical or complementary to a sequence selected from the group consisting of SEQ ID NOs: 1-116. In another aspect, a nucleic acid sequence provided herein is at least 92% identical or complementary to a sequence selected from the group consisting of SEQ ID NOs: 1-116. In another aspect, a nucleic acid sequence provided herein is at least 93% identical or complementary to a sequence selected from the group consisting of SEQ ID NOs: 1-116. In another aspect, a nucleic acid sequence provided herein is at least 94% identical or complementary to a sequence selected from the group consisting of SEQ ID NOs: 1-116. In another aspect, a nucleic acid sequence provided herein is at least 95% identical or complementary to a sequence selected from the group consisting of SEQ ID NOs: 1-116. In another aspect, a nucleic acid sequence provided herein is at least 96% identical or complementary to a sequence selected from the group consisting of SEQ ID NOs: 1-116. In another aspect, a nucleic acid sequence provided herein is at least 97% identical or complementary to a sequence selected from the group consisting of SEQ ID NOs: 1-116. In another aspect, a nucleic acid sequence provided herein is at least 98% identical or complementary to a sequence selected from the group consisting of SEQ ID NOs: 1-116. In another aspect, a nucleic acid sequence provided herein is at least 99% identical or complementary to a sequence selected from the group consisting of SEQ ID NOs: 1-116. In another aspect, a nucleic acid sequence provided herein is 100% identical or complementary to a sequence selected from the group consisting of SEQ ID NOs: 1-116.

In an aspect, an endogenous nucleic acid sequence provided herein is at least 70% identical or complementary to a sequence selected from the group consisting of SEQ ID NOs: 1-116. In another aspect, an endogenous nucleic acid sequence provided herein is at least 75% identical or complementary to a sequence selected from the group consisting of SEQ ID NOs: 1-116. In another aspect, an endogenous nucleic acid sequence provided herein is at least 80% identical or complementary to a sequence selected from the group consisting of SEQ ID NOs: 1-116. In another aspect, an endogenous nucleic acid sequence provided herein is at least 85% identical or complementary to a sequence selected from the group consisting of SEQ ID NOs: 1-116. In another aspect, an endogenous nucleic acid sequence provided herein is at least 88% identical or complementary to a sequence selected from the group consisting of SEQ ID NOs: 1-116. In another aspect, an endogenous nucleic acid sequence provided herein is at least 90% identical or complementary to a sequence selected from the group consisting of SEQ ID NOs: 1-116. In another aspect, an endogenous nucleic acid sequence provided herein is at least 91% identical or complementary to a sequence selected from the group consisting of SEQ ID NOs: 1-116. In another aspect, an endogenous nucleic acid sequence provided herein is at least 92% identical or complementary to a sequence selected from the group consisting of SEQ ID NOs: 1-116. In another aspect, an endogenous nucleic acid sequence provided herein is at least 93% identical or complementary to a sequence selected from the group consisting of SEQ ID NOs: 1-116. In another aspect, an endogenous nucleic acid sequence provided herein is at least 94% identical or complementary to a sequence selected from the group consisting of SEQ ID NOs: 1-116. In another aspect, an endogenous nucleic acid sequence provided herein is at least 95% identical or complementary to a sequence selected from the group consisting of SEQ ID NOs: 1-116. In another aspect, an endogenous nucleic acid sequence provided herein is at least 96% identical or complementary to a sequence selected from the group consisting of SEQ ID NOs: 1-116. In another aspect, an endogenous nucleic acid sequence provided herein is at least 97% identical or complementary to a sequence selected from the group consisting of SEQ ID NOs: 1-116. In another aspect, an endogenous nucleic acid sequence provided herein is at least 98% identical or complementary to a sequence selected from the group consisting of SEQ ID NOs: 1-116. In another aspect, an endogenous nucleic acid sequence provided herein is at least 99% identical or complementary to a sequence selected from the group consisting of SEQ ID NOs: 1-116. In another aspect, an endogenous nucleic acid sequence provided herein is 100% identical or complementary to a sequence selected from the group consisting of SEQ ID NOs: 1-116.

As used herein, the term “polypeptide” refers to a chain of at least two covalently linked amino acids. Polypeptides can be encoded by polynucleotides provided herein. Proteins provided herein can be encoded by nucleic acid molecules provided herein. Proteins can comprise polypeptides provided herein. As used herein, a “protein” refers to a chain of amino acid residues that is capable of providing structure or enzymatic activity to a cell.

Polypeptides can be detected using antibodies. Techniques for detecting polypeptides using antibodies include enzyme linked immunosorbent assays (ELISAs), Western blots, immunoprecipitations and immunofluorescence. An antibody provided herein can be a polyclonal antibody or a monoclonal antibody. An antibody having specific binding affinity for a polypeptide provided herein can be generated using methods well known in the art. An antibody provided herein can be attached to a solid support such as a microtiter plate using methods known in the art.

Detection (e.g., of an amplification product, of a hybridization complex, of a polypeptide) can be accomplished using detectable labels. The term “label” is intended to encompass the use of direct labels as well as indirect labels. Detectable labels include enzymes, prosthetic groups, fluorescent materials, luminescent materials, bioluminescent materials, and radioactive materials.

In an aspect, an amino acid sequence provided herein is at least 70% identical or similar to an amino acid sequence selected from the group consisting of SEQ ID NOs: 117-174. In another aspect, an amino acid sequence provided herein is at least 75% identical or similar to an amino acid sequence selected from the group consisting of SEQ ID NOs: 117-174. In another aspect, an amino acid sequence provided herein is at least 80% identical or similar to an amino acid sequence selected from the group consisting of SEQ ID NOs: 117-174. In another aspect, an amino acid sequence provided herein is at least 85% identical or similar to an amino acid sequence selected from the group consisting of SEQ ID NOs: 117-174. In another aspect, an amino acid sequence provided herein is at least 88% identical or similar to an amino acid sequence selected from the group consisting of SEQ ID NOs: 117-174. In another aspect, an amino acid sequence provided herein is at least 90% identical or similar to an amino acid sequence selected from the group consisting of SEQ ID NOs: 117-174. In another aspect, an amino acid sequence provided herein is at least 91% identical or similar to an amino acid sequence selected from the group consisting of SEQ ID NOs: 117-174. In another aspect, an amino acid sequence provided herein is at least 92% identical or similar to an amino acid sequence selected from the group consisting of SEQ ID NOs: 117-174. In another aspect, an amino acid sequence provided herein is at least 93% identical or similar to an amino acid sequence selected from the group consisting of SEQ ID NOs: 117-174. In another aspect, an amino acid sequence provided herein is at least 94% identical or similar to an amino acid sequence selected from the group consisting of SEQ ID NOs: 117-174. In another aspect, an amino acid sequence provided herein is at least 95% identical or similar to an amino acid sequence selected from the group consisting of SEQ ID NOs: 117-174. In another aspect, an amino acid sequence provided herein is at least 96% identical or similar to an amino acid sequence selected from the group consisting of SEQ ID NOs: 117-174. In another aspect, an amino acid sequence provided herein is at least 97% identical or similar to an amino acid sequence selected from the group consisting of SEQ ID NOs: 117-174. In another aspect, an amino acid sequence provided herein is at least 98% identical or similar to an amino acid sequence selected from the group consisting of SEQ ID NOs: 117-174. In another aspect, an amino acid sequence provided herein is at least 99% identical or similar to an amino acid sequence selected from the group consisting of SEQ ID NOs: 117-174. In another aspect, an amino acid sequence provided herein is 100% identical or similar to an amino acid sequence selected from the group consisting of SEQ ID NOs: 117-174.

In an aspect, a nucleic acid sequence provided herein encodes a polypeptide comprising an amino acid sequence at least 70% identical or similar to an amino acid sequence selected from the group consisting of SEQ ID NOs: 117-174. In an aspect, a nucleic acid sequence provided herein encodes a polypeptide comprising an amino acid sequence at least 75% identical or similar to an amino acid sequence selected from the group consisting of SEQ ID NOs: 117-174. In an aspect, a nucleic acid sequence provided herein encodes a polypeptide comprising an amino acid sequence at least 80% identical or similar to an amino acid sequence selected from the group consisting of SEQ ID NOs: 117-174. In an aspect, a nucleic acid sequence provided herein encodes a polypeptide comprising an amino acid sequence at least 85% identical or similar to an amino acid sequence selected from the group consisting of SEQ ID NOs: 117-174. In an aspect, a nucleic acid sequence provided herein encodes a polypeptide comprising an amino acid sequence at least 88% identical or similar to an amino acid sequence selected from the group consisting of SEQ ID NOs: 117-174. In an aspect, a nucleic acid sequence provided herein encodes a polypeptide comprising an amino acid sequence at least 90% identical or similar to an amino acid sequence selected from the group consisting of SEQ ID NOs: 117-174. In an aspect, a nucleic acid sequence provided herein encodes a polypeptide comprising an amino acid sequence at least 91% identical or similar to an amino acid sequence selected from the group consisting of SEQ ID NOs: 117-174. In an aspect, a nucleic acid sequence provided herein encodes a polypeptide comprising an amino acid sequence at least 92% identical or similar to an amino acid sequence selected from the group consisting of SEQ ID NOs: 117-174. In an aspect, a nucleic acid sequence provided herein encodes a polypeptide comprising an amino acid sequence at least 93% identical or similar to an amino acid sequence selected from the group consisting of SEQ ID NOs: 117-174. In an aspect, a nucleic acid sequence provided herein encodes a polypeptide comprising an amino acid sequence at least 94% identical or similar to an amino acid sequence selected from the group consisting of SEQ ID NOs: 117-174. In an aspect, a nucleic acid sequence provided herein encodes a polypeptide comprising an amino acid sequence at least 95% identical or similar to an amino acid sequence selected from the group consisting of SEQ ID NOs: 117-174. In an aspect, a nucleic acid sequence provided herein encodes a polypeptide comprising an amino acid sequence at least 96% identical or similar to an amino acid sequence selected from the group consisting of SEQ ID NOs: 117-174. In an aspect, a nucleic acid sequence provided herein encodes a polypeptide comprising an amino acid sequence at least 97% identical or similar to an amino acid sequence selected from the group consisting of SEQ ID NOs: 117-174. In an aspect, a nucleic acid sequence provided herein encodes a polypeptide comprising an amino acid sequence at least 98% identical or similar to an amino acid sequence selected from the group consisting of SEQ ID NOs: 117-174. In an aspect, a nucleic acid sequence provided herein encodes a polypeptide comprising an amino acid sequence at least 99% identical or similar to an amino acid sequence selected from the group consisting of SEQ ID NOs: 117-174. In an aspect, a nucleic acid sequence provided herein encodes a polypeptide comprising an amino acid sequence 100% identical or similar to an amino acid sequence selected from the group consisting of SEQ ID NOs: 117-174.

In an aspect, an endogenous nucleic acid sequence provided herein encodes a polypeptide comprising an amino acid sequence at least 70% identical or similar to an amino acid sequence selected from the group consisting of SEQ ID NOs: 117-174. In an aspect, an endogenous nucleic acid sequence provided herein encodes a polypeptide comprising an amino acid sequence at least 75% identical or similar to an amino acid sequence selected from the group consisting of SEQ ID NOs: 117-174. In an aspect, an endogenous nucleic acid sequence provided herein encodes a polypeptide comprising an amino acid sequence at least 80% identical or similar to an amino acid sequence selected from the group consisting of SEQ ID NOs: 117-174. In an aspect, an endogenous nucleic acid sequence provided herein encodes a polypeptide comprising an amino acid sequence at least 85% identical or similar to an amino acid sequence selected from the group consisting of SEQ ID NOs: 117-174. In an aspect, an endogenous nucleic acid sequence provided herein encodes a polypeptide comprising an amino acid sequence at least 88% identical or similar to an amino acid sequence selected from the group consisting of SEQ ID NOs: 117-174. In an aspect, an endogenous nucleic acid sequence provided herein encodes a polypeptide comprising an amino acid sequence at least 90% identical or similar to an amino acid sequence selected from the group consisting of SEQ ID NOs: 117-174. In an aspect, an endogenous nucleic acid sequence provided herein encodes a polypeptide comprising an amino acid sequence at least 91% identical or similar to an amino acid sequence selected from the group consisting of SEQ ID NOs: 117-174. In an aspect, an endogenous nucleic acid sequence provided herein encodes a polypeptide comprising an amino acid sequence at least 92% identical or similar to an amino acid sequence selected from the group consisting of SEQ ID NOs: 117-174. In an aspect, an endogenous nucleic acid sequence provided herein encodes a polypeptide comprising an amino acid sequence at least 93% identical or similar to an amino acid sequence selected from the group consisting of SEQ ID NOs: 117-174. In an aspect, an endogenous nucleic acid sequence provided herein encodes a polypeptide comprising an amino acid sequence at least 94% identical or similar to an amino acid sequence selected from the group consisting of SEQ ID NOs: 117-174. In an aspect, an endogenous nucleic acid sequence provided herein encodes a polypeptide comprising an amino acid sequence at least 95% identical or similar to an amino acid sequence selected from the group consisting of SEQ ID NOs: 117-174. In an aspect, an endogenous nucleic acid sequence provided herein encodes a polypeptide comprising an amino acid sequence at least 96% identical or similar to an amino acid sequence selected from the group consisting of SEQ ID NOs: 117-174. In an aspect, an endogenous nucleic acid sequence provided herein encodes a polypeptide comprising an amino acid sequence at least 97% identical or similar to an amino acid sequence selected from the group consisting of SEQ ID NOs: 117-174. In an aspect, an endogenous nucleic acid sequence provided herein encodes a polypeptide comprising an amino acid sequence at least 98% identical or similar to an amino acid sequence selected from the group consisting of SEQ ID NOs: 117-174. In an aspect, an endogenous nucleic acid sequence provided herein encodes a polypeptide comprising an amino acid sequence at least 99% identical or similar to an amino acid sequence selected from the group consisting of SEQ ID NOs: 117-174. In an aspect, an endogenous nucleic acid sequence provided herein encodes a polypeptide comprising an amino acid sequence 100% identical or similar to an amino acid sequence selected from the group consisting of SEQ ID NOs: 117-174.

### Promoters

As commonly understood in the art, the term “promoter” refers to a DNA sequence that contains an RNA polymerase binding site, a transcription start site, and/or a TATA box and assists or promotes the transcription and expression of an associated transcribable polynucleotide sequence and/or gene (or transgene). A promoter can be synthetically produced, varied, or derived from a known or naturally occurring promoter sequence or other promoter sequence. A promoter can also include a chimeric promoter comprising a combination of two or more heterologous sequences. A promoter of the present application can thus include variants of promoter sequences that are similar in composition, but not identical to, other promoter sequence(s) known or provided herein.

Promoters that drive expression in all or most tissues of the plant are referred to as “constitutive” promoters. In an aspect, a constitutive promoter is selected from the group consisting of a Cauliflower Mosaic Virus 35S promoter, a ubiquitin promoter, an actin promoter, an opine promoter, and an alcohol dehydrogenase promoter.

Promoters that drive expression during certain periods or stages of development are referred to as “developmental” promoters.

Promoters that drive enhanced expression in certain tissues of an organism relative to other tissues of the organism are referred to as “tissue-preferred” promoters. Thus, a “tissue-preferred” promoter causes relatively higher or preferential expression in a specific tissue(s) of a plant, but with lower levels of expression in other tissue(s) of the plant. As a non-limiting example, a root tissue-preferred promoter exhibits higher activity in root tissue, but may also exhibit activity, albeit at lower levels, in additional tissues such as stem, leaves, and floral tissues. A “tissue-specific” promoter causes expression only in a specific tissue. As a non-limiting example, a root tissue-specific promoter drives expression only in root tissue. In an aspect, a tissue-specific promoter is a root tissue-specific promoter. In another aspect, a tissue-preferred promoter is a root tissue-preferred promoter.

In an aspect, a root tissue-preferred promoter is a cassava vein mosaic virus (CsVMV) promoter.

An “inducible” promoter is a promoter that initiates transcription in response to an environmental stimulus such as heat, cold, drought, light, or other stimuli, such as wounding or chemical application.

In an aspect, a promoter provided herein is a constitutive promoter. In another aspect, a promoter provided herein is an inducible promoter. In a further aspect, a promoter provided herein is a developmental promoter. In another aspect, a promoter is a tissue-preferred or tissue-specific promoter. In a further aspect, a promoter is selected from the group consisting of a constitutive promoter, a tissue-preferred promoter, a tissue-specific promoter, and an inducible promoter.

In an aspect, this disclosure provides a heterologous promoter. In another aspect, this disclosure provides a promoter that is operably linked to a heterologous polynucleotide. In another aspect, this disclosure provides a polynucleotide sequence that is operably linked to a heterologous promoter.

As used herein, “operably linked” refers to a functional linkage between two or more elements. For example, an operable linkage between a polynucleotide of interest and a regulatory sequence (e.g., a promoter) is a functional link that allows for expression of the polynucleotide of interest. Operably linked elements may be contiguous or non-contiguous. In an aspect, a promoter provided herein is operably linked to a heterologous nucleic acid molecule.

### Small RNA Molecules

In an aspect, a nucleic acid molecule provided herein is a small RNA molecule. In another aspect, a nucleic acid molecule encodes a small RNA molecule.

As used herein, a “small RNA molecule” refers to a non-coding RNA molecule of between 16 nucleotides and 50 nucleotides in length. In an aspect, a small RNA molecule comprises between 16 nucleotides and 40 nucleotides. In another aspect, a small RNA molecule comprises between 16 nucleotides and 30 nucleotides. In another aspect, a small RNA molecule comprises between 18 nucleotides and 50 nucleotides. In another aspect, a small RNA molecule comprises between 18 nucleotides and 40 nucleotides. In another aspect, a small RNA molecule comprises between 18 nucleotides and 30 nucleotides. In another aspect, a small RNA molecule comprises between 18 nucleotides and 25 nucleotides. In another aspect, a small RNA molecule comprises between 20 nucleotides and 28 nucleotides. In another aspect, a small RNA molecule comprises between 20 nucleotides and 24 nucleotides. In another aspect, a small RNA molecule comprises between 21 nucleotides and 23 nucleotides. In another aspect, a small RNA molecule comprises 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides.

In an aspect, a small RNA molecule is selected from the group consisting of a double-stranded RNA, a small interfering RNA (siRNA), a trans-acting siRNA, and a microRNA (miRNA).

miRNAs are generally of between about 19 to about 25 nucleotides (commonly about 20-24 nucleotides in plants), that guide cleavage in trans of target transcripts, negatively regulating the expression of genes involved in various regulation and development pathways. In some cases, miRNAs serve to guide in-phase processing of siRNA primary transcripts.

It is appreciated in the art that, in plants, miRNAs and targeted nucleic acids often do not share perfect complementarity (although miRNAs and targeted nucleic acids can have perfect complementarity). miRNAs and their targets can have several mismatches between them while still enabling the miRNA to reduce the expression and/or function of the target gene. See, for example, Liu et al., Plant Cell, 26:741-753 (2014) and Wang et al., Curr. Opin. Plant Biol., 27:118-124 (2015).

In an aspect, a small RNA molecule comprises 100% complementarity with a nucleic acid molecule comprising a sequence selected from the group consisting of SEQ ID NOs: 1-58. In an aspect, a small RNA molecule comprises at least 95% complementarity over 21 consecutive nucleotides of a nucleic acid molecule comprising a sequence selected from the group consisting of SEQ ID NOs: 1-58. In an aspect, a small RNA molecule comprises at least 90% complementarity over 21 consecutive nucleotides of a nucleic acid molecule comprising a sequence selected from the group consisting of SEQ ID NOs: 1-58. In an aspect, a small RNA molecule comprises at least 85% complementarity over 21 consecutive nucleotides of a nucleic acid molecule comprising a sequence selected from the group consisting of SEQ ID NOs: 1-58. In an aspect, a small RNA molecule comprises at least 95% complementarity over 20 consecutive nucleotides of a nucleic acid molecule comprising a sequence selected from the group consisting of SEQ ID NOs: 1-58. In an aspect, a small RNA molecule comprises at least 90% complementarity over 20 consecutive nucleotides of a nucleic acid molecule comprising a sequence selected from the group consisting of SEQ ID NOs: 1-58. In an aspect, a small RNA molecule comprises at least 85% complementarity over 20 consecutive nucleotides of a nucleic acid molecule comprising a sequence selected from the group consisting of SEQ ID NOs: 1-58. In an aspect, a small RNA molecule comprises at least 95% complementarity over 19 consecutive nucleotides of a nucleic acid molecule comprising a sequence selected from the group consisting of SEQ ID NOs: 1-58. In an aspect, a small RNA molecule comprises at least 90% complementarity over 19 consecutive nucleotides of a nucleic acid molecule comprising a sequence selected from the group consisting of SEQ ID NOs: 1-58. In an aspect, a small RNA molecule comprises at least 85% complementarity over 19 consecutive nucleotides of a nucleic acid molecule comprising a sequence selected from the group consisting of SEQ ID NOs: 1-58.

Many microRNA genes (MIR genes) have been identified and made publicly available in a database (“miRBase”, available online at microrna[dot]sanger[dot]ac[dot]uk/sequences; also see Griffiths-Jones et al. (2003) Nucleic Acids Res., 31:439-441). MIR genes have been reported to occur in intergenic regions, both isolated and in clusters in the genome, but can also be located entirely or partially within introns of other genes (both protein-coding and non-protein-coding). For a review of miRNA biogenesis, see Kim (2005) Nature Rev. Mol. Cell. Biol., 6:376-385. Transcription of MIR genes can be, at least in some cases, under promotional control of a MIR gene's own promoter. The primary transcript, termed a “pri-miRNA”, can be quite large (several kilobases) and can be polycistronic, containing one or more pre-miRNAs (fold-back structures containing a stem-loop arrangement that is processed to the mature miRNA) as well as the usual 5′ “cap” and polyadenylated tail of an mRNA.

Maturation of a mature miRNA from its corresponding precursors (pri-miRNAs and pre-miRNAs) differs significantly between animals and plants. For example, in plant cells, microRNA precursor molecules are believed to be largely processed to the mature miRNA entirely in the nucleus, whereas in animal cells, the pri-miRNA transcript is processed in the nucleus by the animal-specific enzyme Drosha, followed by export of the pre-miRNA to the cytoplasm where it is further processed to the mature miRNA. Mature miRNAs in plants are typically 21 nucleotides in length.

Transgenic expression of miRNAs (whether a naturally occurring sequence or an artificial sequence) can be employed to regulate expression of the miRNA's target gene or genes. Inclusion of a miRNA recognition site in a transgenically expressed transcript is also useful in regulating expression of the transcript. Recognition sites of miRNAs have been validated in all regions of an mRNA, including the 5′ untranslated region, coding region, and 3′ untranslated region, indicating that the position of the miRNA target site relative to the coding sequence may not necessarily affect suppression. Because miRNAs are important regulatory elements in eukaryotes, transgenic suppression of miRNAs is useful for manipulating biological pathways and responses. Finally, promoters of MIR genes can have very specific expression patterns (e.g., cell-specific, tissue-specific, temporally specific, or inducible), and thus are useful in recombinant constructs to induce such specific transcription of a DNA sequence to which they are operably linked. Various utilities of miRNAs, their precursors, their recognition sites, and their promoters are described in detail in U.S. Patent Application Publication 2006/0200878 A1, incorporated by reference herein. Non-limiting examples of these utilities include: (1) the expression of a native miRNA or miRNA precursor sequence to suppress a target gene; (2) the expression of an artificial miRNA or miRNA precursor sequence to suppress a target gene; (3) expression of a transgene with a miRNA recognition site, where the transgene is suppressed when the mature miRNA is expressed; (4) expression of a transgene driven by a miRNA promoter.

Designing an artificial miRNA sequence can be as simple as substituting sequence that is complementary to the intended target for nucleotides in the miRNA stem region of the miRNA precursor, as demonstrated by Zeng et al. (2002) Mol. Cell, 9:1327-1333. One non-limiting example of a general method for determining nucleotide changes in the native miRNA sequence to produce the engineered miRNA precursor includes the following steps: (a) Selecting a unique target sequence of at least 18 nucleotides specific to the target gene, e.g., by using sequence alignment tools such as BLAST (see, for example, Altschul et al. (1990) J. Mol. Biol., 215:403-410; Altschul et al. (1997) Nucleic Acids Res., 25:3389-3402), for example, of both tobacco cDNA and genomic DNA databases, to identify target transcript orthologues and any potential matches to unrelated genes, thereby avoiding unintentional silencing of non-target sequences; (b) Analyzing the target gene for undesirable sequences (e.g., matches to sequences from non-target species), and score each potential 19-mer segment for GC content, Reynolds score (see Reynolds et al. (2004) Nature Biotechnol., 22:326-330), and functional asymmetry characterized by a negative difference in free energy (“.DELTA..DELTA.G” or “AAG”) (see Khvorova et al. (2003) Cell, 115:209-216). Preferably 19-mers are selected that have all or most of the following characteristics: (1) a Reynolds score>4, (2) a GC content between about 40% to about 60%, (3) a negative AAG, (4) a terminal adenosine, (5) lack of a consecutive run of 4 or more of the same nucleotide; (6) a location near the 3′ terminus of the target gene; (7) minimal differences from the miRNA precursor transcript. Positions at every third nucleotide in an siRNA have been reported to be especially important in influencing RNAi efficacy and an algorithm, “siExplorer” is publicly available at rna[dot]chem[dot]t[dot]u-tokyo[dot]ac[dot]jp/siexplorer.htm (see Katoh and Suzuki (2007) Nucleic Acids Res., 10.1093/nar/gkl1120); (c) Determining the reverse complement of the selected 19-mers to use in making a modified mature miRNA. The additional nucleotide at position 20 is preferably matched to the selected target sequence, and the nucleotide at position 21 is preferably chosen to either be unpaired to prevent spreading of silencing on the target transcript or paired to the target sequence to promote spreading of silencing on the target transcript; and (d) transforming the artificial miRNA into a plant.

Without being limited by any scientific theory, it is appreciated in the art that an RNAi knockdown of a candidate gene (e.g., via the use of an artificial miRNA or an siRNA) and a mutation (e.g., missense or nonsense mutations) in the same candidate gene can both cause reduction of expression and/or decreased protein activity and can cause identical or similar phenotypes in plants. See, for example, Agrawal et al., Microbiology and Molecular Biology Reviews, 67:657-685 (2003).

In an aspect, a small RNA provided herein comprises at nucleic acid sequence at least 75% identical or complementary to a nucleic acid sequence selected from the group consisting of SEQ ID NOs: 1-116. In an aspect, a small RNA provided herein comprises at nucleic acid sequence at least 80% identical or complementary to a nucleic acid sequence selected from the group consisting of SEQ ID NOs: 1-116. In an aspect, a small RNA provided herein comprises at nucleic acid sequence at least 85% identical or complementary to a nucleic acid sequence selected from the group consisting of SEQ ID NOs: 1-116. In an aspect, a small RNA provided herein comprises at nucleic acid sequence at least 90% identical or complementary to a nucleic acid sequence selected from the group consisting of SEQ ID NOs: 1-116. In an aspect, a small RNA provided herein comprises at nucleic acid sequence at least 95% identical or complementary to a nucleic acid sequence selected from the group consisting of SEQ ID NOs: 1-116. In an aspect, a small RNA provided herein comprises at nucleic acid sequence at least 96% identical or complementary to a nucleic acid sequence selected from the group consisting of SEQ ID NOs: 1-116. In an aspect, a small RNA provided herein comprises at nucleic acid sequence at least 97% identical or complementary to a nucleic acid sequence selected from the group consisting of SEQ ID NOs: 1-116. In an aspect, a small RNA provided herein comprises at nucleic acid sequence at least 98% identical or complementary to a nucleic acid sequence selected from the group consisting of SEQ ID NOs: 1-116. In an aspect, a small RNA provided herein comprises at nucleic acid sequence at least 99% identical or complementary to a nucleic acid sequence selected from the group consisting of SEQ ID NOs: 1-116. In an aspect, a small RNA provided herein comprises at nucleic acid sequence 100% identical or complementary to a nucleic acid sequence selected from the group consisting of SEQ ID NOs: 1-116.

In an aspect, a small RNA provided herein comprises at nucleic acid sequence at least 75% complementary to a nucleic acid sequence selected from the group consisting of SEQ ID NOs: 59-116. In an aspect, a small RNA provided herein comprises at nucleic acid sequence at least 80% complementary to a nucleic acid sequence selected from the group consisting of SEQ ID NOs: 59-116. In an aspect, a small RNA provided herein comprises at nucleic acid sequence at least 85% identical or complementary to a nucleic acid sequence selected from the group consisting of SEQ ID NOs: 59-116. In an aspect, a small RNA provided herein comprises at nucleic acid sequence at least 90% identical or complementary to a nucleic acid sequence selected from the group consisting of SEQ ID NOs: 59-116. In an aspect, a small RNA provided herein comprises at nucleic acid sequence at least 95% identical or complementary to a nucleic acid sequence selected from the group consisting of SEQ ID NOs: 59-116. In an aspect, a small RNA provided herein comprises at nucleic acid sequence at least 96% identical or complementary to a nucleic acid sequence selected from the group consisting of SEQ ID NOs: 59-116. In an aspect, a small RNA provided herein comprises at nucleic acid sequence at least 97% identical or complementary to a nucleic acid sequence selected from the group consisting of SEQ ID NOs: 59-116. In an aspect, a small RNA provided herein comprises at nucleic acid sequence at least 98% identical or complementary to a nucleic acid sequence selected from the group consisting of SEQ ID NOs: 59-116. In an aspect, a small RNA provided herein comprises at nucleic acid sequence at least 99% identical or complementary to a nucleic acid sequence selected from the group consisting of SEQ ID NOs: 59-116. In an aspect, a small RNA provided herein comprises at nucleic acid sequence 100% identical or complementary to a nucleic acid sequence selected from the group consisting of SEQ ID NOs: 59-116.

In an aspect, a small RNA provided herein comprises a nucleic acid sequence at least 88.7% identical or complementary to at least 18 contiguous nucleotides of a nucleic acid sequence selected from the group consisting of SEQ ID NOs: 1-116. In another aspect, a small RNA provided herein comprises a nucleic acid sequence at least 94.3% identical or complementary to at least 18 contiguous nucleotides of a nucleic acid sequence selected from the group consisting of SEQ ID NOs: 1-116. In another aspect, a small RNA provided herein comprises a nucleic acid sequence 100% identical or complementary to at least 18 contiguous nucleotides of a nucleic acid sequence selected from the group consisting of SEQ ID NOs: 1-116. In another aspect, a small RNA provided herein comprises a nucleic acid sequence at least 85% identical or complementary to at least 20 contiguous nucleotides of a nucleic acid sequence selected from the group consisting of SEQ ID NOs: 1-116. In another aspect, a small RNA provided herein comprises a nucleic acid sequence at least 90% identical or complementary to at least 20 contiguous nucleotides of a nucleic acid sequence selected from the group consisting of SEQ ID NOs: 1-116. In another aspect, a small RNA provided herein comprises a nucleic acid sequence at least 95% identical or complementary to at least 20 contiguous nucleotides of a nucleic acid sequence selected from the group consisting of SEQ ID NOs: 1-116. In another aspect, a small RNA provided herein comprises a nucleic acid sequence 100% identical or complementary to at least 20 contiguous nucleotides of a nucleic acid sequence selected from the group consisting of SEQ ID NOs: 1-116.

In an aspect, a small RNA molecule provided herein is capable of binding to and reducing the expression of a nucleic acid sequence encoding a polypeptide at least 70% identical or similar to an amino acid sequence selected from the group consisting of SEQ ID NOs: 117-174. In an aspect, a small RNA molecule provided herein is capable of binding to and reducing the expression of a nucleic acid sequence encoding a polypeptide at least 75% identical or similar to an amino acid sequence selected from the group consisting of SEQ ID NOs: 117-174. In an aspect, a small RNA molecule provided herein is capable of binding to and reducing the expression of a nucleic acid sequence encoding a polypeptide at least 80% identical or similar to an amino acid sequence selected from the group consisting of SEQ ID NOs: 117-174. In an aspect, a small RNA molecule provided herein is capable of binding to and reducing the expression of a nucleic acid sequence encoding a polypeptide at least 85% identical or similar to an amino acid sequence selected from the group consisting of SEQ ID NOs: 117-174. In an aspect, a small RNA molecule provided herein is capable of binding to and reducing the expression of a nucleic acid sequence encoding a polypeptide at least 88% identical or similar to an amino acid sequence selected from the group consisting of SEQ ID NOs: 117-174. In an aspect, a small RNA molecule provided herein is capable of binding to and reducing the expression of a nucleic acid sequence encoding a polypeptide at least 90% identical or similar to an amino acid sequence selected from the group consisting of SEQ ID NOs: 117-174. In an aspect, a small RNA molecule provided herein is capable of binding to and reducing the expression of a nucleic acid sequence encoding a polypeptide at least 91% identical or similar to an amino acid sequence selected from the group consisting of SEQ ID NOs: 117-174. In an aspect, a small RNA molecule provided herein is capable of binding to and reducing the expression of a nucleic acid sequence encoding a polypeptide at least 92% identical or similar to an amino acid sequence selected from the group consisting of SEQ ID NOs: 117-174. In an aspect, a small RNA molecule provided herein is capable of binding to and reducing the expression of a nucleic acid sequence encoding a polypeptide at least 93% identical or similar to an amino acid sequence selected from the group consisting of SEQ ID NOs: 117-174. In an aspect, a small RNA molecule provided herein is capable of binding to and reducing the expression of a nucleic acid sequence encoding a polypeptide at least 94% identical or similar to an amino acid sequence selected from the group consisting of SEQ ID NOs: 117-174. In an aspect, a small RNA molecule provided herein is capable of binding to and reducing the expression of a nucleic acid sequence encoding a polypeptide at least 95% identical or similar to an amino acid sequence selected from the group consisting of SEQ ID NOs: 117-174. In an aspect, a small RNA molecule provided herein is capable of binding to and reducing the expression of a nucleic acid sequence encoding a polypeptide at least 96% identical or similar to an amino acid sequence selected from the group consisting of SEQ ID NOs: 117-174. In an aspect, a small RNA molecule provided herein is capable of binding to and reducing the expression of a nucleic acid sequence encoding a polypeptide at least 97% identical or similar to an amino acid sequence selected from the group consisting of SEQ ID NOs: 117-174. In an aspect, a small RNA molecule provided herein is capable of binding to and reducing the expression of a nucleic acid sequence encoding a polypeptide at least 98% identical or similar to an amino acid sequence selected from the group consisting of SEQ ID NOs: 117-174. In an aspect, a small RNA molecule provided herein is capable of binding to and reducing the expression of a nucleic acid sequence encoding a polypeptide at least 99% identical or similar to an amino acid sequence selected from the group consisting of SEQ ID NOs: 117-174. In an aspect, a small RNA molecule provided herein is capable of binding to and reducing the expression of a nucleic acid sequence encoding a polypeptide 100% identical or similar to an amino acid sequence selected from the group consisting of SEQ ID NOs: 117-174.

In an aspect, a small RNA molecule provided herein is capable of binding to and reducing the expression of a nucleic acid sequence at least 70% identical to nucleic acid sequence selected from the group consisting of SEQ ID NOs: 1-116, or an RNA transcribed therefrom. In an aspect, a small RNA molecule provided herein is capable of binding to and reducing the expression of a nucleic acid sequence at least 75% identical to nucleic acid sequence selected from the group consisting of SEQ ID NOs: 1-116, or an RNA transcribed therefrom. In an aspect, a small RNA molecule provided herein is capable of binding to and reducing the expression of a nucleic acid sequence at least 80% identical to nucleic acid sequence selected from the group consisting of SEQ ID NOs: 1-116, or an RNA transcribed therefrom. In an aspect, a small RNA molecule provided herein is capable of binding to and reducing the expression of a nucleic acid sequence at least 85% identical to nucleic acid sequence selected from the group consisting of SEQ ID NOs: 1-116, or an RNA transcribed therefrom. In an aspect, a small RNA molecule provided herein is capable of binding to and reducing the expression of a nucleic acid sequence at least 88% identical to nucleic acid sequence selected from the group consisting of SEQ ID NOs: 1-116, or an RNA transcribed therefrom. In an aspect, a small RNA molecule provided herein is capable of binding to and reducing the expression of a nucleic acid sequence at least 90% identical to nucleic acid sequence selected from the group consisting of SEQ ID NOs: 1-116, or an RNA transcribed therefrom. In an aspect, a small RNA molecule provided herein is capable of binding to and reducing the expression of a nucleic acid sequence at least 91% identical to nucleic acid sequence selected from the group consisting of SEQ ID NOs: 1-116, or an RNA transcribed therefrom. In an aspect, a small RNA molecule provided herein is capable of binding to and reducing the expression of a nucleic acid sequence at least 92% identical to nucleic acid sequence selected from the group consisting of SEQ ID NOs: 1-116, or an RNA transcribed therefrom. In an aspect, a small RNA molecule provided herein is capable of binding to and reducing the expression of a nucleic acid sequence at least 93% identical to nucleic acid sequence selected from the group consisting of SEQ ID NOs: 1-116, or an RNA transcribed therefrom. In an aspect, a small RNA molecule provided herein is capable of binding to and reducing the expression of a nucleic acid sequence at least 94% identical to nucleic acid sequence selected from the group consisting of SEQ ID NOs: 1-116, or an RNA transcribed therefrom. In an aspect, a small RNA molecule provided herein is capable of binding to and reducing the expression of a nucleic acid sequence at least 95% identical to nucleic acid sequence selected from the group consisting of SEQ ID NOs: 1-116, or an RNA transcribed therefrom. In an aspect, a small RNA molecule provided herein is capable of binding to and reducing the expression of a nucleic acid sequence at least 96% identical to nucleic acid sequence selected from the group consisting of SEQ ID NOs: 1-116, or an RNA transcribed therefrom. In an aspect, a small RNA molecule provided herein is capable of binding to and reducing the expression of a nucleic acid sequence at least 97% identical to nucleic acid sequence selected from the group consisting of SEQ ID NOs: 1-116, or an RNA transcribed therefrom. In an aspect, a small RNA molecule provided herein is capable of binding to and reducing the expression of a nucleic acid sequence at least 98% identical to nucleic acid sequence selected from the group consisting of SEQ ID NOs: 1-116, or an RNA transcribed therefrom. In an aspect, a small RNA molecule provided herein is capable of binding to and reducing the expression of a nucleic acid sequence at least 99% identical to nucleic acid sequence selected from the group consisting of SEQ ID NOs: 1-116, or an RNA transcribed therefrom. In an aspect, a small RNA molecule provided herein is capable of binding to and reducing the expression of a nucleic acid sequence 100% identical to nucleic acid sequence selected from the group consisting of SEQ ID NOs: 1-116, or an RNA transcribed therefrom.

As used herein, “capable of binding to” is synonymous with “capable of hybridizing to.” In an aspect, a first nucleic acid molecule that is capable of binding to a second nucleic acid molecule binds to the second nucleic acid molecule. As used herein, a first nucleic acid molecule can “hybridize” a second nucleic acid molecule via non-covalent interactions (e.g., Watson-Crick base-pairing) in a sequence-specific, antiparallel manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine pairing with thymine, adenine pairing with uracil, and guanine (G) pairing with cytosine (C) [DNA, RNA]. In addition, it is also known in the art that for hybridization between two RNA molecules (e.g., dsRNA), guanine base pairs with uracil. For example, G/U base-pairing is partially responsible for the degeneracy (i.e., redundancy) of the genetic code in the context of tRNA anti-codon base-pairing with codons in mRNA. In the context of this disclosure, a guanine of a protein-binding segment (dsRNA duplex) of a subject DNA-targeting RNA molecule is considered complementary to an uracil, and vice versa. As such, when a G/U base-pair can be made at a given nucleotide position a protein-binding segment (dsRNA duplex) of a subject DNA-targeting RNA molecule, the position is not considered to be non-complementary, but is instead considered to be complementary.

Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). The conditions of temperature and ionic strength determine the “stringency” of the hybridization.

Hybridization requires that the two nucleic acids contain complementary sequences, although mismatches between bases are possible. The conditions appropriate for hybridization between two nucleic acids depend on the length of the nucleic acids and the degree of complementation, variables well known in the art. The greater the degree of complementation between two nucleotide sequences, the greater the value of the melting temperature (Tm) for hybrids of nucleic acids having those sequences. For hybridizations between nucleic acids with short stretches of complementarity (e.g. complementarity over 35 or fewer nucleotides) the position of mismatches becomes important (see Sambrook et al.). Typically, the length for a hybridizable nucleic acid is at least about 10 nucleotides. Illustrative minimum lengths for a hybridizable nucleic acid are: at least about 15 nucleotides; at least about 20 nucleotides; at least about 22 nucleotides; at least about 25 nucleotides; and at least about 30 nucleotides). Furthermore, the skilled artisan will recognize that the temperature and wash solution salt concentration may be adjusted as necessary according to factors such as length of the region of complementation and the degree of complementation.

It is understood in the art that the sequence of polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a loop structure or hairpin structure). For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined routinely using BLAST® programs (basic local alignment search tools) and PowerBLAST programs known in the art (see Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

### Reduced Expression/Activity

In an aspect, a small RNA molecule reduces the expression of any nucleic acid sequence to which it is capable of binding. In another aspect, a non-natural mutation provided herein reduces the expression of the mutated nucleic acid sequence as compared to the non-mutated nucleic acid sequence in a control plant grown under comparable conditions.

Reduced expression of an endogenous nucleic acid sequence can be measured using any suitable method known in the art. Non-limiting examples of measuring expression include quantitative reverse transcriptase polymerase chain reaction (qRT-PCR), RNA blot (e.g., a Northern blot), RNA sequencing. Differences in expression can be described as an absolute quantification or a relative quantification. See, for example, Livak and Schmittgen, Methods, 25:402-408 (2001). If an endogenous nucleic acid sequence encodes a protein, changes in expression can be inferred by examining the accumulation of the encoded protein. Non-limiting examples of measuring protein accumulation include Western blots and enzyme-linked immunosorbent assays (ELISAs).

In an aspect, a reduction in expression is measured using qRT-PCR. In another aspect, a reduction in expression is measured using an RNA blot. In another aspect, a reduction in expression is measured using RNA sequencing. In a further aspect, a reduction in expression is measured using a Western blot. In yet a further aspect, a reduction in expression is measured using an ELISA.

In an aspect, a non-natural mutation in a nucleic acid sequence selected from the group consisting of SEQ ID NOs: 1-116 results in a reduced level of expression of the nucleic acid sequence as compared to the nucleic acid sequence lacking the non-natural mutation in a control plant grown under comparable conditions. In an aspect, a non-natural mutation in a nucleic acid sequence encoding an amino acid sequence selected from the group consisting of SEQ ID NOs: 117-174 results in a reduced level of expression of the nucleic acid sequence as compared to the nucleic acid sequence lacking the non-natural mutation in a control plant grown under comparable conditions.

In an aspect, a reduction in expression comprises a reduction of at least 1% as compared to expression in the same tissue of a control plant grown under comparable conditions. In another aspect, a reduction in expression comprises a reduction of at least 5% as compared to expression in the same tissue of a control plant grown under comparable conditions. In another aspect, a reduction in expression comprises a reduction of at least 10% as compared to expression in the same tissue of a control plant grown under comparable conditions. In another aspect, a reduction in expression comprises a reduction of at least 25% as compared to expression in the same tissue of a control plant grown under comparable conditions. In another aspect, a reduction in expression comprises a reduction of at least 50% as compared to expression in the same tissue of a control plant grown under comparable conditions. In another aspect, a reduction in expression comprises a reduction of at least 75% as compared to expression in the same tissue of a control plant grown under comparable conditions. In another aspect, a reduction in expression comprises a reduction of at least 90% as compared to expression in the same tissue of a control plant grown under comparable conditions. In another aspect, a reduction in expression comprises a reduction of at least 95% as compared to expression in the same tissue of a control plant grown under comparable conditions.

In an aspect, a reduction in expression comprises a reduction of between 1% and 99% as compared to expression in the same tissue of a control plant grown under comparable conditions. In another aspect, a reduction in expression comprises a reduction of between 1% and 90% as compared to expression in the same tissue of a control plant grown under comparable conditions. In another aspect, a reduction in expression comprises a reduction of between 1% and 75% as compared to expression in the same tissue of a control plant grown under comparable conditions. In another aspect, a reduction in expression comprises a reduction of between 1% and 50% as compared to expression in the same tissue of a control plant grown under comparable conditions. In another aspect, a reduction in expression comprises a reduction of between 1% and 25% as compared to expression in the same tissue of a control plant grown under comparable conditions. In another aspect, a reduction in expression comprises a reduction of between 25% and 90% as compared to expression in the same tissue of a control plant grown under comparable conditions. In another aspect, a reduction in expression comprises a reduction of between 50% and 90% as compared to expression in the same tissue of a control plant grown under comparable conditions. In another aspect, a reduction in expression comprises a reduction of between 25% and 75% as compared to expression in the same tissue of a control plant grown under comparable conditions.

In an aspect, a reduction in expression comprises a statistically significant reduction as compared to expression in the same tissue of a control plant grown under comparable conditions. One of ordinary skill in the art would recognize that any level of reduction is envisioned, so long as the level of reduction has been determined to be statistically significant using an accepted statistical hypothesis test. As a non-limiting example, a Student's t-test is one statistical hypothesis test that can be used to determine if a reduction in expression between a modified plant and a control plant is statistically significant. As used herein, “statistically significant” refers to a p-value of less than or equal to 0.05.

In an aspect, a non-natural mutation results in a reduced level of activity by a protein or polypeptide encoded by a nucleic acid sequence provided herein as compared to the activity of a control plant grown under comparable conditions. In another aspect, a non-natural mutation in an endogenous nucleic acid sequence selected from the group consisting of SEQ ID NOs: 1-116 reduces the level of activity by a protein or polypeptide encoded by the nucleic acid sequence as compared to activity of a protein or polypeptide encoded by the endogenous nucleic acid sequence in a control tobacco plant when grown under comparable conditions, where the nucleic acid sequence lacks the non-natural mutation in the control tobacco plant. In another aspect, a non-natural mutation in an endogenous nucleic acid sequence, where the endogenous nucleic acid sequence encodes an amino acid sequence selected from the group consisting of SEQ ID NOs: 117-174 reduces the level of activity by a protein or polypeptide encoded by the nucleic acid sequence as compared to activity of a protein or polypeptide encoded by the endogenous nucleic acid sequence in a control tobacco plant when grown under comparable conditions, where the nucleic acid sequence lacks the non-natural mutation in the control tobacco plant.

As used herein, when referring to a protein or polypeptide, “activity” refers to the ability to carry out an enzymatic function.

### Increased Expression/Activity

In an aspect, a non-natural mutation results in increased expression of a nucleic acid sequence. In an aspect, a non-natural mutation results in an increased level of expression of said nucleic acid sequence as compared to expression of said nucleic acid sequence in the same tissue of a control tobacco plant when grown under comparable conditions, wherein said nucleic acid sequence lacks the at least one non-natural mutation in said control tobacco plant.

In an aspect, an increased level of expression comprises an increase of at least 5% as compared to expression in the same tissue of a control plant grown under comparable conditions. In another aspect, an increased level of expression comprises an increase of at least 10% as compared to expression in the same tissue of a control plant grown under comparable conditions. In another aspect, an increased level of expression comprises an increase of at least 25% as compared to expression in the same tissue of a control plant grown under comparable conditions. In another aspect, an increased level of expression comprises an increase of at least 50% as compared to expression in the same tissue of a control plant grown under comparable conditions. In another aspect, an increased level of expression comprises an increase of at least 75% as compared to expression in the same tissue of a control plant grown under comparable conditions. In another aspect, an increased level of expression comprises an increase of at least 100% as compared to expression in the same tissue of a control plant grown under comparable conditions. In another aspect, an increased level of expression comprises an increase of at least 200% as compared to expression in the same tissue of a control plant grown under comparable conditions. In another aspect, an increased level of expression comprises an increase of at least 500% as compared to expression in the same tissue of a control plant grown under comparable conditions.

In an aspect, a non-natural mutation results in an increased level of activity by a protein or polypeptide encoded by said nucleic acid sequence as compared to activity of a protein or polypeptide encoded by said nucleic acid sequence in a control tobacco plant when grown under comparable conditions, wherein said nucleic acid sequence lacks the at least one non-natural mutation in said control tobacco plant.

### Alkaloids

As used herein, “alkaloid” refers to a complex, nitrogen-containing compound that naturally occurs in tobacco, and has pharmacological effects in humans or other animals. Non-limiting examples of alkaloids include nicotine, anabasine, anatabine, and nornicotine.

Nicotine is the primary natural alkaloid in commercialized cigarette tobacco, and it accounts for approximately 90% of the alkaloid content in Nicotiana tabacum. Without being limiting, other major alkaloids in tobacco include cotinine, nornicotine, myosmine, nicotyrine, anabasine and anatabine. Without being limiting, minor tobacco alkaloids include nicotine-n-oxide, N-methyl anatabine, N-methyl anabasine, pseudooxynicotine, 2,3 dipyridyl and others.

In an aspect, an alkaloid is selected from the group consisting of anabasine, anatabine, nicotine, and nornicotine.

Alkaloid levels can be assayed by methods known in the art, for example by quantification based on gas-liquid chromatography, high performance liquid chromatography, radio-immunoassays, enzyme-linked immunosorbent assays, and mass spectrometry. For example, nicotinic alkaloid levels can be measured by a GC-FID method based on CORESTA Recommended Method No. 7, 1987 and ISO Standards (ISO TC 126N 394 E. See also Hibi et al., Plant Physiology 100: 826-35 (1992) for a method using gas-liquid chromatography equipped with a capillary column and an FID detector.

Alkaloid levels can be assayed from any plant tissue. Non-limiting examples include leaf and hairy root cultures. Additionally, alkaloid levels can be assayed in cured plant materials or in plant materials that have not been cured.

Alternatively, tobacco total alkaloids can be measured using a segmented-flow colorimetric method developed for analysis of tobacco samples as adapted by Skalar Instrument Co. (West Chester, PA) and described by Collins et al., Tobacco Science 13:79-81 (1969). In short, samples of tobacco are dried, ground, and extracted prior to analysis of total alkaloids and reducing sugars. The method then employs an acetic acid/methanol/water extraction and charcoal for decolorization. Determination of total alkaloids was based on the reaction of cyanogen chloride with nicotine alkaloids in the presence of an aromatic amine to form a colored complex which is measured at 460 nm.

In an aspect, the level of an individual alkaloid is measured based on a freeze-dried cured leaf sample using liquid chromatography with tandem mass spectrometry (LC/MS/MS). In an aspect, the level of an individual alkaloid is measured based on a freeze-dried hairy root culture sample using liquid chromatography with tandem mass spectrometry (LC/MS/MS).

Unless specified otherwise, the nicotine or alkaloid level (or another leaf chemistry or property characterization) of a tobacco plant is measured after topping in a pooled leaf sample collected from leaf number 3, 4, and 5 after topping. As used herein, whenever a comparison between leaves from two plants (e.g., a mutant plant versus a control plant) is mentioned, leaves from the same or comparable stalk position(s) and developmental stage(s) are intended so that the comparison can demonstrate effects due to genotype differences, not from other factors. As a non-limiting illustration, leaf 3 of a control plant is intended as a reference point for comparing with leaf 3 of a modified plant comprising a non-natural mutation or recombinant DNA construct.

As used herein, leaf numbering is based on the leaf position on a tobacco stalk with leaf number 1 being the youngest leaf (at the top) after topping and the highest leaf number assigned to the oldest leaf (at the bottom).

As used herein, “topping” refers to the removal of the stalk apex, including the shoot apical meristem, flowers, and up to several adjacent leaves, when a tobacco plant is near vegetative maturity and around the start of reproductive growth. Typically, tobacco plants are topped in the button stage (soon after the flower begins to appear). For example, greenhouse or field-grown tobacco plants can be topped when 50% of the plants have at least one open flower. Topping a tobacco plant results in the loss of apical dominance and also induce increased alkaloid production.

Typically, the alkaloid level (or another leaf chemistry or property characterization; e.g., polyamines) of a tobacco plant is measured about 2 weeks after topping. Other time points can also be used. In an aspect, alkaloid level (or another leaf chemistry or property characterization) of a tobacco plant is measured about 1, 2, 3, 4, or 5 weeks after topping. In another aspect, the nicotine, alkaloid, or polyamine level (or another leaf chemistry or property characterization; e.g., polyamines) of a tobacco plant is measured about 3, 5, 7, 10, 12, 14, 17, 19, or 21 days after topping.

In an aspect, a modified tobacco plant, or part thereof, comprising at least one non-natural mutation in an endogenous nucleic acid produces at least one leaf comprising a reduced amount of at least one alkaloid as compared to the amount of the alkaloid in a control tobacco plant lacking the at least one non-natural mutation in the endogenous nucleic acid when grown under comparable conditions.

In an aspect, a modified tobacco plant, or part thereof, comprising a recombinant DNA construct provided herein produces at least one leaf comprising a reduced amount of at least one alkaloid as compared to the amount of the alkaloid in a control tobacco plant lacking the recombinant DNA construct when grown under comparable conditions.

In an aspect, a reduced level of at least one alkaloid comprises a reduction of at least 0.5% as compared to a control tobacco plant when grown under comparable conditions. In an aspect, a reduced level of at least one alkaloid comprises a reduction of at least 1% as compared to a control tobacco plant when grown under comparable conditions. In an aspect, a reduced level of at least one alkaloid comprises a reduction of at least 2% as compared to a control tobacco plant when grown under comparable conditions. In an aspect, a reduced level of at least one alkaloid comprises a reduction of at least 3% as compared to a control tobacco plant when grown under comparable conditions. In an aspect, a reduced level of at least one alkaloid comprises a reduction of at least 4% as compared to a control tobacco plant when grown under comparable conditions. In an aspect, a reduced level of at least one alkaloid comprises a reduction of at least 5% as compared to a control tobacco plant when grown under comparable conditions. In an aspect, a reduced level of at least one alkaloid comprises a reduction of at least 10% as compared to a control tobacco plant when grown under comparable conditions. In an aspect, a reduced level of at least one alkaloid comprises a reduction of at least 15% as compared to a control tobacco plant when grown under comparable conditions. In an aspect, a reduced level of at least one alkaloid comprises a reduction of at least 20% as compared to a control tobacco plant when grown under comparable conditions. In an aspect, a reduced level of at least one alkaloid comprises a reduction of at least 25% as compared to a control tobacco plant when grown under comparable conditions. In an aspect, a reduced level of at least one alkaloid comprises a reduction of at least 35% as compared to a control tobacco plant when grown under comparable conditions. In an aspect, a reduced level of at least one alkaloid comprises a reduction of at least 50% as compared to a control tobacco plant when grown under comparable conditions. In an aspect, a reduced level of at least one alkaloid comprises a reduction of at least 75% as compared to a control tobacco plant when grown under comparable conditions. In an aspect, a reduced level of at least one alkaloid comprises a reduction of at least 90% as compared to a control tobacco plant when grown under comparable conditions.

In an aspect, a reduced level of at least one alkaloid comprises a reduction of at between 1% and 99% as compared to a control tobacco plant when grown under comparable conditions. In an aspect, a reduced level of at least one alkaloid comprises a reduction of at between 1% and 90% as compared to a control tobacco plant when grown under comparable conditions. In an aspect, a reduced level of at least one alkaloid comprises a reduction of at between 1% and 80% as compared to a control tobacco plant when grown under comparable conditions. In an aspect, a reduced level of at least one alkaloid comprises a reduction of at between 1% and 70% as compared to a control tobacco plant when grown under comparable conditions. In an aspect, a reduced level of at least one alkaloid comprises a reduction of at between 1% and 60% as compared to a control tobacco plant when grown under comparable conditions. In an aspect, a reduced level of at least one alkaloid comprises a reduction of at between 1% and 50% as compared to a control tobacco plant when grown under comparable conditions. In an aspect, a reduced level of at least one alkaloid comprises a reduction of at between 1% and 40% as compared to a control tobacco plant when grown under comparable conditions. In an aspect, a reduced level of at least one alkaloid comprises a reduction of at between 1% and 30% as compared to a control tobacco plant when grown under comparable conditions. In an aspect, a reduced level of at least one alkaloid comprises a reduction of at between 1% and 20% as compared to a control tobacco plant when grown under comparable conditions. In an aspect, a reduced level of at least one alkaloid comprises a reduction of at between 1% and 10% as compared to a control tobacco plant when grown under comparable conditions. In an aspect, a reduced level of at least one alkaloid comprises a reduction of at between 1% and 5% as compared to a control tobacco plant when grown under comparable conditions. In an aspect, a reduced level of at least one alkaloid comprises a reduction of at between 10% and 75% as compared to a control tobacco plant when grown under comparable conditions. In an aspect, a reduced level of at least one alkaloid comprises a reduction of at between 10% and 50% as compared to a control tobacco plant when grown under comparable conditions. In an aspect, a reduced level of at least one alkaloid comprises a reduction of at between 10% and 25% as compared to a control tobacco plant when grown under comparable conditions.

In an aspect, a reduced level of at least one alkaloid comprises a reduction of nicotine by at least 0.5% as compared to a control tobacco plant when grown under comparable conditions. In an aspect, a reduced level of at least one alkaloid comprises a reduction of nicotine by at least 1% as compared to a control tobacco plant when grown under comparable conditions. In an aspect, a reduced level of at least one alkaloid comprises a reduction of nicotine by at least 2% as compared to a control tobacco plant when grown under comparable conditions. In an aspect, a reduced level of at least one alkaloid comprises a reduction of nicotine by at least 3% as compared to a control tobacco plant when grown under comparable conditions. In an aspect, a reduced level of at least one alkaloid comprises a reduction of nicotine by at least 4% as compared to a control tobacco plant when grown under comparable conditions. In an aspect, a reduced level of at least one alkaloid comprises a reduction of nicotine by at least 5% as compared to a control tobacco plant when grown under comparable conditions. In an aspect, a reduced level of at least one alkaloid comprises a reduction of nicotine by at least 10% as compared to a control tobacco plant when grown under comparable conditions. In an aspect, a reduced level of at least one alkaloid comprises a reduction of nicotine by at least 15% as compared to a control tobacco plant when grown under comparable conditions. In an aspect, a reduced level of at least one alkaloid comprises a reduction of nicotine by at least 20% as compared to a control tobacco plant when grown under comparable conditions. In an aspect, a reduced level of at least one alkaloid comprises a reduction of nicotine by at least 25% as compared to a control tobacco plant when grown under comparable conditions. In an aspect, a reduced level of at least one alkaloid comprises a reduction of nicotine by at least 35% as compared to a control tobacco plant when grown under comparable conditions. In an aspect, a reduced level of at least one alkaloid comprises a reduction of nicotine by at least 50% as compared to a control tobacco plant when grown under comparable conditions. In an aspect, a reduced level of at least one alkaloid comprises a reduction of nicotine by at least 75% as compared to a control tobacco plant when grown under comparable conditions. In an aspect, a reduced level of at least one alkaloid comprises a reduction of nicotine by at least 90% as compared to a control tobacco plant when grown under comparable conditions.

In an aspect, a reduced level of at least one alkaloid comprises a reduction of nicotine by between 1% and 99% as compared to a control tobacco plant when grown under comparable conditions. In an aspect, a reduced level of at least one alkaloid comprises a reduction of nicotine by between 1% and 90% as compared to a control tobacco plant when grown under comparable conditions. In an aspect, a reduced level of at least one alkaloid comprises a reduction of nicotine by between 1% and 80% as compared to a control tobacco plant when grown under comparable conditions. In an aspect, a reduced level of at least one alkaloid comprises a reduction of nicotine by between 1% and 70% as compared to a control tobacco plant when grown under comparable conditions. In an aspect, a reduced level of at least one alkaloid comprises a reduction of nicotine by between 1% and 60% as compared to a control tobacco plant when grown under comparable conditions. In an aspect, a reduced level of at least one alkaloid comprises a reduction of nicotine by between 1% and 50% as compared to a control tobacco plant when grown under comparable conditions. In an aspect, a reduced level of at least one alkaloid comprises a reduction of nicotine by between 1% and 40% as compared to a control tobacco plant when grown under comparable conditions. In an aspect, a reduced level of at least one alkaloid comprises a reduction of nicotine by between 1% and 30% as compared to a control tobacco plant when grown under comparable conditions. In an aspect, a reduced level of at least one alkaloid comprises a reduction of nicotine by between 1% and 20% as compared to a control tobacco plant when grown under comparable conditions. In an aspect, a reduced level of at least one alkaloid comprises a reduction of nicotine by between 1% and 10% as compared to a control tobacco plant when grown under comparable conditions. In an aspect, a reduced level of at least one alkaloid comprises a reduction of nicotine by between 1% and 5% as compared to a control tobacco plant when grown under comparable conditions. In an aspect, a reduced level of at least one alkaloid comprises a reduction of nicotine by between 10% and 75% as compared to a control tobacco plant when grown under comparable conditions. In an aspect, a reduced level of at least one alkaloid comprises a reduction of nicotine by between 10% and 50% as compared to a control tobacco plant when grown under comparable conditions. In an aspect, a reduced level of at least one alkaloid comprises a reduction of nicotine by between 10% and 25% as compared to a control tobacco plant when grown under comparable conditions.

In an aspect, a modified tobacco plant comprises a nicotine level of less than or equal to 17 milligrams per gram (mg/g) of dry weight. In an aspect, a modified tobacco plant comprises a nicotine level of less than or equal to 16 mg/g of dry weight. In an aspect, a modified tobacco plant comprises a nicotine level of less than or equal to 15 mg/g of dry weight. In an aspect, a modified tobacco plant comprises a nicotine level of less than or equal to 14 mg/g of dry weight. In an aspect, a modified tobacco plant comprises a nicotine level of less than or equal to 13 mg/g of dry weight. In an aspect, a modified tobacco plant comprises a nicotine level of less than or equal to 12 mg/g of dry weight. In an aspect, a modified tobacco plant comprises a nicotine level of less than or equal to 11 mg/g of dry weight. In an aspect, a modified tobacco plant comprises a nicotine level of less than or equal to 10 mg/g of dry weight. In an aspect, a modified tobacco plant comprises a nicotine level of less than or equal to 9 mg/g of dry weight. In an aspect, a modified tobacco plant comprises a nicotine level of less than or equal to 8 mg/g of dry weight. In an aspect, a modified tobacco plant comprises a nicotine level of less than or equal to 7 mg/g of dry weight. In an aspect, a modified tobacco plant comprises a nicotine level of less than or equal to 6 mg/g of dry weight. In an aspect, a modified tobacco plant comprises a nicotine level of less than or equal to 5 mg/g of dry weight. In an aspect, a modified tobacco plant comprises a nicotine level of less than or equal to 4 mg/g of dry weight. In an aspect, a modified tobacco plant comprises a nicotine level of less than or equal to 3 mg/g of dry weight. In an aspect, a modified tobacco plant comprises a nicotine level of less than or equal to 2 mg/g of dry weight. In an aspect, a modified tobacco plant comprises a nicotine level of less than or equal to 1.5 mg/g of dry weight. In an aspect, a modified tobacco plant comprises a nicotine level of less than or equal to 1 mg/g of dry weight.

In an aspect, a reduced level of at least one alkaloid comprises a reduction of anatabine by at least 0.5% as compared to a control tobacco plant when grown under comparable conditions. In an aspect, a reduced level of at least one alkaloid comprises a reduction of anatabine by at least 1% as compared to a control tobacco plant when grown under comparable conditions. In an aspect, a reduced level of at least one alkaloid comprises a reduction of anatabine by at least 2% as compared to a control tobacco plant when grown under comparable conditions. In an aspect, a reduced level of at least one alkaloid comprises a reduction of anatabine by at least 3% as compared to a control tobacco plant when grown under comparable conditions. In an aspect, a reduced level of at least one alkaloid comprises a reduction of anatabine by at least 4% as compared to a control tobacco plant when grown under comparable conditions. In an aspect, a reduced level of at least one alkaloid comprises a reduction of anatabine by at least 5% as compared to a control tobacco plant when grown under comparable conditions. In an aspect, a reduced level of at least one alkaloid comprises a reduction of anatabine by at least 10% as compared to a control tobacco plant when grown under comparable conditions. In an aspect, a reduced level of at least one alkaloid comprises a reduction of anatabine by at least 15% as compared to a control tobacco plant when grown under comparable conditions. In an aspect, a reduced level of at least one alkaloid comprises a reduction of anatabine by at least 20% as compared to a control tobacco plant when grown under comparable conditions. In an aspect, a reduced level of at least one alkaloid comprises a reduction of anatabine by at least 25% as compared to a control tobacco plant when grown under comparable conditions. In an aspect, a reduced level of at least one alkaloid comprises a reduction of anatabine by at least 35% as compared to a control tobacco plant when grown under comparable conditions. In an aspect, a reduced level of at least one alkaloid comprises a reduction of anatabine by at least 50% as compared to a control tobacco plant when grown under comparable conditions. In an aspect, a reduced level of at least one alkaloid comprises a reduction of anatabine by at least 75% as compared to a control tobacco plant when grown under comparable conditions. In an aspect, a reduced level of at least one alkaloid comprises a reduction of anatabine by at least 90% as compared to a control tobacco plant when grown under comparable conditions.

In an aspect, a reduced level of at least one alkaloid comprises a reduction of anatabine by between 1% and 99% as compared to a control tobacco plant when grown under comparable conditions. In an aspect, a reduced level of at least one alkaloid comprises a reduction of anatabine by between 1% and 90% as compared to a control tobacco plant when grown under comparable conditions. In an aspect, a reduced level of at least one alkaloid comprises a reduction of anatabine by between 1% and 80% as compared to a control tobacco plant when grown under comparable conditions. In an aspect, a reduced level of at least one alkaloid comprises a reduction of anatabine by between 1% and 70% as compared to a control tobacco plant when grown under comparable conditions. In an aspect, a reduced level of at least one alkaloid comprises a reduction of anatabine by between 1% and 60% as compared to a control tobacco plant when grown under comparable conditions. In an aspect, a reduced level of at least one alkaloid comprises a reduction of anatabine by between 1% and 50% as compared to a control tobacco plant when grown under comparable conditions. In an aspect, a reduced level of at least one alkaloid comprises a reduction of anatabine by between 1% and 40% as compared to a control tobacco plant when grown under comparable conditions. In an aspect, a reduced level of at least one alkaloid comprises a reduction of anatabine by between 1% and 30% as compared to a control tobacco plant when grown under comparable conditions. In an aspect, a reduced level of at least one alkaloid comprises a reduction of anatabine by between 1% and 20% as compared to a control tobacco plant when grown under comparable conditions. In an aspect, a reduced level of at least one alkaloid comprises a reduction of anatabine by between 1% and 10% as compared to a control tobacco plant when grown under comparable conditions. In an aspect, a reduced level of at least one alkaloid comprises a reduction of anatabine by between 1% and 5% as compared to a control tobacco plant when grown under comparable conditions. In an aspect, a reduced level of at least one alkaloid comprises a reduction of anatabine by between 10% and 75% as compared to a control tobacco plant when grown under comparable conditions. In an aspect, a reduced level of at least one alkaloid comprises a reduction of anatabine by between 10% and 50% as compared to a control tobacco plant when grown under comparable conditions. In an aspect, a reduced level of at least one alkaloid comprises a reduction of anatabine by between 10% and 25% as compared to a control tobacco plant when grown under comparable conditions.

In an aspect, a modified tobacco plant comprises an anatabine level of less than or equal to 1.5 mg/g of dry weight. In an aspect, a modified tobacco plant comprises an anatabine level of less than or equal to 1.4 mg/g of dry weight. In an aspect, a modified tobacco plant comprises an anatabine level of less than or equal to 1.3 mg/g of dry weight. In an aspect, a modified tobacco plant comprises an anatabine level of less than or equal to 1.2 mg/g of dry weight. In an aspect, a modified tobacco plant comprises an anatabine level of less than or equal to 1.1 mg/g of dry weight. In an aspect, a modified tobacco plant comprises an anatabine level of less than or equal to 1.0 mg/g of dry weight. In an aspect, a modified tobacco plant comprises an anatabine level of less than or equal to 0.9 mg/g of dry weight. In an aspect, a modified tobacco plant comprises an anatabine level of less than or equal to 0.8 mg/g of dry weight. In an aspect, a modified tobacco plant comprises an anatabine level of less than or equal to 0.7 mg/g of dry weight. In an aspect, a modified tobacco plant comprises an anatabine level of less than or equal to 0.6 mg/g of dry weight. In an aspect, a modified tobacco plant comprises an anatabine level of less than or equal to 0.5 mg/g of dry weight. In an aspect, a modified tobacco plant comprises an anatabine level of less than or equal to 0.4 mg/g of dry weight. In an aspect, a modified tobacco plant comprises an anatabine level of less than or equal to 0.3 mg/g of dry weight. In an aspect, a modified tobacco plant comprises an anatabine level of less than or equal to 0.25 mg/g of dry weight. In an aspect, a modified tobacco plant comprises an anatabine level of less than or equal to 0.2 mg/g of dry weight.

In an aspect, a reduced level of at least one alkaloid comprises a reduction of anabasine by at least 0.5% as compared to a control tobacco plant when grown under comparable conditions. In an aspect, a reduced level of at least one alkaloid comprises a reduction of anabasine by at least 1% as compared to a control tobacco plant when grown under comparable conditions. In an aspect, a reduced level of at least one alkaloid comprises a reduction of anabasine by at least 2% as compared to a control tobacco plant when grown under comparable conditions. In an aspect, a reduced level of at least one alkaloid comprises a reduction of anabasine by at least 3% as compared to a control tobacco plant when grown under comparable conditions. In an aspect, a reduced level of at least one alkaloid comprises a reduction of anabasine by at least 4% as compared to a control tobacco plant when grown under comparable conditions. In an aspect, a reduced level of at least one alkaloid comprises a reduction of anabasine by at least 5% as compared to a control tobacco plant when grown under comparable conditions. In an aspect, a reduced level of at least one alkaloid comprises a reduction of anabasine by at least 10% as compared to a control tobacco plant when grown under comparable conditions. In an aspect, a reduced level of at least one alkaloid comprises a reduction of anabasine by at least 15% as compared to a control tobacco plant when grown under comparable conditions. In an aspect, a reduced level of at least one alkaloid comprises a reduction of anabasine by at least 20% as compared to a control tobacco plant when grown under comparable conditions. In an aspect, a reduced level of at least one alkaloid comprises a reduction of anabasine by at least 25% as compared to a control tobacco plant when grown under comparable conditions. In an aspect, a reduced level of at least one alkaloid comprises a reduction of anabasine by at least 35% as compared to a control tobacco plant when grown under comparable conditions. In an aspect, a reduced level of at least one alkaloid comprises a reduction of anabasine by at least 50% as compared to a control tobacco plant when grown under comparable conditions. In an aspect, a reduced level of at least one alkaloid comprises a reduction of anabasine by at least 75% as compared to a control tobacco plant when grown under comparable conditions. In an aspect, a reduced level of at least one alkaloid comprises a reduction of anabasine by at least 90% as compared to a control tobacco plant when grown under comparable conditions.

In an aspect, a reduced level of at least one alkaloid comprises a reduction of anabasine by between 1% and 99% as compared to a control tobacco plant when grown under comparable conditions. In an aspect, a reduced level of at least one alkaloid comprises a reduction of anabasine by between 1% and 90% as compared to a control tobacco plant when grown under comparable conditions. In an aspect, a reduced level of at least one alkaloid comprises a reduction of anabasine by between 1% and 80% as compared to a control tobacco plant when grown under comparable conditions. In an aspect, a reduced level of at least one alkaloid comprises a reduction of anabasine by between 1% and 70% as compared to a control tobacco plant when grown under comparable conditions. In an aspect, a reduced level of at least one alkaloid comprises a reduction of anabasine by between 1% and 60% as compared to a control tobacco plant when grown under comparable conditions. In an aspect, a reduced level of at least one alkaloid comprises a reduction of anabasine by between 1% and 50% as compared to a control tobacco plant when grown under comparable conditions. In an aspect, a reduced level of at least one alkaloid comprises a reduction of anabasine by between 1% and 40% as compared to a control tobacco plant when grown under comparable conditions. In an aspect, a reduced level of at least one alkaloid comprises a reduction of anabasine by between 1% and 30% as compared to a control tobacco plant when grown under comparable conditions. In an aspect, a reduced level of at least one alkaloid comprises a reduction of anabasine by between 1% and 20% as compared to a control tobacco plant when grown under comparable conditions. In an aspect, a reduced level of at least one alkaloid comprises a reduction of anabasine by between 1% and 10% as compared to a control tobacco plant when grown under comparable conditions. In an aspect, a reduced level of at least one alkaloid comprises a reduction of anabasine by between 1% and 5% as compared to a control tobacco plant when grown under comparable conditions. In an aspect, a reduced level of at least one alkaloid comprises a reduction of anabasine by between 10% and 75% as compared to a control tobacco plant when grown under comparable conditions. In an aspect, a reduced level of at least one alkaloid comprises a reduction of anabasine by between 10% and 50% as compared to a control tobacco plant when grown under comparable conditions. In an aspect, a reduced level of at least one alkaloid comprises a reduction of anabasine by between 10% and 25% as compared to a control tobacco plant when grown under comparable conditions.

In an aspect, a modified tobacco plant comprises an anabasine level of less than or equal to 0.6 mg/g of dry weight. In an aspect, a modified tobacco plant comprises an anabasine level of less than or equal to 0.5 mg/g of dry weight. In an aspect, a modified tobacco plant comprises an anabasine level of less than or equal to 0.4 mg/g of dry weight. In an aspect, a modified tobacco plant comprises an anabasine level of less than or equal to 0.3 mg/g of dry weight. In an aspect, a modified tobacco plant comprises an anabasine level of less than or equal to 0.25 mg/g of dry weight. In an aspect, a modified tobacco plant comprises an anabasine level of less than or equal to 0.2 mg/g of dry weight. In an aspect, a modified tobacco plant comprises an anabasine level of less than or equal to 0.15 mg/g of dry weight. In an aspect, a modified tobacco plant comprises an anabasine level of less than or equal to 0.1 mg/g of dry weight.

In an aspect, a reduced level of at least one alkaloid comprises a reduction of nornicotine by at least 0.5% as compared to a control tobacco plant when grown under comparable conditions. In an aspect, a reduced level of at least one alkaloid comprises a reduction of nornicotine by at least 1% as compared to a control tobacco plant when grown under comparable conditions. In an aspect, a reduced level of at least one alkaloid comprises a reduction of nornicotine by at least 2% as compared to a control tobacco plant when grown under comparable conditions. In an aspect, a reduced level of at least one alkaloid comprises a reduction of nornicotine by at least 3% as compared to a control tobacco plant when grown under comparable conditions. In an aspect, a reduced level of at least one alkaloid comprises a reduction of nornicotine by at least 4% as compared to a control tobacco plant when grown under comparable conditions. In an aspect, a reduced level of at least one alkaloid comprises a reduction of nornicotine by at least 5% as compared to a control tobacco plant when grown under comparable conditions. In an aspect, a reduced level of at least one alkaloid comprises a reduction of nornicotine by at least 10% as compared to a control tobacco plant when grown under comparable conditions. In an aspect, a reduced level of at least one alkaloid comprises a reduction of nornicotine by at least 15% as compared to a control tobacco plant when grown under comparable conditions. In an aspect, a reduced level of at least one alkaloid comprises a reduction of nornicotine by at least 20% as compared to a control tobacco plant when grown under comparable conditions. In an aspect, a reduced level of at least one alkaloid comprises a reduction of nornicotine by at least 25% as compared to a control tobacco plant when grown under comparable conditions. In an aspect, a reduced level of at least one alkaloid comprises a reduction of nornicotine by at least 35% as compared to a control tobacco plant when grown under comparable conditions. In an aspect, a reduced level of at least one alkaloid comprises a reduction of nornicotine by at least 50% as compared to a control tobacco plant when grown under comparable conditions. In an aspect, a reduced level of at least one alkaloid comprises a reduction of nornicotine by at least 75% as compared to a control tobacco plant when grown under comparable conditions. In an aspect, a reduced level of at least one alkaloid comprises a reduction of nornicotine by at least 90% as compared to a control tobacco plant when grown under comparable conditions.

In an aspect, a reduced level of at least one alkaloid comprises a reduction of nornicotine by between 1% and 99% as compared to a control tobacco plant when grown under comparable conditions. In an aspect, a reduced level of at least one alkaloid comprises a reduction of nornicotine by between 1% and 90% as compared to a control tobacco plant when grown under comparable conditions. In an aspect, a reduced level of at least one alkaloid comprises a reduction of nornicotine by between 1% and 80% as compared to a control tobacco plant when grown under comparable conditions. In an aspect, a reduced level of at least one alkaloid comprises a reduction of nornicotine by between 1% and 70% as compared to a control tobacco plant when grown under comparable conditions. In an aspect, a reduced level of at least one alkaloid comprises a reduction of nornicotine by between 1% and 60% as compared to a control tobacco plant when grown under comparable conditions. In an aspect, a reduced level of at least one alkaloid comprises a reduction of nornicotine by between 1% and 50% as compared to a control tobacco plant when grown under comparable conditions. In an aspect, a reduced level of at least one alkaloid comprises a reduction of nornicotine by between 1% and 40% as compared to a control tobacco plant when grown under comparable conditions. In an aspect, a reduced level of at least one alkaloid comprises a reduction of nornicotine by between 1% and 30% as compared to a control tobacco plant when grown under comparable conditions. In an aspect, a reduced level of at least one alkaloid comprises a reduction of nornicotine by between 1% and 20% as compared to a control tobacco plant when grown under comparable conditions. In an aspect, a reduced level of at least one alkaloid comprises a reduction of nornicotine by between 1% and 10% as compared to a control tobacco plant when grown under comparable conditions. In an aspect, a reduced level of at least one alkaloid comprises a reduction of nornicotine by between 1% and 5% as compared to a control tobacco plant when grown under comparable conditions. In an aspect, a reduced level of at least one alkaloid comprises a reduction of nornicotine by between 10% and 75% as compared to a control tobacco plant when grown under comparable conditions. In an aspect, a reduced level of at least one alkaloid comprises a reduction of nornicotine by between 10% and 50% as compared to a control tobacco plant when grown under comparable conditions. In an aspect, a reduced level of at least one alkaloid comprises a reduction of nornicotine by between 10% and 25% as compared to a control tobacco plant when grown under comparable conditions.

In an aspect, a modified tobacco plant comprises a nornicotine level of less than or equal to 1.0 mg/g of dry weight. In an aspect, a modified tobacco plant comprises a nornicotine level of less than or equal to 0.9 mg/g of dry weight. In an aspect, a modified tobacco plant comprises a nornicotine level of less than or equal to 0.8 mg/g of dry weight. In an aspect, a modified tobacco plant comprises a nornicotine level of less than or equal to 0.7 mg/g of dry weight. In an aspect, a modified tobacco plant comprises a nornicotine level of less than or equal to 0.6 mg/g of dry weight. In an aspect, a modified tobacco plant comprises a nornicotine level of less than or equal to 0.5 mg/g of dry weight. In an aspect, a modified tobacco plant comprises a nornicotine level of less than or equal to 0.4 mg/g of dry weight. In an aspect, a modified tobacco plant comprises a nornicotine level of less than or equal to 0.3 mg/g of dry weight. In an aspect, a modified tobacco plant comprises a nornicotine level of less than or equal to 0.2 mg/g of dry weight.

### Plants

As used herein, a tobacco plant can be from any plant from the Nicotiana genus including, but not limited to Nicotiana tabacum, Nicotiana amplexicaulis PI 271989; Nicotiana benthamiana PI 555478; Nicotiana bigelovii PI 555485; Nicotiana debneyi; Nicotiana excelsior PI 224063; Nicotiana glutinosa PI 555507; Nicotiana goodspeedii PI 241012; Nicotiana gossei PI 230953; Nicotiana hesperis PI 271991; Nicotiana knightiana PI 555527; Nicotiana maritima PI 555535; Nicotiana megalosiphon PI 555536; Nicotiana nudicaulis PI 555540; Nicotiana paniculata PI 555545; Nicotiana plumbaginifolia PI 555548; Nicotiana repanda PI 555552; Nicotiana rustica; Nicotiana suaveolens PI 230960; Nicotiana sylvestris PI 555569; Nicotiana tomentosa PI 266379; Nicotiana tomentosiformis; and Nicotiana trigonophylla PI 555572. In an aspect, a tobacco plant described here is a Nicotiana tabacum plant.

In an aspect, tobacco parts provided include, but are not limited to, a leaf, a stem, a root, a trichome, a seed, a flower, pollen, an anther, an ovule, a pedicel, a fruit, a meristem, a cotyledon, a hypocotyl, a pod, an embryo, endosperm, an explant, a callus, a tissue culture, a shoot, a cell, and a protoplast. In an aspect, tobacco part provided does not include seed. In an aspect, this disclosure provides tobacco plant cells, tissues, and organs that are not reproductive material and do not mediate the natural reproduction of the plant. In another aspect, this disclosure also provides tobacco plant cells, tissues, and organs that are reproductive material and mediate the natural reproduction of the plant. In another aspect, this disclosure provides tobacco plant cells, tissues, and organs that cannot maintain themselves via photosynthesis. In another aspect, this disclosure provides somatic tobacco plant cells. Somatic cells, contrary to germline cells, do not mediate plant reproduction.

Cells, tissues and organs can be from seed, fruit, leaf, cotyledon, hypocotyl, meristem, embryos, endosperm, root, shoot, stem, trichome, pod, flower, inflorescence, stalk, pedicel, style, stigma, receptacle, petal, sepal, pollen, anther, filament, ovary, ovule, pericarp, phloem, vascular tissue. In another aspect, this disclosure provides a tobacco plant chloroplast. In a further aspect, this disclosure provides epidermal cells, stomata cell, leaf or root hairs, a storage root, or a tuber. In another aspect, this disclosure provides a tobacco protoplast.

Skilled artisans understand that tobacco plants naturally reproduce via seeds, not via asexual reproduction or vegetative propagation. In an aspect, this disclosure provides tobacco endosperm.

This disclosure provides cells from tobacco plants provided herein.

As used herein, a “progeny tobacco plant” or “progeny tobacco seed” can be from any filial generation, e.g., F₁, F₂, F₃, F₄, F₅, F₆, F₇, etc.

In an aspect, a tobacco plant, or part thereof, is of a tobacco variety selected from the group consisting of a flue-cured variety, a bright variety, a Burley variety, a Virginia variety, a Maryland variety, a dark variety, a Galpio variety, an Oriental variety, and a Turkish variety. In one aspect, a modified tobacco plant, or part thereof, provided herein is of a tobacco variety selected from the group consisting of a flue-cured variety, a bright variety, a Burley variety, a Virginia variety, a Maryland variety, a dark variety, a Galpio variety, an Oriental variety, and a Turkish variety.

In an aspect, a tobacco cell is of a tobacco variety selected from the group consisting of a flue cured variety, a bright variety, a Burley variety, a Virginia variety, a Maryland variety, a dark variety, a Galpio variety, an Oriental variety, and a Turkish variety. In an aspect, a modified tobacco cell is of a tobacco variety selected from the group consisting of a flue cured variety, a bright variety, a Burley variety, a Virginia variety, a Maryland variety, a dark variety, a Galpio variety, an Oriental variety, and a Turkish variety.

In an aspect, a tobacco leaf is of a tobacco variety selected from the group consisting of a flue cured variety, a bright variety, a Burley variety, a Virginia variety, a Maryland variety, a dark variety, a Galpio variety, an Oriental variety, and a Turkish variety.

In an aspect, a cured tobacco leaf or plant part is of a tobacco variety selected from the group consisting of a flue cured variety, a bright variety, a Burley variety, a Virginia variety, a Maryland variety, a dark variety, a Galpio variety, an Oriental variety, and a Turkish variety. Skilled artisans further understand that cured tobacco does not constitute a living organism and is not capable of growth or reproduction

Flue-cured tobaccos (also called “Virginia” or “bright” tobaccos) amount to approximately 40% of world tobacco production. Flue-cured tobaccos are often also referred to as “bright tobacco” because of the golden-yellow to deep-orange color it reaches during curing. Flue-cured tobaccos have a light, bright aroma and taste. Flue-cured tobaccos are generally high in sugar and low in oils. Major flue-cured tobacco growing countries are Argentina, Brazil, China, India, Tanzania and the United States of America. In one aspect, tobacco plants or seeds or modified tobacco plants or seeds provided herein are of a flue-cured tobacco variety selected from the group consisting of the varieties listed in Table 2, and any variety essentially derived from any one of the foregoing varieties. See WO 2004/041006 A1. In a further aspect, modified tobacco plants or seeds provided herein are in a flue-cured variety selected from the group consisting of K326, K346, and NC 196.

TABLE 2
Flue-cured Tobacco Varieties

| | | |
|---|---|---|
| 400 (TC 225) | K 346 | Reams 134 |
| 401 (TC 226) | K 346 (TC 569) | Reams 158 |
| 401 Cherry Red (TC 227) | K 358 | Reams 713 |
| 401 Cherry Red Free (TC 228) | K 394 (TC 321) | Reams 744 |
| Cash (TC 250) | K 399 | Reams M1 |
| Cash (TI 278) | K 399 (TC 322) | RG 11 (TC 600) |
| CC 101 | K 730 | RG 13 (TC 601) |
| CC 1063 | Lonibow (TI 1573) | RG 17 (TC 627) |
| CC 13 | Lonibow (TI 1613) | RG 22 (TC 584) |
| CC 143 | McNair 10 (TC 330) | RG 8 (TC 585) |
| CC 200 | McNair 135 (TC 337) | RG 81 (TC 618) |
| CC 27 | McNair 30 (TC 334) | RG H51 |
| CC 301 | McNair 373 (TC 338) | RG4H 217 |
| CC 33 | McNair 944 (TC 339) | RGH 12 |
| CC 35 | MK94 (TI 1512) | RGH 4 |
| CC 37 | MS K 326 | RGH 51 |
| CC 400 | MS NC 71 | RGH 61 |
| CC 500 | MS NC 72 | SC 58 (TC 400) |
| CC 600 | NC 100 | SC 72 (TC 403) |
| CC 65 | NC 102 | Sp. G-168 |
| CC 67 | NC 1071 (TC 364) | SPEIGHT 168 |
| CC 700 | NC 1125-2 | Speight 168 (TC 633) |
| CC 800 | NC 12 (TC 346) | Speight 172 (TC 634) |
| CC 900 | NC 1226 | Speight 178 |
| Coker 139 (TC 259) | NC 196 | Speight 179 |
| Coker 139 yb1, yb2 | NC 2326 (TC 365) | Speight 190 |
| Coker 140 (TC 260) | NC 27 NF (TC 349) | Speight 196 |
| Coker 176 (TC 262) | NC 291 | SPEIGHT 220 |
| Coker 187 (TC 263) | NC 297 | SPEIGHT 225 |
| Coker 187-Hicks (TC 265) | NC 299 | SPEIGHT 227 |
| Coker 209 (TC 267) | NC 37 NF (TC 350) | SPEIGHT 236 |
| Coker 258 (TC 270) | NC 471 | Speight G-10 (TC 416) |
| Coker 298 (TC 272) | NC 55 | Speight G-102 |
| Coker 316 (TC 273) | NC 567 (TC 362) | Speight G-108 |
| Coker 319 (TC 274) | NC 60 (TC 352) | Speight G-111 |
| Coker 347 (TC 275) | NC 606 | Speight G-117 |
| Coker 371-Gold (TC 276) | NC 6140 | Speight G-126 |
| Coker 411 (TC 277) | NC 71 | Speight G-15 (TC 418) |
| Coker 48 (TC 253) | NC 72 | Speight G-23 |
| Coker 51 (TC 254) | NC 729 (TC 557) | Speight G-28 (TC 420) |
| Coker 86 (TC 256) | NC 810 (TC 659) | Speight G-33 |
| CU 263 (TC 619) | NC 82 (TC 356) | Speight G-41 |
| CU 561 | NC 8640 | Speight G-5 |
| DH95-1562-1 | NC 89 (TC 359) | Speight G-52 |
| Dixie Bright 101 (TC 290) | NC 92 | Speight G-58 |
| Dixie Bright 102 (TC 291) | NC 925 | Speight G-70 |
| Dixie Bright 244 (TC 292) | NC 95 (TC 360) | Speight G-70 (TC 426) |
| Dixie Bright 27 (TC 288) | NC 98 (TC 361) | Speight G-80 (TC 427) |
| Dixie Bright 28 (TC 289) | NC EX 24 | Speight NF3 (TC 629) |
| GF 157 | NC PY 10 (TC 367) | STNCB |
| GF 318 | NC TG 61 | VA 182 |
| GL 26H | Oxford 1 (TC 369) | VA 45 (TC 559) |
| GL 338 | Oxford 1-181 (TC 370) | Vesta 30 (TC 439) |
| GL 350 | Oxford 2 (TC 371) | Vesta 33 (TC 440) |
| GL 368 | Oxford 207 (TC 632) | Vesta 5 (TC 438) |
| GL 395 | Oxford 26 (TC 373) | Vesta 62 (TC 441) |
| GL 600 | Oxford 3 (TC 372) | Virginia (TI 220) |
| GL 737 | Oxford 414 NF | Virginia (TI 273) |
| GL 939 | PD 611 (TC 387) | Virginia (TI 877) |
| GL 939 (TC 628) | PVH 03 | Virginia 115 (TC 444) |
| Hicks (TC 310) | PVH 09 | Virginia 21 (TC 443) |
| Hicks Broadleaf (TC 311) | PVH 1118 | Virginia Bright (TI 964) |
| K 149 (TC 568) | PVH 1452 | Virginia Bright Leaf (TC 446) |
| K 317 | PVH 1600 | Virginia Gold (TC 447) |
| K 326 | PVH 2110 | White Stem Orinoco (TC 451) |
| K 326 (TC 319) | PVH 2275 | |
| K 340 (TC 320) | R 83 (Line 256-1) (TI 1400) | |

Air-cured tobaccos include “Burley,” “Maryland,” and “dark” tobaccos. The common factor linking air-cured tobaccos is that curing occurs primarily without artificial sources of heat and humidity. Burley tobaccos are light to dark brown in color, high in oil, and low in sugar. Burley tobaccos are typically air-cured in barns. Major Burley growing countries include Argentina, Brazil, Italy, Malawi, and the United States of America.

Maryland tobaccos are extremely fluffy, have good burning properties, low nicotine and a neutral aroma. Major Maryland growing countries include the United States of America and Italy.

In one aspect, tobacco plants or seeds or modified tobacco plants or seeds provided herein are of a Burley tobacco variety selected from the group consisting of the tobacco varieties listed in Table 3, and any variety essentially derived from any one of the foregoing varieties. In a further aspect, modified tobacco plants or seeds provided herein are in a Burley variety selected from the group consisting of TN 90, KT 209, KT 206, KT212, and HB 4488.

TABLE 3
Burley Tobacco Varieties

| | | |
|---|---|---|
| 4407 LC | HB 4108P | KY 54 (TC 71) |
| AA-37-1 | HB 4151P | KY 56 (TC 72) |
| Burley 21 (TC 7) | HB 4192P | KY 56 (TC 72) |
| Burley 49 (TC 10) | HB 4194P | KY 57 (TC 73) |
| Burley 64 (TC 11) | HB 4196 | KY 58 (TC 74) |
| Burley Mammoth KY | HB 4488 | KY 8654 (TC 77) |
| 16 (TC 12) | | |
| Clay 402 | HB 4488P | KY 8959 |
| Clay 403 | HB04P | KY 9 (TC 54) |
| Clay 502 | HB 4488 LC | KY 907 LC |
| Clays 403 | HIB 21 | KY 908 (TC 630) |
| GR 10 (TC 19) | HPB 21 | NBH 98 (Screened) |
| GR 10 (TC 19) | HY 403 | NC 1206 |
| GR 10A (TC 20) | Hybrid 403 LC | NC 129 |
| GR 13 (TC 21) | Hybrid 404 LC | NC 2000 LC |
| GR 14 (TC 22) | Hybrid 501 LC | NC 2002 LC |
| GR 149 LC | KDH-959 (TC 576) | NC 3 LC |
| GR 153 | KDH-960 (TC 577) | NC 5 LC |
| GR 17 (TC 23) | KT 200 LC | NC 6 LC |
| GR 17B (TC 24) | KT 204 LC | NC 7 LC |
| GR 18 (TC 25) | KT 206 LC | NC BH 129 LC |
| GR 19 (TC 26) | KT 209 LC | NC03-42-2 |
| GR 2 (TC 15) | KT 210 LC | Newton 98 |
| GR 24 (TC 27) | KT 212 LC | R 610 LC |
| GR 36 (TC 28) | KT 215 LC | R 630 LC |
| GR 38 (TC 29) | KY 1 (TC 52) | R 7-11 |
| GR 38A (TC 30) | KY 10 (TC 55) | R 59-116 LC |
| GR 40 (TC 31) | KY 12 (TC 56) | RG 17 |
| GR 42 (TC 32) | KY 14 (TC 57) | TKF 1801 LC |
| GR 42C (TC 33) | KY 14 × L8 LC | TKF 2002 LC |
| GR 43 (TC 34) | KY 15 (TC 58) | TKF 4024 LC |
| GR 44 (TC 35) | KY 16 (TC 59) | TKF 4028 LC |
| GR 45 (TC 36) | KY 17 (TC 60) | TKF 6400 LC |
| GR 46 (TC 37) | KY 19 (TC 61) | TKF 7002 LC |
| GR 48 (TC 38) | KY 21 (TC 62) | TKS 2002 LC |
| GR 5 (TC 16) | KY 22 (TC 63) | TN 86 (TC 82) |
| GR 53 (TC 39) | KY 24 (TC 64) | TN 90 LC |
| GR 6 (TC 17) | KY 26 (TC 65) | TN 97 Hybrid LC |
| GR 9 (TC 18) | KY 33 (TC 66) | TN 97 LC |
| GR139 NS | KY 34 (TC 67) | VA 116 |
| GR139 S | KY 35 (TC 68) | VA 119 |
| HB 04P | KY 41A (TC 69) | Virgin A |
| | | Mutante (TI 1406) |
| HB 04P LC | KY 5 (TC 53) | Virginia 509 (TC 84) |
| HB 3307P LC | KY 52 (TC 70) | |

In another aspect, tobacco plants or seeds or modified tobacco plants or seeds provided herein are of a Maryland tobacco variety selected from the group consisting of the tobacco varieties listed in Table 4, and any variety essentially derived from any one of the foregoing varieties.

TABLE 4
Maryland Tobacco Varieties

Maryland 10 (TC 498)
Maryland 14 D2 (TC 499)
Maryland 201 (TC 503)
Maryland 21 (TC 500)
Maryland 341 (TC 504)
Maryland 40
Maryland 402
Maryland 59 (TC 501)
Maryland 601
Maryland 609 (TC 505)
Maryland 64 (TC 502)
Maryland 872 (TC 506)
Maryland Mammoth (TC 507)

Dark air-cured tobaccos are distinguished from other tobacco types primarily by its curing process, which gives dark air-cured tobacco its medium-brown to dark-brown color and a distinct aroma. Dark air-cured tobaccos are mainly used in the production of chewing tobacco and snuff. In one aspect, modified tobacco plants or seeds provided herein are of a dark air-cured tobacco variety selected from the group consisting of Sumatra, Jatim, Dominican Cubano, Besuki, One sucker, Green River, Virginia sun-cured, and Paraguan Passado, and any variety essentially derived from any one of the foregoing varieties.

Dark fire-cured tobaccos are generally cured with low-burning wood fires on the floors of closed curing barns. Dark fire-cured tobaccos are typically used for making pipe blends, cigarettes, chewing tobacco, snuff, and strong-tasting cigars. Major growing regions for dark fire-cured tobaccos are Tennessee, Kentucky, and Virginia in the United States of America. In one aspect, tobacco plants or seeds or modified tobacco plants or seeds provided herein are of a dark fire-cured tobacco variety selected from the group consisting of the tobacco varieties listed in Table 5, and any variety essentially derived from any one of the foregoing varieties.

TABLE 5
Dark Tobacco Varieties

| | | |
|---|---|---|
| Black Mammoth (TC 461) | KY 171 (TC 475) | PD 7309 LC |
| Black Mammoth Small Stalk (TC 641) | KY 171 LC | PD 7312 LC |
| Certified Madole (TC 463) | KY 171 NS | PD 7318 LC |
| D-534-A-1 (TC 464) | KY 180 (TC 573) | PD 7319 LC |
| DAC ULT 302 | KY 190 (TC 574) | Petico M PG04 |
| DAC ULT 303 | Little Crittenden | PY KY 160 (TC 612) |
| DAC ULT 306 | Little Crittenden (TC 476) | PY KY 171 (TC 613) |
| DAC ULT 308 | Little Crittenden LC | Shirey |
| | (certified) | |
| DAC ULT 312 | Little Crittenden PhPh | TI 1372 |
| DF 300 (TC 465) | Lizard Tail Turtle Foot | TN D94 |
| DF 485 (TC 466) | Madole (TC 478) | TN D94 (TC 621) |
| DF 516 (TC 467) | Madole (TC 479) | TN D950 |
| DF 911 (TC 468) | MS KY 171 | TN D950 (PhPh) |
| DT 508 | MS NL Madole LC | TN D950 |
| DT 518 (Screened) | MS TN D950 LC | TN D950 (TC 622) |
| DT 538 LC | Nance (TC 616) | TR Madole (TC 486) |
| DT 592 | Narrow Leaf Madole LC | VA 309 |
| | (certified) | |
| Improved Madole (TC 471) | Neal Smith Madole (TC 646) | VA 309 (TC 560) |
| Jernigan's Madole (TC 472) | Newtons VH Madole | VA 309 LC (certified) |
| KT 14LC | NL Madole | VA 310 (TC 487) |
| KT D17LC | NL Madole (PhPh) | VA 331 (TC 592) |
| KT D4 LC | NL Madole (TC 484) | VA 355 (TC 638) |
| KT D6 LC | NL Madole LC | VA 359 |
| KT D8 LC | NL Madole LC (PhPh) | VA 359 (Screened) |
| KY 153 (TC 216) | NL Madole NS | VA 359 (TC 639) |
| KY 157 (TC 217) | One Sucker (TC 224) | VA 359 LC (certified) |
| KY 160 | OS 400 | VA 403 (TC 580) |
| KY 160 (TC 218) | PD 302H | VA 405 (TC 581) |
| KY 163 (TC 219) | PD 312H | VA 409 (TC 562) |
| KY 165 (TC 220) | PD 318H | VA 510 (TC 572) |
| KY 170 (TC 474) | PD 7302 LC | |
| KY 171 (PhPh) | PD 7305 | |

Oriental tobaccos are also referred to as Greek, aroma and Turkish tobaccos due to the fact that they are typically grown in eastern Mediterranean regions such as Turkey, Greece, Bulgaria, Macedonia, Syria, Lebanon, Italy, and Romania. The small plant size, small leaf size, and unique aroma properties of Oriental tobacco varieties are a result of their adaptation to the poor soil and stressful climatic conditions in which they have been developed. In one aspect, tobacco plants or seeds or modified tobacco plants or seeds provided herein are of an Oriental tobacco variety selected from the group consisting of the tobacco varieties listed in Table 6, and any variety essentially derived from any one of the foregoing varieties.

TABLE 6
Oriental Tobacco Varieties

| | | |
|---|---|---|
| Bafra (TI 1641) | Edirne (TI 1671) | Samsun (TC 536) |
| Bahce (TI 1730) | Ege (TI 1642) | Samsun 959 (TI 1570) |
| Bahia (TI 1416) | Ege-64 (TI 1672) | Samsun Evkaf (TI 1723) |
| Bahia (TI 1455) | Izmir (Akhisar) (TI 1729) | Samsun Holmes NN (TC 540) |
| Baiano (TI 128) | Izmir (Gavurkoy) (TI 1727) | Samsun Maden (TI 1647) |
| Basma | Izmir Ege 64 | Samsun NO 15 (TC 541) |
| Basma (TI 1666) | Izmir-Incekara (TI 1674) | Samsun-BLK SHK Tol (TC 542) |
| Basma Drama | Izmir-Ozbas (TI 1675) | Samsun-Canik (TI 1678) |
| Basma Hybrid (PhPh) | Jaka Dzebel (TI 1326) | Samsun-Maden (TI 1679) |
| Basma Zihna I | Kaba-Kulak | Saribaptar 407 - Izmir Region |
| Bitlis (TI 1667) | Kagoshima Maruba (TI 158) | Smyrna (TC 543) |
| Bitlis (TI 1725) | Katerini | Smyrna No. 23 (TC 545) |
| Bubalovac (TI 1282) | Katerini S53 | Smyrna No. 9 (TC 544) |
| Bursa (TI 1650) | Krumovgrad 58 | Smyrna-Blk Shk Tol (TC 546) |
| Bursa (TI 1668) | MS Basma | Trabzon (TI 1649) |
| Canik (TI 1644) | MS Katerini S53 | Trabzon (TI 1682) |
| Djebel 174 (TI 1492) | Nevrokop 1146 | Trapezund 161 (TI 1407) |
| Djebel 359 (TI 1493) | Ozbas (TI 1645) | Turkish (TC 548) |
| Djebel 81 | Perustitza (TI 980) | Turkish Angshit (TI 90) |
| Dubec 566 (TI 1409) | Prilep (TI 1291) | Turkish Samsum (TI 92) |
| Dubec 7 (TI 1410) | Prilep (TI 1325) | Turkish Tropizoid (TI 93) |
| Dubek 566 (TI 1567) | Prilep 12-2/1 | Turkish Varotic (TI 89) |
| Duzce (TI 1670) | Prilep 23 | Xanthi (TI 1662) |

In an aspect, tobacco plants or seeds or modified tobacco plants or seeds provided herein are of an cigar tobacco variety selected from the group consisting of the tobacco varieties listed in Table 7, and any variety essentially derived from any one of the foregoing varieties.

TABLE 7
Cigar Tobacco Varieties

| | | |
|---|---|---|
| Bahai (TI 62) | Castillo Negro, Blanco, Pina (TI 449) | Enshu (TI 1586) |
| Beinhart 1000 | Caujaro (TI 893) | Florida 301 |
| Beinhart 1000 (TI 1562) | Chocoa (TI 289) | Florida 301 (TC 195) |
| Beinhart 1000-1 (TI 1561) | Chocoa (TI 313) | PA Broadleaf (TC 119) |
| Bergerac C | Connecticut 15 (TC 183) | Pennsylvania Broadleaf |
| Bergerac C (TI 1529) | Connecticut Broadleaf | Pennsylvania Broadleaf (TC 119) |
| Big Cuban (TI 1565) | Connecticut Broadleaf (TC 186) | Petite Havana SR1 |
| Castillo Negro, Blanco, | Connecticut Shade (TC 188) | Petite Havana SR1 (TC 105) |
| Pina (TI 448) | | |
| Castillo Negro, Blanco, | | |
| Pina (TI 448A) | Criollo, Colorado (TI 1093) | |

In an aspect, tobacco plants or seeds or modified tobacco plants or seeds provided herein are of a tobacco variety selected from the group consisting of the tobacco varieties listed in Table 8, and any variety essentially derived from any one of the foregoing varieties.

TABLE 8
Other Tobacco Varieties

Chocoa (TI 319)
Hoja Parada (TI 1089)
Hoja Parado (Galpoa) (TI 1068)
Perique (St. James Parrish)
Perique (TC 556)
Perique (TI 1374)
Sylvestris (TI 984)
TI 179

In an aspect, a tobacco plant, or part thereof, is from a variety selected from the group consisting of the tobacco varieties listed in Table 2, Table 3, Table 4, Table 5, Table 6, Table 7, and Table 8. In another aspect, a tobacco plant, or part thereof, is from a variety listed in Table 2. In another aspect, a tobacco plant, or part thereof, is from a variety listed in Table 3. In another aspect, a tobacco plant, or part thereof, is from a variety listed in Table 4. In another aspect, a tobacco plant, or part thereof, is from a variety listed in Table 5. In another aspect, a tobacco plant, or part thereof, is from a variety listed in Table 6. In another aspect, a tobacco plant, or part thereof, is from a variety listed in Table 7. In another aspect, a tobacco plant, or part thereof, is from a variety listed in Table 8.

In an aspect, a modified tobacco plant, or part thereof, is from a variety selected from the group consisting of the tobacco varieties listed in Table 2, Table 3, Table 4, Table 5, Table 6, Table 7, and Table 8. In an aspect, a modified tobacco plant, or part thereof, is from a variety listed in Table 2. In another aspect, a modified tobacco plant, or part thereof, is from a variety listed in Table 3. In another aspect, a modified tobacco plant, or part thereof, is from a variety listed in Table 4. In another aspect, a modified tobacco plant, or part thereof, is from a variety listed in Table 5. In another aspect, a modified tobacco plant, or part thereof, is from a variety listed in Table 6. In another aspect, a modified tobacco plant, or part thereof, is from a variety listed in Table 7. In another aspect, a modified tobacco plant, or part thereof, is from a variety listed in Table 8.

In an aspect, a tobacco seed is from a variety selected from the group consisting of the tobacco varieties listed in Table 2, Table 3, Table 4, Table 5, Table 6, Table 7, and Table 8. In another aspect, a tobacco seed is from a variety listed in Table 2. In another aspect, a tobacco seed is from a variety listed in Table 3. In another aspect, a tobacco seed is from a variety listed in Table 4. In another aspect, a tobacco seed is from a variety listed in Table 5. In another aspect, a tobacco seed is from a variety listed in Table 6. In another aspect, a tobacco seed is from a variety listed in Table 7. In another aspect, a tobacco seed is from a variety listed in Table 8.

In an aspect, a tobacco cell is from a variety selected from the group consisting of the tobacco varieties listed in Table 2, Table 3, Table 4, Table 5, Table 6, Table 7, and Table 8. In another aspect, a tobacco cell is from a variety listed in Table 2. In another aspect, a tobacco cell is from a variety listed in Table 3. In another aspect, a tobacco cell is from a variety listed in Table 4. In another aspect, a tobacco cell is from a variety listed in Table 5. In another aspect, a tobacco cell is from a variety listed in Table 6. In another aspect, a tobacco cell is from a variety listed in Table 7. In another aspect, a tobacco cell is from a variety listed in Table 8.

All foregoing mentioned specific varieties of flue-cured, dark air-cured, Burley, Maryland, dark fire-cured, cigar, or Oriental type are listed only for exemplary purposes. Any additional flue-cured, dark air-cured, Burley, Maryland, dark fire-cured, cigar, or Oriental varieties are also contemplated in the present application.

In an aspect, a tobacco plant or variety provided herein is an inbred tobacco plant or variety. As used herein, an “inbred” tobacco variety is a variety that has been bred for genetic homogeneity.

As used herein, a “hybrid” is created by crossing two plants from different varieties or species, such that the progeny comprises genetic material from each parent. Skilled artisans recognize that higher order hybrids can be generated as well. For example, a first hybrid can be made by crossing Variety C with Variety D to create a C×D hybrid, and a second hybrid can be made by crossing Variety E with Variety F to create an E×F hybrid. The first and second hybrids can be further crossed to create the higher order hybrid (C×D)×(E×F) comprising genetic information from all four parent varieties. In an aspect, a modified tobacco plant provided herein is a hybrid tobacco plant. In another aspect, a modified tobacco seed provided herein is a hybrid tobacco seed. In an aspect, a tobacco plant or variety provided herein is a hybrid tobacco plant or variety. In another aspect, a modified tobacco plant provided herein is a hybrid tobacco plant.

In an aspect, this disclosure provides a method for producing a modified tobacco plant comprising: (a) crossing at least one tobacco plant of a first tobacco variety with at least one tobacco plant of a second tobacco variety to produce at least one progeny tobacco seed, where the at least one tobacco plant of the first tobacco variety comprises a non-natural mutation in an endogenous nucleic acid sequence, where the endogenous nucleic acid sequence encodes a polypeptide comprising an amino acid sequence at least 80% identical or similar to an amino acid sequence selected from the group consisting of SEQ ID NOs: 117-174, where the non-natural mutation is not present in the endogenous nucleic acid sequence in a control tobacco plant of the first tobacco variety; and (b) selecting for at least one progeny tobacco seed, or a plant germinated therefrom, wherein the at least one tobacco seed or plant germinated therefrom comprises the non-natural mutation. In another aspect, this disclosure provides a method for producing a modified tobacco plant comprising: (a) crossing at least one tobacco plant of a first tobacco variety with at least one tobacco plant of a second tobacco variety to produce at least one progeny tobacco seed, where the at least one tobacco plant of the first tobacco variety comprises a non-natural mutation in an endogenous nucleic acid sequence at least 80% identical to a nucleic acid sequence selected from the group consisting of SEQ ID NOs: 1-58, where the non-natural mutation is not present in the endogenous nucleic acid sequence in a control tobacco plant of the first tobacco variety; and (b) selecting for at least one progeny tobacco seed, or a plant germinated therefrom, wherein the at least one tobacco seed or plant germinated therefrom comprises the non-natural mutation. In another aspect, this disclosure provides a method for producing a modified tobacco plant comprising: (a) crossing at least one tobacco plant of a first tobacco variety with at least one tobacco plant of a second tobacco variety to produce at least one progeny tobacco seed, where the at least one tobacco plant of the first tobacco variety comprises a non-natural mutation in an endogenous nucleic acid sequence at least 80% identical to a nucleic acid sequence selected from the group consisting of SEQ ID NOs: 59-116, where the non-natural mutation is not present in the endogenous nucleic acid sequence in a control tobacco plant of the first tobacco variety; and (b) selecting for at least one progeny tobacco seed, or a plant germinated therefrom, wherein the at least one tobacco seed or plant germinated therefrom comprises the non-natural mutation. In another aspect, this disclosure provides a method for producing a modified tobacco plant comprising: (a) crossing at least one tobacco plant of a first tobacco variety with at least one tobacco plant of a second tobacco variety to produce at least one progeny tobacco seed, where the at least one tobacco plant of the first tobacco variety comprises a non-natural mutation in an endogenous nucleic acid sequence selected from the group consisting of SEQ ID NOs: 1-116, where the non-natural mutation is not present in the endogenous nucleic acid sequence in a control tobacco plant of the first tobacco variety; and (b) selecting for at least one progeny tobacco seed, or a plant germinated therefrom, wherein the at least one tobacco seed or plant germinated therefrom comprises the non-natural mutation. In an aspect, in any of the foregoing methods, the first tobacco variety and second tobacco variety are the same tobacco variety. In another aspect, in any of the foregoing methods, the first tobacco variety and second tobacco variety are two different tobacco varieties.

In an aspect, this disclosure provides a method for producing a modified tobacco plant comprising: (a) crossing at least one tobacco plant of a first tobacco variety with at least one tobacco plant of a second tobacco variety to produce at least one progeny tobacco seed, where the at least one tobacco plant of the first tobacco variety comprises a recombinant DNA construct, where the recombinant DNA construct comprises a heterologous promoter operably linked to a nucleic acid sequence encoding at least one small RNA molecule capable of binding to and reducing the expression of an endogenous nucleic acid sequence encoding a polypeptide at least 80% identical or similar to an amino acid sequence selected from the group consisting of SEQ ID NOs: 117-174, were the recombinant DNA construct is not present in a control tobacco plant of the first tobacco variety; and (b) selecting for at least one progeny tobacco seed, or a plant germinated therefrom, wherein the at least one progeny tobacco seed or plant germinated therefrom comprises the recombinant DNA construct. In another aspect, this disclosure provides a method for producing a modified tobacco plant comprising: (a) crossing at least one tobacco plant of a first tobacco variety with at least one tobacco plant of a second tobacco variety to produce at least one progeny tobacco seed, where the at least one tobacco plant of the first tobacco variety comprises a recombinant DNA construct, where the recombinant DNA construct comprises a heterologous promoter operably linked to a nucleic acid sequence encoding at least one small RNA molecule capable of binding to and reducing the expression of an endogenous nucleic acid sequence at least 80% identical or similar to a nucleic acid sequence selected from the group consisting of SEQ ID NOs: 1-58, were the recombinant DNA construct is not present in a control tobacco plant of the first tobacco variety; and (b) selecting for at least one progeny tobacco seed, or a plant germinated therefrom, wherein the at least one progeny tobacco seed or plant germinated therefrom comprises the recombinant DNA construct. In another aspect, this disclosure provides a method for producing a modified tobacco plant comprising: (a) crossing at least one tobacco plant of a first tobacco variety with at least one tobacco plant of a second tobacco variety to produce at least one progeny tobacco seed, where the at least one tobacco plant of the first tobacco variety comprises a recombinant DNA construct, where the recombinant DNA construct comprises a heterologous promoter operably linked to a nucleic acid sequence encoding at least one small RNA molecule capable of binding to and reducing the expression of an endogenous nucleic acid sequence at least 80% identical or similar to a nucleic acid sequence selected from the group consisting of SEQ ID NOs: 59-116, were the recombinant DNA construct is not present in a control tobacco plant of the first tobacco variety; and (b) selecting for at least one progeny tobacco seed, or a plant germinated therefrom, where the at least one progeny tobacco seed or plant germinated therefrom comprises the recombinant DNA construct. In an aspect, in any of the foregoing methods, the first tobacco variety and second tobacco variety are the same tobacco variety. In another aspect, in any of the foregoing methods, the first tobacco variety and second tobacco variety are two different tobacco varieties.

In an aspect, this disclosure provides a method for producing a modified tobacco plant comprising: (a) crossing at least one tobacco plant of a first tobacco variety with at least one tobacco plant of a second tobacco variety to produce at least one progeny tobacco seed, where the at least one tobacco plant of the first tobacco variety comprises a recombinant DNA construct, where the recombinant DNA construct comprises a heterologous promoter operably linked to a nucleic acid sequence encoding a polypeptide comprising an amino acid sequence at least 80% identical or similar to an amino acid sequence selected from the group consisting of SEQ ID NOs: 117-174, where the recombinant DNA construct is not present in a control tobacco plant of the first tobacco variety; and (b) selecting for at least one progeny tobacco seed, or a plant germinated therefrom, where the at least one progeny tobacco seed or plant germinated therefrom comprises the recombinant DNA construct. In another aspect, this disclosure provides a method for producing a modified tobacco plant comprising: (a) crossing at least one tobacco plant of a first tobacco variety with at least one tobacco plant of a second tobacco variety to produce at least one progeny tobacco seed, where the at least one tobacco plant of the first tobacco variety comprises a recombinant DNA construct, where the recombinant DNA construct comprises a heterologous promoter operably linked to a nucleic acid sequence at least 80% identical to a nucleic acid sequence selected from the group consisting of SEQ ID NOs: 1-58, where the recombinant DNA construct is not present in a control tobacco plant of the first tobacco variety; and (b) selecting for at least one progeny tobacco seed, or a plant germinated therefrom, where the at least one progeny tobacco seed or plant germinated therefrom comprises the recombinant DNA construct. In another aspect, this disclosure provides a method for producing a modified tobacco plant comprising: (a) crossing at least one tobacco plant of a first tobacco variety with at least one tobacco plant of a second tobacco variety to produce at least one progeny tobacco seed, where the at least one tobacco plant of the first tobacco variety comprises a recombinant DNA construct, where the recombinant DNA construct comprises a heterologous promoter operably linked to a nucleic acid sequence at least 80% identical to a nucleic acid sequence selected from the group consisting of SEQ ID NOs: 59-116, where the recombinant DNA construct is not present in a control tobacco plant of the first tobacco variety; and (b) selecting for at least one progeny tobacco seed, or a plant germinated therefrom, where the at least one progeny tobacco seed or plant germinated therefrom comprises the recombinant DNA construct. In an aspect, in any of the foregoing methods, the first tobacco variety and second tobacco variety are the same tobacco variety. In another aspect, in any of the foregoing methods, the first tobacco variety and second tobacco variety are two different tobacco varieties.

As used herein, the term “crossing” refers to the deliberate mating of two plants. In an aspect, crossing comprises pollination and/or fertilization of a first tobacco plant by a second tobacco plant. The two tobacco plants being crossed can be distantly related, closely related, or identical. In an aspect, the two tobacco plants being crossed are both modified tobacco plants. In an aspect, the two tobacco plants being crossed are of the same tobacco variety. In an aspect, the two tobacco plants being crossed are of two different tobacco varieties. In an aspect, one of the two tobacco plants being crossed is male sterile. In an aspect, one of the two tobacco plants being crossed is female sterile. In an aspect, at least one of the two tobacco plants being crossed is a hybrid tobacco plant. In an aspect, at least one of the two tobacco plants being crossed is a modified tobacco plant.

In an aspect, a tobacco plant or variety provided herein is male sterile. In another aspect, a tobacco plant or variety provided herein is cytoplasmic male sterile (CMS). In an aspect, a modified tobacco plant or variety provided herein is male sterile. In another aspect, a modified tobacco plant or variety provided herein is cytoplasmic male sterile (CMS). Male sterile tobacco plants can be produced by any method known in the art. Methods of producing male sterile tobacco are described in Wernsman, E. A., and Rufty, R. C. 1987. Chapter Seventeen. Tobacco. Pages 669-698 In: Cultivar Development. Crop Species. W. H. Fehr (ed.), MacMillan Publishing Go., Inc., New York, N.Y. 761 pp.

In another aspect, a tobacco plant or variety provided herein is female sterile. In another aspect, a modified tobacco plant or variety provided herein is female sterile. As a non-limiting example, female sterile plants can be made by mutating the STIG1 gene. See, for example, Goldman et al. 1994, EMBO Journal 13:2976-2984. In an aspect, a modified tobacco plant provided herein is female sterile.

Unless specified otherwise, all comparisons to control plants require similar growth conditions or comparable growth conditions for the two plants being compared. As used herein, “grown under comparable conditions,” “similar growth conditions” or “comparable growth conditions” refer to similar environmental conditions and/or agronomic practices for growing and making meaningful comparisons between two or more plant genotypes so that neither environmental conditions nor agronomic practices would contribute to or explain any difference observed between the two or more plant genotypes. Environmental conditions include, for example, light, temperature, water (humidity), and nutrition (e.g., nitrogen and phosphorus). Agronomic practices include, for example, seeding, clipping, undercutting, transplanting, topping, and suckering. See Chapters 4B and 4C of Tobacco, Production, Chemistry and Technology, Davis & Nielsen, eds., Blackwell Publishing, Oxford (1999), pp 70-103. As used herein, a “control plant” refers to a plant of identical, or nearly identical, genetic makeup as the modified plant being compared, except for the non-natural mutation or recombinant DNA construct provided herein that was introduced to the modified plant.

In an aspect, a modified tobacco plant described herein is a low-alkaloid variety or low-alkaloid plant. As a non-limiting example, LA Burley 21 (LA BU21) is a low-alkaloid variety of tobacco. LA BU21 is produced by incorporation of a low alkaloid gene(s) from a Cuban cigar variety into Burley 21 through several backcrosses. It has approximately 0.2% total alkaloids (dry weight) compared to the about 3.5% (dry weight) of its parent, Burley 21. LA BU21 has a leaf grade well below commercially acceptable standards. LA BU21 also exhibits other unfavorable leaf phenotypes characterized by lower yields, delayed ripening and senescence, higher susceptibility to insect herbivory, and poor end-product quality after curing. LA BU21 leaves further exhibit traits such as higher polyamine content, higher chlorophyll content and more mesophyll cells per unit leaf area. See US2019/0271000 for more characterization of LA BU21 leaf phenotypes.

In an aspect, the present disclosure provides tobacco plants, or part thereof, comprising a low nicotine or low alkaloid-conferring mutation or transgene (e.g., a genetic modification in or targeting one or more ADC, AO, or ODC) and capable of producing a leaf comprising a comparable level of one or more polyamines relative to a comparable leaf of a control plant not comprising the same mutation or transgene. In one aspect, a comparable level of one or more polyamines is within 20%, 17.5%, 15%, 12.5%, 10%, 7.5%, 5%, 2.5%, or 1% of the level in a comparable leaf of a control plant not comprising the same mutation or transgene. In an aspect, a comparable level of one or more polyamines is between 0.5% and 1%, between 1% and 2%, between 2% and 3%, between 3% and 4%, between 4% and 5%, between 5% and 6%, between 6% and 7%, between 7% and 8%, between 8% and 9%, between 9% and 10%, between 11% and 12%, between 12% and 13%, between 13% and 14%, between 14% and 15%, between 15% and 16%, between 16% and 17%, between 17% and 18%, between 18% and 19%, or between 19% and 20% of the level in a comparable leaf of a control plant not comprising the same mutation or transgene. In a further aspect, a comparable level of one or more polyamines is between 0.5% and 5%, between 5% and 10%, or between 10% and 20% of the level in a comparable leaf of a control plant not comprising the same mutation or transgene.

In an aspect, the present disclosure provides ADC mutant or transgenic tobacco plants or parts thereof, AO mutant or transgenic tobacco plants or parts thereof, or ODC mutant or transgenic tobacco plants or parts thereof, capable of producing a leaf comprising a comparable chlorophyll level relative to a comparable leaf of a control plant not comprising the same mutation or transgene. In one aspect, a comparable chlorophyll level is within 20%, 17.5%, 15%, 12.5%, 10%, 7.5%, 5%, 2.5%, or 1% of the level in a comparable leaf of a control plant not comprising the same mutation or transgene. In an aspect, a comparable chlorophyll level is between 0.5% and 1%, between 1% and 2%, between 2% and 3%, between 3% and 4%, between 4% and 5%, between 5% and 6%, between 6% and 7%, between 7% and 8%, between 8% and 9%, between 9% and 10%, between 11% and 12%, between 12% and 13%, between 13% and 14%, between 14% and 15%, between 15% and 16%, between 16% and 17%, between 17% and 18%, between 18% and 19%, or between 19% and 20% of the level in a comparable leaf of a control plant not comprising the same mutation or transgene. In a further aspect, a comparable chlorophyll level is between 0.5% and 5%, between 5% and 10%, or between 10% and 20% of the level in a comparable leaf of a control plant not comprising the same mutation or transgene.

In an aspect, the present disclosure provides ADC mutant or transgenic tobacco plants or parts thereof, AO mutant or transgenic tobacco plants or parts thereof, or ODC mutant or transgenic tobacco plants or parts thereof, capable of producing a leaf comprising a comparable number of mesophyll cell per unit of leaf area relative to a comparable leaf of a control plant not comprising the same mutation or transgene. In one aspect, a comparable number of mesophyll cell per unit of leaf area is within 20%, 17.5%, 15%, 12.5%, 10%, 7.5%, 5%, 2.5%, or 1% of the level in a comparable leaf of a control plant not comprising the same mutation or transgene. In an aspect, a comparable number of mesophyll cell per unit of leaf area is between 0.5% and 1%, between 1% and 2%, between 2% and 3%, between 3% and 4%, between 4% and 5%, between 5% and 6%, between 6% and 7%, between 7% and 8%, between 8% and 9%, between 9% and 10%, between 11% and 12%, between 12% and 13%, between 13% and 14%, between 14% and 15%, between 15% and 16%, between 16% and 17%, between 17% and 18%, between 18% and 19%, or between 19% and 20% of the level in a comparable leaf of a control plant not comprising the same mutation or transgene. In a further aspect, a comparable number of mesophyll cell per unit of leaf area is between 0.5% and 5%, between 5% and 10%, or between 10% and 20% of the level in a comparable leaf of a control plant not comprising the same mutation or transgene.

In an aspect, the present disclosure provides ADC mutant or transgenic tobacco plants or parts thereof, AO mutant or transgenic tobacco plants or parts thereof, or ODC mutant or transgenic tobacco plants or parts thereof, capable of producing a leaf comprising a comparable epidermal cell size relative to a comparable leaf of a control plant not comprising the same mutation or transgene. In one aspect, a comparable epidermal cell size is within 20%, 17.5%, 15%, 12.5%, 10%, 7.5%, 5%, 2.5%, or 1% of the level in a comparable leaf of a control plant not comprising the same mutation or transgene. In an aspect, a comparable epidermal cell size is between 0.5% and 1%, between 1% and 2%, between 2% and 3%, between 3% and 4%, between 4% and 5%, between 5% and 6%, between 6% and 7%, between 7% and 8%, between 8% and 9%, between 9% and 10%, between 11% and 12%, between 12% and 13%, between 13% and 14%, between 14% and 15%, between 15% and 16%, between 16% and 17%, between 17% and 18%, between 18% and 19%, or between 19% and 20% of the level in a comparable leaf of a control plant not comprising the same mutation or transgene. In a further aspect, a comparable epidermal cell size is between 0.5% and 5%, between 5% and 10%, or between 10% and 20% of the level in a comparable leaf of a control plant not comprising the same mutation or transgene.

In an aspect, the present disclosure provides ADC mutant or transgenic tobacco plants or parts thereof, AO mutant or transgenic tobacco plants or parts thereof, or ODC mutant or transgenic tobacco plants or parts thereof, capable of producing a leaf comprising a comparable leaf yield relative to a comparable leaf of a control plant not comprising the same mutation or transgene. In one aspect, a comparable leaf yield is within 20%, 17.5%, 15%, 12.5%, 10%, 7.5%, 5%, 2.5%, or 1% of the level in a comparable leaf of a control plant not comprising the same mutation or transgene. In an aspect, a comparable leaf yield is between 0.5% and 1%, between 1% and 2%, between 2% and 3%, between 3% and 4%, between 4% and 5%, between 5% and 6%, between 6% and 7%, between 7% and 8%, between 8% and 9%, between 9% and 10%, between 11% and 12%, between 12% and 13%, between 13% and 14%, between 14% and 15%, between 15% and 16%, between 16% and 17%, between 17% and 18%, between 18% and 19%, or between 19% and 20% of the level in a comparable leaf of a control plant not comprising the same mutation or transgene. In a further aspect, a comparable leaf yield is between 0.5% and 5%, between 5% and 10%, or between 10% and 20% of the level in a comparable leaf of a control plant not comprising the same mutation or transgene.

In an aspect, the present disclosure provides ADC mutant or transgenic tobacco plants or parts thereof, AO mutant or transgenic tobacco plants or parts thereof, or ODC mutant or transgenic tobacco plants or parts thereof, exhibiting a comparable insect herbivory susceptibility relative to a comparable leaf of a control plant not comprising the same mutation or transgene. In one aspect, a comparable insect herbivory susceptibility is within 20%, 17.5%, 15%, 12.5%, 10%, 7.5%, 5%, 2.5%, or 1% of the level in a comparable leaf of a control plant not comprising the same mutation or transgene. In an aspect, a comparable insect herbivory susceptibility is between 0.5% and 1%, between 1% and 2%, between 2% and 3%, between 3% and 4%, between 4% and 5%, between 5% and 6%, between 6% and 7%, between 7% and 8%, between 8% and 9%, between 9% and 10%, between 11% and 12%, between 12% and 13%, between 13% and 14%, between 14% and 15%, between 15% and 16%, between 16% and 17%, between 17% and 18%, between 18% and 19%, or between 19% and 20% of the level in a comparable leaf of a control plant not comprising the same mutation or transgene. In a further aspect, a comparable insect herbivory susceptibility is between 0.5% and 5%, between 5% and 10%, or between 10% and 20% of the level in a comparable leaf of a control plant not comprising the same mutation or transgene.

Any genetic modification provided herein can be introduced into any ADC mutant or transgenic tobacco plants or parts thereof, AO mutant or transgenic tobacco plants or parts thereof, or ODC mutant or transgenic tobacco plants or parts thereof.

Insect herbivory susceptibility level can be assayed by methods known in the art, for example, in an insect feeding assay. In short, a quarter inch layer of 0.7% agar in water is added to a 100 mm Petri dish and allowed to solidify. Leaf discs are cut from the petri dish lid, placed in the plates and pushed gently into the agar. Leaf discs are taken from plants at the 4-5 leaf stage. Discs were taken from lamina only to exclude major midribs. A single disc is taken from each of the four largest leaves of the plant generating 4 replicates per plant. Four plants are sampled for a total of 16 biological replicates test line. A single budworm (e.g., Heliothis sp., Helicoverpa sp.) at the second instar stage is added to the leaf and allowed to feed for 48 hours at ambient temperature. After 48 hours the budworm larvae are weighed and final larval weights are recorded.

In an aspect, a tobacco plant, or part thereof, comprises relative to a control tobacco plant: a first genome modification providing a lower level of nicotine or total alkaloid (e.g., in or targeting one or more ADC, AO, or ODC genes), and a second genome modification providing a comparable level of one or more traits selected from the group consisting of total leaf polyamine level, total root polyamine level, total leaf chlorophyll level, mesophyll cell number per leaf area unit, and leaf epidermal cell size; and where the control plant does not have both the first and the second genome modifications. In one aspect, a tobacco plant, or part thereof, comprises relative to a control tobacco plant: a first genome modification providing a lower level of nicotine or total alkaloid (e.g., in or targeting one or more ADC, AO, or ODC genes), and a second genome modification providing a comparable level of total leaf polyamine level, where the control plant does not have both the first and the second genome modifications. In an aspect, a tobacco plant, or part thereof, comprises relative to a control tobacco plant: a first genome modification providing a lower level of nicotine or total alkaloid (e.g., in or targeting one or more ADC, AO, or ODC genes), and a second genome modification providing a comparable level of total root polyamine level, where the control plant does not have both the first and the second genome modifications. In one aspect, a tobacco plant, or part thereof, comprises relative to a control tobacco plant: a first genome modification providing a lower level of nicotine or total alkaloid (e.g., in or targeting one or more ADC, AO, or ODC genes), and a second genome modification providing a comparable level of total leaf chlorophyll level, where the control plant does not have both the first and the second genome modifications. In an aspect, a tobacco plant, or part thereof, comprises relative to a control tobacco plant: a first genome modification providing a lower level of nicotine or total alkaloid (e.g., in or targeting one or more ADC, AO, or ODC genes), and a second genome modification providing a comparable level of mesophyll cell number per leaf area unit, where the control plant does not have both the first and the second genome modifications. In one aspect, a tobacco plant, or part thereof, comprises relative to a control tobacco plant: a first genome modification providing a lower level of nicotine or total alkaloid (e.g., in or targeting one or more ADC, AO, or ODC genes), and a second genome modification providing a comparable level of leaf epidermal cell size, where the control plant does not have both the first and the second genome modifications. In an aspect, a second genome modification is in or targeting an ADC, AO, or ODC gene.

In an aspect, a first genome modification, a second genome modification, or both comprise a transgene, a mutation, or both. In one aspect, a genome modification, a second genome modification, or both comprise a transgene. In an aspect, a first genome modification, a second genome modification, or both comprise a mutation. In one aspect, a first genome modification, a second genome modification, or both are not transgene-based. In an aspect, a first genome modification, a second genome modification, or both are not mutation-based.

In an aspect, tobacco plants provided herein comprise a reduced amount of total conjugated polyamines in leaves relative to the control tobacco plant. In one aspect, tobacco plants provided herein comprise a reduced amount of total conjugated polyamines in roots relative to the control tobacco plant. Used here, conjugated polyamines include, but are not limited to, soluble conjugated polyamines such as phenolamides containing a backbone consisting of a free polyamine (e.g., putrescine, spermine, and/or spermidine) conjugated with one or more phenylpropanoids such as ferulic, caffeic and courmaric acids. Conjugated polyamines also include, but are not limited to, insoluble conjugated polyamines incorporated into structural polymers such as lignin. In an aspect, tobacco plants provided herein comprise a reduced amount of total free polyamines (e.g., putrescine, spermine, and spermidine) in leaves relative to the control tobacco plant. In one aspect, tobacco plants provided herein comprise a reduced amount of total conjugated polyamines in roots relative to the control tobacco plant. In an aspect, tobacco plants provided herein comprise a reduced amount of total conjugated form of one or more polyamines selected from the group consisting of putrescine, spermidine and spermine in leaves relative to the control tobacco plant. In one aspect, tobacco plants provided herein comprise a reduced amount of total conjugated form of one or more polyamines selected from the group consisting of putrescine, spermidine and spermine in roots relative to the control tobacco plant. In an aspect, tobacco plants provided herein comprise a reduced amount of total free form of one or more polyamines selected from the group consisting of putrescine, spermidine and spermine in leaves relative to the control tobacco plant. In one aspect, tobacco plants provided herein comprise a reduced amount of total conjugated form of one or more polyamines selected from the group consisting of putrescine, spermidine and spermine in roots relative to the control tobacco plant.

In an aspect, a characteristic or a trait of a tobacco plant described here are measured at a time selected from the group consisting of immediately before flowering, at topping, 1 week-post-topping (WPT), 2 WPT, 3 WPT, 4 WPT, 5 WPT, 6 WPT, 7 WPT, 8 WPT, and at harvest. In one aspect, tobacco plants provided herein comprising a first and a second genome modification are capable of producing a leaf with a leaf grade comparable to that of a leaf from a control plant. In an aspect, tobacco plants provided herein comprising a first and a second genome modification have a total leaf yield comparable to a control plant.

In one aspect, a tobacco plant of the present disclosure comprises a nic1 mutation, a nic2 mutation, or both.

In an aspect, a modified tobacco plant provided herein further comprises a transgene or mutation directly suppressing the expression or activity of one or more, two or more, three or more, four or more, five or more, six or more, seven or more, eight or more, nine or more, ten or more, eleven or more, twelve or more, thirteen or more, fourteen or more, fifteen or more, sixteen or more, or seventeen or more genes or loci encoding a protein selected from the group consisting of agmatine deiminase (AIC), arginase, diamine oxidase, methylputrescine oxidase (MPO), NADH dehydrogenase, phosphoribosylanthranilate isomerase (PRAI), putrescine N-methyltransferase (PMT), quinolate phosphoribosyl transferase (QPT), S-adenosyl-methionine synthetase (SAMS), A622, NBB1, berberine bridge enzyme-like (BBL), MYC2, Nic1_ERF, Nic2_ERF, ethylene response factor (ERF) transcription factor, nicotine uptake permease (NUP), and MATE transporter. See Dewey and Xie, Molecular genetics of alkaloid biosynthesis in Nicotiana tabacum, Phytochemistry 94 (2013) 10-27.

In an aspect, a modified tobacco plant provided herein further comprises a mutation in an ERF gene of Nic2 locus (Nic2_ERF). In an aspect, a modified tobacco plant provided herein further comprises one or more mutations in one or more, two or more, three or more, four or more, five or more, six or more, seven or more, eight or more, nine or more, or all ten genes selected from the group consisting of ERF32, ERF34, ERF39, ERF189, ERF115, ERF221, ERF104, ERF179, ERF17, and ERF168. See Shoji et al., Plant Cell, (10):3390-409 (2010); and Kajikawa et al., Plant physiol. 2017, 174:999-1011. In one aspect, a modified tobacco plant provided herein further comprises one or more mutations in ERF189, ERF115, or both. In an aspect, a modified tobacco plant provided herein further comprises one or more transgenes targeting and suppressing a gene encoding one or more, two or more, three or more, four or more, five or more, six or more, seven or more, eight or more, nine or more, or all ten proteins selected from the group consisting of ERF32, ERF34, ERF39, ERF189, ERF115, ERF221, ERF104, ERF179, ERF17, and ERF168.

In an aspect, a modified tobacco plant provided herein further comprises a mutation in an ERF gene of Nic1 locus (Nic1_ERF) (or Nic1b locus as in WO/2019/140297). See also WO/2018/237107. In an aspect, a modified tobacco plant provided herein further comprises one or more mutations in two or more, three or more, four or more, five or more, six or more, or seven or more genes selected from the group consisting of ERF101, ERF110, ERFnew, ERF199, ERF19, ERF130, ERF16, ERF29, ERF210, and ERF91L2. See WO/2019/140297 and Kajikawa et al., Plant physiol. 2017, 174:999-1011. In an aspect, a modified tobacco plant provided herein further comprises one or more mutations in one or more, two or more, three or more, four or more, five or more, or all six genes selected from the group consisting of ERFnew, ERF199, ERF19, ERF29, ERF210, and ERF91L2. In an aspect, a modified tobacco plant provided herein further comprises one or more transgenes targeting and suppressing a gene encoding one or more, two or more, three or more, four or more, five or more, six or more, or seven or more genes selected from the group consisting of ERF101, ERF110, ERFnew, ERF199, ERF19, ERF130, ERF16, ERF29, ERF210, and ERF91L2.

In an aspect, a modified tobacco plant provided herein further comprise a first genetic modification comprising a mutation in a gene or locus encoding a protein selected from the group consisting of aspartate oxidase, agmatine deiminase (AIC), arginase, diamine oxidase, arginine decarboxylase (ADC), methylputrescine oxidase (MPO), NADH dehydrogenase, ornithine decarboxylase (ODC), phosphoribosylanthranilate isomerase (PRAI), putrescine N-methyltransferase (PMT), quinolate phosphoribosyl transferase (QPT), and S-adenosyl-methionine synthetase (SAMS), A622, NBB1, BBL, MYC2, Nic1_ERF, Nic2_ERF, ethylene response factor (ERF) transcription factor, nicotine uptake permease (NUP), and MATE transporter, and further comprises a second genetic modification targeting one or more amino acid sequences at least 80% identical or similar to an amino acid sequence selected from the group consisting of SEQ ID NOs: 117-174. In one aspect, a modified tobacco plant provided herein comprises a first genetic modification comprises a transgene targeting and suppressing a gene or locus encoding a protein selected from the group consisting of aspartate oxidase, agmatine deiminase (AIC), arginase, diamine oxidase, arginine decarboxylase (ADC), methylputrescine oxidase (MPO), NADH dehydrogenase, ornithine decarboxylase (ODC), phosphoribosylanthranilate isomerase (PRAI), putrescine N-methyltransferase (PMT), quinolate phosphoribosyl transferase (QPT), and S-adenosyl-methionine synthetase (SAMS), A622, NBB1, BBL, MYC2, Nic1, Nic2, ethylene response factor (ERF) transcription factor, nicotine uptake permease (NUP), and MATE transporter, and further comprises a second genetic modification targeting one or more amino acid sequences at least 80% identical or similar to an amino acid sequence selected from the group consisting of SEQ ID NOs: 117-174.

### Cured Tobacco/Tobacco Products

In an aspect, this disclosure provides a method comprising preparing a tobacco product using cured tobacco material from a modified tobacco plant, where the modified tobacco plant comprises a non-natural mutation in an endogenous nucleic acid sequence encoding a polypeptide comprising an amino acid sequence at least 80% identical or similar to an amino acid sequence selected from the group consisting of SEQ ID NOs: 117-174. In another aspect, this disclosure provides a method comprising preparing a tobacco product using cured tobacco material from a modified tobacco plant, where the modified tobacco plant comprises a non-natural mutation in an endogenous nucleic acid sequence at least 80% identical to a nucleic acid sequence selected from the group consisting of SEQ ID NOs: 1-58. In another aspect, this disclosure provides a method comprising preparing a tobacco product using cured tobacco material from a modified tobacco plant, where the modified tobacco plant comprises a non-natural mutation in an endogenous nucleic acid sequence at least 80% identical to a nucleic acid sequence selected from the group consisting of SEQ ID NOs: 59-116.

In an aspect, this disclosure provides a method comprising preparing a tobacco product using cured tobacco material from a modified tobacco plant, where the modified tobacco plant comprises a recombinant DNA construct, where the recombinant DNA construct comprises a heterologous promoter operably linked to a nucleic acid sequence encoding at least one small RNA molecule capable of binding to and reducing the expression of an endogenous nucleic acid sequence encoding a polypeptide at least 80% identical or similar to an amino acid sequence selected from the group consisting of SEQ ID NOs: 117-174. In another aspect, this disclosure provides a method comprising preparing a tobacco product using cured tobacco material from a modified tobacco plant, where the modified tobacco plant comprises a recombinant DNA construct, and where the recombinant DNA construct comprises a heterologous promoter operably linked to a nucleic acid sequence encoding at least one small RNA molecule capable of binding to and reducing the expression of an endogenous nucleic acid sequence at least 80% identical to a nucleic acid sequence selected from the group consisting of SEQ ID NOs: 1-58. In another aspect, this disclosure provides a method comprising preparing a tobacco product using cured tobacco material from a modified tobacco plant, where the modified tobacco plant comprises a recombinant DNA construct, and where the recombinant DNA construct comprises a heterologous promoter operably linked to a nucleic acid sequence encoding at least one small RNA molecule capable of binding to and reducing the expression of an endogenous nucleic acid sequence at least 80% identical to a nucleic acid sequence selected from the group consisting of SEQ ID NOs: 59-116.

In an aspect, this disclosure provides a method comprising preparing a tobacco plant using cured tobacco material from a modified tobacco plant, where the modified tobacco plant comprises a recombinant DNA construct, and where the recombinant DNA construct comprises a heterologous promoter operably linked to a nucleic acid sequence encoding a polypeptide comprising an amino acid sequence at least 80% identical or similar to an amino acid sequence selected from the group consisting of SEQ ID NOs: 117-174. In another aspect, this disclosure provides a method comprising preparing a tobacco plant using cured tobacco material from a modified tobacco plant, where the modified tobacco plant comprises a recombinant DNA construct, and where the recombinant DNA construct comprises a heterologous promoter operably linked to a nucleic acid sequence at least 80% identical to a nucleic acid sequence selected from the group consisting of SEQ ID NOs: 1-58. In another aspect, this disclosure provides a method comprising preparing a tobacco plant using cured tobacco material from a modified tobacco plant, where the modified tobacco plant comprises a recombinant DNA construct, and where the recombinant DNA construct comprises a heterologous promoter operably linked to a nucleic acid sequence at least 80% identical to a nucleic acid sequence selected from the group consisting of SEQ ID NOs: 59-116.

“Curing” is the aging process that reduces moisture and brings about the destruction of chlorophyll giving tobacco leaves a golden color and by which starch is converted to sugar. Cured tobacco therefore has a higher reducing sugar content and a lower starch content compared to harvested green leaf. In one aspect, tobacco plants or plant components provided herein can be cured using conventional means, e.g., flue-cured, barn-cured, fire-cured, air-cured or sun-cured. See, for example, Tso (1999, Chapter 1 in Tobacco, Production, Chemistry and Technology, Davis & Nielsen, eds., Blackwell Publishing, Oxford) for a description of different types of curing methods. Cured tobacco is usually aged in a wooden drum (e.g., a hogshead) or cardboard cartons in compressed conditions for several years (e.g., two to five years), at a moisture content ranging from 10% to about 25%. See, U.S. Pat. Nos. 4,516,590 and 5,372,149. Cured and aged tobacco then can be further processed. Further processing includes conditioning the tobacco under vacuum with or without the introduction of steam at various temperatures, pasteurization, and fermentation.

Information regarding the harvesting of burley and dark tobacco varieties can be found in the 2019-2020 Burley and Dark Tobacco Production Guide (December 2018) published by the University of Kentucky, The University of Tennessee, Virginia Polytechnic Institute and State University, and North Carolina State University, which is incorporated herein by reference in its entirety.

In an aspect, this disclosure provides cured tobacco material from any tobacco plant, or part thereof, provided herein. In an aspect, this disclosure provides cured tobacco material from any modified tobacco plant, or part thereof, provided herein.

In an aspect, cured tobacco material comprises tobacco material selected from the group selected from cured leaf material, cured stem material, cured bud material, cured flower material, and cured root material. In another aspect, cured tobacco material comprises cured leaf material, cured stem material, or both. In a further aspect, cured tobacco material comprises cured leaf material. In yet another aspect, cured tobacco material comprises cured stem material.

In an aspect, cured tobacco material comprises flue-cured tobacco material. In another aspect, cured tobacco material comprises air-cured tobacco material. In another aspect, cured tobacco material comprises fire-cured tobacco material. In another aspect, cured tobacco material comprises sun-cured tobacco material. In another aspect, cured tobacco material provided herein is selected from the group consisting of air-cured tobacco material, fire-cured tobacco material, sun-cured tobacco material, and flue-cured tobacco material. In another aspect, cured tobacco material is from a tobacco variety selected from the group consisting of a flue-cured variety, a bright variety, a Burley variety, a Virginia variety, a Maryland variety, a dark variety, an Oriental variety, and a Turkish variety.

In an aspect, cured tobacco leaf provided herein is selected from the group consisting of air-cured tobacco leaf, fire-cured tobacco leaf, sun-cured tobacco leaf, and flue-cured tobacco leaf. In an aspect, cured tobacco leaf is from a tobacco variety selected from the group consisting of a flue-cured variety, a bright variety, a Burley variety, a Virginia variety, a Maryland variety, a dark variety, an Oriental variety, and a Turkish variety.

Fermentation typically is characterized by high initial moisture content, heat generation, and a 10 to 20% loss of dry weight. See, for example, U.S. Pat. Nos. 4,528,993, 4,660,577, 4,848,373, 5,372,149; U.S. Publication No. 2005/0178398; and Tso (1999, Chapter 1 in Tobacco, Production, Chemistry and Technology, Davis & Nielsen, eds., Blackwell Publishing, Oxford). Cured, aged, and fermented tobacco can be further processed (e.g., cut, shredded, expanded, or blended). See, for example, U.S. Pat. Nos. 4,528,993; 4,660,577; and 4,987,907. In an aspect, this disclosure provides fermented tobacco material from any tobacco plant, or part thereof, provided herein. In another aspect, this disclosure provides fermented tobacco material from any modified tobacco plant, or part thereof, provided herein.

Tobacco material obtained from the tobacco lines, varieties or hybrids of the present disclosure can be used to make tobacco products. As used herein, “tobacco product” is defined as any product made or derived from tobacco that is intended for human use or consumption. In an aspect, this disclosure provides a tobacco product comprising plant material from a tobacco plant provided herein. In another aspect, this disclosure provides a tobacco product comprising plant material from a modified tobacco plant provided herein. In another aspect, this disclosure provides a tobacco product comprising cured tobacco material. In another aspect, this disclosure provides a tobacco product comprising fermented tobacco material. In another aspect, this disclosure provides a tobacco product comprising a tobacco blend.

Tobacco products include, without limitation, cigarette products (e.g., cigarettes and bidi cigarettes), cigar products (e.g., cigar wrapping tobacco and cigarillos), pipe tobacco products, products derived from tobacco, tobacco-derived nicotine products, oral nicotine products, smokeless tobacco products (e.g., moist snuff, dry snuff, and chewing tobacco), films, chewables, tabs, shaped parts, gels, consumable units, insoluble matrices, hollow shapes, reconstituted tobacco, expanded tobacco, and the like. See, e.g., U.S. Patent Publication No. US 2006/0191548.

As used herein, “cigarette” refers a tobacco product having a “rod” and “filler”. The cigarette “rod” includes the cigarette paper, filter, plug wrap (used to contain filtration materials), tipping paper that holds the cigarette paper (including the filler) to the filter, and all glues that hold these components together. The “filler” includes (1) all tobaccos, including but not limited to reconstituted and expanded tobacco, (2) non-tobacco substitutes (including but not limited to herbs, non-tobacco plant materials and other spices that may accompany tobaccos rolled within the cigarette paper), (3) casings, (4) flavorings, and (5) all other additives (that are mixed into tobaccos and substitutes and rolled into the cigarette).

In an aspect, a tobacco product comprises reconstituted tobacco. In another aspect, this disclosure provides reconstituted tobacco comprising cured tobacco material. As used herein, “reconstituted tobacco” refers to a part of tobacco filler made from tobacco dust and other tobacco scrap material, processed into sheet form and cut into strips to resemble tobacco. In addition to the cost savings, reconstituted tobacco is very important for its contribution to cigarette taste from processing flavor development using reactions between ammonia and sugars.

In an aspect, a tobacco product comprises expanded tobacco. As used herein, “expanded tobacco” refers to a part of tobacco filler which is processed through expansion of suitable gases so that the tobacco is “puffed” resulting in reduced density and greater filling capacity. It reduces the weight of tobacco used in cigarettes.

Tobacco products derived from plants of the present disclosure also include cigarettes and other smoking articles, particularly those smoking articles including filter elements, where the rod of smokable material includes cured tobacco within a tobacco blend. In an aspect, a tobacco product of the present disclosure is selected from the group consisting of a cigarillo, a non-ventilated recess filter cigarette, a vented recess filter cigarette, a cigar, snuff, pipe tobacco, cigar tobacco, cigarette tobacco, chewing tobacco, leaf tobacco, hookah tobacco, shredded tobacco, and cut tobacco. In another aspect, a tobacco product of the present disclosure is selected from the group consisting of a cigarette, a heated tobacco product, a kretek, a bidi cigarette, a cigar, a cigarillo, a non-ventilated cigarette, a vented recess filter cigarette, pipe tobacco, snuff, snus, chewing tobacco, moist smokeless tobacco, fine cut chewing tobacco, long cut chewing tobacco, pouched chewing tobacco product, gum, a tablet, a lozenge, and a dissolving strip.

In another aspect, a tobacco product of the present disclosure is a smokeless tobacco product. In an aspect, a smokeless tobacco product is selected from the group consisting of loose leaf chewing tobacco, plug chewing tobacco, moist snuff, nasal snuff, dry snuff, and snus.

Smokeless tobacco products are not combusted and include, but not limited to, chewing tobacco, moist smokeless tobacco, snus, and dry snuff. Chewing tobacco is coarsely divided tobacco leaf that is typically packaged in a large pouch-like package and used in a plug or twist. Moist smokeless tobacco is a moist, more finely divided tobacco that is provided in loose form or in pouch form and is typically packaged in round cans and used as a pinch or in a pouch placed between an adult tobacco consumer's cheek and gum. Snus is a heat-treated smokeless tobacco. Dry snuff is finely ground tobacco that is placed in the mouth or used nasally.

In yet another aspect, a tobacco product of the present disclosure is selected from the group consisting of an electronically heated cigarette, an e-cigarette, an electronic vaporing device, and an inhalable nicotine product.

In an aspect, a tobacco product of the present disclosure can be a blended tobacco product.

In another aspect, this disclosure provides a tobacco blend comprising cured tobacco material. A tobacco blend can comprise any combination of cured tobacco, uncured tobacco, fermented tobacco, unfermented tobacco, expanded tobacco, and reconstituted tobacco.

In an aspect, a tobacco blend comprises at least 5% cured tobacco by weight. In an aspect, a tobacco blend comprises at least 10% cured tobacco by weight. In an aspect, a tobacco blend comprises at least 15% cured tobacco by weight. In an aspect, a tobacco blend comprises at least 20% cured tobacco by weight. In an aspect, a tobacco blend comprises at least 25% cured tobacco by weight. In an aspect, a tobacco blend comprises at least 30% cured tobacco by weight. In an aspect, a tobacco blend comprises at least 35% cured tobacco by weight. In an aspect, a tobacco blend comprises at least 40% cured tobacco by weight. In an aspect, a tobacco blend comprises at least 45% cured tobacco by weight. In an aspect, a tobacco blend comprises at least 50% cured tobacco by weight. In an aspect, a tobacco blend comprises at least 55% cured tobacco by weight. In an aspect, a tobacco blend comprises at least 60% cured tobacco by weight. In an aspect, a tobacco blend comprises at least 65% cured tobacco by weight. In an aspect, a tobacco blend comprises at least 70% cured tobacco by weight. In an aspect, a tobacco blend comprises at least 75% cured tobacco by weight. In an aspect, a tobacco blend comprises at least 80% cured tobacco by weight. In an aspect, a tobacco blend comprises at least 85% cured tobacco by weight. In an aspect, a tobacco blend comprises at least 90% cured tobacco by weight. In an aspect, a tobacco blend comprises at least 95% cured tobacco by weight.

In an aspect, a tobacco blend comprises at least 5% cured tobacco by volume. In an aspect, a tobacco blend comprises at least 10% cured tobacco by volume. In an aspect, a tobacco blend comprises at least 15% cured tobacco by volume. In an aspect, a tobacco blend comprises at least 20% cured tobacco by volume. In an aspect, a tobacco blend comprises at least 25% cured tobacco by volume. In an aspect, a tobacco blend comprises at least 30% cured tobacco by volume. In an aspect, a tobacco blend comprises at least 35% cured tobacco by volume. In an aspect, a tobacco blend comprises at least 40% cured tobacco by volume. In an aspect, a tobacco blend comprises at least 45% cured tobacco by volume. In an aspect, a tobacco blend comprises at least 50% cured tobacco by volume. In an aspect, a tobacco blend comprises at least 55% cured tobacco by volume. In an aspect, a tobacco blend comprises at least 60% cured tobacco by volume. In an aspect, a tobacco blend comprises at least 65% cured tobacco by volume. In an aspect, a tobacco blend comprises at least 70% cured tobacco by volume. In an aspect, a tobacco blend comprises at least 75% cured tobacco by volume. In an aspect, a tobacco blend comprises at least 80% cured tobacco by volume. In an aspect, a tobacco blend comprises at least 85% cured tobacco by volume. In an aspect, a tobacco blend comprises at least 90% cured tobacco by volume. In an aspect, a tobacco blend comprises at least 95% cured tobacco by volume.

### Transformation

In an aspect, this disclosure provides a method of producing a modified tobacco plant comprising: (a) inducing a non-natural mutation in at least one tobacco cell in an endogenous nucleic acid sequence encoding a polypeptide comprising an amino acid sequence at least 80% identical or similar to an amino acid sequence selected from the group consisting of SEQ ID NOs: 117-174; (b) selecting at least one tobacco cell comprising the non-natural mutation from step (a); and (c) regenerating at least one modified tobacco plant from the at least one tobacco cell selected in step (b). In another aspect, this disclosure provides a method of producing a modified tobacco plant comprising: (a) inducing a non-natural mutation in at least one tobacco cell in an endogenous nucleic acid sequence at least 80% identical to a nucleic acid sequence selected from the group consisting of SEQ ID NOs: 1-58; (b) selecting at least one tobacco cell comprising the non-natural mutation from step (a); and (c) regenerating at least one modified tobacco plant from the at least one tobacco cell selected in step (b). In another aspect, this disclosure provides a method of producing a modified tobacco plant comprising: (a) inducing a non-natural mutation in at least one tobacco cell in an endogenous nucleic acid sequence at least 80% identical to a nucleic acid sequence selected from the group consisting of SEQ ID NOs: 59-116; (b) selecting at least one tobacco cell comprising the non-natural mutation from step (a); and (c) regenerating at least one modified tobacco plant from the at least one tobacco cell selected in step (b). In another aspect, this disclosure provides a method of producing a modified tobacco plant comprising: (a) inducing a non-natural mutation in at least one tobacco cell in an endogenous nucleic acid sequence selected from the group consisting of SEQ ID NOs: 1-116; (b) selecting at least one tobacco cell comprising the non-natural mutation from step (a); and (c) regenerating at least one modified tobacco plant from the at least one tobacco cell selected in step (b). In an aspect, any of the foregoing methods further comprises (d) growing the modified tobacco plant regenerated in step (c). In another aspect, any of the foregoing methods further comprises: (e) crossing the modified tobacco plant grown in step (d) with a second tobacco plant; and (f) obtaining at least one seed from the crossing in step (e).

In an aspect, this disclosure provides a method of producing a modified tobacco plant comprising: (a) introducing a recombinant DNA construct to at least one tobacco cell, where the recombinant DNA construct comprises a heterologous promoter operably linked to a nucleic acid encoding at least one small RNA molecule capable of binding to and reducing the expression of an endogenous nucleic acid sequence encoding a polypeptide at least 80% identical or similar to an amino acid sequence selected from the group consisting of SEQ ID NOs: 117-174; (b) selecting at least one tobacco cell comprising the recombinant DNA construct; and (c) regenerating at least one modified tobacco plant from the at least one tobacco cell selected in step (b). In another aspect, this disclosure provides a method of producing a modified tobacco plant comprising: (a) introducing a recombinant DNA construct to at least one tobacco cell, where the recombinant DNA construct comprises a heterologous promoter operably linked to a nucleic acid encoding at least one small RNA molecule capable of binding to and reducing the expression of an endogenous nucleic acid sequence at least 80% identical to a nucleic acid sequence selected from the group consisting of SEQ ID NOs: 1-58; (b) selecting at least one tobacco cell comprising the recombinant DNA construct; and (c) regenerating at least one modified tobacco plant from the at least one tobacco cell selected in step (b). In another aspect, this disclosure provides a method of producing a modified tobacco plant comprising: (a) introducing a recombinant DNA construct to at least one tobacco cell, where the recombinant DNA construct comprises a heterologous promoter operably linked to a nucleic acid encoding at least one small RNA molecule capable of binding to and reducing the expression of an endogenous nucleic acid sequence at least 80% identical to a nucleic acid sequence selected from the group consisting of SEQ ID NOs: 59-116; (b) selecting at least one tobacco cell comprising the recombinant DNA construct; and (c) regenerating at least one modified tobacco plant from the at least one tobacco cell selected in step (b). In an aspect, any of the foregoing methods further comprises (d) growing the modified tobacco plant regenerated in step (c). In another aspect, any of the foregoing methods further comprises: (e) crossing the modified tobacco plant grown in step (d) with a second tobacco plant; and (f) obtaining at least one seed from the crossing in step (e).

In an aspect, this disclosure provides a method of producing a modified tobacco plant comprising: (a) introducing a recombinant DNA construct to at least one tobacco cell, where the recombinant DNA construct comprises a heterologous promoter operably linked to a nucleic acid sequence encoding a polypeptide comprising an amino acid sequence at least 80% identical or similar to an amino acid sequence selected from the group consisting of SEQ ID NOs: 117-174; (b) selecting at least one tobacco cell comprising the recombinant DNA construct; and (c) regenerating at least one modified tobacco plant from the at least one tobacco cell selected in step (b). In another aspect, this disclosure provides a method of producing a modified tobacco plant comprising: (a) introducing a recombinant DNA construct to at least one tobacco cell, where the recombinant DNA construct comprises a heterologous promoter operably linked to a nucleic acid sequence at least 80% identical to a nucleic acid sequence selected from the group consisting of SEQ ID NOs: 1-58; (b) selecting at least one tobacco cell comprising the recombinant DNA construct; and (c) regenerating at least one modified tobacco plant from the at least one tobacco cell selected in step (b). In another aspect, this disclosure provides a method of producing a modified tobacco plant comprising: (a) introducing a recombinant DNA construct to at least one tobacco cell, where the recombinant DNA construct comprises a heterologous promoter operably linked to a nucleic acid sequence at least 80% identical to a nucleic acid sequence selected from the group consisting of SEQ ID NOs: 59-116; (b) selecting at least one tobacco cell comprising the recombinant DNA construct; and (c) regenerating at least one modified tobacco plant from the at least one tobacco cell selected in step (b). In an aspect, any of the foregoing methods further comprises (d) growing the modified tobacco plant regenerated in step (c). In another aspect, any of the foregoing methods further comprises: (e) crossing the modified tobacco plant grown in step (d) with a second tobacco plant; and (f) obtaining at least one seed from the crossing in step (e).

In an aspect, this disclosure provides a method comprising transforming a tobacco cell with a recombinant DNA construct, where the recombinant DNA construct comprises a heterologous promoter operably linked to a nucleic acid sequence encoding at least one small RNA molecule capable of binding to and reducing the expression of an endogenous nucleic acid sequence encoding a polypeptide at least 80% identical or similar to an amino acid sequence selected from the group consisting of SEQ ID NOs: 117-174. In another aspect, this disclosure provides a method comprising transforming a tobacco cell with a recombinant DNA construct, where the recombinant DNA construct comprises a heterologous promoter operably linked to a nucleic acid sequence encoding at least one small RNA molecule capable of binding to and reducing the expression of an endogenous nucleic acid sequence at least 80% identical a nucleic acid sequence selected from the group consisting of SEQ ID NOs: 1-58. In another aspect, this disclosure provides a method comprising transforming a tobacco cell with a recombinant DNA construct, where the recombinant DNA construct comprises a heterologous promoter operably linked to a nucleic acid sequence encoding at least one small RNA molecule capable of binding to and reducing the expression of an endogenous nucleic acid sequence at least 80% identical a nucleic acid sequence selected from the group consisting of SEQ ID NOs: 59-116. In another aspect, the foregoing methods further comprise regenerating a modified tobacco plant from the transformed tobacco cell.

In an aspect, this disclosure provides a method comprising transforming a tobacco cell with a recombinant DNA construct, where the recombinant DNA construct comprises a heterologous promoter operably linked to a nucleic acid sequence encoding a polypeptide comprising an amino acid sequence at least 80% identical or similar to an amino acid sequence selected from the group consisting of SEQ ID NOs: 117-174. In another aspect, this disclosure provides a method comprising transforming a tobacco cell with a recombinant DNA construct, where the recombinant DNA construct comprises a heterologous promoter operably linked to a nucleic acid sequence at least 80% identical to a nucleic acid sequence selected from the group consisting of SEQ ID NOs: 1-58. In another aspect, this disclosure provides a method comprising transforming a tobacco cell with a recombinant DNA construct, where the recombinant DNA construct comprises a heterologous promoter operably linked to a nucleic acid sequence at least 80% identical to a nucleic acid sequence selected from the group consisting of SEQ ID NOs: 59-116. In another aspect, the foregoing methods further comprise regenerating a modified tobacco plant from the transformed tobacco cell.

Numerous methods for introducing a recombinant DNA construct to a plant cell are known in the art, which can be used according to methods of the present application to produce a transgenic plant cell and plant. Any suitable method or technique for transformation of a plant cell known in the art can be used according to present methods. Effective methods for transformation of plants include bacterially mediated transformation, such as Agrobacterium-mediated or Rhizobium-mediated transformation and microprojectile bombardment-mediated transformation. A variety of methods are known in the art for transforming explants with a transformation vector via bacterially mediated transformation or microprojectile bombardment and then subsequently culturing, etc., those explants to regenerate or develop transgenic plants. Other methods for plant transformation, such as microinjection, electroporation, vacuum infiltration, pressure, sonication, silicon carbide fiber agitation, polyethylene glycol (PEG)-mediated transformation, etc., are also known in the art. Transgenic plants produced by these transformation methods can be chimeric or non-chimeric for the transformation event depending on the methods and explants used.

Methods of transforming plant cells are well known by persons of ordinary skill in the art. For instance, specific instructions for transforming plant cells by microprojectile bombardment with particles coated with recombinant DNA (e.g., biolistic transformation) are found in U.S. Pat. Nos. 5,550,318; 5,538,880 6,160,208; 6,399,861; and 6,153,812 and Agrobacterium-mediated transformation is described in U.S. Pat. Nos. 5,159,135; 5,824,877; 5,591,616; 6,384,301; 5,750,871; 5,463,174; and 5,188,958, all of which are incorporated herein by reference. Additional methods for transforming plants can be found in, for example, Compendium of Transgenic Crop Plants (2009) Blackwell Publishing. Any appropriate method known to those skilled in the art can be used to transform a tobacco cell with any of the nucleic acid molecules provided herein.

In an aspect, a method of introducing a nucleic acid molecule to a tobacco cell comprises Agrobacterium-mediated transformation. In another aspect, a method of introducing a nucleic acid molecule to a cell comprises PEG-mediated transformation. In another aspect, a method of introducing a nucleic acid molecule to a cell comprises biolistic transformation. In another aspect, a method of introducing a nucleic acid molecule to a cell comprises liposome-mediated transfection (lipofection). In another aspect, a method of introducing a nucleic acid molecule to a cell comprises lentiviral transfection.

Lipofection is described in e.g., U.S. Pat. Nos. 5,049,386, 4,946,787; and 4,897,355) and lipofection reagents are sold commercially (e.g., Transfectam™ and Lipofectin™) Cationic and neutral lipids that are suitable for efficient receptor-recognition lipofection of polynucleotides include those of WO 91/17424 and WO 91/16024. Delivery can be to cells (e.g. in vitro or ex vivo administration) or target tissues (e.g. in vivo administration).

Any tobacco cell from which a fertile tobacco plant can be regenerated is contemplated as a useful recipient cell for practice of this disclosure. In an aspect, a recombinant DNA construct is introduced to a tobacco cell. In an aspect, a recombinant DNA construct is introduced to a tobacco protoplast cell. In another aspect, a recombinant DNA construct is introduced to a tobacco callus cell. In an aspect, a recombinant DNA construct is introduced to a tobacco cell selected from the group consisting of a seed cell, a fruit cell, a leaf cell, a cotyledon cell, a hypocotyl cell, a meristem cell, an embryo cell, an endosperm cell, a root cell, a shoot cell, a stem cell, a flower cell, an inflorescence cell, a stalk cell, a pedicel cell, a style cell, a stigma cell, a receptacle cell, a petal cell, a sepal cell, a pollen cell, an anther cell, a filament cell, an ovary cell, an ovule cell, a pericarp cell, and a phloem cell.

Callus can be initiated from various tissue sources, including, but not limited to, immature embryos or parts of embryos, seedling apical meristems, microspores, and the like. Those cells which are capable of proliferating as callus can serve as recipient cells for transformation. Practical transformation methods and materials for making transgenic plants of this disclosure (e.g., various media and recipient target cells, transformation of immature embryos, and subsequent regeneration of fertile transgenic plants) are disclosed, for example, in U.S. Pat. Nos. 6,194,636 and 6,232,526 and U. S. Patent Application Publication 2004/0216189, all of which are incorporated herein by reference.

### Leaf Grade

As used herein, “USDA leaf grade index” refers to a subdivision of a leaf type according to group, quality, and color. In one aspect, a USDA grade quality score is quantified as a 0-100 numerical representation of the grade as determined by a certified tobacco leaf grader, and is a weighted average of all stalk positions. A higher grade index indicates higher quality. As used below, a “point” refers to each whole number numerical representation of the USDA leaf grade score. For example, the difference between a USDA leaf grade index score of 90 and a score of 85 is 5 points.

Alternatively, leaf grade can be determined via hyper-spectral imaging. See e.g., WO 2011/027315 (published on Mar. 10, 2011, and incorporated by reference in its entirety).

As used herein, a “certified tobacco leaf grader” refers to a person trained to grade tobacco leaves in accordance with USDA Official Standards Grades defined by the United States Department of Agriculture (USDA), Agricultural Marketing Systems as published in 7 CFR § 29. A USDA leaf grade index score may be assigned by an employee, a past employee, or a person otherwise trained to grade tobacco leaves in accordance with USDA Official Standards Grades. Exemplary steps of a standard operation for commercial inspection service begins with a grower delivering tobacco to market after which the tobacco is arranged on flat baskets as lots. Each lot is weighed and then inspected by a certified tobacco leaf grader. After examination, the grader assigns a grade to each lot which becomes a certificate of grade indicating group, quality, and color. The steps for grading experimental lots is similar; however, experimental tobacco is not taken to market or otherwise used for commercial purposes.

Tobacco grades are evaluated based on factors including, but not limited to, the leaf stalk position, leaf size, leaf color, leaf uniformity and integrity, ripeness, texture, elasticity, sheen (related with the intensity and the depth of coloration of the leaf as well as the shine), hygroscopicity (the faculty of the tobacco leaves to absorb and to retain the ambient moisture), and green nuance or cast. Leaf grade can be determined, for example, using an Official Standard Grade published by the Agricultural Marketing Service of the US Department of Agriculture (7 U.S.C. § 511). See, e.g., Official Standard Grades for Burley Tobacco (U.S. Type 31 and Foreign Type 93), effective Nov. 5, 1990 (55 F.R. 40645); Official Standard Grades for Flue-Cured Tobacco (U.S. Types 11, 12, 13, 14 and Foreign Type 92), effective Mar. 27, 1989 (54 F.R. 7925); Official Standard Grades for Pennsylvania Seedleaf Tobacco (U.S. Type 41), effective Jan. 8, 1965 (29 F.R. 16854); Official Standard Grades for Ohio Cigar-Leaf Tobacco (U.S. Types 42, 43, and 44), effective Dec. 8, 1963 (28 F.R. 11719 and 28 F.R. 11926); Official Standard Grades for Wisconsin Cigar-Binder Tobacco (U.S. Types 54 and 55), effective Nov. 20, 1969 (34 F.R. 17061); Official Standard Grades for Wisconsin Cigar-Binder Tobacco (U.S. Types 54 and 55), effective Nov. 20, 1969 (34 F.R. 17061); Official Standard Grades for Georgia and Florida Shade-Grown Cigar-Wrapper Tobacco (U.S. Type 62), Effective April 1971. A USDA grade index value can be determined according to an industry accepted grade index. See, e.g., Bowman et al, Tobacco Science, 32:39-40(1988); Legacy Tobacco Document Library (Bates Document #523267826-523267833, Jul. 1, 1988, Memorandum on the Proposed Burley Tobacco Grade Index); and Miller et al., 1990, Tobacco Intern., 192:55-57 (all foregoing references are incorporated by reference in their entirety).

Unless specified otherwise, measurements of leaf grade index values, alkaloid, or nicotine levels mentioned herein for a tobacco plant, variety, cultivar, or line refer to average measurements, including, for example, an average of multiple leaves of a single plant or an average measurement from a population of tobacco plants from a single variety, cultivar, or line. A population of tobacco plants or a collection of tobacco leaves for determining an average measurement (e.g., leaf grading or alkaloid or nicotine level) can be of any size, for example, 2, 5, 10, 15, 20, 25, 30, 35, 40, 50, or more. A population of at least 5 or more tobacco plants is used to determine standard deviation. Industry-accepted standard protocols are followed for determining average measurements or grade index values.

As used herein, “USDA graded leaf group”, “leaf group”, or “group” is a division of a type covering closely related grades based on certain characteristics which are related to stalk position, body, or general quality. Group is the first factor of a USDA grade. Group determination is part of the grading procedure and is assigned by a certified tobacco leaf grader.

In an aspect, a modified tobacco plant comprising a non-natural mutation comprises a comparable or higher USDA leaf grade index as compared to a control tobacco plant lacking the non-natural mutation when grown under comparable conditions. In another aspect, a modified tobacco plant comprising a recombinant DNA construct comprises a comparable or higher USDA leaf grade index as compared to a control tobacco plant lacking the recombinant DNA construct when grown under comparable conditions.

As used herein, a “comparable” USDA leaf grade index refers to within 15%. For example, if a control plant has a USDA leaf grade index of 100, a comparable USDA leaf grade index would be between 85 and 100.

In an aspect, a modified tobacco plant comprises a USDA leaf grade index at least 1% higher than the USDA leaf grade index of a control tobacco plant when grown under comparable conditions. In another aspect, a modified tobacco plant comprises a USDA leaf grade index at least 5% higher than the USDA leaf grade index of a control tobacco plant when grown under comparable conditions. In another aspect, a modified tobacco plant comprises a USDA leaf grade index at least 10% higher than the USDA leaf grade index of a control tobacco plant when grown under comparable conditions. In another aspect, a modified tobacco plant comprises a USDA leaf grade index at least 20% higher than the USDA leaf grade index of a control tobacco plant when grown under comparable conditions. In another aspect, a modified tobacco plant comprises a USDA leaf grade index at least 30% higher than the USDA leaf grade index of a control tobacco plant when grown under comparable conditions. In another aspect, a modified tobacco plant comprises a USDA leaf grade index at least 40% higher than the USDA leaf grade index of a control tobacco plant when grown under comparable conditions. In another aspect, a modified tobacco plant comprises a USDA leaf grade index at least 50% higher than the USDA leaf grade index of a control tobacco plant when grown under comparable conditions. In another aspect, a modified tobacco plant comprises a USDA leaf grade index at least 75% higher than the USDA leaf grade index of a control tobacco plant when grown under comparable conditions. In another aspect, a modified tobacco plant comprises a USDA leaf grade index at least 100% higher than the USDA leaf grade index of a control tobacco plant when grown under comparable conditions.

In an aspect, a modified tobacco plant comprises a USDA leaf grade index between 1% higher and 100% higher than the USDA leaf grade index of a control tobacco plant when grown under comparable conditions. In an aspect, a modified tobacco plant comprises a USDA leaf grade index between 1% higher and 75% higher than the USDA leaf grade index of a control tobacco plant when grown under comparable conditions. In an aspect, a modified tobacco plant comprises a USDA leaf grade index between 1% higher and 50% higher than the USDA leaf grade index of a control tobacco plant when grown under comparable conditions. In an aspect, a modified tobacco plant comprises a USDA leaf grade index between 1% higher and 40% higher than the USDA leaf grade index of a control tobacco plant when grown under comparable conditions. In an aspect, a modified tobacco plant comprises a USDA leaf grade index between 1% higher and 30% higher than the USDA leaf grade index of a control tobacco plant when grown under comparable conditions. In an aspect, a modified tobacco plant comprises a USDA leaf grade index between 1% higher and 20% higher than the USDA leaf grade index of a control tobacco plant when grown under comparable conditions. In an aspect, a modified tobacco plant comprises a USDA leaf grade index between 1% higher and 10% higher than the USDA leaf grade index of a control tobacco plant when grown under comparable conditions. In an aspect, a modified tobacco plant comprises a USDA leaf grade index between 10% higher and 75% higher than the USDA leaf grade index of a control tobacco plant when grown under comparable conditions. In an aspect, a modified tobacco plant comprises a USDA leaf grade index between 10% higher and 50% higher than the USDA leaf grade index of a control tobacco plant when grown under comparable conditions. In an aspect, a modified tobacco plant comprises a USDA leaf grade index between 1% higher and 30% higher than the USDA leaf grade index of a control tobacco plant when grown under comparable conditions.

In an aspect, a modified tobacco plant comprises a USDA leaf grade index at least 1 point higher than the USDA leaf grade index of a control tobacco plant when grown under comparable conditions. In another aspect, a modified tobacco plant comprises a USDA leaf grade index at least 2 points higher than the USDA leaf grade index of a control tobacco plant when grown under comparable conditions. In another aspect, a modified tobacco plant comprises a USDA leaf grade index at least 3 points higher than the USDA leaf grade index of a control tobacco plant when grown under comparable conditions. In another aspect, a modified tobacco plant comprises a USDA leaf grade index at least 4 points higher than the USDA leaf grade index of a control tobacco plant when grown under comparable conditions. In another aspect, a modified tobacco plant comprises a USDA leaf grade index at least 5 points higher than the USDA leaf grade index of a control tobacco plant when grown under comparable conditions. In another aspect, a modified tobacco plant comprises a USDA leaf grade index at least 6 points higher than the USDA leaf grade index of a control tobacco plant when grown under comparable conditions. In another aspect, a modified tobacco plant comprises a USDA leaf grade index at least 7 points higher than the USDA leaf grade index of a control tobacco plant when grown under comparable conditions. In another aspect, a modified tobacco plant comprises a USDA leaf grade index at least 8 points higher than the USDA leaf grade index of a control tobacco plant when grown under comparable conditions. In another aspect, a modified tobacco plant comprises a USDA leaf grade index at least 9 points higher than the USDA leaf grade index of a control tobacco plant when grown under comparable conditions. In another aspect, a modified tobacco plant comprises a USDA leaf grade index at least 10 points higher than the USDA leaf grade index of a control tobacco plant when grown under comparable conditions. In another aspect, a modified tobacco plant comprises a USDA leaf grade index at least 11 points higher than the USDA leaf grade index of a control tobacco plant when grown under comparable conditions. In another aspect, a modified tobacco plant comprises a USDA leaf grade index at least 12 points higher than the USDA leaf grade index of a control tobacco plant when grown under comparable conditions. In another aspect, a modified tobacco plant comprises a USDA leaf grade index at least 13 points higher than the USDA leaf grade index of a control tobacco plant when grown under comparable conditions. In another aspect, a modified tobacco plant comprises a USDA leaf grade index at least 14 points higher than the USDA leaf grade index of a control tobacco plant when grown under comparable conditions. In another aspect, a modified tobacco plant comprises a USDA leaf grade index at least 15 points higher than the USDA leaf grade index of a control tobacco plant when grown under comparable conditions. In another aspect, a modified tobacco plant comprises a USDA leaf grade index at least 16 points higher than the USDA leaf grade index of a control tobacco plant when grown under comparable conditions. In another aspect, a modified tobacco plant comprises a USDA leaf grade index at least 17 points higher than the USDA leaf grade index of a control tobacco plant when grown under comparable conditions. In another aspect, a modified tobacco plant comprises a USDA leaf grade index at least 18 points higher than the USDA leaf grade index of a control tobacco plant when grown under comparable conditions. In another aspect, a modified tobacco plant comprises a USDA leaf grade index at least 19 points higher than the USDA leaf grade index of a control tobacco plant when grown under comparable conditions. In another aspect, a modified tobacco plant comprises a USDA leaf grade index at least 20 points higher than the USDA leaf grade index of a control tobacco plant when grown under comparable conditions. In another aspect, a modified tobacco plant comprises a USDA leaf grade index at least 25 points higher than the USDA leaf grade index of a control tobacco plant when grown under comparable conditions. In another aspect, a modified tobacco plant comprises a USDA leaf grade index at least 30 points higher than the USDA leaf grade index of a control tobacco plant when grown under comparable conditions. In another aspect, a modified tobacco plant comprises a USDA leaf grade index at least 35 points higher than the USDA leaf grade index of a control tobacco plant when grown under comparable conditions. In another aspect, a modified tobacco plant comprises a USDA leaf grade index at least 40 points higher than the USDA leaf grade index of a control tobacco plant when grown under comparable conditions. In another aspect, a modified tobacco plant comprises a USDA leaf grade index at least 50 points higher than the USDA leaf grade index of a control tobacco plant when grown under comparable conditions.

In an aspect, a modified tobacco plant comprises a USDA leaf grade index between 1 point higher and 100 points higher than the USDA leaf grade index of a control tobacco plant when grown under comparable conditions. In another aspect, a modified tobacco plant comprises a USDA leaf grade index between 1 point higher and 75 points higher than the USDA leaf grade index of a control tobacco plant when grown under comparable conditions. In another aspect, a modified tobacco plant comprises a USDA leaf grade index between 1 point higher and 50 points higher than the USDA leaf grade index of a control tobacco plant when grown under comparable conditions. In another aspect, a modified tobacco plant comprises a USDA leaf grade index between 1 point higher and 25 points higher than the USDA leaf grade index of a control tobacco plant when grown under comparable conditions. In another aspect, a modified tobacco plant comprises a USDA leaf grade index between 1 point higher and 10 points higher than the USDA leaf grade index of a control tobacco plant when grown under comparable conditions. In another aspect, a modified tobacco plant comprises a USDA leaf grade index between 1 point higher and 5 points higher than the USDA leaf grade index of a control tobacco plant when grown under comparable conditions. In another aspect, a modified tobacco plant comprises a USDA leaf grade index between 10 points higher and 50 points higher than the USDA leaf grade index of a control tobacco plant when grown under comparable conditions. In another aspect, a modified tobacco plant comprises a USDA leaf grade index between 10 points higher and 25 points higher than the USDA leaf grade index of a control tobacco plant when grown under comparable conditions.

In an aspect, a comparable USDA leaf grade index between a modified tobacco plant and a control tobacco plant grown under comparable conditions is within 1 point. In another aspect, a comparable USDA leaf grade index between a modified tobacco plant and a control tobacco plant grown under comparable conditions is within 2 points. In another aspect, a comparable USDA leaf grade index between a modified tobacco plant and a control tobacco plant grown under comparable conditions is within 3 points. In another aspect, a comparable USDA leaf grade index between a modified tobacco plant and a control tobacco plant grown under comparable conditions is within 4 points. In another aspect, a comparable USDA leaf grade index between a modified tobacco plant and a control tobacco plant grown under comparable conditions is within 5 points. In another aspect, a comparable USDA leaf grade index between a modified tobacco plant and a control tobacco plant grown under comparable conditions is within 6 points. In another aspect, a comparable USDA leaf grade index between a modified tobacco plant and a control tobacco plant grown under comparable conditions is within 7 points. In another aspect, a comparable USDA leaf grade index between a modified tobacco plant and a control tobacco plant grown under comparable conditions is within 8 points. In another aspect, a comparable USDA leaf grade index between a modified tobacco plant and a control tobacco plant grown under comparable conditions is within 9 points. In another aspect, a comparable USDA leaf grade index between a modified tobacco plant and a control tobacco plant grown under comparable conditions is within 10 points. In another aspect, a comparable USDA leaf grade index between a modified tobacco plant and a control tobacco plant grown under comparable conditions is within 11 points. In another aspect, a comparable USDA leaf grade index between a modified tobacco plant and a control tobacco plant grown under comparable conditions is within 12 points. In another aspect, a comparable USDA leaf grade index between a modified tobacco plant and a control tobacco plant grown under comparable conditions is within 13 points. In another aspect, a comparable USDA leaf grade index between a modified tobacco plant and a control tobacco plant grown under comparable conditions is within 14 points. In another aspect, a comparable USDA leaf grade index between a modified tobacco plant and a control tobacco plant grown under comparable conditions is within 15 points.

### Aroma/Flavor

In an aspect, a modified tobacco plant provided herein comprises a similar level of one or more tobacco aroma compounds selected from the group consisting of 3-methylvaleric acid, valeric acid, isovaleric acid, a labdenoid, a cembrenoid, a sugar ester, and a reducing sugar, compared to a control tobacco plant when grown under comparable conditions. As used herein, a “similar” level refers to within 20%.

As used herein, “tobacco aroma compounds” are compounds associated with the flavor and aroma of tobacco smoke. These compounds include, but are not limited to, 3-methylvaleric acid, valeric acid, isovaleric acid, cembrenoid and labdenoid diterpenes, and sugar esters. Concentrations of tobacco aroma compounds can be measured by any known metabolite profiling methods in the art including, without limitation, gas chromatography mass spectrometry (GC-MS), Nuclear Magnetic Resonance Spectroscopy, liquid chromatography-linked mass spectrometry. See The Handbook of Plant Metabolomics, edited by Weckwerth and Kahl, (Wiley-Blackwell) (May 28, 2013).

As used herein, “reducing sugar(s)” are any sugar (monosaccharide or polysaccharide) that has a free or potentially free aldehyde or ketone group. Glucose and fructose act as nicotine buffers in cigarette smoke by reducing smoke pH and effectively reducing the amount of “free” unprotonated nicotine. Reducing sugars balances smoke flavor, for example, by modifying the sensory impact of nicotine and other tobacco alkaloids. An inverse relationship between sugar content and alkaloid content has been reported across tobacco varieties, within the same variety, and within the same plant line caused by planting conditions. Reducing sugar levels can be measured using a segmented-flow colorimetric method developed for analysis of tobacco samples as adapted by Skalar Instrument Co (West Chester, PA) and described by Davis, Tobacco Science 20:139-144 (1976). For example, a sample is dialyzed against a sodium carbonate solution. Copper neocuproin is added to the sample and the solution is heated. The copper neocuproin chelate is reduced in the presence of sugars resulting in a colored complex which is measured at 460 nm.

### TSNAs

In an aspect, a modified tobacco plant provided further comprises one or more mutations in one or more loci encoding a nicotine demethylase (e.g., CYP82E4, CYP82E5, CYP82E10) that confer reduced amounts of nornicotine (See U.S. Pat. Nos. 8,319,011; 8,124,851; 9,187,759; 9,228,194; 9,228,195; 9,247,706) compared to a control tobacco plant lacking one or more mutations in one or more loci encoding a nicotine demethylase when grown under comparable conditions. In an aspect, a modified tobacco plant described further comprises reduced nicotine demethylase activity compared to a control plant when grown and cured under comparable conditions. In a further aspect, a tobacco plant provided further comprises one or more mutations or transgenes providing an elevated level of one or more antioxidants (See U.S. Patent Application Publication No. 2018/0119163 and WO 2018/067985). In another aspect, a tobacco plant provided further comprises one or more mutations or transgenes providing a reduced level of one or more tobacco-specific nitrosamines (TSNAs). In an aspect, a TSNA is selected from the group consisting of N′-nitrosonornicotine (NNN), 4-methylnitrosoamino-1-(3-pyridyl)-1-butanone (NNK), N′-nitrosoanatabine (NAT), and N′-nitrosoanabasine (NAB).

### EMBODIMENTS

The following non-limiting embodiments are envisioned:
1. A modified tobacco plant, or part thereof, comprising at least one non-natural mutation in an endogenous nucleic acid sequence, wherein the endogenous nucleic acid sequence encodes a polypeptide comprising an amino acid sequence at least 80% identical or similar to an amino acid sequence selected from the group consisting of SEQ ID NOs: 117-174.
2. A modified tobacco plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 80% identical or similar to an amino acid sequence selected from the group consisting of SEQ ID NOs: 117-174.
3. A modified tobacco plant, or part thereof, comprising a recombinant DNA construct comprising a heterologous promoter operably linked to a nucleic acid sequence encoding a polypeptide comprising an amino acid sequence at least 80% identical or similar to an amino acid sequence selected from the group consisting of SEQ ID NOs: 117-174.
4. The modified tobacco plant, or part thereof, of embodiment 1, wherein said tobacco plant produces at least one leaf comprising a reduced amount of at least one alkaloid as compared to the amount of said alkaloid in a control tobacco plant lacking said at least one non-natural mutation in said endogenous nucleic acid sequence when grown under comparable conditions.
5. The modified tobacco plant, or part thereof, of embodiment 2 or 3, wherein said tobacco plant produces at least one leaf comprising a reduced amount of at least one alkaloid as compared to the amount of said alkaloid in a control tobacco plant lacking said recombinant DNA construct when grown under comparable conditions.
6. The modified tobacco plant, or part thereof, of any one of embodiments 1, 2, or 4, wherein said endogenous nucleic acid sequence comprises a nucleic acid sequence at least 80% identical or complementary to a sequence selected from the group consisting of SEQ ID NOs: 1-58.
7. The modified tobacco plant, or part thereof, of embodiment 3, wherein said nucleic acid sequence comprises a nucleic acid sequence at least 80% identical or complementary to a sequence selected from the group consisting of SEQ ID NOs: 1-58.
8. The modified tobacco plant, or part thereof, of any one of embodiments 1, 2, or 4, wherein said endogenous nucleic acid sequence comprises a nucleic acid sequence at least 80% identical or complementary to a sequence selected from the group consisting of SEQ ID NOs: 59-116.
9. The modified tobacco plant, or part thereof, of embodiment 3, wherein said nucleic acid sequence comprises a nucleic acid sequence at least 80% identical or complementary to a sequence selected from the group consisting of SEQ ID NOs: 59-116.
10. The modified tobacco plant, or part thereof, of embodiment 1 or 4, wherein said tobacco plant is homozygous for said at least one non-natural mutation.
11. The modified tobacco plant, or part thereof, of embodiment 1 or 4, wherein said tobacco plant is heterozygous for said at least one non-natural mutation.
12. The modified tobacco plant, or part thereof, of any one of embodiments 4-6, 8, 10, or 11, wherein said at least one alkaloid is selected from the group consisting of anabasine, anatabine, nicotine, and nornicotine.
13. The modified tobacco plant, or part thereof, of any one of embodiments 4-6, 8, or 10-12, wherein said reduced amount of at least one alkaloid comprises a reduction of at least 1%.
14. The modified tobacco plant, or part thereof, of any one of embodiments 1, 4, 10, or 11, wherein said at least one non-natural mutation comprises a mutation selected from the group consisting of an insertion, a deletion, a substitution, a duplication, and an inversion.
15. The modified tobacco plant, or part thereof, of any one of embodiments 1, 4, 10, or 11, wherein said at least one non-natural mutation comprises at least one mutation selected from the group consisting of a nonsense mutation, a missense mutation, a frameshift mutation, and a splice-site mutation.
16. The modified tobacco plant, or part thereof, of any one of embodiments 1, 4, 10, 11, 14, or 15, wherein said at least one non-natural mutation comprises a null mutation.
17. The modified tobacco plant, or part thereof, of any one of embodiments 1, 4, 10, 11, or 14-16, wherein said at least one non-natural mutation results in a truncation of said polypeptide.
18. The modified tobacco plant, or part thereof, of any one of embodiments 1, 4, 10, 11, or 14-17, wherein said at least one non-natural mutation comprises a mutation in a sequence region selected from the group consisting of a promoter, a 5′-untranslated region (UTR), an exon, an intron, a 3′-UTR, and a terminator.
19. The modified tobacco plant, or part thereof, or part thereof, of any one of embodiments 1, 4, 10, 11, or 14-18, wherein said at least one non-natural mutation results in a reduced level of expression of said nucleic acid sequence as compared to expression of said nucleic acid sequence in the same tissue of a control tobacco plant when grown under comparable conditions, wherein said nucleic acid sequence lacks the at least one non-natural mutation in said control tobacco plant.
20. The modified tobacco plant, or part thereof, of any one of embodiments 1, 4, 10, 11, or 14-19, wherein said at least one non-natural mutation results in a reduced level of activity by a protein or polypeptide encoded by said nucleic acid sequence as compared to activity of a protein or polypeptide encoded by said nucleic acid sequence in a control tobacco plant when grown under comparable conditions, wherein said nucleic acid sequence lacks the at least one non-natural mutation in said control tobacco plant.
21. The modified tobacco plant, or part thereof, of any one of embodiments 2, 3, 5, 7, or 9, wherein said promoter comprises a promoter selected from the group consisting of a constitutive promoter, a tissue-preferred promoter, a tissue-specific promoter, and an inducible promoter.
22. The modified tobacco plant, or part thereof, of embodiment 21, wherein said tissue-preferred promoter comprises a root-preferred promoter.
23. The modified tobacco plant, or part thereof, of embodiment 21, wherein said tissue-specific promoter comprises a root-specific promoter.
24. The modified tobacco plant, or part thereof, of embodiment 21, wherein said constitutive promoter is selected from the group consisting of a Cauliflower Mosaic Virus (CaMV) 35S promoter, a ubiquitin promoter, an actin promoter, an opine promoter, and an alcohol dehydrogenase promoter.
25. The modified tobacco plant, or part thereof, of embodiment 2, wherein the at least one small RNA molecule is selected from the group consisting of a double-stranded RNA, a small interfering RNA (siRNA), a trans-acting siRNA, and a microRNA.
26. The modified tobacco plant, or part thereof, of embodiment 2, wherein said at least one small RNA molecule comprises between 18 nucleotides and 30 nucleotides.
27. The modified tobacco plant, or part thereof, of embodiment 2, wherein said at least one small RNA molecule comprises a nucleic acid sequence at least 90% complementary to a sequence selected from the group consisting of SEQ ID NOs: 59-116.
28. The modified tobacco plant, or part thereof, of any one of embodiments 1-27, wherein the modified tobacco plant is of a tobacco variety selected from the group consisting of a flue-cured variety, a bright variety, a Burley variety, a Virginia variety, a Maryland variety, a dark variety, a Galpio variety, an Oriental variety, and a Turkish variety.
29. The modified tobacco plant, or part thereof, of any one of embodiments 1-116, wherein the modified tobacco plant is of a variety selected from the group consisting of the tobacco varieties listed in Tables 2-8.
30. The modified tobacco plant, or part thereof, of any one of embodiments 1-29, wherein the modified tobacco plant is a hybrid.
31. The modified tobacco plant, or part thereof, of any one of embodiments 1-30, wherein the modified tobacco plant is male sterile or cytoplasmically male sterile.
32. The modified tobacco plant, or part thereof, of any one of embodiments 1-30, wherein the modified tobacco plant is female sterile.
33. Cured tobacco material from the modified tobacco plant, or part thereof, of any one of embodiments 1-32.
34. The cured tobacco material of embodiment 33, wherein said cured tobacco material comprises cured leaf material, cured stem material, or both.
35. The cured tobacco material of embodiment 33 or 34, wherein said cured tobacco material comprises flue-cured tobacco material, air-cured tobacco material, fire-cured tobacco material, and sun-cured tobacco material.
36. A tobacco blend comprising the cured tobacco material of any one of embodiments 33-35.
37. The tobacco blend of embodiment 36, wherein said tobacco blend comprises at least 10% cured tobacco by weight.
38. The tobacco blend of embodiment 36, wherein said tobacco blend comprises at least 10% cured tobacco by volume.
39. A tobacco product comprising the tobacco blend of any one of embodiments 36-38.
40. A tobacco product comprising the cured tobacco material of any one of embodiments 33-35.
41. The tobacco product of embodiment 39 or 40, wherein said tobacco product is selected from the group consisting of a cigarette, a heated tobacco product, a kretek, a bidi cigarette, a cigar, a cigarillo, a non-ventilated cigarette, a vented recess filter cigarette, pipe tobacco, snuff, snus, chewing tobacco, moist smokeless tobacco, fine cut chewing tobacco, long cut chewing tobacco, pouched chewing tobacco product, gum, a tablet, a lozenge, and a dissolving strip.
42. The tobacco product of embodiment 39 or 40, wherein said tobacco product is a smokeless tobacco product.
43. The tobacco product of embodiment 42, wherein said smokeless tobacco product is selected from the group consisting of loose leaf chewing tobacco, plug chewing tobacco, moist snuff, nasal snuff, dry snuff, and snus.
44. A reconstituted tobacco comprising the cured tobacco material of any one of embodiments 33-35.
45. A method of producing a modified tobacco plant comprising:
   (a) inducing a non-natural mutation in at least one tobacco cell in an endogenous nucleic acid sequence encoding a polypeptide comprising an amino acid sequence at least 80% identical or similar to an amino acid sequence selected from the group consisting of SEQ ID NOs: 117-174;
   (b) selecting at least one tobacco cell comprising said non-natural mutation from step (a); and
   (c) regenerating at least one modified tobacco plant from said at least one tobacco cell selected in step (b).
46. A method of producing a modified tobacco plant comprising:
   (a) introducing a recombinant DNA construct to at least one tobacco cell, wherein said recombinant DNA construct comprises a heterologous promoter operably linked to a nucleic acid encoding at least one small RNA molecule capable of binding to and reducing the expression of an endogenous nucleic acid sequence encoding a polypeptide at least 80% identical or similar to an amino acid sequence selected from the group consisting of SEQ ID NOs: 117-174;
   (b) selecting at least one tobacco cell comprising said recombinant DNA construct; and
   (c) regenerating at least one modified tobacco plant from said at least one tobacco cell selected in step (b).
47. A method of producing a modified tobacco plant comprising:
   (a) introducing a recombinant DNA construct to at least one tobacco cell, wherein said recombinant DNA construct comprises a heterologous promoter operably linked to a nucleic acid sequence encoding a polypeptide comprising an amino acid sequence at least 80% identical or similar to an amino acid sequence selected from the group consisting of SEQ ID NOs: 117-174;
   (b) selecting at least one tobacco cell comprising said recombinant DNA construct; and
   (c) regenerating at least one modified tobacco plant from said at least one tobacco cell selected in step (b).
48. The method of embodiment 45, wherein said at least one modified tobacco plant comprises a reduced amount of at least one alkaloid as compared to a control tobacco plant lacking said mutation when grown under comparable conditions.
49. The method of embodiment 46 or 47, wherein said at least one modified tobacco plant comprises a reduced amount of at least one alkaloid as compared to a control tobacco plant lacking said recombinant DNA construct when grown under comparable conditions.
50. The method of any one of embodiments 45, 46, 48, or 49, wherein said endogenous nucleic acid sequence is at least 80% identical to a sequence selected from the group consisting of SEQ ID NOs: 1-58.
51. The method of embodiment 47 or 49, wherein said nucleic acid sequence is at least 80% identical to a sequence selected from the group consisting of SEQ ID NOs: 1-58.
52. The method of any one of embodiments 45, 46, 48, or 49, wherein said endogenous nucleic acid sequence is at least 80% identical to a sequence selected from the group consisting of SEQ ID NOs: 59-116.
53. The method of embodiment 47 or 49, wherein said nucleic acid sequence is at least 80% identical to a sequence selected from the group consisting of SEQ ID NOs: 59-116.
54. The method of any one of embodiments 48-53, wherein said at least one alkaloid is selected from the group consisting of anabasine, anatabine, nicotine, and nornicotine.
55. The method of any one of embodiments 48-53, wherein said reduced amount of at least one alkaloid comprises a reduction of at least 1%.
56. The method of any one of embodiments 45, 48, 50, 52, 54, or 55, wherein said non-natural mutation comprises a mutation selected from the group consisting of an insertion, a deletion, a substitution, a duplication, and an inversion.
57. The method of any one of embodiments 45, 48, 50, 52, or 54-56, wherein said non-natural mutation comprises a mutation selected from the group consisting of a nonsense mutation, a missense mutation, a frameshift mutation, and a splice-site mutation.
58. The method of any one of embodiments 45, 48, 50, 52, or 54-57, wherein said non-natural mutation comprises a null mutation.
59. The method of any one of embodiments 45, 48, 50, 52, or 54-58, wherein said non-natural mutation results in a truncation of said polypeptide.
60. The method of any one of embodiments 45, 48, 50, 52, or 54-59, wherein said non-natural mutation comprises a mutation in a sequence region selected from the group consisting of a promoter, a 5′-untranslated region (UTR), an exon, an intron, a 3′-UTR, and a terminator.
61. The method of any one of embodiments 45, 48, 50, 52, or 54-60, wherein said inducing comprises the use of an agent selected from the group consisting of: a chemical mutagen, irradiation, a transposon, Agrobacterium, and a nuclease.
62. The method of embodiment 61, wherein said nuclease is selected from the group consisting of a meganuclease, a zinc-finger nuclease, a transcription activator-like effector nuclease, a CRISPR/Cas9 nuclease, a CRISPR/Cpf1 nuclease, a CRISPR/CasX nuclease, a CRISPR/CasY nuclease, a Csm1 nuclease, or any combination thereof.
63. The method of embodiment 61, wherein said chemical mutagen comprises ethyl methanesulfonate.
64. The method of embodiment 61, wherein said irradiation comprises gamma rays, X-rays, ionizing radiation, or fast neutrons.
65. The method of embodiment 46, wherein said small RNA molecule is selected from the group consisting of a double-stranded RNA, a small interfering RNA (siRNA), a trans-acting siRNA, and a microRNA.
66. The method of embodiment 46, wherein said at least one small RNA molecule comprises between 18 nucleotides and 30 nucleotides.
67. The method of embodiment 46, wherein said at least one small RNA molecule comprises a nucleic acid sequence at least 90% complementary to a sequence selected from the group consisting of SEQ ID NOs: 59-116.
68. The method of any one of embodiments 46, 47, or 49-53, wherein said promoter comprises a promoter selected from the group consisting of a constitutive promoter, a tissue-preferred promoter, a tissue-specific promoter, and an inducible promoter.
69. The method of embodiment 68, wherein said tissue-preferred promoter comprises a root-preferred promoter.
70. The method of embodiment 68, wherein said tissue-specific promoter comprises a root-specific promoter.
71. The method of embodiment 68, wherein said constitutive promoter is selected from the group consisting of a Cauliflower Mosaic Virus (CaMV) 35S promoter, a ubiquitin promoter, an actin promoter, an opine promoter, and an alcohol dehydrogenase promoter.
72. The method of any one of embodiments 45-71, wherein said at least one tobacco cell is a tobacco protoplast cell.
73. The method of any one of embodiments 45-71, wherein said at least one tobacco cell is a tobacco callus cell.
74. The method of any one of embodiments 45-71, wherein said at least one tobacco cell is selected from the group consisting of a seed cell, a fruit cell, a leaf cell, a cotyledon cell, a hypocotyl cell, a meristem cell, an embryo cell, an endosperm cell, a root cell, a shoot cell, a stem cell, a flower cell, an inflorescence cell, a stalk cell, a pedicel cell, a style cell, a stigma cell, a receptacle cell, a petal cell, a sepal cell, a pollen cell, an anther cell, a filament cell, an ovary cell, an ovule cell, a pericarp cell, and a phloem cell.
75. The method of any one of embodiments 45-47, wherein said method further comprises:
   (d) growing said modified tobacco plant regenerated in step (c).
76. The method of embodiment 75, wherein said method further comprises:
   (e) crossing said modified tobacco plant grown in step (d) with a second tobacco plant; and
   (f) obtaining at least one seed from said crossing in step (e).
77. The method of embodiment 45 or 48, wherein said at least one non-natural mutation results in a reduced level of expression of said nucleic acid sequence as compared to expression of said nucleic acid sequence in the same tissue of a control tobacco plant when grown under comparable conditions, wherein said nucleic acid sequence lacks the at least one non-natural mutation in said control tobacco plant.
78. The method of embodiment 77, wherein said reduced level of expression comprises a reduction of at least 5%.
79. The method of embodiment 45 or 48, wherein said at least one non-natural mutation results in an increased level of expression of said nucleic acid sequence as compared to expression of said nucleic acid sequence in the same tissue of a control tobacco plant when grown under comparable conditions, wherein said nucleic acid sequence lacks the at least one non-natural mutation in said control tobacco plant.
80. The method of embodiment 79, wherein said increased level of expression comprises an increase of at least 5%.
81. The method of embodiment 45 or 48, wherein said at least one non-natural mutation results in a reduced level of activity by a protein or polypeptide encoded by said nucleic acid sequence as compared to activity of a protein or polypeptide encoded by said nucleic acid sequence in a control tobacco plant when grown under comparable conditions, wherein said nucleic acid sequence lacks the at least one non-natural mutation in said control tobacco plant.
82. The method of embodiment 45 or 48, wherein said at least one non-natural mutation results in an increased level of activity by a protein or polypeptide encoded by said nucleic acid sequence as compared to activity of a protein or polypeptide encoded by said nucleic acid sequence in a control tobacco plant when grown under comparable conditions, wherein said nucleic acid sequence lacks the at least one non-natural mutation in said control tobacco plant.
83. The method of any one of embodiments 45-82, wherein said modified tobacco plant is of a tobacco variety selected from the group consisting of a flue-cured variety, a bright variety, a Burley variety, a Virginia variety, a Maryland variety, a dark variety, a Galpio variety, an Oriental variety, and a Turkish variety.
84. The method of any one of embodiments 45-83, wherein said modified tobacco plant is of a variety selected from the group consisting of the varieties listed in Tables 2-8.
85. The method of any one of embodiments 45-84, wherein said modified tobacco plant is a hybrid.
86. The method of any one of embodiments 45-85, wherein said modified tobacco plant is male sterile or cytoplasmically male sterile.
87. The method of any one of embodiments 45-85, wherein said modified tobacco plant is female sterile.
88. The method of embodiment 45 or 48, wherein said modified tobacco plant comprises a comparable or higher USDA leaf grade index as compared to a control tobacco plant lacking said non-natural mutation when grown under comparable conditions.
89. The method of embodiment 46 or 47, wherein said modified tobacco plant comprises a comparable or higher USDA leaf grade index as compared to a control tobacco plant lacking said recombinant DNA construct when grown under comparable conditions.
90. A method comprising preparing a tobacco product using cured tobacco material from a modified tobacco plant, wherein said modified tobacco plant comprises a non-natural mutation in an endogenous nucleic acid sequence, wherein the endogenous nucleic acid sequence encodes a polypeptide comprising an amino acid sequence at least 80% identical or similar to an amino acid sequence selected from the group consisting of SEQ ID NOs: 117-174.
91. A method comprising preparing a tobacco product using cured tobacco material from a modified tobacco plant, wherein said modified tobacco plant comprises a recombinant DNA construct, and wherein said recombinant DNA construct comprises a heterologous promoter operably linked to a nucleic acid sequence encoding at least one small RNA molecule capable of binding to and reducing the expression of an endogenous nucleic acid sequence encoding a polypeptide at least 80% identical or similar to an amino acid sequence selected from the group consisting of SEQ ID NOs: 117-174.
92. A method comprising preparing a tobacco product using cured tobacco material from a modified tobacco plant, wherein said modified tobacco plant comprises a recombinant DNA construct, and wherein said recombinant DNA construct comprises a heterologous promoter operably linked to a nucleic acid sequence encoding a polypeptide comprising an amino acid sequence at least 80% identical or similar to an amino acid sequence selected from the group consisting of SEQ ID NOs: 117-174.
93. The method of any one of embodiments 90-92, wherein said cured tobacco material comprises cured leaf material, cured stem material, or both.
94. The method of any one of embodiments 90-92, wherein said cured tobacco material comprises flue-cured tobacco material, air-cured tobacco material, fire-cured tobacco material, and sun-cured tobacco material.
95. The method of any one of embodiments 90-92, wherein said tobacco product is selected from the group consisting of a cigarette, a kretek, a bidi cigarette, a cigar, a cigarillo, a non-ventilated cigarette, a vented recess filter cigarette, pipe tobacco, snuff, snus, chewing tobacco, moist smokeless tobacco, fine cut chewing tobacco, long cut chewing tobacco, pouched chewing tobacco product, gum, a tablet, a lozenge, and a dissolving strip.
96. The method of any one of embodiments 90-92, wherein said tobacco product is a smokeless tobacco product.
97. The method of embodiment 96, wherein said smokeless tobacco product is selected from the group consisting of loose leaf chewing tobacco, plug chewing tobacco, moist snuff, nasal snuff, dry snuff, and snus.
98. The method of any one of embodiments 90-92, wherein said cured tobacco material is of a tobacco variety selected from the group consisting of a flue-cured variety, a bright variety, a Burley variety, a Virginia variety, a Maryland variety, a dark variety, a Galpio variety, an Oriental variety, and a Turkish variety.
99. The method of any one of embodiments 90-98, wherein said endogenous nucleic acid sequence comprises a sequence at least 80% identical to a sequence selected from the group consisting of SEQ ID NOs: 1-116.
100. The method of any one of embodiments 92-98, wherein said nucleic acid sequence comprises a sequence at least 80% identical to a sequence selected from the group consisting of SEQ ID NOs: 1-116.
101. A method comprising transforming a tobacco cell with a recombinant DNA construct, wherein said recombinant DNA construct comprises a heterologous promoter operably linked to a nucleic acid sequence encoding at least one small RNA molecule capable of binding to and reducing the expression of an endogenous nucleic acid sequence encoding a polypeptide at least 80% identical or similar to an amino acid sequence selected from the group consisting of SEQ ID NOs: 117-174.
102. A method comprising transforming a tobacco cell with a recombinant DNA construct, wherein said recombinant DNA construct comprises a heterologous promoter operably linked to a nucleic acid sequence encoding a polypeptide comprising an amino acid sequence at least 80% identical or similar to an amino acid sequence selected from the group consisting of SEQ ID NOs: 117-174.
103. A method for producing a modified tobacco plant comprising:
   (a) crossing at least one tobacco plant of a first tobacco variety with at least one tobacco plant of a second tobacco variety to produce at least one progeny tobacco seed, wherein said at least one tobacco plant of said first tobacco variety comprises a non-natural mutation in an endogenous nucleic acid sequence, where the endogenous nucleic acid sequence encodes a polypeptide comprising an amino acid sequence at least 80% identical or similar to an amino acid sequence selected from the group consisting of SEQ ID NOs: 117-174, wherein said non-natural mutation is not present in said endogenous nucleic acid sequence in a control tobacco plant of said first tobacco variety; and
   (b) selecting for at least one progeny tobacco seed, or a plant germinated therefrom, wherein said at least one tobacco seed or plant germinated therefrom comprises said non-natural mutation.
104. A method for producing a modified tobacco plant comprising:
   (a) crossing at least one tobacco plant of a first tobacco variety with at least one tobacco plant of a second tobacco variety to produce at least one progeny tobacco seed, wherein said at least one tobacco plant of said first tobacco variety comprises a recombinant DNA construct, wherein said recombinant DNA construct comprises a heterologous promoter operably linked to a nucleic acid sequence encoding at least one small RNA molecule capable of binding to and reducing the expression of an endogenous nucleic acid sequence encoding a polypeptide at least 80% identical or similar to an amino acid sequence selected from the group consisting of SEQ ID NOs: 117-174, wherein said recombinant DNA construct is not present in said endogenous nucleic acid sequence in a control tobacco plant of the same variety; and
   (b) selecting for at least one progeny tobacco seed, or a plant germinated therefrom, wherein said at least one tobacco seed or plant germinated therefrom comprises said recombinant DNA construct.
105. A method for producing a modified tobacco plant comprising:
   (a) crossing at least one tobacco plant of a first tobacco variety with at least one tobacco plant of a second tobacco variety to produce at least one progeny tobacco seed, wherein said at least one tobacco plant of said first tobacco variety comprises a recombinant DNA construct, wherein said recombinant DNA construct comprises a heterologous promoter operably linked to a nucleic acid sequence encoding a polypeptide comprising an amino acid sequence at least 80% identical or similar to an amino acid sequence selected from the group consisting of SEQ ID NOs: 117-174, wherein said recombinant DNA construct is not present in said nucleic acid sequence in a control tobacco plant of the first tobacco variety; and
   (b) selecting for at least one progeny tobacco seed, or a plant germinated therefrom, wherein said at least one tobacco seed or plant germinated therefrom comprises said recombinant DNA construct.
106. The method of any one of embodiments 103-105, wherein said plant germinated in step
   (b) comprises a reduced amount of at least one alkaloid as compared to said control tobacco plant when grown under comparable conditions.
107. The method of embodiment 106, wherein said at least one alkaloid is selected from the group consisting of anabasine, anatabine, nicotine, and nornicotine.
108. The method of embodiment 106 or 107, wherein said reduced amount of at least one alkaloid comprises a reduction of at least 1%.
109. The method of embodiment 103 or 104, wherein said endogenous nucleic acid sequence comprises a sequence at least 80% identical to a sequence selected from the group consisting of SEQ ID NOs: 1-116.
110. The method of embodiment 105, wherein said nucleic acid sequence comprises a sequence at least 80% identical to a sequence selected from the group consisting of SEQ ID NOs: 1-116.
111. A modified tobacco plant, or part thereof, comprising at least one non-natural mutation in an endogenous nucleic acid sequence that modulates the expression or functional activity of a gene, wherein said gene encodes a polypeptide comprising an amino acid sequence at least 80% identical or similar to an amino acid sequence selected from the group consisting of SEQ ID NOs: 117-174.
112. The modified tobacco plant, or part thereof, of embodiment 111, wherein said endogenous nucleic acid sequence encodes a transcription factor that is capable of binding to a nucleic acid sequence encoding said gene.
113. The modified tobacco plant, or part thereof, of embodiment 111, wherein said endogenous nucleic acid sequence encodes a repressor that is capable of binding to a nucleic acid sequence encoding said gene.
114. The modified tobacco plant, or part thereof, of embodiment 111, wherein said endogenous nucleic acid sequence encodes a protein that generates a precursor or substrate required for said polypeptide to function.
115. The modified tobacco plant, or part thereof, of any one of embodiments 111-114, wherein said expression or functional activity is increased expression or activity as compared to a control tobacco plant lacking said at least one non-natural mutation when grown under comparable conditions.
116. The modified tobacco plant, or part thereof, of any one of embodiments 111-114, wherein said expression or functional activity is decreased expression or activity as compared to a control tobacco plant lacking said at least one non-natural mutation when grown under comparable conditions.
117. The modified tobacco plant, or part thereof, of any one of embodiments 111-116, wherein said tobacco plant produces a leaf comprising a reduced amount of at least one alkaloid as compared to the amount of said alkaloid in a control tobacco plant lacking said at least one non-natural mutation in said endogenous nucleic acid sequence when grown under comparable conditions.
118. Cured tobacco material from the modified tobacco plant, or part thereof, of any one of embodiments 111-117.
119. A tobacco blend comprising the cured tobacco material of embodiment 118.
120. A tobacco product comprising the tobacco blend of embodiment 119.
121. A tobacco product comprising the cured tobacco material of embodiment 118.
122. A reconstituted tobacco comprising the cured tobacco material of embodiment 118.
123. A modified tobacco plant, or part thereof, comprising: (a) a genetic modification in a gene; or (b) a genetic modification targeting said gene; wherein said genetic modification downregulates the expression or activity of said gene, wherein said gene encodes a nucleic acid sequence having at least 80% identity to a polynucleotide sequence selected from the group consisting of SEQ ID NOs: 1-116.

Having now generally described the disclosure, the same will be more readily understood through reference to the following examples that are provided by way of illustration, and are not intended to be limiting of the present disclosure, unless specified.

### EXAMPLES

### Example 1. RNA Sequencing

RNA is isolated from TN90 tobacco roots at three time points: before topping, three-days after topping, one-week after topping, and two-weeks after topping. Three technical replicates from three biological replicates are collected for each time point. The isolated RNA is sequenced using Illumina® technology.

Sequenced reads are assessed for quality, and then mapped to a proprietary tobacco reference genome using CLC Genomics Workbench version 11.0.1 software (QIAGEN) using default parameter settings. A read count matrix including all samples is generating by aggregating the raw counts of the mapped reads for each gene in each sample against a total of 98,753 tobacco genes using the Expression Browser Tool in CLC Genomics Workbench.

RNA sequences are mapped to 57,595 tobacco genes. The 41,158 tobacco genes that do not exhibit expression are removed from further consideration. For the tobacco genes that exhibit expression, gene expression values are normalized by library size in terms of reads mapped per kilobase per million reads (RPKM). In order to reduce noise from tobacco genes with consistent expression across all four time points, or that have consistently low expression, tobacco genes that exhibit a fold change in expression across all samples of less than two log are removed. Following the removal of these tobacco genes, a total of 21,025 tobacco genes exhibit dynamic expression across the three measured time points.

### Example 2. WGCNA Analysis

The R package wgcna is used to perform weighted gene co-expression network analysis (WGCNA) on the 21,025 genes exhibiting dynamic expression. See Langfelder & Horvath, “WGCNA: an R package for weighted correlation network analysis,” BMC Bioinformatics, 9:559 (2008). A matrix of all tobacco genes with their RPKM values across nine samples (all technical and biological replicates) is used as input for WGCNA. An adjacency matrix is generated to determine similarity (e.g., correlation for every tobacco gene pair across the nine samples) between tobacco genes. Next, the results are transformed through a soft thresholding procedure in wgcna using the function pickSoftThreshold, where a soft power of 10 is chosen for module detection.

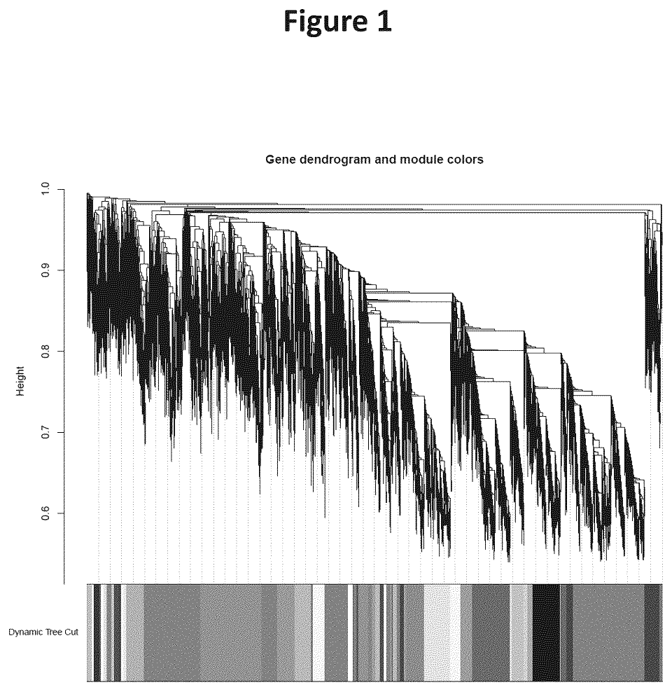

A topological overlap measure (TOM) is calculated from the adjacency matrix to estimate network interconnectedness. The dissimilarity of TOM (1-TOM) is used as the input for average linkage hierarchical clustering to identify co-expression modules. The clustering identifies 54 gene modules, and the 34 known nicotine biosynthetic pathway genes are present among these modules. See FIG. 1.

wgcna is also used to determine the interconnectedness of genes within, and between, modules. A strength value (e.g., weight) is assigned to the connection (edge) between each gene (node) and every other gene in the network. An integrated co-expression network is generating after filtering gene-to-gene connections having connectivity (edge weight >0.02).

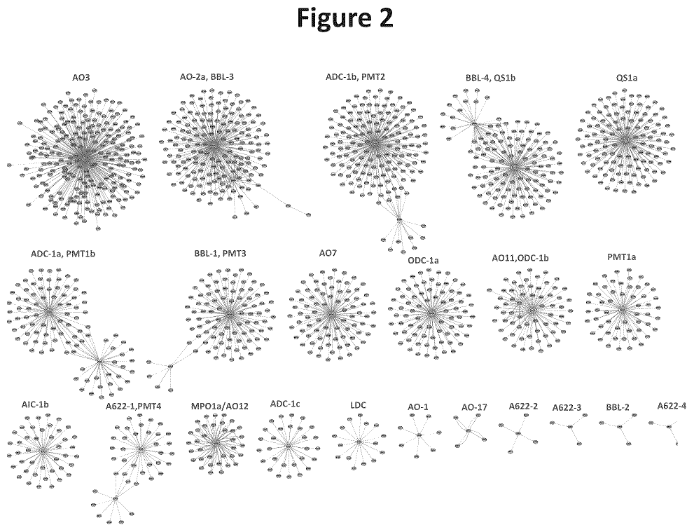

In order to define sub-networks related to specific developmental processes, and to identify novel regulatory factors, the co-expression networks are filtered for genes having strong connects to known genes of interest within a module. Co-expression modules are extracted for all of the known genes in the nicotine biosynthetic pathway and used to construct sub-networks. Gene-to-gene connections are filtered using an edge weight parameter of >0.08 to reduce complexity, and the edge weight parameter is subjected to increased stringency within sub-networks as necessary. See FIG. 2.

The sub-networks comprise 1452 tobacco genes, of which 1394 are annotated. The genes in the sub-networks include 30 nicotine biosynthesis-related genes and their homologs, plus an 58 unknown tobacco genes. The 58 unknown tobacco genes are candidates for further study. See Table 9.

TABLE 9
Candidate gene nucleic acid and amino acid sequences.

| Gene | Genomic DNA | cDNA | Amino Acid |
|---|---|---|---|
| Identifier | SEQ ID NO | SEQ ID NO | SEQ ID NO |
| g12941 | 1 | 59 | 117 |
| g14663 | 2 | 60 | 118 |
| g20986 | 3 | 61 | 119 |
| g20989a | 4 | 62 | 120 |
| g20989b | 5 | 63 | 121 |
| g22648 | 6 | 64 | 122 |
| g23499 | 7 | 65 | 123 |
| g26597 | 8 | 66 | 124 |
| g31004 | 9 | 67 | 125 |
| g31698 | 10 | 68 | 126 |
| g31724 | 11 | 69 | 127 |
| g44455 | 12 | 70 | 128 |
| g44587 | 13 | 71 | 129 |
| g47147 | 14 | 72 | 130 |
| g49713 | 15 | 73 | 131 |
| g52130 | 16 | 74 | 132 |
| g57292 | 17 | 75 | 133 |
| g59464 | 18 | 76 | 134 |
| g59465 | 19 | 77 | 135 |
| g60786 | 20 | 78 | 136 |
| g62380 | 21 | 79 | 137 |
| g62763 | 22 | 80 | 138 |
| g66085 | 23 | 81 | 139 |
| g66301 | 24 | 82 | 140 |
| g68242 | 25 | 83 | 141 |
| g69182 | 26 | 84 | 142 |
| g69391 | 27 | 85 | 143 |
| g71598 | 28 | 86 | 144 |
| g74738 | 29 | 87 | 145 |
| g74782 | 30 | 88 | 146 |
| g74912 | 31 | 89 | 147 |
| g75157 | 32 | 90 | 148 |
| g75446 | 33 | 91 | 149 |
| g75500 | 34 | 92 | 150 |
| g76096 | 35 | 93 | 151 |
| g77018 | 36 | 94 | 152 |
| g77780 | 37 | 95 | 153 |
| g78265 | 38 | 96 | 154 |
| g78408 | 39 | 97 | 155 |
| g79607 | 40 | 98 | 156 |
| g79774 | 41 | 99 | 157 |
| g82124 | 42 | 100 | 158 |
| g82252 | 43 | 101 | 159 |
| g82744 | 44 | 102 | 160 |
| g83936 | 45 | 103 | 161 |
| g86315 | 46 | 104 | 162 |
| g86407 | 47 | 105 | 163 |
| g86563 | 48 | 106 | 164 |
| g89013 | 49 | 107 | 165 |
| g91123 | 50 | 108 | 166 |
| g91334 | 51 | 109 | 167 |
| g91336 | 52 | 110 | 168 |
| g91411 | 53 | 111 | 169 |
| g92376 | 54 | 112 | 170 |
| g92662 | 55 | 113 | 171 |
| g93191 | 56 | 114 | 172 |
| g95317 | 57 | 115 | 173 |
| g95400 | 58 | 116 | 174 |

### Example 3. Candidate Gene Expression

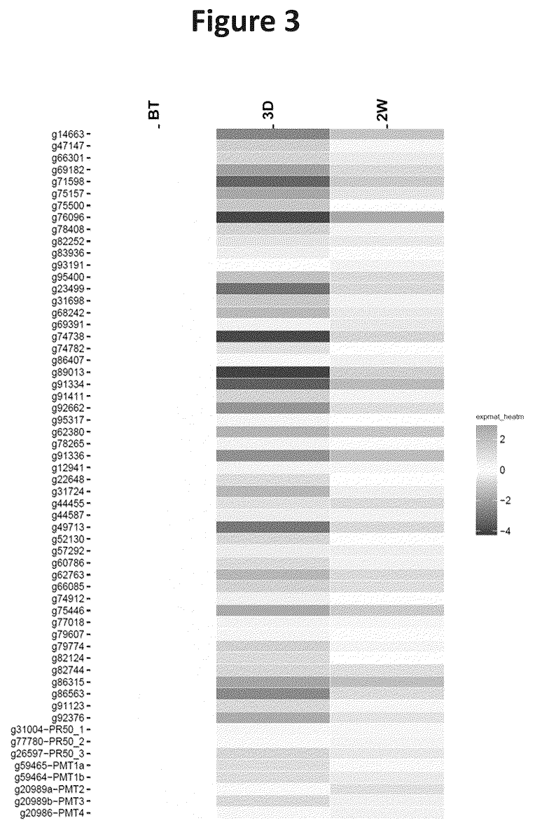

Of the 58 unknown tobacco genes ultimately identified in Example 2, 50 are considered for further evaluation. RNA expression data from Example 1 are used to compare the expression of the 50 candidate genes against the expression of known genes (e.g., PR50_1, PR50_2, PR50_3, PMT1a, PMT1b, PMT2, PMT3, PMT4). See FIG. 3.

To validate the RNA sequencing data from Example 1, expression of the candidate genes listed in Table 9 is examined in root samples from two tobacco lines: TN90 and CS118 (e.g., a PMT knockout line). See U.S. Patent Application Publication No. 2020/0029522 for additional information about CS118. Three independently collected samples are obtained at three time points: before topping (e.g., at flowering stage), three days after topping, and one week after topping.

RNA is extracted from the root samples, and cDNA transcripts are generated from the RNA samples, using methods standard in the art. The resulting cDNA is used to measure the relative expression of each candidate gene with quantitative RT-PCR (qRT-PCR) using gene-specific primers.

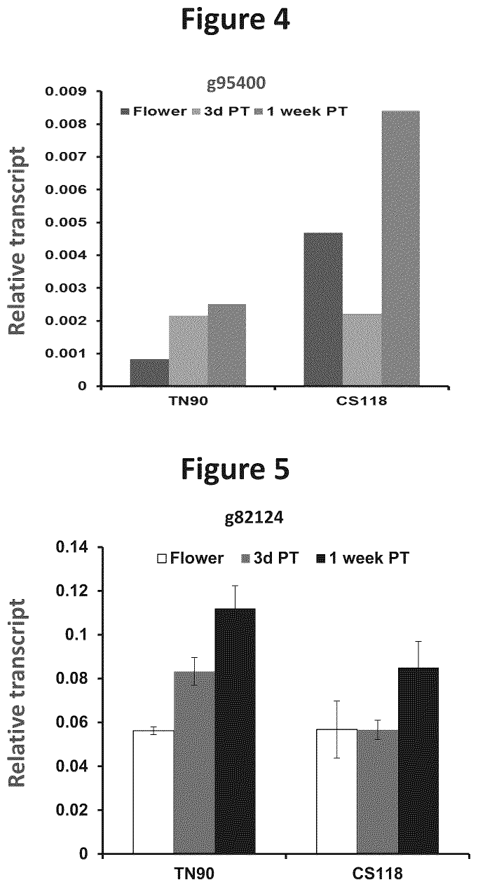

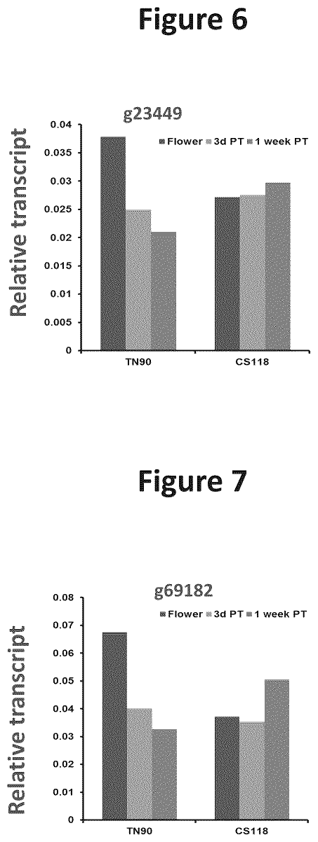

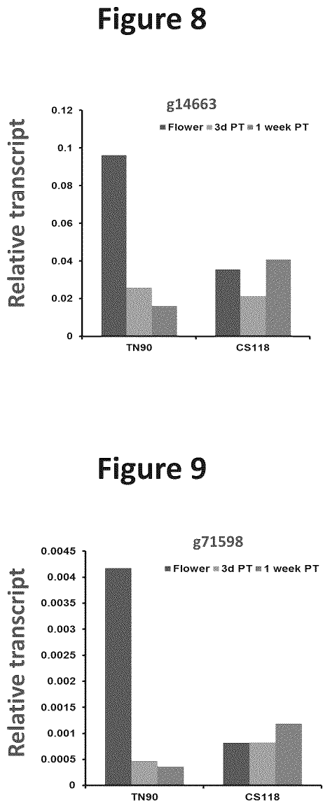

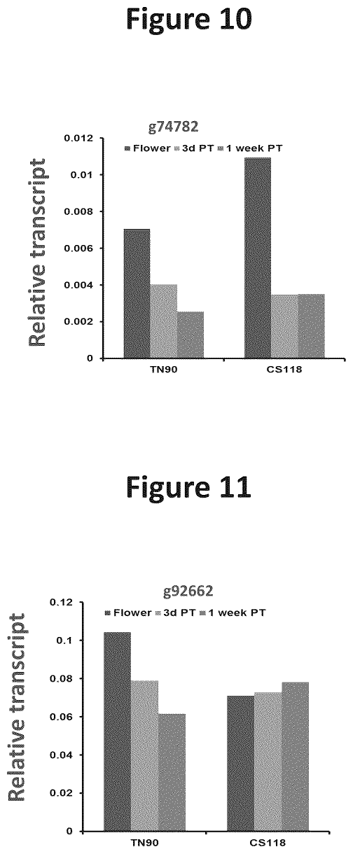

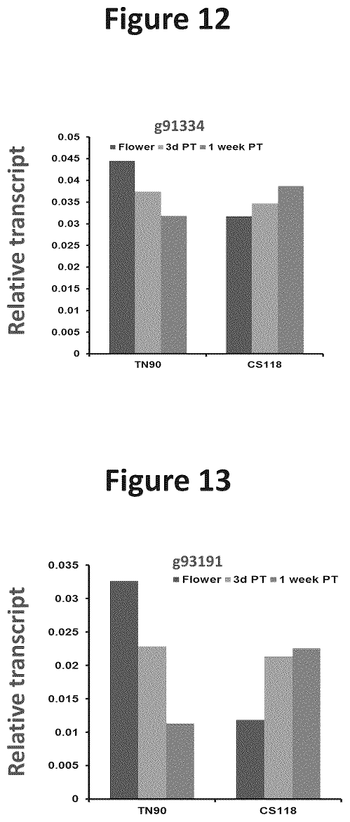

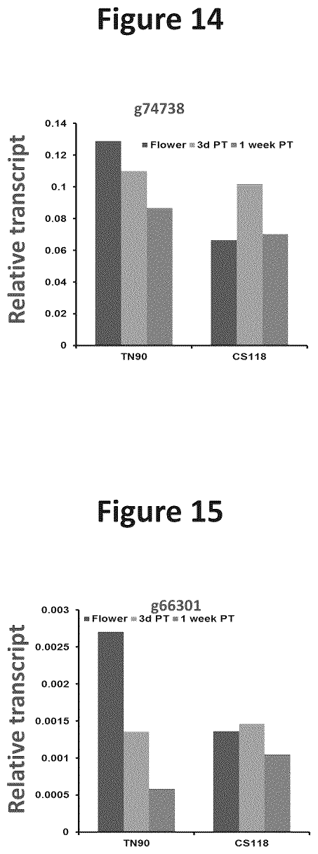

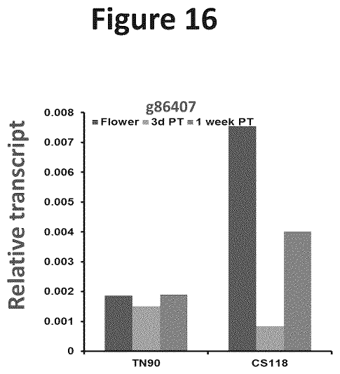

Relative expression of each candidate gene, as compared to a control (tobacco EF1α; SEQ ID NO: 176) is measured. See, e.g., FIGS. 4-16.

### Example 4. Transformation and Regeneration of Modified Tobacco Plants

An expression vector is used as a backbone to generate multiple transformation vectors comprising recombinant DNA constructs (SEQ ID NO: 175). The expression vector contains a Cassava Vein Mosaic Virus (CsVMV) promoter, a NOS terminator, and a cassette comprising a kanamycin selection marker (NPT II) operably linked to an Actin2 promoter and a NOS terminator. Nucleic acid vectors comprising transgenes of interest (e.g., SEQ ID NOs: 1-116) are introduced into tobacco leaf discs via Agrobacterium transformation. See, for example, Mayo et al., 2006, Nat Protoc. 1:1105-11 and Horsch et al., 1985, Science 227:1229-1231.

TN90 tobacco plants are grown in Magenta™ GA-7 boxes and leaf discs are cut and placed into Petri plates. Agrobacterium tumefaciens cells comprising a transformation vector are collected by centrifuging a 20 mL cell suspension in a 50 mL centrifuge tube at 3500 RPM for 10 minutes. The supernatant is removed and the Agrobacterium tumefaciens cell pellet is re-suspended in 40 mL liquid re-suspension medium. Tobacco leaves, avoiding the midrib, are cut into eight 0.6 cm discs with a #15 razor blade and placed upside down in a Petri plate. A thin layer of Murashige & Skoog (MS) with B5 vitamin liquid re-suspension medium is added to the Petri plate and the leaf discs are poked uniformly with a fine point needle. About 25 mL of the Agrobacterium tumefaciens suspension is added to the Petri plate and the leaf discs are incubated in the suspension for 10 minutes.

Leaf discs are transferred to co-cultivation Petri plates (½ MS medium) and discs are placed upside down in contact with filter paper overlaid on the co-cultivation TOM medium (MS medium with 20 g/L sucrose; 1 mg/L indole-3-acetic acid; and 2.5 mg/L 6-benzyl aminopurine (BAP)). The Petri plate is sealed with parafilm prior to incubation in dim light (60-80 mE/ms) with 18 hours on, 6 hours off photoperiods at 24° C. for three days. After incubation, leaf discs are transferred to regeneration/selection TOM K medium Petri plates (TOM medium plus 300 mg/L kanamycin). Leaf discs are sub-cultured bi-weekly to fresh TOM K medium in dim light with 18 hours on, 6 hours off photoperiods at 24° C. until shoots become excisable. Shoots from leaves are removed with forceps and inserted in MS basal medium with 100 mg/L kanamycin. Shoots on MS basal medium with 100 mg/L kanamycin are incubated at 24° C. with 18 hours on, 6 hours off photoperiods with high intensity lighting (6080 mE/ms) to induce rooting.

When plantlets comprising both shoots and roots grow large enough (e.g., over half the height of a Magenta™ GA-7 box), they are transferred to soil for acclimatization. Once established, seedlings are transferred to a greenhouse for further growth, breeding, and analysis.

### Example 5. Measuring Alkaloid Levels in Tobacco

Modified tobacco plants from Example 4, along with controls, are grown in 25 centimeter pots in a greenhouse with 75 parts per million fertilizer. At flowering stage, plants are topped. Two weeks after topping, lamina samples are collected from the third, fourth, and fifth leaves from the top of the plant, and alkaloid levels are measured using a method in accordance with CORESTA Method No 62, Determination of Nicotine in Tobacco and Tobacco Products by Gas Chromatographic Analysis, February 2005, and those defined in the Centers for Disease Control and Prevention's Protocol for Analysis of Nicotine, Total Moisture and pH in Smokeless Tobacco Products, as published in the Federal Register Vol. 64, No. 55 Mar. 23, 1999 (and as amended in Vol. 74, No. 4, Jan. 7, 2009).

Briefly, approximately 0.5 grams (g) of tobacco is extracted using liquid/liquid extraction into an organic solvent containing an internal standard and analyzed using gas chromatography (GC) with flame ionization detection (FID). Results can be reported as weight percent (Wt %) on either an “as is” or a “dry weight” basis. Reporting data on a dry weight basis requires an oven volatiles (OV) determination. Unless specified otherwise, total or individual alkaloid levels or nicotine levels provided herein are on a milligrams/gram dry weight basis.

Plants are also planted in the field, harvested, and tested for alkaloids and TSNA levels in cured tobacco. Both leaf yield and leaf grade are also assessed for modified plants.

### Example 6. Overexpression of Candidate Genes in Tobacco Hairy Root Cultures or Tobacco Plants

Separate transformation vectors comprising one each of SEQ ID NOs: 59-116 under the control of a CsVMV promoter are constructed using the vector described in Example 4. The vectors are used to (a) transform dark tobacco hairy root cultures using the Agrobacterium rhizogenes line ATCC15834 and/or (b) generate modified tobacco plants as described in Example 4. Control hairy root cultures and control modified tobacco plants are transformed with an “empty” vector that lacks the overexpressed gene (termed “vector controls” or “VC”). Alkaloid levels of tobacco plants are measured as described in Example 5. Alkaloid levels of tobacco root cultures are measured as described in Example 7.

Hairy root cultures can serve as an alternative to plant cell suspensions and serve as useful proxies for whole plants when studying secondary metabolites. See, for example, Zhou et al., Appl. Microbiol Biotechnol., 90:1229-1239 (2011) and Hidalgo et al., Scientific Reports, 7:45331 (2017) for additional information regarding hairy root cultures.

Transformation and hairy root induction is performed as described in Hidalgo et al., Scientific Reports, 7:17976 (2017). Briefly, leaf discs from tobacco plantlets are grown in vitro on MS medium. The leaf discs are co-infected with Agrobacterium rhizogenes line ATCC15834 to introduce the transformation vectors described above and to induce hairy root formation. Approximately two to four weeks after transformation, hairy roots appear in leaf discs grown on solid MS medium comprising 30 grams per liter (g/L) sucrose and 500 milligrams per liter (mg/L) cefotaxime. Hairy roots are then excised and individually cultured on solid MS medium comprising 30 g/L sucrose, 500 mg/L cefotaxime, and 50 mg/L kanamycin to select for hairy root cultures that are successfully transformed. Transformed hairy root cultures are grown in the dark at 25° C. Approximately two to three weeks later, liquid cultures are inoculated with transgenic roots and are grown at 25° C. until they are used in further analyses.

FIGS. 17, 29, 41, and 45 depict the relative expression of g22648, g12941, g14663, and g71598, respectively, as compared to the expression of EF1α (SEQ ID NO: 176) in dark tobacco hairy root cultures in lines over-expressing each gene. FIGS. 18, 30, 42, and 46 depict relative expression of the tobacco quinolinate phosphoribosyltransferase (QPT) and putrescine N-methyltransferase (PMT) gene families as compared to EF1α in the hairy root culture lines overexpressing g22648, g12941, g14663, and g71598, respectively.

### Example 8. Measuring Alkaloid Content in Transformed Tobacco Hairy Root Cultures Overexpressing Candidate Genes

Alkaloid levels are also measured in the hairy root cultures that are overexpressing a candidate gene discussed above in Example 6 and control hairy root cultures comprising an empty vector.

Briefly, to measure alkaloids, hairy root culture tissues are frozen in liquid nitrogen and ground to a fine powder. Approximately 50 mg of the fine powder (per sample) is extracted twice with two volumes of 100% methanol, sonicated for 30 minutes, and then the supernatant is collected and evaporated. Alkaloid levels are quantified via gas chromatography-mass spectrometry (GC/MS) using a QP2010SE quadrupole mass spectrometer (Shimadzu, Japan) following separation using a Zebron ZB-5 (Phenomenex, USA) 30 m stationary phase column with 0.25 mm internal diameter and 0.25 μm film thickness. For each sample, 1 μL of sample is injected into a column with the following settings: split mode 1:10, injection port temperature 250° C. The ZB5 column is maintained at 45° C. for one minute followed by a gradient of 10° C. per minute until the temperature reaches 300° C., which is held for a further seven minutes. Helium inlet pressure is checked by electronic pressure control to achieve a constant column flow rate of 1.0 milliliter per minute. Alkaloids are identified by comparison with reference spectra and the NIST library. Quantities of alkaloids detected are provided as milligrams per gram (mg/g).

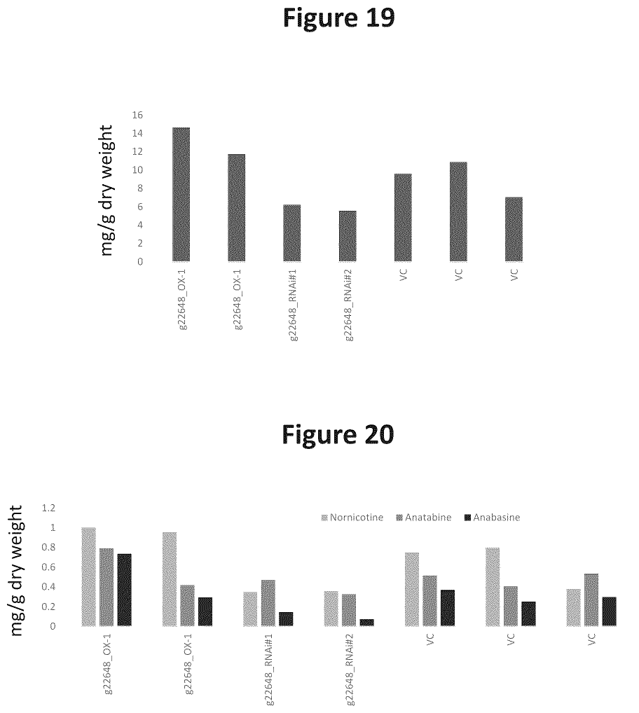

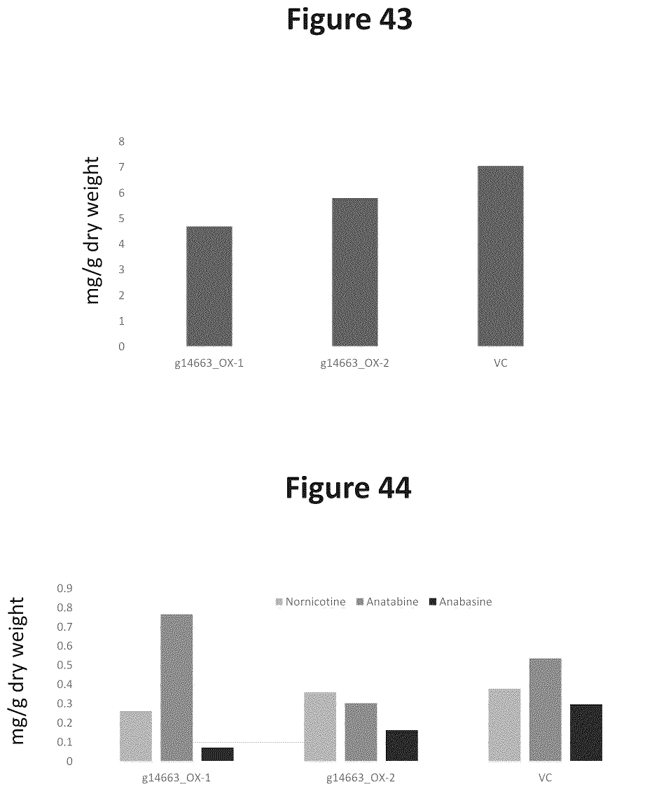

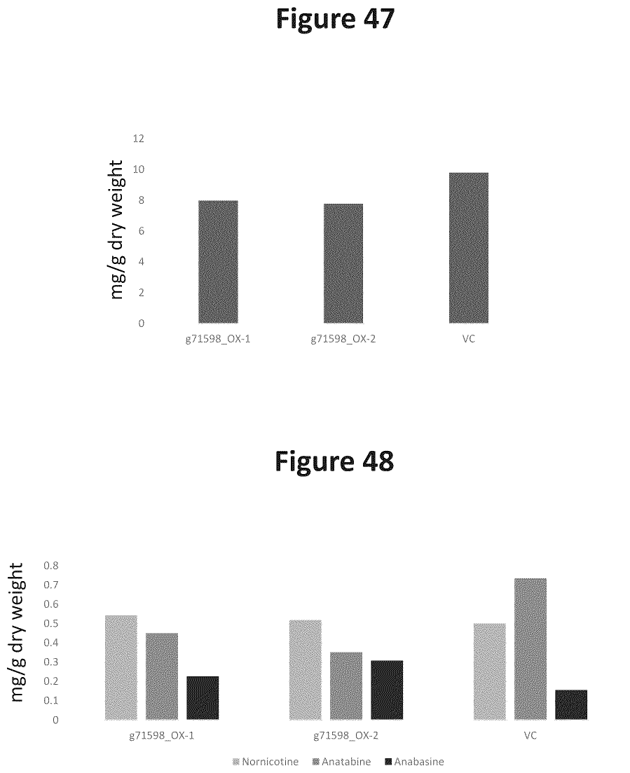

FIGS. 19, 31, 43, and 47 depict average nicotine levels (using three biological replicates per line) in hairy root culture lines overexpressing g22648, g12941, g14663, and g71598, respectively, and corresponding vector controls. FIGS. 20, 32, 44, and 48 depict average nornicotine, anatabine, and anabasine levels (using three biological replicates per line) in hairy root culture lines overexpressing g22648, g12941, g14663, and g71598, respectively, and corresponding vector controls. Table 10 provides average alkaloid levels as shown in FIGS. 19, 20, 31, 32, 43, 44, 47, and 48. Table 11 provides percentage change for each overexpression line as compared to the vector control average (or single vector control if only one vector control is available) for each alkaloid. Percent change compared to control in Table 11 is calculated by using the formula: ((Overexpression Line Level−Control Level)/(Control Level))×100 for each measured alkaloid.

TABLE 10
Alkaloid levels in tobacco plants overexpressing genes of interest.

| Gene | | Alkaloids (mg/g dry weight) | | | |
|---|---|---|---|---|---|
| ID | Expression | Nicotine | Nornicotine | Anatabine | Anabasine |
| g22648 | OX-1 | 14.66 | 1.002 | 0.789 | 0.735 |
| | OX-2 | 11.75 | 0.956 | 0.419 | 0.294 |
| | VC | 10.88 | 0.796 | 0.408 | 0.252 |
| | VC | 9.60 | 0.751 | 0.517 | 0.370 |
| | VC | 7.05 | 0.378 | 0.536 | 0.297 |
| | OX Average | 13.2 | 0.979 | 0.604 | 0.515 |
| | VC Average | 9.17 | 0.642 | 0.487 | 0.306 |
| g12941 | OX-1 | 8.41 | 0.488 | 0.750 | 0.397 |
| | OX-2 | 13.24 | 0.855 | 0.753 | 0.582 |
| | VC | 9.80 | 0.501 | 0.735 | 0.155 |
| | VC | 12.61 | 0.746 | 0.803 | 0.194 |
| | VC | 7.05 | 0.378 | 1.205 | 0.132 |
| | OX Average | 10.825 | 0.672 | 0.752 | 0.49 |
| | VC Average | 9.82 | 0.542 | 0.914 | 0.16 |
| g14663 | OX-1 | 4.70 | 0.264 | 0.766 | 0.072 |
| | OX-2 | 5.80 | 0.360 | 0.303 | 0.163 |
| | VC | 7.05 | 0.378 | 0.536 | 0.297 |
| | OX Average | 5.25 | 0.312 | 0.535 | 0.118 |
| g71958 | OX-1 | 7.99 | 0.544 | 0.450 | 0.227 |
| | OX-2 | 7.78 | 0.520 | 0.352 | 0.308 |
| | VC | 9.80 | 0.501 | 0.735 | 0.155 |
| | OX Average | 7.89 | 0.532 | 0.401 | 0.268 |

OX = overexpression line; VC = vector control.

TABLE 11
Percent change in alkaloids as compared to controls.

| Gene | | Alkaloids (% change compared to control) | | | |
|---|---|---|---|---|---|
| ID | Expression | Nicotine | Nornicotine | Anatabine | Anabasine |
| g22648 | OX-1 | +59.87% | +56.07% | +62.01% | +140.2% |
| | OX-2 | +28.14% | +48.91% | −13.96% | −3.92% |
| g12941 | OX-1 | −14.36% | −9.96% | −17.94% | +148.1% |
| | OX-2 | +34.83% | +57.75% | −17.61% | +263.8% |
| g14663 | OX-1 | −33.33% | −30.16% | +42.91% | −75.76% |
| | OX-2 | −17.73% | −4.76% | −43.47% | −45.12% |
| g71958 | OX-1 | −18.47% | +8.58% | −38.78% | +46.45% |
| | OX-2 | −20.61% | +3.79% | −52.11% | +98.71% |

OX = overexpression line.

### Example 9. Knockdown of Candidate Genes Using Small RNA Molecules in Tobacco

Reducing the expression of genes identified in Table 9 is tested for their effect on the levels of specific alkaloids such as nicotine, nornicotine, anatabine, and anabasine.

Separate transformation vectors comprising an artificial miRNA designed to reduce the transcription or translation of one each of SEQ ID NOs: 59-116 driven by CsVMV are constructed using the vector described in Example 4. Tobacco lines (plants or hairy root cultures) expressing these RNAi constructs can be collectively referred to as “knockdown lines.”

The vectors are used to (a) transform dark tobacco hairy root cultures using the Agrobacterium rhizogenes line ATCC15834 as described in Example 6 and/or (b) generate modified tobacco plants as described in Example 4. Control hairy root cultures and control modified tobacco plants are transformed with an “empty” vector that lacks the miRNA constructs (referred to as “vector controls” or “VC”). Alkaloid levels of transformed tobacco hairy root cultures are measured as described in Example 8. Alkaloid levels of transformed tobacco plants are measured as described in Example 5.

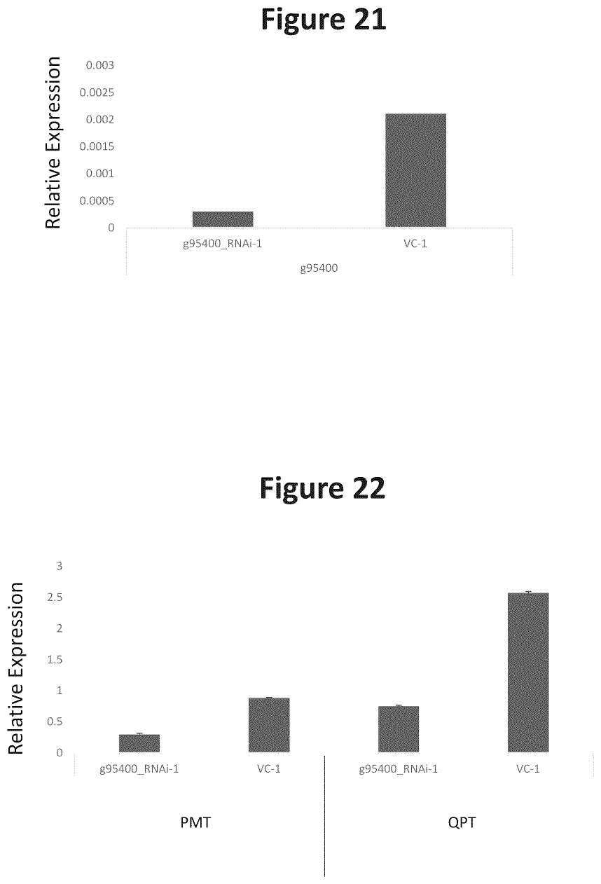

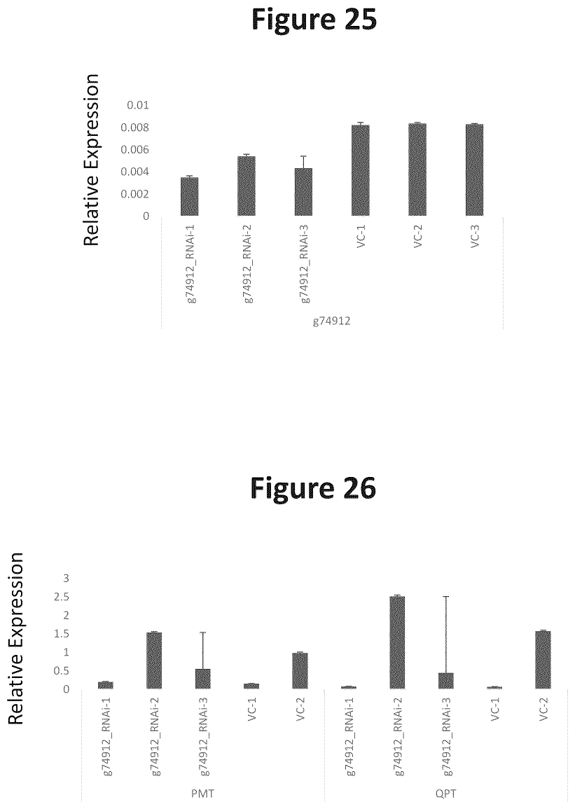

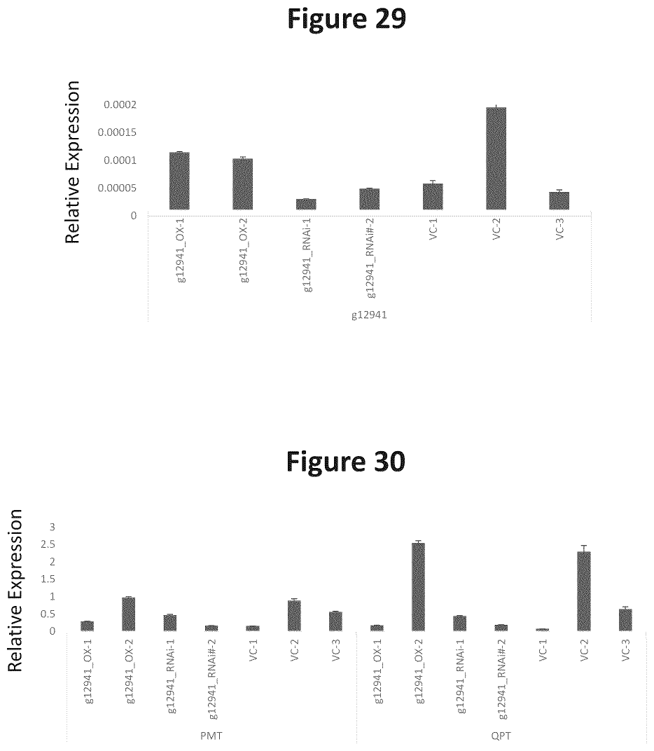

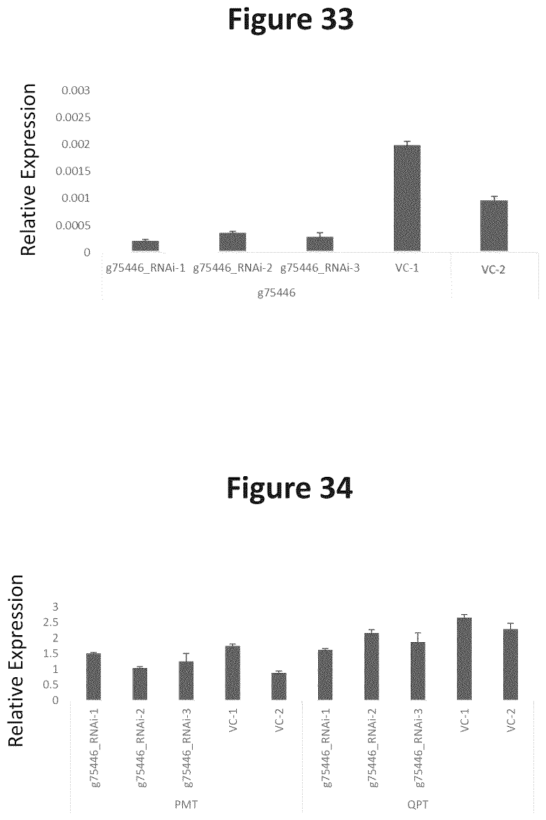

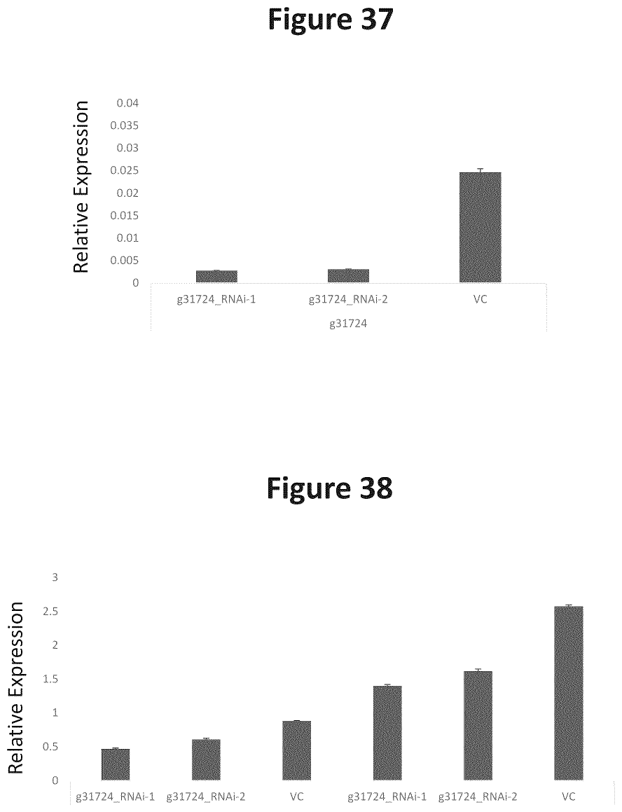

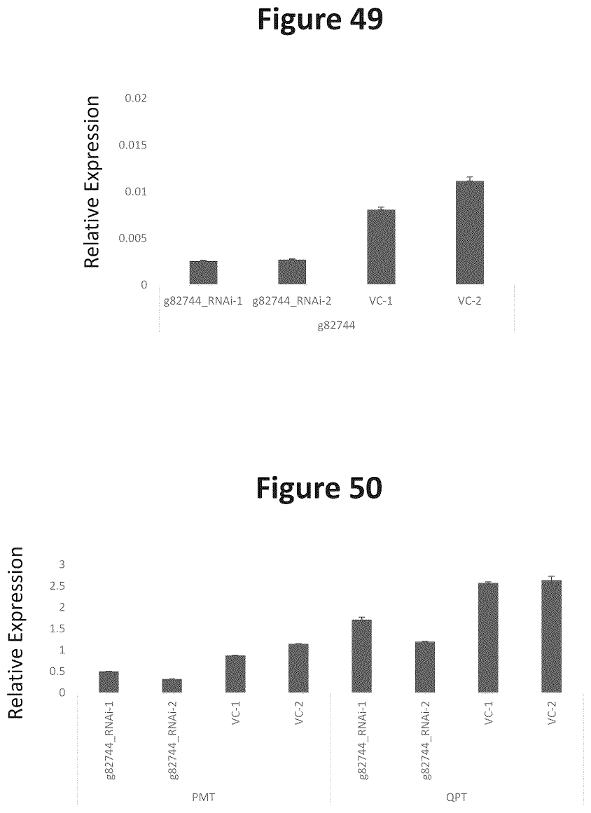

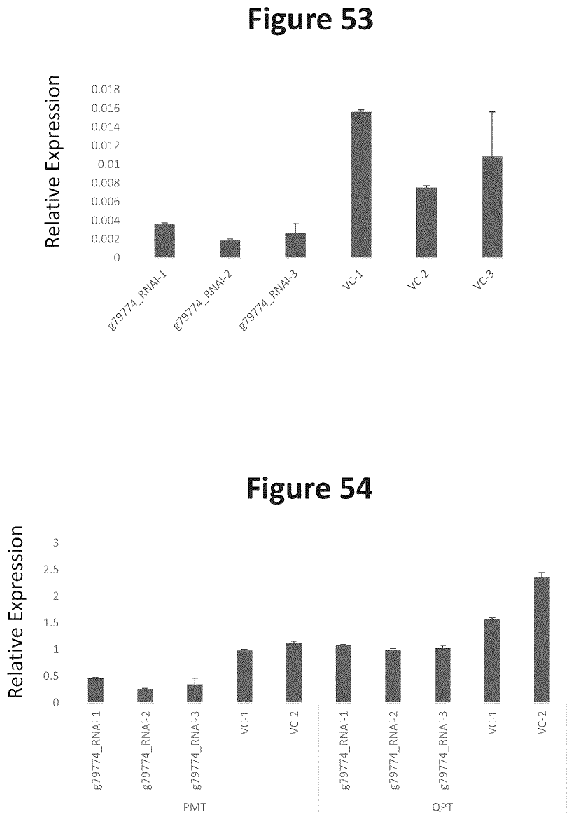

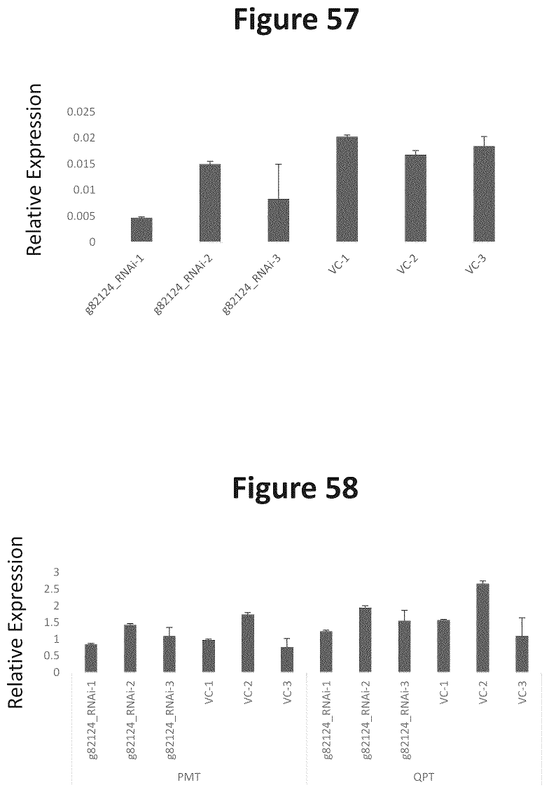

FIG. 17 depicts relative expression of g22648 relative to EF1α in hairy root culture knockdown lines as compared to vector control lines. FIG. 21 depicts relative expression of g95400 relative to EF1α in a hairy root culture knockdown line as compared to a vector control line. FIG. 25 depicts relative expression of g74912 relative to EF1α in hairy root culture knockdown lines as compared to vector control lines. FIG. 29 depicts relative expression of g12941 relative to EF1α in hairy root culture knockdown lines as compared to vector control lines. FIG. 33 depicts relative expression of g75446 relative to EF1α in hairy root culture knockdown lines as compared to vector control lines. FIG. 37 depicts relative expression of g31724 relative to EF1α in hairy root culture knockdown lines as compared to vector control lines. FIG. 49 depicts relative expression of g82744 relative to EF1α in hairy root culture knockdown lines as compared to vector control lines. FIG. 53 depicts relative expression of g79774 relative to EF1α in hairy root culture knockdown lines as compared to vector control lines. FIG. 57 depicts relative expression of g82124 relative to EF1α in hairy root culture knockdown lines as compared to vector control lines.

The relative expression of both PMT and QPT gene families is also measured in each of the knockdown lines. FIG. 18 depicts relative expression of PMT and QPT gene families relative to EF1α in hairy root culture g22648 knockdown lines as compared to vector control lines. FIG. 22 depicts relative expression of PMT and QPT gene families relative to EF1α in a hairy root culture g25400 knockdown line as compared to a vector control line. FIG. 26 depicts relative expression of PMT and QPT gene families relative to EF1α in hairy root culture g74912 knockdown lines as compared to vector control lines. FIG. 30 depicts relative expression of PMT and QPT gene families relative to EF1α in hairy root culture g12941 knockdown lines as compared to vector control lines. FIG. 34 depicts relative expression of PMT and QPT gene families relative to EF1α in hairy root culture g75446 knockdown lines as compared to vector control lines. FIG. 38 depicts relative expression of PMT and QPT gene families relative to EF1α in hairy root culture g31724 knockdown lines as compared to vector control lines. FIG. 50 depicts relative expression of PMT and QPT gene families relative to EF1α in hairy root culture g82744 knockdown lines as compared to vector control lines. FIG. 54 depicts relative expression of PMT and QPT gene families relative to EF1α in hairy root culture g79774 knockdown lines as compared to vector control lines. FIG. 58 depicts relative expression of PMT and QPT gene families relative to EF1α in hairy root culture g82124 knockdown lines as compared to vector control lines.

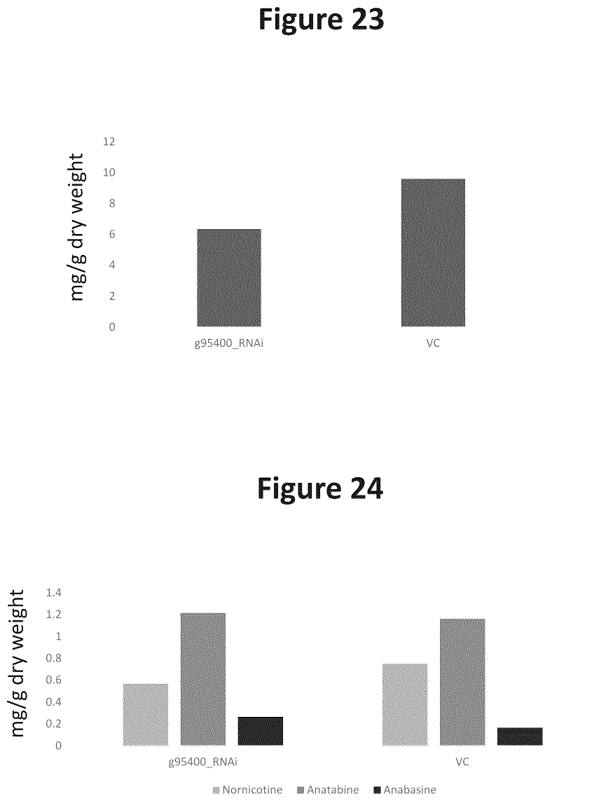

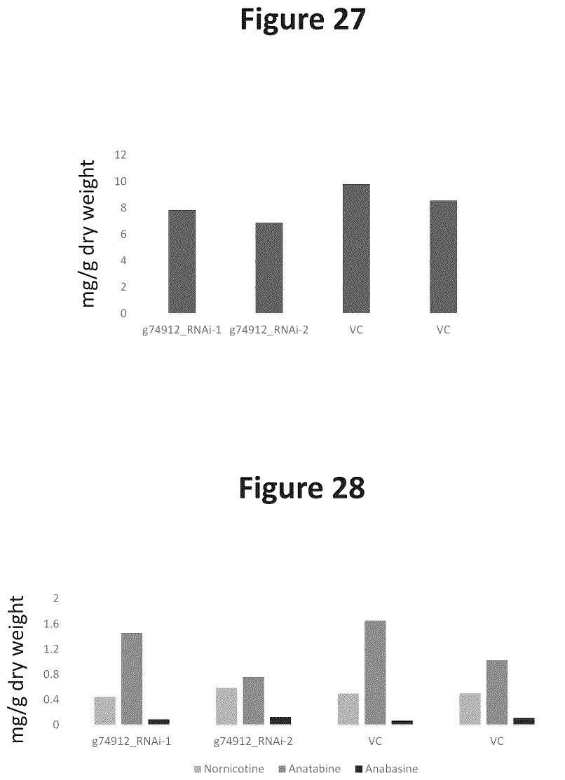

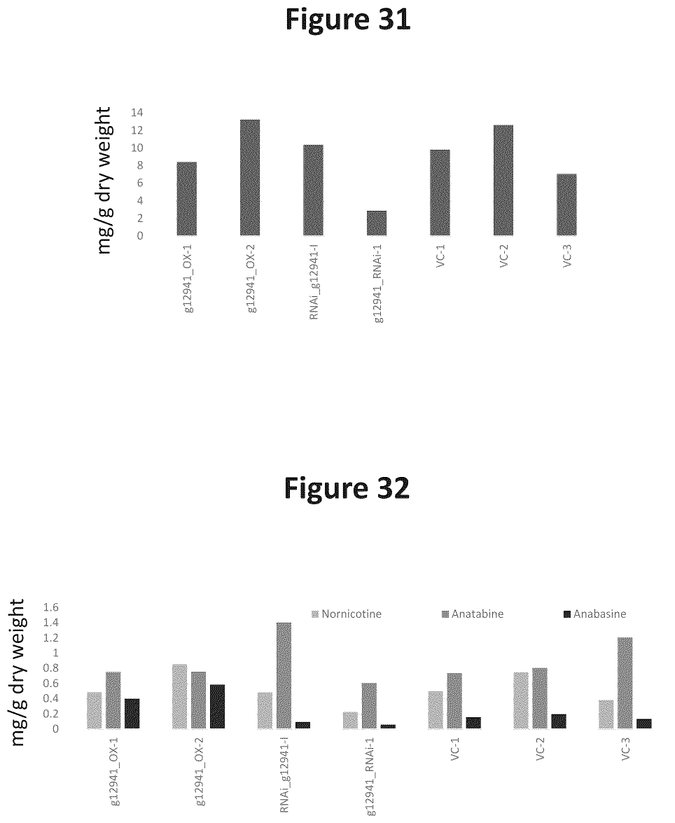

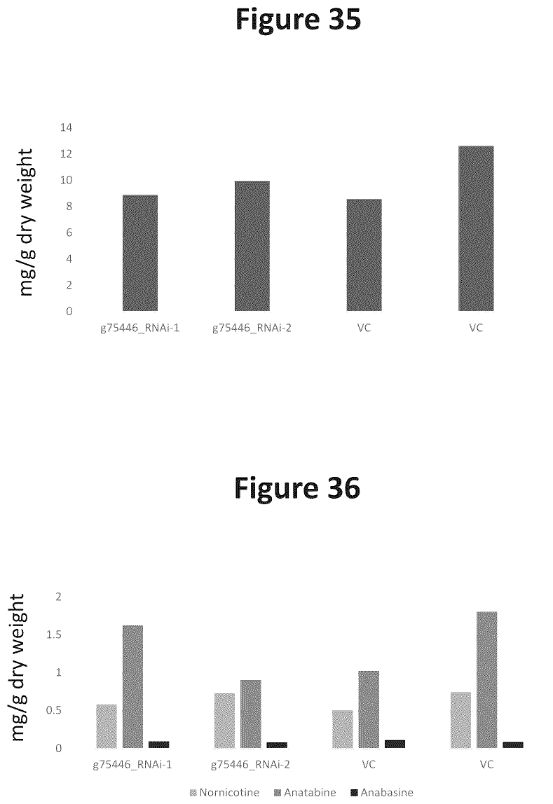

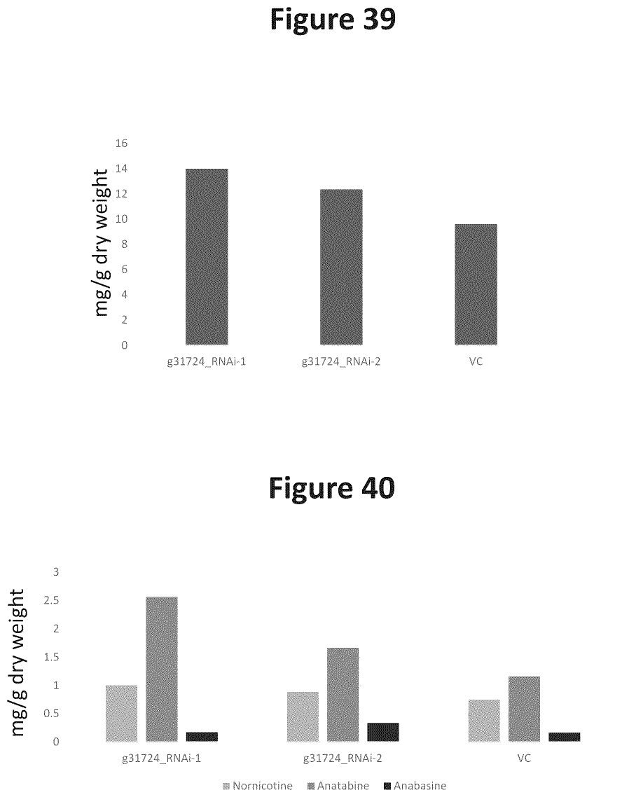

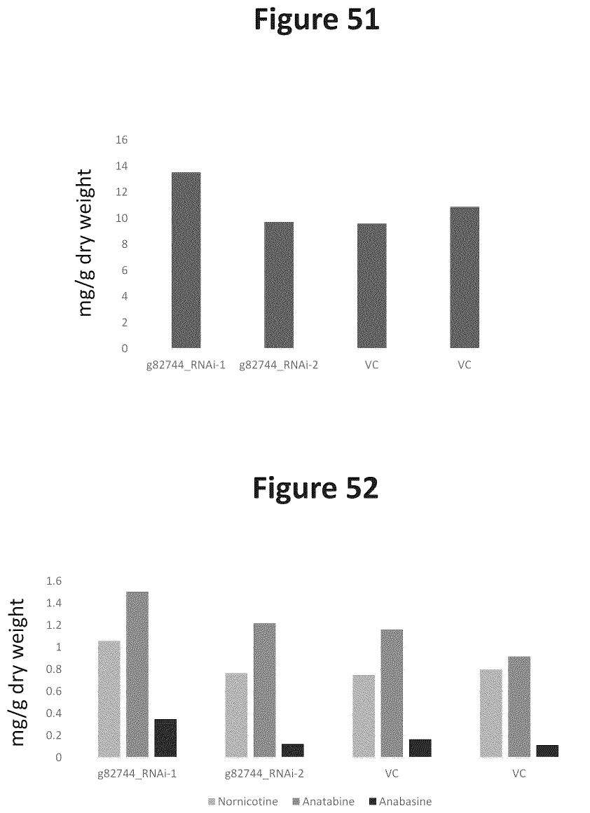

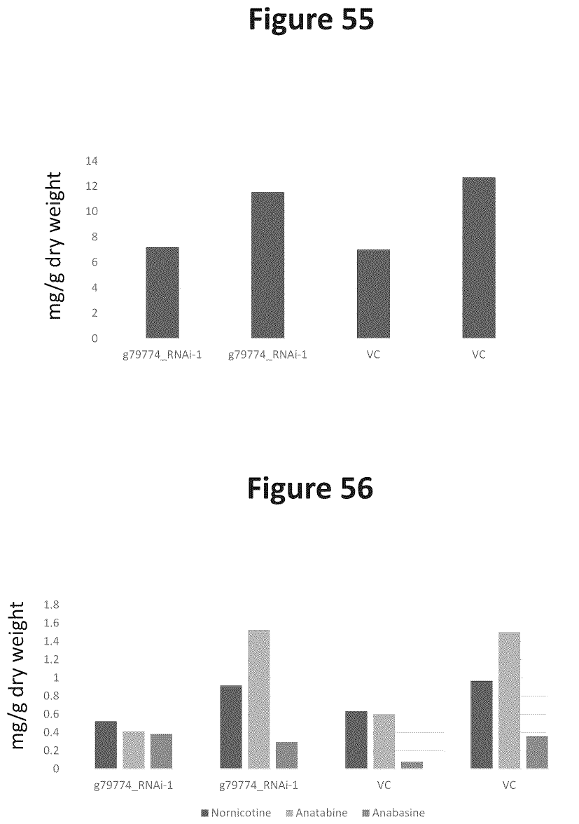

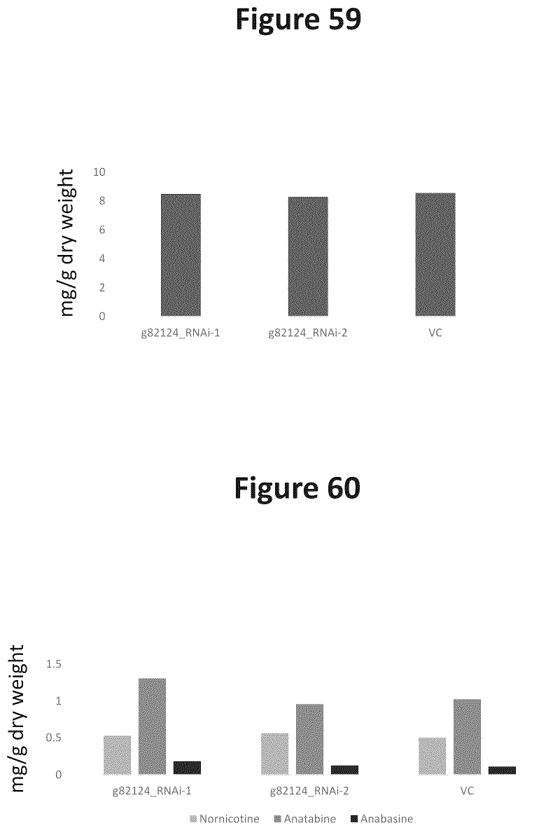

Alkaloid levels (e.g., nicotine, nornicotine, anatabine, anabasine) are measured in hairy root culture knockdown lines as described in Example 8. FIG. 19 depicts nicotine levels in hairy root culture g22648 knockdown lines as compared to vector control lines, and FIG. 20 depicts nornicotine, anatabine, and anabasine levels in hairy root culture g22648 knockdown lines as compared to vector control lines. FIG. 23 depicts nicotine levels in a hairy root culture g95400 knockdown line as compared to a vector control line, and FIG. 24 depicts nornicotine, anatabine, and anabasine levels in a hairy root culture g95400 knockdown line as compared to a vector control line. FIG. 27 depicts nicotine levels in hairy root culture g74912 knockdown lines as compared to vector control lines, and FIG. 28 depicts nornicotine, anatabine, and anabasine levels in hairy root culture g74912 knockdown lines as compared to vector control lines. FIG. 31 depicts nicotine levels in hairy root culture g12941 knockdown lines as compared to vector control lines, and FIG. 32 depicts nornicotine, anatabine, and anabasine levels in hairy root culture g12941 knockdown lines as compared to vector control lines. FIG. 35 depicts nicotine levels in hairy root culture g75446 knockdown lines as compared to vector control lines, and FIG. 36 depicts nornicotine, anatabine, and anabasine levels in hairy root culture g75446 knockdown lines as compared to vector control lines. FIG. 39 depicts nicotine levels in hairy root culture g31724 knockdown lines as compared to vector control lines, and FIG. 40 depicts nornicotine, anatabine, and anabasine levels in hairy root culture g31724 knockdown lines as compared to vector control lines. FIG. 51 depicts nicotine levels in hairy root culture g82744 knockdown lines as compared to vector control lines, and FIG. 52 depicts nornicotine, anatabine, and anabasine levels in hairy root culture g82744 knockdown lines as compared to vector control lines. FIG. 55 depicts nicotine levels in hairy root culture g79774 knockdown lines as compared to vector control lines, and FIG. 56 depicts nornicotine, anatabine, and anabasine levels in hairy root culture g79774 knockdown lines as compared to vector control lines. FIG. 59 depicts nicotine levels in hairy root culture g82124 knockdown lines as compared to vector control lines, and FIG. 60 depicts nornicotine, anatabine, and anabasine levels in hairy root culture g82124 knockdown lines as compared to vector control lines.

Table 12 provides average alkaloid levels as shown in FIGS. 19, 20, 23, 24, 27, 28, 31, 32, 35, 36, 39, 40, 51, 52, 55, 56, 59, and 60. Table 13 provides percentage change for each overexpression line as compared to the vector control average (or single vector control if only one vector control is available) for each alkaloid. Percent change compared to control in Table 13 is calculated by using the formula: ((Overexpression Line Level−Control Level)/(Control Level))×100 for each measured alkaloid.

TABLE 12
Alkaloid levels in tobacco knockdown lines.

| Gene | | Alkaloids (mg/g dry weight) | | | |
|---|---|---|---|---|---|
| ID | Expression | Nicotine | Nornicotine | Anatabine | Anabasine |
| g22648 | RNAi-1 | 6.22 | 0.351 | 0.471 | 0.144 |
| | RNAi-2 | 5.57 | 0.357 | 0.327 | 0.073 |
| | VC | 10.88 | 0.796 | 0.408 | 0.252 |
| | VC | 9.60 | 0.751 | 0.517 | 0.370 |
| | VC | 7.05 | 0.378 | 0.536 | 0.297 |
| | RNAi | 5.9 | 0.354 | 0.399 | 0.109 |
| | Average | | | | |
| | VC Average | 9.17 | 0.642 | 0.487 | 0.306 |
| g95400 | RNAi-1 | 6.35 | 0.568 | 1.215 | 0.266 |
| | VC | 9.60 | 0.751 | 1.161 | 0.165 |
| g74912 | RNAi-1 | 7.84 | 0.448 | 1.460 | 0.087 |
| | RNAi-2 | 6.89 | 0.589 | 0.759 | 0.126 |
| | VC | 9.80 | 0.501 | 1.652 | 0.069 |
| | VC | 8.57 | 0.505 | 1.026 | 0.111 |
| | RNAi | 7.365 | 0.519 | 1.11 | 0.107 |
| | Average | | | | |
| | VC Average | 9.19 | 0.503 | 1.339 | 0.09 |
| g12941 | RNAi-1 | 10.38 | 0.484 | 1.401 | 0.093 |
| | RNAi-2 | 2.90 | 0.227 | 0.606 | 0.057 |
| | VC | 9.80 | 0.501 | 0.735 | 0.155 |
| | VC | 12.61 | 0.746 | 0.803 | 0.194 |
| | VC | 7.05 | 0.378 | 1.205 | 0.132 |
| | RNAi | 6.64 | 0.356 | 1.00 | 0.075 |
| | Average | | | | |
| | VC Average | 9.82 | 0.542 | 1.248 | 0.16 |
| g75446 | RNAi-1 | 8.89 | 0.584 | 1.625 | 0.094 |
| | RNAi-2 | 9.92 | 0.733 | 0.905 | 0.082 |
| | VC | 8.57 | 0.505 | 1.026 | 0.111 |
| | VC | 12.61 | 0.746 | 1.804 | 0.086 |
| | RNAi | 9.405 | 0.659 | 1.265 | 0.088 |
| | Average | | | | |
| | VC Average | 10.59 | 0.626 | 1.415 | 0.099 |
| g31724 | RNAi-1 | 13.98 | 1.001 | 2.565 | 0.172 |
| | RNAi-2 | 12.36 | 0.890 | 1.666 | 0.335 |
| | VC | 9.60 | 0.751 | 1.161 | 0.165 |
| | RNAi | 13.17 | 0.946 | 2.116 | 0.254 |
| | Average | | | | |
| g82744 | RNAi-1 | 13.53 | 1.060 | 1.505 | 0.348 |
| | RNAi-2 | 9.71 | 0.764 | 1.218 | 0.123 |
| | VC | 9.60 | 0.751 | 1.161 | 0.165 |
| | VC | 10.88 | 0.796 | 0.915 | 0.112 |
| | RNAi | 11.62 | 0.912 | 1.362 | 0.236 |
| | Average | | | | |
| | VC Average | 10.24 | 0.774 | 1.038 | 0.139 |
| g79774 | RNAi-1 | 7.20 | 0.523 | 0.416 | 0.381 |
| | RNAi-2 | 11.55 | 0.915 | 1.528 | 0.296 |
| | VC | 7.02 | 0.633 | 0.601 | 0.078 |
| | VC | 12.71 | 0.965 | 1.502 | 0.358 |
| | RNAi | 9.375 | 0.719 | 0.972 | 0.339 |
| | Average | | | | |
| | VC Average | 9.865 | 0.799 | 1.052 | 0.218 |
| g82124 | RNAi-1 | 8.50 | 0.533 | 1.308 | 0.183 |
| | RNAi-2 | 8.30 | 0.567 | 0.957 | 0.125 |
| | VC | 8.57 | 0.505 | 1.026 | 0.111 |
| | RNAi | 8.40 | 0.55 | 1.133 | 0.154 |
| | Average | | | | |

RNAi = knockdown line; VC = vector control.

TABLE 13
Percent change in alkaloids as compared to controls.

| Gene | Expres- | Alkaloids (% change compared to control) | | | |
|---|---|---|---|---|---|
| ID | sion | Nicotine | Nornicotine | Anatabine | Anabasine |
| g22648 | RNAi-1 | −32.17% | −45.33% | −3.29% | −52.94% |
| | RNAi-2 | −39.26% | −44.39% | −32.85% | −76.14% |
| g95400 | RNAi-1 | −33.85% | −24.37% | +4.65% | +61.21% |
| g74912 | RNAi-1 | −14.69% | −10.93% | +9.04% | −3.33% |
| | RNAi-2 | −25.03% | +17.10% | −43.32% | +40% |
| g12941 | RNAi-1 | +8.76% | −10.70% | +12.26% | −41.88% |
| | RNAi-2 | −70.47% | −58.12% | −51.44% | −64.38% |
| g75446 | RNAi-1 | −16.05% | −6.71% | +14.84% | −5.05% |
| | RNAi-2 | −6.33% | −17.09% | −36.04% | −17.17% |
| g31724 | RNAi-1 | +45.63% | +33.29% | +120.93% | +4.24% |
| | RNAi-2 | +28.75% | +18.51% | +43.50% | +103.03% |
| g82744 | RNAi-1 | +32.13% | +36.95% | +44.99% | +150.36% |
| | RNAi-2 | −5.18% | −1.29% | +17.34% | −11.51% |
| g79774 | RNAi-1 | −27.01% | −34.54% | −60.46% | +74.77% |
| | RNAi-2 | +17.08% | +14.52% | +45.25% | +35.78% |
| g82124 | RNAi-1 | −0.82% | +5.54% | +27.49% | +64.86% |
| | RNAi-2 | −3.15% | +12.28% | −6.73% | +12.61% |

RNAi = knockdown line.

### Example 10. Generating Mutations in Candidate Genes

Mutations are produced in each of the genes identified in Table 9 by specifically editing SEQ ID NOs: 1-58, separately and individually, in the tobacco genome. Tobacco protoplasts are transfected using polyethylene glycol (PEG) with plasmids encoding a CRISPR protein or a CRISPR protein and specific guide RNA (gRNA) targeting individual genes at desired positions.

Transfected protoplasts are then immobilized in 1% agarose beads and subjected to tissue culture. When calli grow to approximately 1 millimeter in diameter, they are spread on TOM2 plates. Calli are screened for mutations (e.g., insertions or deletions (indels)) at the target positions using fragment analysis. Candidates, showing size shifts compared to wildtype control, are selected for further culture and the consequent shoots are tested by fragment analysis again to confirm the presence of mutations.

Modified tobacco plants (TO generation) are grown as described in Example 4. Then, plants are topped, and alkaloid levels are measured as described in Example 5.

1. A modified tobacco plant, or part thereof, comprising at least one non-natural mutation in an endogenous nucleic acid sequence, wherein the endogenous nucleic acid sequence encodes a polypeptide comprising an amino acid sequence at least 80% identical or similar to an amino acid sequence selected from the group consisting of SEQ ID NOs: 117-174.

2. (canceled)

3. A modified tobacco plant, or part thereof, comprising a recombinant DNA construct comprising a heterologous promoter operably linked to a nucleic acid sequence encoding a polypeptide comprising an amino acid sequence at least 80% identical or similar to an amino acid sequence selected from the group consisting of SEQ ID NOs: 117-174.

4. The modified tobacco plant, or part thereof, of claim 1, wherein said tobacco plant produces at least one leaf comprising a reduced amount of at least one alkaloid as compared to the amount of said alkaloid in a control tobacco plant lacking said at least one non-natural mutation in said endogenous nucleic acid sequence when grown under comparable conditions.

5. The modified tobacco plant, or part thereof, of claim 3, wherein said tobacco plant produces at least one leaf comprising a reduced amount of at least one alkaloid as compared to the amount of said alkaloid in a control tobacco plant lacking said recombinant DNA construct when grown under comparable conditions.

6. The modified tobacco plant, or part thereof, of claim 1, wherein said endogenous nucleic acid sequence comprises a nucleic acid sequence at least 80% identical or complementary to a sequence selected from the group consisting of SEQ ID NOs: 1-58.

7. The modified tobacco plant, or part thereof, of claim 3, wherein said nucleic acid sequence comprises a nucleic acid sequence at least 80% identical or complementary to a sequence selected from the group consisting of SEQ ID NOs: 1-58.

8. The modified tobacco plant, or part thereof, of claim 1, wherein said endogenous nucleic acid sequence comprises a nucleic acid sequence at least 80% identical or complementary to a sequence selected from the group consisting of SEQ ID NOs: 59-116.

9. The modified tobacco plant, or part thereof, of claim 3, wherein said nucleic acid sequence comprises a nucleic acid sequence at least 80% identical or complementary to a sequence selected from the group consisting of SEQ ID NOs: 59-116.

10. (canceled)

11. (canceled)

12. The modified tobacco plant, or part thereof, of claim 4, wherein said at least one alkaloid is selected from the group consisting of anabasine, anatabine, nicotine, and nornicotine.

13.-18. (canceled)

19. The modified tobacco plant, or part thereof, or part thereof, of claim 1, wherein said at least one non-natural mutation results in a reduced level of expression of said nucleic acid sequence as compared to expression of said nucleic acid sequence in the same tissue of a control tobacco plant when grown under comparable conditions, wherein said nucleic acid sequence lacks the at least one non-natural mutation in said control tobacco plant.

20. (canceled)

21. The modified tobacco plant, or part thereof, of claim 3, wherein said promoter comprises a promoter selected from the group consisting of a constitutive promoter, a tissue-preferred promoter, a tissue-specific promoter, and an inducible promoter.

22.-27. (canceled)

28. The modified tobacco plant, or part thereof, of claim 1, wherein the modified tobacco plant is of a tobacco variety selected from the group consisting of a flue-cured variety, a bright variety, a Burley variety, a Virginia variety, a Maryland variety, a dark variety, a Galpão variety, an Oriental variety, and a Turkish variety.

29.-32. (canceled)

33. Cured tobacco material from the modified tobacco plant, or part thereof, of claim 1.

34. (canceled)

35. The cured tobacco material of claim 33, wherein said cured tobacco material comprises flue cured tobacco material, air cured tobacco material, fire cured tobacco material, and sun cured tobacco material.

36. A tobacco blend comprising the cured tobacco material of claim 33.

37. (canceled)

38. (canceled)

39. A tobacco product comprising the tobacco blend of claim 36.

40. A tobacco product comprising the cured tobacco material of claim 33.

41. The tobacco product of claim 40, wherein said tobacco product is selected from the group consisting of a cigarette, a heated tobacco product, a kretek, a bidi cigarette, a cigar, a cigarillo, a non-ventilated cigarette, a vented recess filter cigarette, pipe tobacco, snuff, snus, chewing tobacco, moist smokeless tobacco, fine cut chewing tobacco, long cut chewing tobacco, pouched chewing tobacco product, gum, a tablet, a lozenge, and a dissolving strip.

42. The tobacco product of claim 40, wherein said tobacco product is a smokeless tobacco product.

43. The tobacco product of claim 42, wherein said smokeless tobacco product is selected from the group consisting of loose leaf chewing tobacco, plug chewing tobacco, moist snuff, nasal snuff, dry snuff, and snus.

44.-123. (canceled)


SEQUENCE LISTING (178 sequences)

SEQ ID NO: 1 - DNA, 1140 bp, synthetic construct
  source 1..1140: mol_type = other DNA
atgtcacatctcactgcagctccgccatcagaaccaccgtcggcggcggtgaattcattgtacaaacagaaatcgtggtcgccggacacgtttcgcgatgaggcgtggcagcggcggaagggtacccatggaagccgcttaaagcagcggagcaagagcgttaccgatgaagacctcgatgagctcaaggcttgtattgagttagggtttggatttgactcgcctgaaatagatcaacgattgtctgatactttcccggcttatggccttttttacgctgttaataaacaatatgccgacaccctttcgaagacttcttctttatcgtcggtcatctcctattgcgagtcacccgctcctcccggcagtccccacaccattgtcgatcaaggtaattcgataaaagcctctcctttacaatttttattcgtactcttttattttagaatgttctgaaatgcttaacacaaattccatttcttttgaactatccttcactcagaaaatccaaattcattcatctaatcaaaatgaaatttgattttaaattctaatatgaaatcctctatgtgaaattaactttcaattataatgtcttttacaatcagtaaatactactcctatataagttaagttctatatatttaacttattgccctgtcataaaaaaagagattaccagataatttaaccttaaaagtttgttaatgtaaagaacagttgagtgcctatgggaactctccgcccttaactaaaggtcttgagtttaagccctatgaaagaagaaactcttgatagagaatgcttccctcatagtggttctacgggcggctaatctagattagttggacaaatgaatttagattaccggatgaatagatgaaaaaagaaaaaaaggatgcctatgttttatcttttgttcaatgcacaatttagttttgcctacaccccttccccattttttttgggggggatgagtggtggtggggacaagattcatataaagaactatagtgttgaacgtatggttgctaatatttgcaaaaaatgaatgtaggagataatcctcagacagtgaagacaaggttgcggcagtgggcacaagtggtcgcttggtcagttcgtcaatcttcacattaa

SEQ ID NO: 2 - DNA, 2245 bp, synthetic construct
  source 1..2245: mol_type = other DNA
atggagaaaaagcagctggatttcaatgcaccacttttatcagtgaggaaaatttcatcatctttatcccctcatgagagagcaaataaaaagattatagaaagggcgccaccgaatcgacaacaatcacttcctgtcaaaaaatcagattgggaattgagtgaggtgactaaaccagtagctgttccatttatgtgggaacagatccctggaagacgaaaaggtgacaatgaagcccgagctaaccttcgagtggagtggtcaagtagtcctaggctgcccccgggaagattgccagagactattcggttctattcgggtgaaaggcctcgtactcaaaacatttacaagtctccagctgaaggacttccttggattgatcatgcagctttacttgatagcctagcggaaagtatatatacaagaggagacagagcaagtgaggatgatgccaattctgatgctcctgaaacattgtcacctaccgaatccttatccttggactgcagtgtcagtggtttgagcggacatcaaagttcagattcaaaaccatctggaaccttttccattgactcccaaactagagactttatgatgagtcgattcttacctgcagcaaaagctgttgttttggagacgcctcagtatgttcaaaaaaagcaagttccagtcagtactgaacaacccaagccagtacctgtggaacgaaagccaatagttaagcagatggagtctaaacctgtctcatattatagcggctatccagacgatgtaggaagtgaaattgaagatgatgtgtctgagaatcagcacaaaagaccaagtaaagggtggaaatttttccctcggatttgtgtgaagaattctttatgcctattaaatccactgccaggactaaaagtgaagacacatgttcccacaccctcagctcaagcagtaaagagagtttcaggaatgaaaccaaagacgccacagagccccacatcttatgctcatgaagtcaagagattagctagaaaagcttatagtggacctcttgagaaggtaataattataattttcctcagtttctttttttctgtcctaagattaataagatttatgtttgtttctaacaatcatgttctttgcagaatacttgtgatactataaataagcaaagatttcactctggagtactgtctcgcgagctgtacaaagctgaaaatagaagtttttccggccaactgcctaaccctagtgattcatgtaagctagttggaatctctcctggaagacgttcaagaagtggggctatatctccctgtcgaaatgtagcacccccatctccattcaatgaaggtactaggtttcttggtgtgccaaaggaaatggagagtctttgggctagccgctttgattcattccggaaaggttgctacactgtcaaggacaaagtaccacagcagattggcacaggaaggttttctgattcaccaagtgaagtagttgagaaaacattgtacatagattctgtagataatgtgcaaatttcagcccgtaattctgcttcttcaaagcccaaggaattggtgaactcctctggtaagaatatgaagccactggttaaaagcagtaaagtactagaaaatatggatgccaccgctcgtactcaaggtgccaaagaccggaatgtttcagagaaagagagcaaacaggtttttgagaaggagtcccttgatttggtggaagcttctcccatttccatatcaacacgcaaaggccctgctgatcatgagtctttgaaactaaagcaaaatcttgatacactatctggagccttagagtcttcaaaagtgcatccctatggaaatttaggtacagaaaatgaagataaccaaaatgcaaatggcccaaaagattctaaccttacctccttggaatctcctataccaccacccttacctaaatctccctctgaatcttggctttggcgcacgctgccttccatccctttgagaactccgttctcaagtttgagctccaagaagcagaataagaagtcccatgctgatggtagtaagtgggagactattgtgaaaacttctaacttgcataaggatcatgtccgttactccgaggtaacctcttttttcttttataccttttgaagttttctgttgcaaattttgatgtttacttctgctccattttgtttgaacaggaactatatactcttggttcttgtcagcagagcaaagcttga

SEQ ID NO: 3 - DNA, 2431 bp, synthetic construct
  source 1..2431: mol_type = other DNA
atggaagtcatatctaccaacacaaatggctcgaccatcttcaagaatggtgccattcccatgaatggccaccagagtggcacttccaaacacctcaacggctaccagaacggcacttccaaacaccaaaacggccaccataatggcacttccgaacatcggaacggccaccagaatgggatttccgaacaccaaaacggccaccagaatgggacttccgaacatcggaacggccaccagaatgggatttccgaacaccaaaacggccaccagaatgggacttccgaacaccaaaacggccaccagaatgggacttccgaacaacagaacgggacaatcagccatgacaatggcaacgagctactgggaaactccaactctattaagcttggttggttttcagagtttagcgcattatggccaggttagtactgagaaagaaactcaaattcatatttaaagttaaaattgttaggctaatataagaagttgattttcttttagtgattaattaaaaaaggaaagagtatcaaataaattccaaaaaatgaccagtaacttcgcatattattctacacattaatttgaaataaatcgaattttgcaggtgaagcattctcccttaaggttgagaagttactatttcaggggaagtctgactaccaagatgtcatgctctttgaggtaaataatattctaatacacatgctttaatatgaataaatacttttaatttacttttagtttattgcacgtgtacgtacagtcagcaacatatgggaaggttttgactttggatggagcaattcaacacacagagaatggtggatttccatacactgaaatgattgttcatcttccacttggttccatcccaaacccaaaaaaggttttgatcatcggcggaggaattggttttacattattcgaaatgcttcgttatcctacaatcgaaaaaattgacattgttgaaatcgatgacgtggtagttgatgtaagtcaaatttcttttactcacataaaaaaatgatttagattgcttctttttatttttctaaaagaataaatatattctctcttagttttaaacaaaattctctttcttacaggtatctagaaaatctttcccttatctcgcagctaattttaatgatcctcgtgtaaccctcgttctcggagatggtgcgtatttataatctcgtttttgttttatcttttatttttatttcatttaatttacctttttgtgtgtggttaatttacccgtcattggttctctttcatttcaggggctgcatttgtaaaggctgcacaagcaggatattatgatgctattatagtggactcttctgatcccattggtactctattactacttaataccaagactaatcttattgaataagctactaataaactgtaattgatttctaaaataatataatttcaggtccagcaaaagatttgtttgagaggccattctttgaggcagtagccaaagccctaaggccaggaggagttgtatgcacacaggccgaaagcatttggcttcatatgcatattattaagcaaatcattgctaactgtcgtcaagtctttaagggctctgtcaactacgcttggactactgttccaacatatcccacgtattttctctctctctctcttcatctttgaaaattgaaaatcctgactactttccttcctttgattcctcggttaaaggggcgtagatcataagattttcaagaaatagataatgacgtccaagaaaaactaacttcttttcatttactattctttttggtgacaaactttatttattatttcgttctaaagagaaaatttatttttatattttaaaataattttgttttaaacttttatttttacttattatatctttaataaaaaaattatagtcaaataaatattatggccacactaaacatccaagtttttgaaaccataagttttagagccaaatgagttaatttgtttttggtatgcgggtgcggagtcaaattatgtcacaaaaattgtaatggagtgagcaaatttttatttcgagtaaactttcaaggtattgtgttaaagttttttcaactgatactaatcaattatgtctcaaccattttgatttcagtggtgtaattgggtatatgctctgctctactgaagggccagaagttgacttcaagaatccaataaatccaattgacaaagagacaactcaagtcaagtccaaattagcacctctcaagttttacaattctgatgtaacttcatatctaacaatttctttttctgttttactgtatcttcattgtcaaaattttataattaactcttctcaaattatctttttttttagattcacaaagcagcattcattttgccatctttcgccagaagtatgatcgagtcttaa

SEQ ID NO: 4 - DNA, 1894 bp, synthetic construct
  source 1..1894: mol_type = other DNA
atggaagtcatatctaccaacacaaatggctctaccatcttcaagagtggtgccattcccatgaatggccaccataatggcacttccaaacaccaaaacggccacaagaatgggacttccgaacaacagaacgggacaatcagccttgataatggcaacgagctactgggaaactccaattgtattaagcctggttggttttcagagtttagcgcattatggccaggttagtactgagaaagaaactcaaatgcatatttaaagttaaaattgttaggctaatataaggagttgatattcttttagtgattaattaaaaaggaaaaagtatcaaataaattcaaaaaatggatagtaacttcgcatattactctacacattaatttgaaataaatcgaattttgcaggtgaagcattctcacttaaggttgagaagttactgttccaggggaagtctgactaccaagatgtcatgctctttgaggtaaataatattttaatacacatgcttccatttaaattgatacttttaatttacttttactttattgcatgtgtacgtacagtcagcaacttatgggaaggttctgactttggatggagcaattcaacacacagagaatggtggatttccatacactgaaatgattgttcatcttccacttggttccatcccaaacccaaaaaaggttttgatcatcggcggaggaattggttttacattattcgaaatgcttcgttatcctacaatcgaaaaaattgacattgttgagatcgatgacgtggtagttgatgtaagtcaaacttcttttactcacataaaaaaatggtttagattgcttcttgttatttttctaaaagaatactatttttttaaaacaaaattttcttttttacaggtatctagaaaatttttcccttatctcgctgctaattttaacgatcctcgtgtaaccctagtccttggagatggtgcgtatttgataatctcgcttttgttttatcttttatttttattgcatttaatttttaccttttggtgtgtggttaattcacctgccattggttctctttcatttcaggggctgcatttgtaaaggctgcacaagcagaatattatgatgctattatagtggactcttctgatcccattggtactctattacttcttaataccaagactaatcttattgaataagctactaataaacggtaattgatttctaaaacaatataatttcaggtccagcaaaagatttgtttgagaggccattctttgaggcagtagctaaagccctaaggccaggaggagttgtatgcacacaggctgaaagcatttggcttcatatgcatattattaagcaaatcattgctaactgtcgtcaagtctttaagggctctgtcaactatgcttggactactgttccaacatatccaacgtatttttctctctctctctcttcctataaaattggaagttttgattctataattgtcaagaaatggagaatcagttccaagaaaaaccaacttcttttcttttactcttcaaggtattgtgtttaattttttttcaactgatatgatcaattattttgatttcagcggtgtgattggttatatgctctgctctactgaaggaccagaaattgacttcaagaatccagtaaatccaattgacaaagagacagctcaagtcaagtccaaattagcacctctcaagttctacaactctgatgtaacttcatatctcacaatttcttttttcctattgtactttatgttcttcgtcaaattttataattaactcttttcaaattgtctttttttttttcagattcacaaagcagcattcattttgccatctttcgccagaagtatgatcgagtcttaa

SEQ ID NO: 5 - DNA, 2035 bp, synthetic construct
  source 1..2035: mol_type = other DNA
atggaagtcatatctaccaacacaaatggctctactatcttcaagaatggtgccattcccatgaacggttaccagaatggcacttccaaacaccaaaacggccaccagaatggcacttccgaacatcggaacggccaccagaatgggatttccgaacaccaaaacggccaccagaatggcacttccgagcatcagaacggccatcagaatgggacaatcagccatgacaacggcaacgagctacagctactgggaagctccaactctattaagcctggttggttttcagagtttagcgcattatggccaggttagtactaagaaagaaactcaaatgcatcgtactcttgtattctgctttgcgtataatttagatgatggtgtttgactaagcactgagtttaaaaataaaaagtttaaagttaaattgttactatagagagctatatctttaggaactaactaaaaaggaaaaattatcacataaaattgggatgaagtaagcagttaacttcgcatattattcgacacattaatttgaaataaatcgaattttgcaggtgaagcattctcacttaaggttgagaagttactattccaggggaagtctgattaccaagatgtcatgctctttgaggtaattaattaatactaatagtcaagctcatgtatgattatatttaaagtggtatttttcgtttatttttaatttattgcacgtgtacgtacagtcagcaacatatgggaaggttctgactttggatggagcaattcaacacacagagaatggtggatttccatacactgaaatgattgttcatcttccacttggttccatcccaaaccctaaaaaggttttgatcatcggcggaggaattggttttacattattcgaaatgcttcgttatcctacaatcgaaaaaattgacattgttgagatcgatgacgtggtagttgatgtaagtcaaacttcttttactcacataaaaaaatgatttagattcttatttttctaaaagaattaaaacaaaattttccgttttacaggtatctagaaaatttttcccttatcttgctgctaattttagcgatcctcgtgtaaccctagtccttggagatggtgcgtatttgataatctcgtttttattttatcttttacttttattttatttaatttttacctttttgtgtgtggttaattcacctgccattggttctttttatttcaggggctgcatttgtaaaggccgcacaagcaggatattatgatgctattatagtggactcttctgatcccattggtactctattactacttaataccaagactattcttattaaataagctactaataaacgtaactctgatagttttctaaaataatataatttcaggtccagcaaaagacttgtttgagaggccattctttgaggcagtagccaaagccctaaggccaggaggagttgtatgcacacaggctgaaagcatttggcttcatatgcatattattaagcaaatcattgctaactgtcgtcaagtctttaagggctctgtcaactatgcttggactactgttccaacatatccaacgtatttttctctctctcttcctataaaattggaagttttgattctataattgtcaagaaatggagaatcagttccaagaaaaaccaaattcttttcttttactcttcaaggtgtgtttaagttttttaaactgatactgatcaattattttgatttcagcggtgtgattggttatatgctctgttctactgaaggaccagaagttgacttcaagaatccagtaaatccaattgacaaagagacaactcaagtcaagtccaaattagcacctctcaagttctacaactctgatgtaacttcatatctcaatttcttttttcttattgtactttatgttcttagtcaaattttataattaactcttttcaaattgtctttttttttcagattcacaaagcagcattcattttgccatctttcgccagaagtatgatcgagtcttaa

SEQ ID NO: 6 - DNA, 1732 bp, synthetic construct
  source 1..1732: mol_type = other DNA
atgccaaatcaaaagcaaggaagtgaacagctacaaacactgatgcaatcaggtcaaatttcggggtctttgagttttaatggggctttaactaaagaagatgaagagatgtctaggtctgctctttctactttcaaagctaaagaagaagaaattgagaaaaagaaaatggaagtcaaagaaagagttcaagctcagttgggtcgggtcgaagaagaaactcggcgtttagccattattcgtgaggtatctctcctttttcttccttattttctcttttgtttgttttttaccagttcctttttctcaacattaatgctaattttcttgttggggttttaacgtaatactggaatttaatttttaaagttgatttttttttggtgagatcttgtgcattttttggaaatacacatatgtgttggtttttcttttgattttttgcttttaggtaggggtttccattttggaccttgacagtagaacatctttagactactactgctacgttatgaacacagatttgggtttgcatatttttataaagttagttttaatttttaattagggttaatggataattcaagatagattaagttatgccagattatcttatcaccatcttgaaattgggctcaaccttttgaccagctaaaaaagaaatacaaatttctctattcttttctatttgctttatggtttttcctccttttttggttgttattaaaaaaaggtttattgaggatgaaacaggaaggaggtggggggggggggttggaattatcctgaagctaaaagatctctgatctgtcaaaattggtcgttttcaatgagggttttatgctttgctattgtttttgccatttaatagttgaaaatgagtatgagttttggaaattgaagtatttgagaaggttaaaatcaagatttatctcagtttccatccaacttatgccttatttaaattcaacttaattatcaggagctagaagcattagctgatccaaagaagaaagatgttcaaatcgttcggaagaagattgatgctgtcaacaaagagttaaaacccctgggacagacctgccagaagaaggtgaaatatatatgcctctattttcttgttttttctttgttttttccccttcattagattgtatatcatataataagtacaatcctagtaataataacccttgggggtcgtttggtacaatggtgggatatcccacgggattagtatcccaccttttatatggaagctaatcccaccattttagtgtaaaagttatcccgggattagctaataccccaaaccaaacatgggataaagttgatcccaaattctatcccgggattattatccttatcccatgtaccaaacgaccccttagagttagttaatcccaatatgtggttgaagtgttaacttatccatgaagctcattaacgagactttttggatctgtgaatgcaggaaaaagagtacaaagaagctcttgaggcgttcaatgaaaagcacaaggaaaaagtacagctaataacaagattgatggaggtgagcatgagtttataatttaaaaccagtttgtatttggaacttgtctcaacatatttgatttgcagaagctcactgaatcttgtgtgttcaatgtacgtgcagttggtgggtgaaagcgagaaactgcgactgaagaagttggagctgagcaagagcatagaaacaatacgctga

SEQ ID NO: 7 - DNA, 1713 bp, synthetic construct
  source 1..1713: mol_type = other DNA
atggatccgtgtccttttgtgagaatagttatcggaaacttagccttgaagtttccggcggaacataaactctcctcaagtacggtttttgattgtaaattcaagcttagaggtttctccacacaagtttcaaccatctcagcttttgttcaagatccaattttggacaacagaactcactcttgtttcaacttgaacaaaacagaacttgacaaactggtcgaaaaatcaactgccaaaggaaaaacttgcagcttaaagattgaaatttttgcacggaagaaggggattggttgtgggttattgaatggtgggaagctattggggagtgtaatagtggatttaaaggggttggaaagtgatggtcggagaggagttgtgattcagaatgaatgggttttggttggtggatctagtactgcagcacaattgcatttgaatgtgaaagctgaacctgatccaagatttgttttccagtttgatggtgaacctgagtgtagtcctcaagtttttcaagtcaatggcaatgttaaacaacctgtttttacttgcaagttcagtttcaggaataatggtgacaggaatttgagatcaaggttagttttatcttctacaattgaagttgattttctaataattagtagaaaaactgatctttcttgactaaacaaattcattccctatagtcacatggcgaacctagcgtaaacatacttgatacgcagaccttgcccttcctcgtggggtagagatgctattttcaataatcgctccctaaccggaacacttataggagctcctgtgtcaatcacttccattcctgtccttagaccgtcccctcgcctatatacatgtataacgaattgaattcatcgagtctaaatcccaacttcatccttcttgttggtgattatccagctaactaacacattagttttagtctatgttgattcatatttttcttgaactctgcatctaaatcttggttgttatttgtacagatcttcactatcaaaaccaagtacatcaacaagctgctttaattcttgcacatctgataaagaatcatcgccgctaaaagagcgaaagggatggtcaatcacaattcatgatctctctggctcaccaattgccgcagcatctatggttacaccatttgtcccatcacagggttcaagtaatgtcagcagatcaaaccccggggcgtggctcatccttcgcccaggcaacggtacatggaagccgtggggccgcttagaagcatggcaagagcgcggtggtgacctcggttatcgctttgaaatcatccctgatggtgctactgatgccataactttgtccaattcaactatcagcaccaagaatggtgggaaatttggtgtagacatcaccaatggtgctaccccattgactagtccaaacagcagctttgacttatgctcgggttctggatccgggtcggatttcgggtcagcacccggatccgggtcgtgggcacaactcttgtaccgtggtttcgtgatgtcgtccacggtggagggtgagggaaaatgcagcaagccagaggtggttgtcggggtgcagcacgtgaactgcgtggaggatgctgcagctttcgttgcattggcagctgccatggatcttagtatggatgcttgtcagtcttttactaagaagctccggaaagagttgagacagtcggatcaagaatga

SEQ ID NO: 8 - DNA, 2086 bp, synthetic construct
  source 1..2086: mol_type = other DNA
atgtcagggtaagtggaattttatgaattcccttaattttattgtcaaattctagtttacttgattataaataaatgttcctgtcgatgtcatttctgttggtaatgatgtagacacaagtgaaaactcagaagtgcacaacttaatataactatttgatcgctttacgagtagttattgcaacttagtctgcaattatttttgtatgtgtagtacatagtctatcagagtgctatttgaattattcttctacgcttaaattaggtcctatctattggaaacagcctctctatccacaaggatggggtaaggtctgcgtacacactaccctccccagaccctacggtgtgggattatactaggtatgttgttgttgcttaaattaggttctgtgtttgcttggaatctttttgggttggttagcgatttgtatgtaagtgtcggataagtttggagaatctatagttttagggaacctatcaagttcaattcttgataatgacttgagatggtcatcagctctccaggaaattcttcttccattctcttttcctctcctccctcatcttcatccttccaagttccatagcatctctttatcagctccttcctttgtttaacatgaatatcatgaaatctccttccatctttttattaagtagatgtaaaacacacgaagtagaaagactcaccatgaaggctttggtttatatggagtggcattcctttccttgtttccgaaaattgtacaatgagccatagataacagttctcgactaacaataaaaaaaatagttctcagccagaggcggatgtagagtaccggtaccagctgaacccgctaaaattacaacaaatagtagatataaacccataactttaaaaatattgtgggttcaatgctaaaagtcttaaagatcccataaatgctggatcaacttctgttctcagctggtatcagacattgttacatatcatgtaaatatttttgtttagtcctaaattttgtgaatcagcttatgttctgaaacttaaagtcaagggaggtatctttattccctggcaaaccttacattggacttggtattctaagttaaactgtctcccaagtttcatgtaatgtagcacttaatggttagggatatcccttccagtttgccttcgcggaaactggacatcggttttttacttttcttccattattcatgccacattggaaagttattagttacttaatcaattttgctcttttagtttttcacttcctttaacttacccataaggttctctacttaatattcccttcgattgtagcgaggatgttgcagttgctgttgccgagactcctgctccagcacttggagagcccatggacatcatgactgctttgcagctggtgctgaggaagtctaaggctcatggaggcctagctcggggactacatgaaggtgcaaaggtgattgagaagcatgcagcacagctttgtgtgttagcagaggactgcgaccagccagattatgtcaaattggtcaaagcactctgtgctgatcacaatgtcagcttgatcacagttccgaatgcaaaaactcttggcgaatgggctggtgtaagtttactttgtttgccacttgctactttttagctctaagtggcttagaggggtgataaaatattaacttccgacttctcttgatgtgcattttcatgcgtcatgaatcgttggagtatgaacgttctgtccttttctaattcttatttttgggcccattttacagttgtgtaagattgactctgaagggaaagcaaggaaagtggttggctgcggctgtgttgttgtgaaggtatatccatgaactactaagcatcataatcttgttctagcatgtatagttcggctttatatattaaaggaaaatgactcgggctggatttctgtcttaaaaatgctgaaatataaatagaacctaataagttgtggaaacagaggagattttccgtcttagttaatcttactatcattttgcaggattatggggaagagactgagggtctgcatattgtccaggagtacgtaaagtcccattaa

SEQ ID NO: 9 - DNA, 1717 bp, synthetic construct
  source 1..1717: mol_type = other DNA
atgtcagggtaagcttggtgatttgtaagctttctcattaaattcagttttttaattcattatagtgttcaagttgctgtcttttttatttttagtaatctagacacgtgataaattttgtatggttttgctgaattgcttgatccacgttaagattagactttgcaagttcaatgttcaaaattttctgtacctctattagaaagagctaggcagaagtgtttcctgtctgtagaatcatgaaacaaatacattccctggaattaaactaatatgtcattatttcaagggaacccaaccagtagaaccatccaaaaattgattctttttgtaggtcatttgaagcagaaccaaatacaattagcgtatgagagtttcttaattctatgtttcagctgcctattacatatctatagcctttttatatatatgttgtttgttgttagaattacaaaaccctaaattgtcatggtactgatgcagaactgtcatatagcaaatgttttatccaattggttgaccactttattttcatggaataatggacatgctactgtgtgtagacaaagtatcatgaaaaaatggattggcttagctttgatattccgaggtttgatgagaaagaaaaatgttatgatgtcaacgttctgaattagtcgagtttgatgttcttgtggcattggctttaataccattataatgtgacactgcatggtgatttcttgcttcaccaatctggtccttgatgtgtgcctatctttatctttagtataaggtgtactcttacctgacagttgtatatctgtagagaggatgctgctgttcctgttgttgctgccgagactcctgctccagcacttggggagcccatggacatcatgaccgctctgcagctggtgctcaggaagtctaaagctcatggaggacttgctcgaggactccatgaaggtgctaaggtgattgagaagcatgctgcgcagctttgtgtgctagcagaggactgcgaccagccagactatgtcaagctggtcaaagctctttgtgctgatcacaatgtcagtttgattacagttcccaatgcaaaaactcttggcgaatgggctggtgtaagtaattgatcattgcttatctatggccccaatcacagtatctcagttgttactatacatacttgagtagctggttctgatgtgaaaatgaaattggaatgtgcgtctttactagatactccatcttttaataggtgaagggttttagggacttgtgataaataatactcagaatcccatgactgaagccttaactttatgttggacctatgttattgtgttttttgcacttgtattggagttgaggggtatatgagtcttctgatttaacttgttgactcctctttgaagtggtctgtagttctcatttttgtagaagtgtatcatgtaaatcgaaatataatcactcatcgactatgatctttcttcaattacagttatgcaaaattgattctgaagggaaagcaaggaaagttgttggttgtggctgtgttgtcgtgaaggtacgtcctacgtatcctctggctttaaatgaatagtacagttgataagttttccaatctggaatttatactccactgtaatgaattttctaattgattctgcaggattatggggaagagactgagggtctccatatcgtccaggagtacgtgaagtctcattaa

SEQ ID NO: 10 - DNA, 1451 bp, synthetic construct
  source 1..1451: mol_type = other DNA
atggatcctcaggctttcattaggctgtcaatagggtcgctggggttgagactgtctggaacaactactttgaacagtacaaaaacagggataagtgcagtctcttctccctgtgtgtgcgagatccgtcttcgaggtttccctgtgcagacatcatctattccctatatttcctcacctgaagctacaccggatattcacaacgttgcatccagcttttatcttgaagaatctgatttgaaagctttactggcacctgccccctgtttttatgcggctcatgcatgtctagagatagttgtttacacaggacgcaaaggaggccactgtggtgttggtattaagaggcagcaagttgggacatttaagttggaagtaggtcccgaatggggtgaaggaaaaccagccattctgtttaatggctggataggaattggcaagaacaagctggagactggaaaacctggagcggagcttcatttgagagtgaagctggaccctgatccaagatatgttttccagtttgaagataaaacgaaactaagccctcaaatagtgcagcttcagggaaccatcaagcaacctattttcagttgcgagtttagtcaggacaggtaaatcattgttttgctcttttaccctttcctagggatgagtagcgtataagtagaattgaagttctactgttatagtctactttttccgggaaacttttaagttgcttcagttctaattctaagagttgtgtgttatgctgaacttacagggtatctccggtagatccattaaataatttttggtcaagttcatttgacggttctgaactagaggtagagaaaagagagaggaagggatggaaggtgaagatacacgatctctctgggtcggctgttgcagcagccttcataacaactccgtttgtgccatcaacaggttgtgattgggtggccaaatccaacccaggagcttggttgattgttcgtcctgatatttgcaggcccgaaagttggcagccatggggaaagcttgaagcatggcgtgaacgtgggatcagagattccatttgctgtcgcttccatcttctatcagaggggcaggaatgtggtggtgatcttctcatgtctgaaatcttgatcagtgcagagaagggtggtgagttctacattgacacggacagacaggtacaagcagcagcgagtccactgcccagtccaagaagcagcggagactttgcagcactaagtcctgttgcaggcggttttgtcatgagctgtcgagtgcaaggggaagggaaatgcagcaaacccctcgtgcaacttgccatgcgacacatcacctgtgtggaagatgcagccattttcatggcgcttgctgctgcagttgatcttagcattgaggcatgcaggccttttcgcagaaagctacggagaagtactcgccattcctggtga

SEQ ID NO: 11 - DNA, 2282 bp, synthetic construct
  source 1..2282: mol_type = other DNA
atggctaaggtgtgcttttacaatgtgaagatgaatatgcaagtgatgttttgcttgtttttggtgtcactatgtcgtgatgatgcagctgctgaagctaaacaggggatgtctcttgctgcccaaaagttcgaggttcaaaagcacttgaatcgcttaaacaaacctgcaatcaaatccataaaggttaaaattttatacccctttttttgctccttttttttgtgtgtctttttgtattgatggtttcttttggaaaaaaataacagagcccagatggtgatattattgattgtgttcatatgtctcaccaaccagcttttgatcatcctttgctcaagaatcacactgtacaggtcagcctcttcccaaaaatagaaccccttattctgtttgtaaaaattaaacataattcttgtgataatcttaatgggaaaaaagttgatttttttgggttatagatgatgccaaattatcacccagaagggctatttcgtgatggaaaattgtctacagcaaaaaccaagaatgaaggttcaaaatcaattactcaactgtggcatttgaatggaaaatgtccagaaggaacaattccaattagaagaaccaaaaaagatgatattttaagagcaagttccatgaaaagctatggtaggaagaaaaaacattctactattcctaaaccagagtctggtagacctggccttgttagccaaactggtcatcaggtacaccaaattgttcagatttgtatagaaggaaaatcagaagtttcattgaattaatggttttgttatctgttggagttattttagcatgcaatagcctatgttgaaggagacaaatattatggtgcaaaagcaaccataaatgtttgggaacctaaaattcagcaacccaatgagtttagcttgtctcagctttggattcttggaggttcttttgattcagatcttaatagcattgaagctggatggcaggtactgctacttatcttttccaaattatttgcttgtttgaacaattagagagtgatattattgtcatattatatgtctgtatttggttgtcttgtgacttcttcttttttacttttcttgtctttttggggataaaaacaggttagcccagatttgtatggagataacaacacaagactttttacctactggactgtaagttaaatatttttccttctaaatacagctagcatacatgcataaaaaatctaaaatcaaagagtgtgtgctatataaagttcatgtttcaaaattgaaattttggaaaattattttttgtacagagtgatgcttatcaagccacaggctgctacaatctgttatgttcaggatttattcaaatcaataatgaaatagcaatgggggccaccatttcccctctttccagctatcatggttcccaatatgatataagcattcttgtctggaaggtaatttaatttattggattcacatatcatggtttgacccttttcagtttacttaattcttatcaacaattatacctcatattgtattgaaaaatacacccttgtatgttccacagcatattttattgaggtgtccattatttgcatgtgaatgcataattgattaattaacttatgagaagacagacagaatctctgacacttgctgttgagcccatgctcttctttctctatttcataaaaatctgttctataaaggggagattgtaacttgtaaagttgcggtcatgtgttcaagccgtggtacaataggctcttatgttccggcccttccctggaaccccctcttaacggaagcttattataccggaccgtcctttaataaaaatctgttctttagcttttgttatttatcttgaaaatattttaaagtttgatttggcttaacttataggatccaaaagagggaaactggtggatgcaatttgggaatgactatgtattgggatattggccaggctttttattttcatatttaacagacagtgcttcaatgattgaatggggtggggaagtggtgaattcagaatcagatggacttcacaccacaactcaaatggggagtggccattttccagatgaaggttttgggaaatcaagctatttcaggaatatacaagtagttgatggttcaaataatttgagagctcctcaagatcttgggatttatactgaggataacaattgttatgatgttcaactaggaaaaaataatgactgggggaactacttttactatggtggacctggcagaaatcctaattgtccatga

SEQ ID NO: 12 - DNA, 2377 bp, synthetic construct
  source 1..2377: mol_type = other DNA
atgtcaatagacaaaagctcaatgcaatcaaaccttgattgcttcttggaatgcaccacaccgttagtcccttcccagtttctaccccaggtattttttcttcatttttcatatttataattcaatggatgaatatgaaatgtgatttgttattgtatgattgttttatgcgcagagtgagattaggaatctgaacaggttatggcatccatgggaaagggagaaggttgaatattttactttggctgatctttggaattgttacgacgaattgagtgcttatggggctggagttcctattaaaatggatacaggcgaaactttagttcaatattacgtgccttatctttcagctattcaaatctttaccagcaggtcttctgttaattttctgaggtaaaattcttttggtgctacacttttttgggtatctcaatttgatcattgattttagggtcttttcttgtcaaatgaatttcttggttttttttgaaagtaatgcagcattgcttgtttaccaagttgagccggtcaaagtttgtgatggcagcaaaaattctgctacttaaagctaaataggctattacatagtttatgttagtaatatacgtttcccagaagttagattattcaaagtttgttagttgcttgttagaaactatgcttcatagttgtggactggagataatgaagttgttcaacaatttaataattttgtagggaagagactgagtctgtttgcgagacgagggattcgttcagtgattcgttcagcgacgagagtgaaagtgaaaagctatcaagatgggatggttgttcttctgaggaaggagatagtctttggcaaatgaatgatagatggggctatctttattttcagtactttgagagatctactccttatggaagagtccctttaatggacaaggttttcctctttagacttgttacacctttttttgtcccctttagtttctattcccctgcttcttaaagtcttcttttgaaccaaaatttatactggtagctagtgctttaggaaatgtatgtggtagcaagttttagttgtatatttccaattagttttatctcttgtttcctttgttatcaacccctttctttttatgaacttcatattcgaaagaaaaaggactaactggggtttgtttctgttaatagggatgataggaatagttcttagtttgtgacttctaagtacaaatttacaggccttaattgtgctttttagtcccccttctccctaaaatcattggagataaatctattagtattgttctttattctgtattctagcctaatccggacattatggttgacttaatcacccactcatctttgtttatgcaactcagagaaaatttgaagcatttattctttctttatgcagtagagttctctaatgattctttatttgataagatatataagattccatatgaaacatgtggtttgaactttttagcatatattgttttgttttgaacgtttatttgacactaactctgattctttttgctcgcagattagtggtttagctgaaagataccccggattaatgtcattgagaagtgtagatctttcaccagctagttggatgtcagttgcttggtatgccagttcttccgttctgtgcacttatggtcatgaaactaatggttaactgaagtagattttatcaagttaatgaaatttatctgggacatgtaaaatattgtttacttgctccttgatgggtattaattggatcatattttccttgtaatgcaaccaattttataccacttaagttgatcctgcatccttttggtattgctgacttgcttggtcgccacagtgagtaatgaaaggggaataaagacggattttgttactaacccttttgacctggacattataggtatccgatatatcacattcctatgggaagaaccattaaagacttgtcagcatgctttctcacattccacaccctttcttcttctttccaaggtattgtttgatctatccgttggacattgaatgcttttccctttttaattgctatcttctgagcgaacgagtgatgaagtacagaatatttttacactagctaatcttttggtcgtctcgtgcagatatggaccttgaagatgacacggagaatggtaaacggaaacgaaaggaaggagaaagcatcccacttccacctttcggtttgggcacttacaagatgcagggtgatgtgtggctttcagacaggaaaggaagggaccaagagaggctggcatcactttttagcgtggcggattcttggctaaagcagttgggtgtccagcaccatgacttcaactatttcatgggtattcgtcgtggctga

SEQ ID NO: 13 - DNA, 951 bp, synthetic construct
  source 1..951: mol_type = other DNA
atgacaaaaactagcaaggtgcagcgggtacctggtttgctatcttgccgctcaaaggtgtgcaaaaagattcaaaaggagaaacgaccgaaatcatcagagaagaaagaatgggaaggggcaacttgctcagtttgcatggagcatcctcacaatgcagtgctgctgctgtgctcttcttatgacaagggatgtcgtccttatatgtgtgccactagctgtcgtttgtcgaattgtcttgagcagtacaagaaagcctatgcgaaagtcacttcaattgagggctctgaaccagagttgctgtcaattgatgatccaaattgcttgtcaggagcaggttcttgcgctgggaagtcggtagttgaacttctatgcccactctgtcgtgggcaggtgaagggttggacggttgttgaacctgcacgcaagtatctaaatgcaaagaagagaacatgcatgcatgaaaattgctcatttgttgggacatacaaaaagttaaggaaacatgtaaggcgggagcatccatcagcacgccccctcgaagtcgacccttcacatgcagagaaatggaagaagcttgaacacgagagagagttgaacgatgtatttagcacaatcagatcggccatgcctggggcaattgtaatgggagattatgtaatagagggtaactttggaggcttccacagaaattttggattagatgatcacctcggtgacactctttttaggtcggaatctaatggcattccctggaatgatattgtccactcagatgatgttttcgatgatggctatcattcgtttgatgaggatgattttttcgtcaatcattctggcagtggggctgcatctaatgttttcaataggatttctcgacttcgtagcaggctcttattgggaagatcaaggaggcggcaacgacatcgagcgagtagcagaattcggtga

SEQ ID NO: 14 - DNA, 3966 bp, synthetic construct
  source 1..3966: mol_type = other DNA
atgaggacattgtgctccaacttggatagagaagatgggctggaaacagtgctggaagtccccatcccagaagaaatgtttgcttccaacaagcacagggcacggcagagcaagaattctggggtcaaatctcacatggacaagtctgctgcttccgtttttggtagccgaaatgctgagattcaactcttgcttggcgttgttggagctccgttgatccctcatcctatccgttgcgactactctctcaacacaaaaatcaacgatcatcccatagtaagtcggagtattgtggcagcattattttaaagcttaaactattaggggtcctttggttgctggttagagttatgcagctattaataaagtagggagtagttatacatgatttagctatgtaggaattggttatgcataatttaattatatatgcagggtttagtcatgcacgatttaattatgaagaggatatgttatttatggattagtaatttatgaattgtaatgtagagattattttttgttcaatatttacttttttttaaatatacaatgtgtaatttaaaatatatatatatgcgtggtcattttgattgtttaattcatatattataaatacatgtattaattgttccacattctaccttgcataactaatacatagatttgttggttaacttatccatgtattacttatacctctaataaaagcaaccaaacatgatattagagggagtttagacataagaattgtaaaattcccaaaaaaaaagtgaaaaaaaatttaaagtaaaatggtatttaaaaattagagttgtgtttggacatgaacattattttttgttttttatgaagttttgtgagtaattagagtgaaaattttgaaaaaacacatttttggagtttttcaaacttccgaaaagttccaaaatgcattttcaagtgaaaaataaaaattttatgaccaaacgatgagttcgaaaaaattgaaaaaatttcggaaaaaagtaaaaaatttcttatgtccaagcgggctccgtattagttatattactcctaatgcatgaataatttaccttgtaatcagctaaccccttagagagtaaattttttctttagttaattcaacatgctctctcacaaccggattcttttttatggatgattagcaacagggagattagaacataaaacctctacctgtccgatatcatattggattgtatgaccatatcatctaaaaagtttagttgaacatatttttatttatttaattggtatatttttaacatgttcaggaagcttcagtggcgaaatatatagtacaacagtacatagcagcagcagggggagaacatgctctaaattccattgatagtatgtatgcaatggggaaaataaagatggtggcgtctgagtttattgctggagatggaatgggattgaacaacggtaatatgatgaagattaagagtgtgaaaaatgggcctggtgaaatgggaggatttgtgttgtggcaaaagagacctgacctgtggagcatagagctggtggtttcagggtgtaaaattagtgctggaagtgatggtaaagtagcttggaggcaaactccatggcataattcccatgcatctcgtggtcctgctaggcctcttcgtcgttctttacaggtactttatttcaaatcaatgggttcaattgaatttaatacttccagtatggtggagcactatgagaatagtgcaaaataaggtgtattagtaatgcatgtgttgataatgcaagcattagttatacagatattattttttatctactgtttgatgtggtgtattaaagttataatgcattgcataatttttaagagaaatagttgtttaaaaaatgtcatccatattctctagctttaagggtctttaaggacaattttatttttgaccttactaatgcatgcattaatagccttggtattactaatgtcatagttttctattccttacttatacataggataatatgtatagtttagttatacacagattgaaaaaatataccaaataaggtattaataatacatagaactaatgcttgcattattttttttctaataccctatcccaaaacaaccccgaaaagaattaaggtatatagtgctaacaactttttaatcgttgaatctatcaaagtatatatcataaattctctcactcaatccttgaataatggtcaattttccttttattagtcatgatctaaaacaagcttataacaagtgaacaggccgacaatttctccacatgaaaatgtatccttcttctgaagggacaataattgccaaatacgtcgtttttttctttgccacatgatgagtgggaaggccaagattcgttgactgttcctactgacaactatgtatgttcttttactttgtgtgggtcctcgtgtctgattaatctatttaactggtacttgggttttgggaagtgtgaaatcataatcaagaagatcaaaattctctttgacgttcttttttactttactttggaggtgtaggacctacttatttccatggtcattgacagctttagttttatcttttttctttatttctggctctaacgggacatatccccatgcggacctgagttgtccttccaccaaatcaatgattgattttatgagtactcaaattggcatctattctgaatgtgtaagaatcttagcctcaacctctatgatcagcaatctagaccaagaaatcagttctccgttgtgaatgcgcacaaatcttagcctccattttgaataaagagttattaaacttaatctcatatatgtgttgagatttatattatgtttggttttcctttttctcgagtgtattttataaagacaaatcacatgacccatcaaaatggtcagtagggcaagcttgaaaaatgtctaataatcccttggtttcattttatatgagactatacacttgaaaacttttaacatttatgtctactatatttgcgatatctacttaataaaatgcattatagacacataaatgtctactatatttttggatgataagttttaagggtacctttttatatctatcaaattatgtcttcttttataaaattcttgcatagtaaaatacgtcatataagataaaattagaaggagtaacaaactagaagccaatatatataaattttttctgaagatggtaaattcctgaactaaaaacccccaggaaaaaataaagataagaaatatggaacgagtaaagtttgcttttgtagaacaaattaaactggttgaaattgaacatgttaacagggtcttgatcccaaatccgttgcgaatttattctcgaattccatttgtattggagagaagacggtgaatatagaagactgttttgtactaaagcttgaaacagagccctcaactctgaaagcaagaagcagcagcaacgttgaagtaatgaggcacactgtttggggttacttcagccaacgaacagggctcttactccagcttgaagacactcatcttcttagaatcaaagccccaggaaatgatgtcttttgggaaacaacaatggactcattaatactcgattatcgaactctcgacggtgttaatattgcacatggtggaagaacatctgtttctttatttaggtttggtaagaactcagaaggactcactaggacaagaatggaagaggtttggaccattgaagaaatcgattttaacataaagggactatctgcggattgtttcttaccacctagtgacttgaaaaaggaagatgaagtgagtcatgatgtgaacaagaagtcaaggttggggttcaaggctcgtactaataatgctaaactaagaactgctaggaaaggtgcgtctaaaatcatgtctattgacgaagaagatcttgaagattacgaagcagatgaagaatcttga

SEQ ID NO: 15 - DNA, 1022 bp, synthetic construct
  source 1..1022: mol_type = other DNA
atgattattggctctagcttctcagaaaagtaagttattcatttctttactttttatttaattttttgctattggtatgttgtgtcttcatgttatggtttattgggtattgggggggggagggggggggggtgttgttttataagggtatatagatatatgcaaagcaccagacaccttaaagatttgatttttatttctgggtagttgtgaaatttggattttgctatcatatattataaggttatatagatatatgcaaaagagagcactagataccttaaaagtttgaatatttctcccttggtagttttcaagattagaagtggagtattggcgtcacgggtaaattgatgcccgtgaccacgaggttactagttagagccggtaaaatagcctctcgcagaaagtcatgttaagattgcgtacaatagacttttgtgatccggctctttttcggacccgtgcatagcaaaaacctagtgaaccgagctttggtagttttcaagattggattttgatatcatatgtttatatagatatatttatgcaaaagagagcaccagaaacattaaagattggatttttattttttgggtaattttaagaattggattttgtttatcatataagtttttttaaagactgaaatagcttagtgggttgtgaaaagattgatgggaaagaagaaaattttgaagaagacaaaggagttatcagtagcaatagcagaatcatcagcaatgagtggagactctcaacaacaacaacaacagattactcctagaaaaagaggaagaccaaggaaaattattgtaaaagatgaagaagaggctactggagaaataaagaaactcaaaataaatgaaggtgaagaattagaaaataaagaagcagcagaaggagaaaagaaagaagcagaagaacaatctgaagaggaaaaaaatttacagccacaaaagcagcaacctgtagcaaagagcagagctagaagaaaaagcaagccaagaaaaagctgctaa

SEQ ID NO: 16 - DNA, 3324 bp, synthetic construct
  source 1..3324: mol_type = other DNA
atgatggcacagcatattgagattagcccactgcttgatcaaactcctccgaatgaagctacgaaagatgaggtctctctcctctctcttcacaatcaaacacacacatatatataagcattacctctttgttatccacatcatgtaacccaaattaattaggacaatactaaaaatcttgagataattttattgaatacctcaataattatagctttttcattagttaaagtcttcaataaatatagcttttttcattagaaaaaagaatcttagcttagcggacttctctctttttggcaaccccttaaatgataattgaacgagattaaaaggagaaaaataatcctaaaacacacataacacctagacataattctcacttctgactcgtctctttcgacagtcgccagctcgcccgcggttgctaaagccttcacccactgccgctgcttccttcttcgcctttgccgtcgccagtcatcaagtggttagttctctctccctcccttcatctttctctctcttagggtccttttggtatgagttataagtacaaatagtgttgagataaaaatttaatatcacctggaataaattattacgggattaaaattagtatcgagataacttataccttatagatggtgggtaattagtgccgggataacatataatttcttcttagaaattatgcaattgtcatttttaatacaacataccaaacaatggataaaaaacaataccaaacatgaaagtttttcaaaaaatattattttggatagaagtttcattttataagaatgtcaaatgtttcaattttgaaaaataaagatatatctgtaactaagtacattaaaatataatactccctataagacaaattaaccaaaaacatgaaacttcaattttattcatacctttgatcaaaaagaaaataattttatttcaaaaacatgtatatttcaaaccttttgaataagaaataatcacaatttcttaataatcaattactagtaataacattttttgtgtttcgaaaataaatgctatttagattttaagaaaattataatattttagcttgcttgttattattctgcatcaaattcatttagatgaaaatttattcgtggaatttccattatataaaggggtatgcaaaggagtgtatccgtagttgagttatattatagggatgtttaaagtgctttactctaatgtatttgctttatataattgtgttgaattaactaaatcatgttactactgattttagaagaatgaatttgatgataaacgtaagctcgatttaaataagcattaatttattaattataataacaatcatttagatttcttttttatgttatcttgattatacaaatgatatacttggttaaataaaattttccgtttaaccaaaaaaaataatcacctttctcaacattcacctaaatagttctctttccttttgtttgtttgggcttttaaggctacttaattagcttaatttgatgtatggttaagtatatattgtccaaaaattccaggtctgtaaacgtttcaagatcgtttgatcttcttcatttcacgtttcttaagatagtaactatagagttgctatctattactgactgcttagtcacaagtccatgtgattagaaacgaaaacacccaaaaaaatggggaaaaagggccgcccggtgcactaagctcccgctatgcgcggggtccgggtaaggtcgaaccacaagggtctattgtacgtagtcttaccctgtatttctgcaagaggctgtttccacggctcgaaccggtgatcttcacaactttaccagttacaccaagacttcccttcaaaagaaaaactaaaaagaaaccatgtttcactaattacagatacaagaaggcgggaaagcagattatataatcgagataaggacagcagatcaaacaaatgaatcagcaaatcaagtctttgatgaaggatcatctaccatatttaaagtaaatgtggggctatctgaatcaaatccagatgcttataccccaaaattgatctccattggtccttaccataaccaaaatccgagacttcgctcgatggaaaaatataaactgcgttaccgacaacggtttctgcagaggaaagcgtggagagatgtggagtattactttagtgaaatggagaaactaaaggatgaagcactaaagtgttacgatgatataagggaccttgacagtaatattgttagcaaatttttagatttgctgttacttgatggctgcttcgtggttgagtatatacgagagttttacgaaggggtgccagaaggagaagacaagattatcaacgcggcttggatggaatctctggtagatcgtgacttgttgttactagaaaaccaacttcctttctttatcctcgccaaacttcatgagatgactaaggatcctacagacgccccattcatacagatggtgaaatacaactttggtagtagtttaccaaaagtgactcctaaattcataaatgctactgatgatgatgccgaagaaatcaaacatttacttcaagtagtacacatgtgttgttgcccttcagagatgaatactggcctaactaggaatagcagaaagaaaaaaggaagtagcaagaaaagttgtaactggaatcctttgcgaataggaaagtccaaaaagaagtttaaaactaaggatggtgacttatggcacgatcgcatgcgaagtgcaacagaacttgatgaagctggaattcgcttctctaatgttgggaaaatttatagaaagttgaataaaaacaacaaggaggatgctataagtttatttgatatcaaattcaataagggattgcttgaaatcccttgttttgaagtcattaatagtacggagactatcctgagaaatctcattgcctacgaacaacactcctctgatgtgcatccaaaatatttcacggattatataatttttatggaccatcttattaactcaggcaaagatgtgaatttgcttcggttgaacggaattatcagaaataggataggagatgacgaagaagtggctattatgttcaacaaactaggagaaggtgtcatcccttcaactgatttcttttacaaagtggaatgcagaaaagtgattgagcattgtgaaaaaccatggaatgaaaacatggcaagtttgaggcacaattatcttaatagcccttgggcgaaaatttcaactgcggcagccatcattctccttctgctcacagttgcacaaacagttctagctttcatcagtactttaaagtag

SEQ ID NO: 17 - DNA, 909 bp, synthetic construct
  source 1..909: mol_type = other DNA
atgagggacttcccatcttgttttggtgaaaatggggttcaagttgctgatgcttcttgttcagctgttgggggtgtgactaaaaccccacagaatttagtcacatgtgtttatcagtgtaaactacttgggaaatcttgtctgatcactattgtttggaccaagagtttgatgggtcaatgccttagtgttgaaattgatgatatgtctcatcagtgtctttgtaaagttgatgtaaagccttctctcttctccaaaagaaaagggtcaaggtctttagaagtaaattcttgtaaaattgacctacactgggacttttcactaggcaagtttggatctgggccagagcctatagaagggtattatttagctctagtttgcaaaggacaaatggttttggtcattggggatctgaggaaagaagcatttaaaaagactaatgcaactcctagtttttcaaatgcaatgttcatttctaaaagagagcacatatttgggaagagggtatttggtacaaaggctcaattttgttatacaggtccaattcatgacattacaattgaatgtgactctaatggcattgacgatccgtgtcttttggtccgtattgactctaaaactgtaatgcaagtgaagcatttgcggtggaagtttcgcggtaactatactgttttaattgatggactccctgttgaagtcttttgggatgttcataactggttgttcagtagcaattttgggaatgcagtgttcatgtttcaaacttgtttatcggccgagaagttgtggaccacacaaactttgtcggatctctctgtgatgccttggtcttacacagagagtttgagcaattccaaatcatctggtttgggtttttctttggttttgtatgtttggaagaacgagtaa

SEQ ID NO: 18 - DNA, 2210 bp, synthetic construct
  source 1..2210: mol_type = other DNA
atggaagtcatatctaccaacacaaatggctctaccatcttcaagaatggtaccattcccatgaacggccaccaaaatggctcttccgaacacctcaacggctaccagaatggcatttccaaacaccaaaacgggcaccagaatggcacttccgaacatcggaacggccaccagaatgggacatccgaacaacagaacgggacaatcagccatgacaatggcaacgagctactgggaagctccaactctattaagcctggttggttttcagagtttagcgcattatggccaggttagtattaagaaagaaactcaaatgcatcgtactcttgtattttggtttgtgtataatttataatatggataaattatgacgaagtaaactcgcatacattaatttgaaataatctaattttgcaggtgaagcattctcacttaaggtcgagaagttactattccaggggaaatctgattaccaagatgtcatgctctttgaggtaattaatattctgatacacatgctttaatctaaagtgatacttttaatttacttttagtttattgcatgtgcacgtacagtcagcaacttatgggaaggttctgactttggatggagcaattcaacatacagagaatggtggatttccatacactgaaatgattgttcatctaccacttggttccatcccaaacccaaaaaaggttttgatcatcggcggaggaattggttttacattattcgaaatgcttcgttatccttcaatcgaaaaaattgacattgttgagatcgatgacgtggtagttgatgtaagtcaaacttcttttacccacataaagaaaatgatttagattgcaattctttttatttttctaaaagaataaatatattctctctttttttttttaaaacaaaattctctttcttacaggtatccagaaaatttttcccttatctggcagctaattttaacgatcctcgtgtaaccctagttctcggagatggtgcgtatatgatagtctcgttttatattttatttcacttgatttttacctttttttgtggttaattaatcatctaccattggttctctttaccttcaggagctgcatttgtaaaggctgcacaagcgggatattatgatgctattatagtggactcttctgatcccattggtacgctattactatttaataccaagactattcttattatataagctactaagaaactaattgaataattaataaacgtaactgtaattgatttctaaaataatatatataatttcaggtccagcaaaagatttgtttgagaggccattctttgaggcagtagccaaagcccttaggccaggaggagttgtatgcacacaggctgaaagcatttggcttcatatgcatattattaagcaaatcattgctaactgtcgtcaagtctttaagggttctgtcaactatgcttggacaaccgttccaacatatcccacgtattctttttctctctctcttcctgtctttttcgatgcaatgtaaatttataaaattggaagtccgttttacttttctatagacgtagatcctaaaattgtcaagaaatggagaattgacttacaagaaaaatcaacttcttttcatttactattctttttggtgacaaactttacttattatttcgttctaaaatgaaaatttatttttatattttaaaataatttagctttaaacttttaattttacttgttatatttttaataaaaaagatttattgtcaaataaatgttgtggccatacaataagtttcaaattatgtcacaaaaattgaaacagagtgagcaaatttttatttcaagtaaactttcaaggaattgtgtttaagttttctcaactgatactgatcaatgatgtcttaaatattttgatttcagcggtgtgattggttatatgctctgctctactgaagggccagaagttaacttcaagaatccagtaaatccaattgacaaagagacaactcaagtcaagtccaaattaggacctctcaagttctacaactctgatgtaacttcatatctcacaatttctttttccgttttactgtatgttcttcatcaaattttataactaactcttttcatattgtctttttttttcagattcacaaagcagcattcattttgccatctttcgcccgaagtatgatcgagtcttaa

SEQ ID NO: 19 - DNA, 2317 bp, synthetic construct
  source 1..2317: mol_type = other DNA
atggaagtcatatctaccaacacaaatggctctaccatcttcaagaatggtgccattcccatgaacggccaccaaaatggcacttctgaacacctcaacggctaccagaatggcacttccaaacaccaaaacgggcaccagaatggcactttcgaacatcggaacggccaccagaatgggacatccgaacaacagaacgggacaatcagccatgacaatggcaacgagctactgggaagctccgactctattaagcctggctggttttcagagtttagcgcattatggccaggttagtactaagaaagcaactcaaatgcatcggcctcttgttgctactaaatatagagagctatcatacttttagggactaactaaaaaggaaagattatcacagggacgaagtgagcagttaacttcgcatattatcagacgcattaatttgaaataatcgaattttgcaggtgaagcattctcacttaaggttgagaagttactattccaggggaagtctgattaccaagatgtcatgctctttgaggtaattaatattctaatacacatgctttaatttaaagtgatacttttaatttacttttagtttattgcatgtgcacgtacagtcagcaacttatgggaaggttctgactttggatggagcaattcaacatacagagaatggtggatttccatacactgaaatgattgttcatctaccacttggttccatcccaaacccaaaaaaggttttgatcatcggcggaggaattggttttacattattcgaaatgcttcgttatccttcaatcgaaaaaattgacattgttgagatcgatgacgtggtagttgatgtaagtcaaacttcttttacccacataaagaaaatgatttagattgcaattctttttatttttctaaaagaataaatatattctctttttttttttaaaacaaaattctctttcttacaggtatccagaaaatttttcccttatctggcagctaattttaacgatcctcgtgtaaccctagttctcggagatggtgcgtatatgatagtctcgttttatattttatttcacttgatttttacctttttttgtggttaattaatcatctaccattggttctctttaccttcaggagctgcatttgtaaaggctgcacaagcgggatattatgatgctattatagtggactcttctgatcccattggtacgctattactatttaataccaagactattcttattaaataagctactaagaaactaattgaataattaataaacgtaactgtaattgatttctaaaataatatatataatttcaggtccagcaaaagatttgtttgagaggccattctttgaggcagtagccaaagcccttaggccaggaggagttgtatgcacacaggctgaaagcatttggcttcatatgcatattattaagcaaatcattgctaactgtcgtcaagtctttaagggttctgtcaactatgcttggacaaccgttccaacatatcccacgtattctttttctctctctctcttcctgtctttttcgatgcaatgtaaatttataaaattggaagtccgttttacttttctatagacgtagatcctaaaattgtcaagaaatggagaattgacttacaagaaaaatcaacttcttttcatttactattctttttggtgacaaactttacttattatttcgttctaaaatgaaaatttatttttatattttaaaataatttagctttaaacttttaattttacttgttatatttttaataaaaaagatttatagtcaaataaatgttgtgaccatataaaaacctccgcatttttaagatcataagtttcagagtcaaacgagttaatttatttttagtatgccggtgcggagtcaaattatgtcataaaaattgaaacggagtgagaacatttttatttcgagtaaactttcaaggtattgtgtttaatttcaagtgatactgatcaatgatgtcttaaatattttgatttcagcggtgtgatcggttatatgctctgctctactgaagggccagaagttgacttcaagaatccagtaaatccaattgacaaagagacaactcaagtcaagtccaaattaggacctctcaagttctacaactctgatgtaacttcatatctcacaatttctttttccgttttactgtatgttcttcgtcaaattttataactaactcttttcatattgtcttttttttcagattcacaaagcagcattcattttaccatctttcgccagaagtatgatcgagtcttaa

SEQ ID NO: 20 - DNA, 1443 bp, synthetic construct
  source 1..1443: mol_type = other DNA
atggccaacaaggtctccaatttctccgatctaatccaacgtgtcactgcttcttgcttgcttcatccactttcctctggccgtattgacggcaatatctccgatgctcgttatagcgatgaatctgaagatgacaagtacagtaccgaagacgaagaagaaaaagaagacccaccttatgtggagactcgggaaaaattccagaatgttgaggtcacaaaggaggagataagaactgagaaagtaactgaaatggagatgcttttaggggaagtttttgacgcggtgtcggcagtgaagacggcttacgttagtctacaggaggcgcattgtccttgggatcaggataaaatgcgtgtggctgacgtggctgtgatatctgaactgcggaggcttggggtgttgagggaacgatttcgaaggaatgttggtggtggaattagaaaaggggactggagagttggcgccgctacgctgagagaagtggtggcaccatatgaagccaccgtggaggaactgaggcaagaagtgaaggccaaagaaatcgagattgataatttgagggagaagctgaaaacagcaacaagtcttagcagcagtggtggaaagaaaagcaagtcaaaacgaaaagttagcggtagcagtcaagctccagttacaatgtcgccggcacctgacgtattcgaaactacagtgagcttagtaaaagaagcttcaaaatccttcacgagtttgctcctctctttgatgcgctctgctcactgggacattgcagcggcggtgagatccatcgaagctgcttcatccaacacaaacacacccacggcagattctatcgttggagccaaccatgccaaatacgcactggaatcttatgtgaaccgcaaaatgttccaaggattcgatcacgagacgttctacatggatggaagcttatcatcactaatccacccagatcaacaccggcgcgattgttttacacagtacagggacatgaaagcaatggacccaatggaacttctgggaattctccccacttgcagtttcgggaacttctgtttcaagaagtatttggccattgtgcacccaaaaatggaggagtctctgtttggtgatttggagcagcggcgacaggtgttggctgggaaccatccgaggagtcagttttatggggagtttcttgggctagcaaaggccgtttggctgctgcatttgctaaccttttctttggatcctccgccaagtcatttcgaggcgagcaaaggatctgagtttcacccgcagtacatggaaagtgtggtgaagtattccatttccatgggcataggagggagaatgggtatgggtctagttgtcggatttccagttagtcccgggtttaagcttggaaatggatcggttgttaaggctagagtttatttagtccccaaaaatgggttttaa

SEQ ID NO: 21 - DNA, 324 bp, synthetic construct
  source 1..324: mol_type = other DNA
atggatcgtctaatcaactctgacaaagatgtgaagttgtttcgcttgaagggaatcatcagaaaccggataggcgatgacaatgaagcagctagcatctttaacagacttggagaaggagtgatcccttcaactcagttctattacaaagaagcatgcaaaaaggtggttgagcattgtgaaaaaccatggagtgaaaggaaggcaagtttttggcacaattactttaatggtccttgggtgggactttcaactgcagctgcagtaatcctcctttcgctcacagttatgcaaacagttctaactttcataagtactctttaa

SEQ ID NO: 22 - DNA, 953 bp, synthetic construct
  source 1..953: mol_type = other DNA
atggcgatgggaccagaaagatcgaagccgcctctacacaatttcaccttaccgtgtgggctcaagtgggggaaccaaaagttcttaaggtgcgctaaggttgaatccgacggacaaatctccgccattcatcgcaggtcatttgggtctgagttgatcggacggcgccgatcaaatgatcgaaaattcaggccaccggaaaagcaagatgccggtgaaggaatcggagctgtgagagagaaactcatgtttgatctccaaacagaagctgataagataaaagacgcaatttttagagaaggtttggaggagcaacaactgtctccggcgccggcgaaaactgcgactgcggctgtatcatatgccggtgaattgtccaggccgtggaacttgaggactcggcgtgcggcttgtaaggagcctaatggattcgttgccggcgccggtgccgccggaagtggtggtggtggatcgaaaggagggttgaagattgatgcttatagaactaatgctccgtcgccgttaaggacggaaaacaaatctccgacacttcgaagtgattttgccggtggagcggccgccggagcttctgccagcggcgagaagagacagagagtgaagttttcggtacccctttcgcgaggagagatcgaggaggatttcatggcgatggtgggacatagaccccctcgtagacccaagaaacgagctaaatttgttcaaaagaatttggatgtaagtcaaaaaatttcctctctaaattgaatcttttgatttatgataatcggactaaatagatggaacagatataaaagattgtaatttgcgcatagcagtagctgtaattgataatcattttgatagtgaatttatctattttgcagacgttatttccggggttgtggttgacggaaattacacctgacttatacaaagttcctgaagatcagtag

SEQ ID NO: 23 - DNA, 2877 bp, synthetic construct
  source 1..2877: mol_type = other DNA
atggaaattatcagtgagagaagaagaacccgatctcaagcggctgctattctagtggagctaaaatccatcaattttcaatcagaaaaaagacccaaagtacatgaaaaagatggtgtgaaaaattttgggaaaaggaagcgtcgaaagggtagtaatgtcaggagaaaaaagaagggaaaatatgtacaagttgactctgatgatgatgaagactatgtgtttgataaaagtcccaaaaggagaaagctggattatggacacaaggggaagagttccagctcggatttgaaggtactttccagttttctttcgtttttttattactctttctgctccattgttatgtgatgttgtttggctggacatggagttagagacatgtacaaactataagttaattatgtcattaaaggtagaatgagaaagtttaaagttaaattatttctaaatataaaaagatttcattctttgtgggacactctaaaaaggaaaaaatgtctcatgaattgggacggagacagtaatatataatgtatattatgtgtatatagctctatgtctgcgcatatacatagtatacggtgtatatgtttagtatactgtgtatatatatggtatgttatgtgtattacagtgtatatgatgtgtgtgtgtgtatagtagatgatgtacaaacggtatatatgtactttttgattgtataatatatttgacaaccttctggtgtattaacagtatagttgtatcattgttatgtagatttgggatttttatgttcaaccgagaaagcatttcacagcaagaattgggacacacactaatgttgatgttgttaaattgttgaatgcgaagttggatgataggcagattcagatgttcagagaaacatgcttcgggcattttcttgatttgcccgatgtattggttcagcctcaattgatacactcgctattgctgagggaggtggttcacgagagagaggatgagttgtggatttcagtgaatgatgtcaggctgcgttttgggcttgttgagtttgggattatcaccgggttaaagtgtacgggtgatgctgatatgtgccttgatttcgacggaacgaatcggttgtttgatacgtacttttctgaacttaccagggtacctaagcaatccttaattgactgcttccttaataagagatggaaatccgatgaggatgcagtcaacattgctgtcctatatttcatacacacattcttgctctccactctgaatcgtaattatatcacgagggatgactttgggctcgttgagagcggtgcatatcaaacgtacccttggggaaaagttgtctttaaagccacactgaagtctattaagggtaggttgcttgggaagccctcgatgtacaggcttcgtggattaccattggcattccaatgttggttttacgagtgctgcccttatgttaataagaagattgctttccgggttgatgacaaagtgccccgcatacttagttggaaagttacaaagcagccaaacctcaagaagttgtcgaatgggattttcatgcagaggcgggatcaggtaattgggaagcagtcattttttttatactgtgttaacatatacacttgatattccattaacacacacactaatgtaggatatattttgcactttaaaagtgtatattaatggtattattctagtatattatattgtaacattttgctatacatgtaaaaccattactataccatctcagaatggtatatctgtggtgtaattattgtttattacacattattagttatgaatcgacgtacgtttaatctcttttcatgctaatgtttttttatgtagtttatgctgaggaatatttctccaacagaatttgagcaaacaacactgaatttgcctgaatcatttgaaaatgaaagggataatgaggtggcatctggagatggcgctgaagttcatcttagtgatgatgactttagcggttcgccgcctcaaacaagtaggaagcagcctaaaaccaaacctgatcatcccttgaacaatgatgactggagcatcgagttaaaaagactgagtgatggacagtcggagttgaagagtgagattcagatggtgcgaccacttgagctattttagtttaacatttgaatttttatacacaacagttttttaactttgtttttttcccctttcactagctcaacaaggaggttgcatcgctcaaggactgcatggtagcatcctttgcagatgcttttaaagccatcaagtctctgtcgaagaaacaaagagaaaagattgcttccgaggtgatgcacaaagtaggggttcctttgaatttgtgtgtgctatttcagtatatacctgaaaataatgatgtttttcatttttgaacagcttgatggagaaaatggccaccatgacagacatggcgaaagcgattctagtgactttagtaatagtgaagacaatggttgtgaaggtcaagagcatggcaaggactctatgggcgacaaggacaatagtgagaaagggaatgaagtagctttgggcgatgctgagggccgggagacagttagagatcctgtggaggagggtgaaaggatggacgtatctgcgtctccgattcttccagatacatgtgaggtttcagaccacattacaccggaacaaagtcatccgacaccaattctagagtcaccatatgtgaaccaaggcgacactggtgttgagggttcttccgagaagtccattttaaaaggagagcaccctttagagagcaaaattgttgataatgttgattatagtttgttgtctgagtttgataagtgggttgacgaagggatgaagaaagaaagcaagtaa

SEQ ID NO: 24 - DNA, 3307 bp, synthetic construct
  source 1..3307: mol_type = other DNA
atgagtagtcacgaacgccgaggcccggtggccgtcgtcggcagcggcggaggtggaggtggaggtggagaactgtattggacagccggaaattggctcaatagaaccggcagcggcggcggaggcggttacagtcacgagagcgaacctgacttggctgctatggttagtgattttttggaaagtagtagtgtcggtgcggagtctcggtatagcagtgataatgactctggtttctccgatctcgctcttctcgctgataggatttcggtgagtattcttttgtttccgtgtatgcatattatgcttatcggattttaatttgcattctttgtgagacacatactgcatacatagctagcttgaatagggattttctccagagcaattaaagtgtgaaatggagtaatttttgtctctgtgtatagacatgtttataattatgcttatcaaattctgattttgcatggtttttagaggttttagctatgaaattgatcgagtattttggtctaggtgtatatagggagagcttgaatagcgcagataacaattttatccagagcagttaaagtttcagcggagcaatctttttctctgtgtatagacatgtttaggctgatcggcgatccatgagttgaagtttatgagttcctaaagctacctcaagttaatatacaataataattgagttcatagtcaattattgatagatattttgtgatttcttaaaacatgtatagagtatgggcaaaagctattgggttcaggtaggttacaagctaaatccgcctctgtgtatgttgagctttagttcaagtatttcggtctaggtgtatacatagcgaccttgggtagtacaaataaggatcttattctgagaaattaatgtttgaaacggagtaatccttatatctgtatacacatatgtttatgctcatcagatcggattataatgttgcctgtcgatgagagatgctagagttgatattgctcaagtagtttgatcttggtttatgtagcgagcttggatatcgttatagcacatacgggattttttccagaggattaatgtttgagatagaatagtcggagttccacacatatgacggtgatggttttcccccataggaataaagtttgaattggaatgtacaaatttcttttattctttttagtaattttttgataaggtaaaaagctgtccataactggaaccaatttgatgaattagggattcttttagcatgacattaaagataggaaagaggggccataagtggtttctgcctttgcaggtaatcaagaaaaaaatgatttgtctgaatatctagtatttgattgctgagtaacaagctatgtatggttggatggagggatcctgcgatggtcatatgatggtgaaattctttctgtgtgacacaatattggagattttgagtacagaattcaaaagcttttggtgagtgttagggagtgaaaatttggggataatgaggagaaagctaatggattgggaaaaggattatgcggagagtaggaaggaaagctaagaggttgagagctcgcatttagcaatagggtatttaatttttatcaggtgtgatacaagggtagtctatgtagaagcaagattgtgcgcacactacatgggcatatgcttcatctcagttttggtggttgttttaattagagaaggtttctcgataagcaatagtttaaaattgcattttatggtgggtgagcacaagctcctgaaaacgcaaatttggataacccgccggcattagcaaacctactatcctgcactaaattcagggcgggcagtactacctttcttccttggacatatggtatcaacctgccatgacttttctaagtaaaagcatcactatttgtagtaggctccaatcttgatttctccttttttcaataactatgaactaggagatggaaatgactaggagttttataattatagctgttggaatatgttcgtatagcaaattttggagtaaaacaagagctatttctatgtccctttctgcttaccatgcctgttgcagacatcttctgctgaaaagacttgtcaatgcaacgaacttccaaatgaaagagtcacttagagctcctagcataagaaaacttaagtactagctctcatatatttgtctgctgcttcttgctgaatcttgttacctgcaaatttgaaccattatgtagttacacaagcactcagtggaccagtatgaaagtgatctaacaatggtggttcattcgctgattctttcgttgggcgagtcctgccatcttagcaagcctgagacttgcaatgcgagctgcatcagatctaatctagtgaagctcctacagtcttgcggttatgatgcagctttgtgttcaaccaagtggcagggttgtggaaagattcctggaggtatgctttcaaagattattacttatctgaattcgtttcctgcatgtttgctaccttaagatgtctaactatgaatatttctttggcttataaagaaagtctttcacggtgctacggtgttttgtattacttccaatatatctatatgctgggttccaacttagttctagtgtcaatgtgtgcaaaatatgtggcattgtctaccatatatattagtagattcaacaagataatgttcccatggaactttatcttttgattaaaataattaacttttcaattttgagcgaaaaatttaccgtggtaataacgtctgctgaaataaatatgagagctggagttctgcctgacattgtgctacttttcctatgttatgaaggtgaacatgagtatattgaggtgatctcacgtggaaatgatggatgctctgaaaggtacatcattgacatcgacttcaggagccactttgagattgcaagagctgtcaaatcctacaatgtggtcttgagttgtcttccaccagtgtatgttggcacagtaagaaagcttaagcaatatcttcagactatggtagaagcagcgagatgttcgctcaaacagaactcaatgcctcttcctccgtggcgatcccttgcttatctggaagccaagtgggaatcatccagtcaaagagtagccaactttcaggtccaaagcagcattggcccctctaattcctctcatcagcattgtactgagctgttgtggaggataaaatccagtattgggtctgaaatcaacgccaaaagtttcttggttcctaaaagctgtagaaagaagcagggactaaagattgataaattgaggcattcttcaccagtgacaccgtga

SEQ ID NO: 25 - DNA, 840 bp, synthetic construct
  source 1..840: mol_type = other DNA
atgcaggaagaagaatttttctgccctagctttagcagctattcctcaaatagagcggcggagattgccgctaaaatctccgatgaaattgaacgcaattctaaggtggcggagcaggcggaggtggaagcgaacgctggcgaggatgattttgaattctctttggtatgtgaaaatccagaagcttcggttggggaattcttctacgaccgtcaaatccagcctgttttccccgttttcaaccgcgatctgttactaaacgttgtttcttatgacgtagatcacaaaggcggcgttgacggcgaatcgtcggaaaatgccaatagttcaatccaggtttcgttaaaggatttgtttttagaagatcgcgaaccgccgtcgtcgtcgtcatcggaggtcgatgagttggaaagtgtacctccgggaacctactgtgtatggaaaccgcagataaccgagccttcaccgagcagatgtaagaagagtaattcaacaggatcggcgtttaagcggtggagtattcgagatttgatgcgtcggagtaatagcgacgggaaggacagttttgtatttttaacgcaggaaaaaggaacgaaaaacgaaacttccaaaacaaaggactcagttgaggcatcgaaagtaaccggaaaattaaaggcaaagggaagtagtaacgtcggagagaaggcatcttcgtcggcggctgtttatttacggaaccaagcggcggcaaaggaaatggataagaataagaggaaatcatacttaccgtatcggcaagatctggtaggtattttcgcaaatgttaacagtttagggagaacttttccacctttctag

SEQ ID NO: 26 - DNA, 2254 bp, synthetic construct
  source 1..2254: mol_type = other DNA
atggagaaaaagcagctggatttcaatgcaccacttttatcagtgaggaaaatttcatcatctttatcccctcatgagagagcaaataaaaagattatagaaagggcgccaccgaatcgacaacaatcacttcctgtcaaaaaatcagattgggaattgagtgaagtgactaaaccagtagctgttccatttatgtgggaacaaatccctggtagacgaaaaggtaacaatgatgcccgagctaaccttcgagtggagtgctcaagtagtcctaggctgccccccggaagattgccagagactattcggttctattcgggtgaaaggcctcgtgctcaaaacatttacaagtctccagctgaaggacttccttggattgatcatgcagctttactggatagcctagtggaaagtatatatacaagaggagacagagaaagtgaggatgatgcttattctgatgctcctgaaaccttgtcacctaccgaatccttatccttggactgcagtgtcagtggtctgagcggacatcaaagttcggattcaaaaccatctggaaccttttccattgactcccaaactagagactttatgatgagccgattcttacctgcagcaaaagctgttgttttggagacgcctcagtatgttcaaaaaaagcaagttccagtcagtactgaacaaccgaagccagtacccgtggaacgaaagccaacagttaagcggatggagtctaaacctgtctcatattatagcggctatccagacgatgtaggaagtgaaattgaagatgatgtgtctgagaatcagcacaaaaggccaagtaaagggtggaaatttttccctcggatttgtgtgaagaattctttatgcctattaaatccactgccaggactgaaagtgaaaacacacgttcccacaccctcatctcaagcagtaaagagagtttcaggattgaaaccaaagacgccccagagtcccacatcttatgctcatgaagtcaaaagattagctagaaaagcttatagtggtcctcttgagaaggtaatcattatagttttcctcagttatttttttctgtcctaagattaataagatttatgcttgtttctaacaataatgttctttgcagaattcttgtgatactataaataagcaaagatttcactctggagtactgtctcgcgagctttacaaagctgataatagaagtttttccggccaactacctaaccctagtgattcatgtaagctagttggaatctctcctggaagacgttcaagaagtggggctatatctccctatcgaaatgtagcacccccatctccattcaatgaaggtacaaggtttcttggtgtgccaaaggaaatggagagtctttgggctagtcgctttgattcattccggaaaggttgctacactgtcaaggacaaagtaccacagcagattggcacaggaaggttttctgattcaccaagtgaagtagttgagaaaacattgtacatagattctgtagataatgtgcaaatttcagcccgtaattctgcttcttcaaaacccaagggattagtgaactcctctagtaagaatatgaagacactggttaaaagcagtgtagtactagaaaatatggatgccacggctcgtactcaaggtgccaagaactggaatgtttcagagaaagagagcaaacagattgctgaaaaggagtcccttgatttggtggaagcttctcccgtttccatatcaactctcaaaggccctgctgatcaggagtctttgaaactaaagcaaaatcttgatacactatctggagccttagagtcttcaaaagtgcatccctatggaaatttaggtacagaaaatgaagataacaaaaatgcaaatgacccaaaagattctaaccttacctccgtggaatctcctataccaccacccttgcctaaatctccctctgaatcttggctttggcgcacgttgccttccattcctttgagaaccccattctcaagtttgagctccaagaagcagaacaagaagtcccacactgatggtaccaagtgggagactattgtgaaaacttctaacttgcataaggatcatgtccgttactccgaggtaacctctccttattttttgaagttttctgttgcaaattttgatgtttgagaaagtggaattatttacttctgctccattttgtttgaacaggaactatatactcttggttcttgtcagcagagcaaagcttga

SEQ ID NO: 27 - DNA, 2770 bp, synthetic construct
  source 1..2770: mol_type = other DNA
atggaaatagctcggagctcaagaagatgcaaatgggttacagcatttcttgttctactagcttgctcagctgctggacaagttgaagatggtacgccccttcttcttccctttctattttaccatttcccactttgaagatcatattattttcataaacaaaaaatgaaatttatgcttgatgggatttaccaacttggtatgagatctgatgaacaatttcttttgtcttcattttcagcttcaatgtatgttaatttaagcatataacacgatgatgtcatctgaagactcaaattctaacattggtagaaatatttaattttaaacaatggagtagtgtatttgttcgtaaattttgttcctctgtctcaaacaggattattttcctccaatttccggaagcaacattttaatatgtttgtatcacatgaaaatattgtaaagttaaattgttttttttaaatcttaaaataacaaataaaagttatattaataagtaattatgtagttgatgttgatgatcatgaggtgttattgcgacaccctcaccctataagaatcatgacaatgcaggggttatggtcctcactccacacaattagaccgctatttctattttgatattttgtgtacgtttgatttacatattataagtttattttaatttcttaatgtcgtacacattcattaggagtgagttggtgggggagctatagtatcattactaatttcaataattaccattggttacatttttaggcctacagagttactacacaggacttatttgcataaatattccttcataatttgtgaaagtgattacctcagttgagtgccgtgtgaagtgaatgttccttcatgtcatacactaatttttggtacgattttgaagtgggctcaataataaatgctaccttttctttatatgggtctttttcacataatgtgtgcataaatattttttcatgtataatagccactaagcttaatttatttccaaaaattagcttatgaagctaagattttaaaaaagagacaatattggaaacaaagacaacgaaatcttaactacaatgagttgatattgcccaagtgaaaatatattttagttatatatcttagtaaaacatttactagtggaaaccttctctcagtgctgatagtaaagaaaaacaaaaacaaccccatatctttattaaattatcaatccaaccttgtggttttgcaggtccgttagttaacggtgattttgagacacctccgtcaggcggtttctcttccggcgacggattctctgatggccctctcgtactccctagctggaaaacaaacggcaccgtcgagctagtggaatcagggcaaaaacagggtgggatgatcctcatcgtaccacaaggtagacacgcagttcggctcggaaacgacgccgagataagtcaagagctcaaagtcgagaaaggctccatttattcagtcacgttcagcgcggctcgcacctgcgcccagctagagtcactgaacgtgtcggttcctccggcgtcacagaccatcgatcttcagactctgtataatgtccagggctgggattcctacgcgtgggcctttcaagccgaggaagatgacgtgcgtgccgttttcacaaatcctggcatggaagatgaccctacttgtgggcccattattgatgatattgctatcaagaagcttttcactccagataagtccaaaggtaaggtcacttctgtttctttactttcttatttgggcccatttccagctgtttatcacaccacgttttggtcaaagatttgattctaattgaaatatctacttgggttttgagaaaaatttgaatggcaaaaagaataacttaaataaggtttggcatagaagtactgcaaatctcaaaattagttttgatgttttcataacttctcatattatggagaattaggaaaaaattcagatgatcaacatgaagccgagaaataggttcattcaccttctagctagtttttcgacgactttaatgcatttctcgcatggcttgtacatgaaactcgtgatggatgcatgaacaaaagacactcttttgatgttaaaaaaacctgaagcagaatgcaacatgtaaataactacaaatgttgctaacttgttccttatgttggctatcggaacagataatgcagtacttaatggcgactttgaagaaggtccatggatgttcaggaatgcttccctcggtgttctgcttccaaccaacctcgacgaggaaacatcatcgctacctggttggatagttgaatcaaatcgagcggttcggtatattgataattatcactttacagttccagaagggaaaagagctatagaattgctttcaggaaaagaaggcattatatctcagatggttgaaaccaagcccaacaagccatacagattgacatttttgttgggccatgcaggggactcatgcaagcagccactggctgttatggcgttcgccggtgatcaggcccaaaacatccattacactcccaattccaattcttcatttcagattgctaatctgaatttcacggctaaggcggacaggacacgtgttgcgttctatagtatctattacaacacaaggagtgatgatatgagctctctttgtggacctgttgtggatgatgtgagggttgaagtatctgggtctagcacagtcaaggttttggggttcgggtttatgttgtggctgttagtcttagtattggtttaa

SEQ ID NO: 28 - DNA, 1808 bp, synthetic construct
  source 1..1808: mol_type = other DNA
atgagaacattgtgtccaaatttggacaaggcagatgggttggagacagttttggaggtccccataccagaggagatgtttaacaatatgggtagcaatggtgccttacaatggcggaatatgcgcaatcttatgagggctcaaagtgcagacagatattcttcttcttctcatcatgccgctgtatcaagtaatgatcaattcatgttcttgtttaaaatcgttggctctgctcttgtcccttttcaagttcaattggaccatattgctgataacatgcctgttaggaatggttcaatggtaagcactctattccttgtttccttttcttattgaacattcttgaatgcgattaaatcttaaaattggataaaatctagcaatataacttgttatttttggtaaaccattctccaaatattgacaattacaggctttaaaaatgtaaactgtatttgttgtttcaagagttttagagaaacaatgtctagttcctattttcagtgctgctaccattttcttgttttgtacttgtatctaaaattttgccctggccagatgctcttgttcaactccttagatatgaataaggttcaaggaaaaaagacaaaagaaaatgttttaactgatttagatttcagtttgaaaaactatatatgttgaactcataactcgtacgttatggtcatcaggaggctgctactgctaaatacatagtacaacaatatttggcagcatgtggagggcaggcggcgttaaattcgctaaatagcatgtgtgcagtagggcaggtgaagatggctacatgggacatccatcaaagtggtggtgacgccaactcgaaacgccattgtgaggttggagcctttgtgctttggcagaagaaccctgacctgtgggttttagaattggttgtttcaggttgcaagataagtgcaggaagtaatgggaaggttgcttggagtcagtcttcctctacttctagtgcttcaaaaggtcctccaagaccccttagaaggtttttccaggtatccttttgctgatgaaagtgttacatacacagcatttctgtaattaaatcatgcctatagtctttgcttcaacagaatggagacatagttggtacaaccaaatgcaatgaaatgagaattctttaatatatttcggttgcttacctagaatgcatgcataattttgatggcaaaataaatcatgtatcttctaatacatattcatgactttttgcatgctcatccaactgcttaaattgactttaagggtcattcaaatgcgagtttatgtattttgatgaacagggcttggaccctagatcaacggccaatttgttcttgaatgctatttgcgttggagaaaagactattaaggatgaagaatgttttattttaaaactagaatcaagcatagacatgctcaaggcacaaagtacggcaaatacagaagttgtccatcacacaatattgggatacttcagccaaaggacagggcttctgattcaatttgaagacacaaaattagtaagattgaagtcaccaaagggggatagcaatgtcttttgggaaacaagtatggaatctatgcttgaagattatagatatattgaaggtatcaacattgctcacagtgggaagactgcagcaacaatttacagatatggaaagaacatagactatagagcaaagattgaggagacttggatgattgaagagattgatttcaatatatctgatttgccaatggactgcttcttgcctcctgctgaatttgaagagcaaggaatgggttgctga

SEQ ID NO: 29 - DNA, 1076 bp, synthetic construct
  source 1..1076: mol_type = other DNA
atgctgcaacatctttatcttggtcaattttctatgatgtttcttcttctattatgttctatttttgcagtattaacgactgcagaatcatctatatacgaagtgcttgaatcccatggattgccaatgggtttacttccaaaaggtgtgaagaatttcacattagacaattcggggaaatttgtagtccatttggatcaagcttgcaatgctaaattcgagaatgagtttcactatgataggaatgtatcgggtacaataagttacggacagatccatgcactttcaggaattgaggctcaagatttgtttctatggtttccagtgaaggatattcgggttgatatacccagttctggtttgatttacttcaacgttggcgttgtatctaagcaattctctttgtcttcatttgagactcctagggattgtactgctgttcaactcacggatcttcaggatgacatgcacatagctgacgctatttctaaggtcttttctcttccttgattcagtcttttctaagttgaccattttctgctctagtttctatgaaacttttgcttattttgtttggaacaattgaagacacgttgtccagtaatttggctacgtctgtaagtcccctgtaacacaggaacttgtaccaaggtttgaacttcttcaagaacaattattccccaaatggatgggtgaatctaagcttggttattttagttctgattattcaagtgtatagttctttgcattatttggtttctgcttcatttgttgtccaaattgcagtactagtatttgcaaaaattgcaggcagattattgcaagcactgtccttttcttgttcaatttggatgttgtatataaatattttgaagctaatgaaatgttcacataatgtgctttaagtagtagtagtatatatgatatctgcttctgcattttgaagttcatttttgtgaaagtgttccctgtttgtttattaattcttggaatggttatttgttgagcagagtcaatcaggggagcttcggtacaagtttgatcagggcaatatcgggaggaaggctatattgtag

SEQ ID NO: 30 - DNA, 918 bp, synthetic construct
  source 1..918: mol_type = other DNA
atgcaagcttatggtagaatgaagagagtaactgacccgcttgacgacaaagtgaaggctcggatcattgggcgtgaccagcaggaactcggttacctcagcagcggaagcgaacacagcgcccacgccgatgatgatgcttcctgtagcttttctactctcatatttggtttgcctgatgatgtggcggccgaaaatatgtcgtcggagaatgattcagactccgacgacggagatgtgtcaatgtacgagtcgacggacgtgattgaagatttgccaaagccagtgttttatagcgatctggatttgtttcgtaattcgctggcgactaagattactaaggcactggaaatgttttccttttcgaaatcgaataagccaatcctgaggcgaaatgtaatgacctatcttagggattttggttataatgccgctatatgcaagacgaaatgggagagttccggtggacttaaggccggaaactacgagttccttgatgtgatccgatcggattccggtaaccggatcactcgttactttatcgacctcgattttgcggcggagttcgagatagcaaggcctacgaatcactacgagcgtttatcgcaatcgttgccgagggttttcgtaggtaaaagcgaagagttaaagaagatgttgaaggtaatgagcgacgccggtaggcgttcgctgaaaagcaaagacctcagtattcctccgtggagaaaacaccggttcatgcagaataagtggttaggtgcttacaaacggacaaccaacgttctcccgtcggcaaactcatcggcgttgttgtcgccgtcgaagcagactaacgtcgctaagtgccggtccgttgggtttagcgctgtcgccgttaacggccgtttactctttcccgcggcaacccgtacaagatga

SEQ ID NO: 31 - DNA, 1086 bp, synthetic construct
  source 1..1086: mol_type = other DNA
atggctagtaggaaacgaagcatctcaaatgatgtagatatgcatgtcctttacaaggaattggatgaagcttcatgccctgtatgcatggatcatccacacaatgctgttcttctcatttgtagctctcatgataagggatgccggtcatacatttgtgatacaagttatcgacattcaaattgcctggatcgtttcaaaaaactcagagccgaaaatagggacagtcctcctattatgacacagggaaacctggacattgctgttgagaaccccgatgagcagttgagaaatttaagcgatcgttctgttgttcatggaaatagtaacagagataatcacatggagatgcatgaaggtactgtacaaacttcaggtgctgctactgtatggggaagtagtcaagaaacagcgagtgctgacggctcatctgactcaaaattgaaattgaagtgtcctatgtgccgtggagatgtattaggctggaaagtagtggaagaagccagaaagtatctgaatttgaagaatagaagttgctctcgtgaatcatgctcattcgttggtaactacagagaattgcgccggcatgctaggagagttcacccgacagcacgccctgctgatattgacccttccagacagcgagcttggagacggcttgaaagccaaagagagtatgatgacattgtcagtgctgttcgctctgccatgcctggtgccgtcgtgcttggagattatgtgattgagagtggagacaggctatccggtgaaagagaacgaggcgcgggtggaaacagcaggtggttgagtaccttcttcttgtttcagatgattggttcaatggacccaatatctgaagcaagaggtggcaggtcaagagctttgtcaaggcaccgtcggtccactggacctttatctaggcgccgctatctttggggtgaaaacctgctaggtctacaagacgatgacgaggatgaagatgagcgtgatcttaacatattgagtgacatgagtggtgacatacctacaaatccaagaaggcgtaggcggttgatgcggtcgaggtcagatgaagatcagcaatag

SEQ ID NO: 32 - DNA, 2143 bp, synthetic construct
  source 1..2143: mol_type = other DNA
atgccttttccgatgaagattcaacctgttgatttcaataccgtggaagaaccgagtcgatatgactcgtttaagccggttcaaaagtcacggttcaagcggctattcgagaggcaattttcaggacttttgagatcatcggcgccgctggagaagctggtggccggcgaagagttgatttgtaataagaaggatgttgcgtccgacgagttcgagccgagctccgtttgtttggctaaaatggtacagactttcattgaagaaggtgaggccgataaacatcggtgcaatcggaaccgaaaatgcaattgctttaactgtaatggtactgacagctcagaggaggaaactgattcactcagttgcttcggtgaatcgaatcatacttgttgcaacgatgcttgtgaaatcctcaaggtattgcatgttccattaatatgcaattttaccttaatttgttgttaaaattcttcttagagttaaattaacagtgtctgttctttgcgtgtttctcaaaataactccctttttggtgtagagtttggtgccgtgtccttccgttattgaaaggaacgttttagctgatattacaaagatcattgagaaaaacaagatgggaaagcgaaaggacaatttcatccgaaagatagttgttgatggcctcttagctgcgggatatgatgcttcaatctgcgagtctcgctgggacaaaactccttccactcctgccggtaaactcacaatttcaacaacaaagatgtgctggatttttttgcttaaatctgaattggaaactattaacttcatgctcatatgtgtactgacatataaatctctttagttttgtatggttaaagatctatatatattccagtaactgtgagatgacatataaatctctttagttttgtatggttaaagatctactatatatattccagtaactgtgagagtgagaatattaattttaaagtcgtcaaattattgctaaaggtattgcaatgcatctgaccgaaaatagagcagaatggatacaaaaacttacaatgaagtcaattggttcattcatcatataatcgatcctattatttcgacacagaagacacatataattgatcccaattagttccagattaaggcctaatagcaagaaaacttggtctgcaacatggcaagttacaacttgcattttgttttatattcaggctagcttaaatggagaaacatgaacaatgaagattcatatagccaataccaacgtgtttgggactgaggcatagttgttggctatctgcttaggctgcctttttttctttcttgctattatggggctaagaattcaacaaaaagtctgaatcttttctacaggagcgtatgaatatgttgatgtttgctattatttttgggttaagatttcaacaagactctatcttttcgacaggatcgtatgaatatattgaagttgttgtgtatgaatatgttgatgttgtttgctatttattgggttaagagttcaacagaagtctgaaaattttctacaggaacgtacgagtatgttgatgtggtgattgaaggagaaagactactaattgacattgatttcagatcagagtttgaaatagcaaggtcgaccaggtcctacaaatttctcctccaattgctgcccaacacttttgtaggcaaagcggatcgccttcagaagattgttcatttgctaacagaggctgcaaagcagagcttgaagaagaaaggaatgccttttcctccatggcgtaaagctgaatacgtgaaagccaaatggctgtcaccttacacccgtatcaaaccaactctaatgggggcaccaaccgtgtcaaactctgctcccgaaactggcactgcttgcaaaacctatcaacaagcggtaaaggaggagtcttcagaggattcttgcggggagctgaatctgatttttggagagagcagccaactcttggagaataacgccaagagcgtgattacctatcctctatctgcttgtgatgacgaagaaaaggatgtgatggtgcagcaaggcaaaccacctgacactaaacacaaggattcaagcaatggtgccaggaagataactggtttaacttctctgattgaagaccacacatga

SEQ ID NO: 33 - DNA, 495 bp, synthetic construct
  source 1..495: mol_type = other DNA
atgccaatgtctttaacaagcattctctgcttcttctttctcttcttttcagccacaacagcttcaagaaatgagaagccatcagcctatgaagagctgcagcgttatgacttcccaatggggattcttccaaaaggggtaaaagactataaattaaacaccaaaacaggtgaattctcagcttatcttaattccacatgcagcttcagattggaaaactcgtatcagctaaattacaagcctgttataaaaggggttatatcaaaaggcaggcttacaaaactgagtggtgtaagtgttaaagtggtgttgctatggcttaacattgttgaagttaggcgtaaaggtgagaatcttgagttctcagttgggctcacatcggcgaattttccgattgagaacttcgaggaatgcccacaatgtgggtgtggattggattgtgttagtaaagaggagagaaagattagacagaaggtctttgtgtcttcttcttag

SEQ ID NO: 34 - DNA, 419 bp, synthetic construct
  source 1..419: mol_type = other DNA
atggaggagctccgcccgagtttccggtgctccggcgaccgaagactagagatcgtaagcggaaaagggttcagcagcaaccaggtaagcgttacgcatattaaatttcgaactcataatttttaagaaccttaaagttgaactattaagtttaaattttagattcgcctcagcagggaagtcgggctcgctcaccggatctaccagtagtaacgagcaagggaacttggccgagccaggtggcagcagcaacttcatcgtcagctaagccctggggattcaatgatccagagatgaagagacgaaagagaatagcaaagtacaaagtgtatactattgaaggaaagttcaagacttccattaggaatggactgaggtggttcaagaataaatgctctgaaatcatccatggctattaa

SEQ ID NO: 35 - DNA, 3589 bp, synthetic construct
  source 1..3589: mol_type = other DNA
atggggttttcggagaacaatagaggattagttctagcaatggtgtccagcttgttcattggaacgagttttatattgaagaagaaaggtctccggcgtgctgctgctgctggcactcgtgcaggtctgaaacgtctctatgtgtttgtgtgtgtggtgtgttagatctttacttcggtttattagtcaatttactatatgaaaaggcacaatttgttcgctagcttaaacttgaagtacttgtgtagatgcacaattccagcaacttgtgttgaaaacgctttatgtgtgatattagtgacaaaatatatcaatcaactgcgcatttggtaccaaattagttggccttcgacttaatgaaccctctatcgaggcctgtttaatattcaaaagaatcatcacaggcagattatttttagctgacattagttctttatcatgacttaggaccaaaaaagctttgcgaaactcatgtagacaaagttaccttagtgaaagaatatactgcattaattattctttatttagtgaatattgctattttacatatctaatatctaagcatgttaaggacattgtatctcaacttaggtgaaaattgttctgttcaatttgttgtatttatgttggttcttctttgtttgatcaagacaatatttttttgggtgtgtaattctttgttcattagggtctataggcaccatctggtagactatgtgatagtagcacataattactcaattagagccaagcatactaggatttttaatcatttaggtggtggaaacaatatccttcaggatcactttcagttaggtattacactgttcaggcctgtgttggaatacataaatgatgtgggggatgggcacgagaaaagagaagagaaaatgtagaaatctattatttggatatgtagagggaactgctgaagtgaaaaatatgaggtctaaaggcaaacaaatcaacttcatgtttttgtgctgagtctattgacaacttttctagccccattaggtccagccttcttctggtagcactggaatagccgaattgggttcttgacatttagttttcttcttcttcctgttgtcattttctttaatttaaaccatgagtagaagattgacaaataatagttgtctagcagactaaaacattgcagccccatgtgtatttactagtagtttttattcttattcatgatcttgcaaacccatacataatatgagaagaactatctttggttggacattctagtgggagctgaagcttttatccctgaagtacttgtgagtttcacatgcagtaatctattccagtacttctagctgatgatagtttgcttggaagtaaatgtagttttgatttttagatgtagtctagtgtgccaattttctatttagtacttcaattgatagcagaacatcaaacatcgtatttagcatcctttatcctcattatttactcagtcaacttcaattcacctggctaaacagttgttgtagttttcactgcaggagttggagattatacttatttgcttgaacccctttggtgggcaggcatgattacaagtaagttgacataagctgtctccataagttaacctctttccaggattgtgttacattgttctggtgttaatgtcatttggatttggacatgcagtgattgttggggaggcttccaattttgtggcttatatttatgctccggcggttcttgtgacccctcttggcgcgttgagtataattataaggtatgattttgaagtcttatgagctgttttataactctgataaggaactcctggtgatgtttgtgtaactgactttggtgaatctgaatagtgctcttttggcacactttatgttgagggaacgattacaacggttgggtgtggtaggatgcatattgtgcattgtaggatctgtggtaatttttatacatgcacctcaggagcatatgccaacttctgtacaagaaatctggattttggcaattcaaccaggtctggactttttgtaggttgtaagatatttctccctcttgcagagcttattagtaatttctccttgcttcttttgtcagcagcatttatgatttatatagcagcaacagtatccacagtagtagctttgatgttgcattttgggcctcgttatgggcagacgaacttactggtctatttggtaatatgttccctaatgggtgcacttacggtaattatgttttgttctagtctacttccgtcatctccttttattctttggtggaatttgtacaactacattcttgttggacatgatacagttttgcacatgtgaacattttgctagcctgttaagatgttctgtaaaattttattgtattttgcaggttgtcagcataaaggctattggaattgcaataaaacttactttggaaggaattagtcagattgcctatccgcagacttggttttttctagctgttgcagtaatctgtgtaatcacacagttgaattatctaaacaaggtaatgtctgtatggtactttcccagctcggctgagactttcagtaatccaatgatcactcgattacattaccttgactagcgtgaagtctcatttctctgaaataataacatgcctgtcctttacgttaaaattcagaatgcatctatattgatgtcgtcctggcaaagcatccaggacatgcatcatgcatatatttggtgtcagttcctctgaactaaaatattggagtagttatttggatttatgctcttcatgtaatgatatcaattcttgcacaaggtgtcttttgttaatgacatgaagataaaacatagaatcaatacttgatgctaactatgctgcaatatttttctgaagtctttaaattatgtttctgtttttaacttttgtgatttggcaataaatgctctaatttggatcattatgtcacagacaaataagtagagtactatgaaactgaagccgtccttaaatgattcatagctctactcctaagttgagatgaaggatactgcagttcagattctcgtttcctttgttgctctttcttgcatgcttgatatgtgagcagttttaatatctgaaacgagtgctttggttggtaatctaggaaaaagaagtttctacaatagttggtacgacttcatatcaatatttcccttatcacatgaaccttgagatgctgcttgaccattatagagcgcggttaatatgaactgcatgctattatgcctaattttggttttgcagttcagcaatatgcattatatttaggggaattcacattgcttttctttagggtgtctgaaccattttctaacgtttatgcgtcatttggataggcactggatacattcaatgctgcaattgtttccccaatatattatgtaatgttcaccactttgaccatcattgcaagcgcaataatgttcaaggtatgttgtcagtttgaactattcttaattgtggtctcctatcaatatgttcacggagtaaaatgtgaacccatcatttgcttattgctttgcaggactgggcgggacaagatgctagcagcatagtatag

SEQ ID NO: 36 - DNA, 1777 bp, synthetic construct
  source 1..1777: mol_type = other DNA
atgggtacagatacagaggtagcagaaatgagttcaacactaaagcttcctctgttttcagtagagtcatcacaagaggatttagggatgctaactcctcttcacactttagcttcaatacctttcaaatgggaagaagaacctggaaaacctcgtccatgcactgatcttatccctcttccaaactccatatgtttagaaccacctcctaggctttacaatatggaattttccaagacatcttcttctcctacaactgtatttgatggaccttatattaccaacaagcctaggttttcttcgtttaggttgttgaggagggatcgtcgtcatcgtcgacaagggtcttttgatagtactactagctctgaaacaggacaacttagtactacacttgttcttggtaacaagaaatcggaaagcaaaagctggtggcgtcggccgactactcatcatgttaaacataatggctgtgagattaatagtggttttgtgtttccatcttctatagattattcaacagattgtgttgaagaatgcagcagttccagtataaaaatggaaacatttagaagaagtggaagcttctcaggtcactcgcaagctaagactcacatctgggtaagtgtctttacaactccactaattgttcgttcgtaatcaatttattatttatatgtagattttaatttcattaatgaattttgctattactactgtttttcttctgtccgtctcatgtcaaattgctctatctaagtcatgattcgtactctgcctttaacccccttgtaattaacgtactatgatctcaatgaacttaggaacattgaaagaaagatgtacttactatatagagtctctaccctagattctttaagaagattggccgccccaccgtaggccttttggcatgtgtcgaggggaattaaaatataatatataaaagtataaactgatcttttgaatcggcgaatgattctgcccctaaatacgcccttgcgccccaccctttcctttaagggatcaactgtttgttctttgactataaaactttcaatcaatatatttttagtaaaattcatgttacatagtttccatttttgtttaaataaaacttgaagaataatgactgaattaataagaatcaactaactagaaataccttacagttcctttttgtaatgagggactaatatgaacagagaaagtacagaactagtggggtgatagagtaatgagaaagcttaattattggggcaggtgaggttgaagaagacggcagtggggctaaggaacgcctagttaacttcactttttaggcaatgggggtaggtattacacaaacccacaaatagctgtaagcaattatgtatcaaaatgttaaaagctactactgtattaatagatggggttgaatgagtaggtagatgctgtcccaagttatagggcgtccattgtcattgcagtaaataggaaaggggcgaaggatatgggctgtctgattgaagctgcttttgtaaaggattgggagatatctggagacttttctccttagcatattttctatccttgctctgctatcattatcagcatttaaaatcatttgcgtgattcgttggatttcataattaaaactcttctatttttctatcagcatttactaaactcaagtattggtcttgttgaaatcttacaggcagctatatacgagggtttcaagcaagtaataccatggaagaacagaaagtctaaaaaggaagcgctaatcggctaa

SEQ ID NO: 37 - DNA, 1737 bp, synthetic construct
  source 1..1737: mol_type = other DNA
atgtcagggtaagcttggtgatttgtaagctttctcgttaatttctgtttttttaatttcttatggtgttcaagttgctgtctttttttatttttagtagtctgcacgttgtagaatttgtatggtttgctgaattgcttgatccacagtaagattagtctttgcaagttcaatgttcaaaattgtctgtatctctattagaaagtgctagccagaagtatttcctgttgcgatgcattccctggaattaaattaatctgtcattattttgattgcttcaaaggaacccaaccagtagaaccatccaaaacctggcatttcgtaggtcatttgaagcagaaccaaagacaattagccgtatgacagtttcttaactctatcttttggccacctattatgtctctgtagcctatttatatacatattatttgttgttcgaattacaaaacccctaaattgtcatgctccggatgcagagccgacatatagctagtgttttatccaattagtggaccactttattttcatgaaaaaatggacatgttactggatgtagacaaagtatcatgaaaaaatagattggcttagcttgcatatggacatatttaaggtggatttgattatttgctttgatataccgagcttttctgagaaagaaacttgttgtgatgtcaacattctgaattattcgagtttgaggtttttgtggcattggcttttataccattgtaatgtgacattggatggtgatttcttgcttcgccaatctggtctttgatgtgtgcctatctttaactttagtgtaaggtgttctctagctgacaattgtgtaactgtagagaggatgctgctgttcctgttgtcgctgctgccgagactcctgctccagcacttggggagcccatggacatcatgaccgcactacagctggtgctaaagaagtctaaagctcatggaggacttgctcgaggactccatgaaggtgctaaggtgattgagaagcatgctgcacagctttgtgtgctagctgaggactgtgaccagccagattacgtcaaactggtgaaagcactttgtgctgatcacaatgtcagtttaattacagttcccaatgcaaaaactcttggcgaatgggctggtgtaagtaattggtcattgcttatctatgaccccagttacagtatctcagttttttactatacacgagtagctgttttctgatgtgaaaatgaaattggtatgtgcctttactagatactccatcttttagaaggtgaagtgttttagggacttgtgataaataatactcagaatgccatgactgaagccttaacctcatgttggactgtttttcgcacttgtaatggagtggaggggtacatgtgggttctgactattaacttgttgactcctctttgaagtgttctgtatttctcatttttgttgaagtgtatcatgtaagtcgaaatataatcactcatcgactatgatctttcttcgattacagttatgcaaaattgattctgaagggaaagcaaggaaggttgttggttgtggctgtgttgtcgtgaaggtacatcctatttatcctttggctttacatgagtagtatagctgataagtttccaaactggaatttatattccattgtaatgatttttctgattgattctgcaggattatggtgaagagactgagggtctccatatcgtccaagagtacgtgaagtctcattaa

SEQ ID NO: 38 - DNA, 2237 bp, synthetic construct
  source 1..2237: mol_type = other DNA
atggggaacagaagttcaaatcttgataatgacaacaatatttcacgtccaattgatactccatttaagcttccttctccgttgcccacttggccctcaggtactttctgtctatcaatgtgtaatcttccattatcttatcatttttgctttactagtatctgatcacttctggtgttgttaaaggctcgcttaaagcgcgcttaattctaaagctcggtaaagcccaagttgtgtgatttgcctcgcttagttgacgcgtgcctattttcattgatgggctatgtcttaagataacaacactagacaataaatatatttggcaaatagaaaaaaaaaaggcagtccgatgcacaaaatatcccgttcatgtacggtcgggggaaagaccgcaccttaaggctatgatgttgacagtctgtcctaatacaagcattagagggttcgagccgtgaaaccaatcattaatacttgcattagaaaaagtcatggctgaaattttagtgattattgtttgtgttatagttatattttcttgcaatacatgtttatctttttgtaaatattttgttcattgtcgcttttctctgctaaagcccacgctttagttgtgctttgcgcttaaagatttaatagactttggccctttttgtgcttttctcatttggctactttgatcaattgttctgtttattttgattgtttaaatattaggtcaagcctttgctactggagttattgatcttggaggtttagaagtgtctcaaacatcatcattggctaaagtttgggctactcaagaaagtggaccagatgatcttggagctacattctttgaaccatcaaatttaccaaatggcttctttatgcttggatcctatagccaacctaacaatttacccctcttcggatcggttcttgttggaaaagattcaaaaggagacgcgctaaagattccaattgactatacacttgtatggagtagtgagaacttgaatatcaagcaggatagtgttggctatatttggctgccaattcctcctgaaggctataaagccgtaggccacgttgtaacaacgtcgcctcaaaagccttctcttgacaaaattcgttgtgttcgttctgatttaaccgatgtgtctgaaagtgatgattggatttggggcaataatggattgaatgtgtattcttcaagaccaagagacagaggaatcaatgctttaggagtctctactggtgcttttgtggcttcaaataatggaactgcagattcattagcttgtttgaaaaatgtcaatgctaatttatctgctatgccaaatttgaaccaagttaaagcactatttcaagcctactctcctttgtactacttccatcctgatgaacaatattatccttcttctgtcacttggttttttcagaatggagcattattatataccaaaggtcaagaatctgcaccagttgctattgagtcaaatggttcaaatcttcctcaaggtggttcaaatgatggtgctttttggttggatttgccaactgatgattccgcgaaagatcaagtcaaaaaaggagatttgcaggctgctacagcctacttacacattaaaccaatgtttggcgcgacgtatactgatattgctctgtggctattttacccctttaatggccctgctagagcaaaaattgaattcatgactattccattagggaaaattggacaacatgtcggcgattgggaacatgttacgttaaggattagcaacttcaatggggagttacaaggtgtgtacttttctcaacacagtggaggaaattgggtaagtgcttctcagcttgaattccaaaatggtaacaaacccgtggcctattcatcgttgcacggtcacgcttcttatgcccaaccaggaaaaaatctgcaggggaataacaacataggcttaagaaatgacacagcaaaagggcaaatgatggacactggagcaaactattcaatagttgctgctgaatacttaacaattgttgagccagcatggttgaactatgctagagaatggggtctgaaaatcagttatgatattgcaaatgaactccaaaaagtggagagatttttgccaggaaaactgaagaatgctgttgagaaacttgtgaaaagtctaccaaatgaggtgttgggtgaggaaggaccaactggacccaagtttaaggacatgtggaatggggatgaaaggggttaa

SEQ ID NO: 39 - DNA, 492 bp, synthetic construct
  source 1..492: mol_type = other DNA
atggctttctctcaaaagtcacaactcttgttttgctctctcctcttcatcttaatctccctttctctctccttcaatcctagccgtccaaataaagatgatgatgtccatgatctccttccttcctacaatcttccaattggacttctcccaaataatgtgaaatcctacactctttctcctaaagacaactcttttacagtccaactcacacacccttgttatgtacagttccaagatcaacttgtttactaccaaaaagaaatcaaagggaaaatgagttatggtgaggtttctgatgtctctggaatccaagctaagaaactgttcgtgtgggtccctgtaacagggattaatgttgatgaggagtctcatatgattgaattccatgttgggtttttgtctgagaagttgcctgctaaggattttgagactattcccacttgtaagaataataaggggtgccaagattctctcctttcttcaatatga

SEQ ID NO: 40 - DNA, 902 bp, synthetic construct
  source 1..902: mol_type = other DNA
atgttgcttggaaagaggccaaggccaccaatcaaaagaacaacaagcatgaaagaattcaccttggatctcaacataaacgatccttctgtagccgttataaattatcagccctttgatcctctcaacccctacaacttcactggtcccgtcccaatgccctccaacggcctagatcaacgcttaatcttgactcgcagaagatcagctgatttcgttggaactgcccatttccttagagcttgctccctctgcaaacgccagttaatccctggtcgtgacatctacatgtacaggtatacagatgtatacgatttaacttatataccctgagaaggagagccttggcgcagttggtaaagttgttaccatatgtgatcaggaggtcgcaggttcgagccgtggaaacggcatcttgcataaatgctgtgtaagactgcgtccaatagacaccttctgatcgaatccttctttgaacctcgcgcatagcaagagcttagtgcactgggctgccatttttaacttatacttgttttctatgttactaatttcatttattattgcctatcacatgtagttattttctaggcaatcagataataaaattaatttattctatcaatttataggggttgaactctttacatatatgtagctagctagttatttctgtattcttttttgttgaaatctataataaacttatttgtggttgttttttttttttcagaggagatagtgctttctgcagcctagactgtagagaaaagcagatgaagcaagatgaaaagaaagagaagtatccgaaggttgcaactaggaaaaaagttactaactcagccgccatggccgccgtggcaagcggaacaggatccgacgctccacctcaggccagaccgtaa

SEQ ID NO: 41 - DNA, 1443 bp, synthetic construct
  source 1..1443: mol_type = other DNA
atggccaacaaggtctccaatttctccgatctaatccaacgtgtcactgcttcttgcttgctccatccactttcctctggccgtattgacagcaatatctccgatgctcattacagcgatgaatctgaagatgacaagtacagtaccgaagatgaagaagaaaaaggagacctaccttatgtggagactcaggaaaaattccagaacgttgaggtcacaaaggaggagataaaaactgagaaagtaactgaaatggagatgcttttaggggaagtgttcgacgtggtgtctgcagtgaagacggcttatgttagtctccaagaggcgcattgtccttgggatcaggataaaatgcgtgtggctgacgtggctgtgatatctgaactgcggaggcttggggtgttgagggaacgatttcgaaggaatattggtggtggaattagaaaaggggactggagagtcggcgccgctacgttgagagaagtggtggcaccatatgaagctaccatggaggaactgaggcaagaagtgaaggccaaagaaatcgagattgataatttgagggagaagctgaaaatggcaacaagtcttagcagcagtggtggaaagaaaagcaagtcaaagcgaaaaattagttgtagcagtcaagctccagttgcaatgtcgccagcacctgacgtatttgaaactacagtgagcttagtaaaagaagcctcaaaatccttcacgagtttgctgctctccttgatgcgctctgcccactgggacattgcagcggctgtgagatccatcgaagctgcttcatccaacacaaacacacccacagcagattcaattgtcggatccaaccatgccaaatacgcactggaatcttacgtgaaccacaaaatgttccaaggattcgatcacgagacgttctacttggacggaagcttgtcatcactgctccacccagatcaacaccggcgcgattgttttacacagtacagggacatgaaagcaatggacccaatggaacttctgggaattctcccaacttgcagtttcgggaacttctgtttcaagaagtacttggccattgtgcacccaaaaatggaggagtctctgtttggtgatttggagcagcgacgacaggtgttggcagggaaccatccgaggagtcagttctatggggagtttctggggctggcaaaggccgtttggctgctgcatttgctaaccttttctttggatcctccgccaagtcatttcgaggcaagcaaaggatcggagtttcacccgcagtacatggagagtgtggtgaagtattcaatttccatggggataggagggagaatgggtatgggtctagttgttggatttccagttagtcccgggtttaagctgggaaatggatcagttgttaaggctagagtttatttagtccccaaaaatgggttttaa

SEQ ID NO: 42 - DNA, 6827 bp, synthetic construct
  source 1..6827: mol_type = other DNA
atgatggggagtggattacagtttaatcggagttttaacggtgaagatcggttttatagtgcggggaagtttcgccggaatgttaatcggagtttttctaccggcagtgaggataatctccggcgagctaagagtgatgtcgccgttattcattccccgacgaagccgaaaatgaaagttgccgccgatgagtcgcctgatgagccggtatctgccgtgccgccgacggatgttctgcccccgccgctttgtaatcttcagcggtttttgaaatctgtttctccttcagtacctgctcaatatctatctaaggtcttaataaattatgaaactcttttatgggtttttgattgaagatttaactattgtttgtcggtttttgatatgtgttttggttgaaaatgtttgtgtttgtatgggttattcaagtgtttaaacgattggattagtataaaattgtacttccctcgtttgtccgaaacaaaataatacctttttaaatttagaaataatttaactcttaaacttttttttacaaaaatgtataggttgttttagatcacaagtttcagtttcaaaagtacgcttttctttcgtaaactctgtgcgcatcaaattgagactgagggaatattactttagttggtattttcacggagaatataactgttgtttgttgttaagattataggtttgacggaaaatggttgcattagtttaaagttgtaatctttttgctgggtatgttgataaactgttgtttattgggtttaaatgaaagtttaactgtggtttgttggtaagattataggttttgattgtaaaggttgtattagtttaaagttataatctttactgggtatggttgaaaatttaactgttctttattgggtatggttgaaaacttaagttctgtttgttgctaagattataggtttgattgaaaatggttgtattagtttaaatttaaatcatttattgggtatggttgacaatttaacttctgtttgttgctaagattttaggtttgattgaaagtggttgttttagtataaatatataatctttaactgctattattgggtttgaatgaaagtttaactgttgttcgttgttaagattataggttttgattgaaaaatggttgtatttatataaatttctaatattttaccgggtatgactaataatttaactgttttttattggtatggttgaaaatttaactgttgtttgttgttaatattataggttttgattgaaaagtggttgtattgatatacatttttatcttttactggatatgattgataattagctgttgtcagtagggtatggttgaaaattttattatttttattgttaagaatataggttttgagcgacaaatgtttgtgtttgtaacagacaactatgaggggttggaggacgtgtgatgtggagtttcagccatactttgttcttggtgacttatgggagtcttttagagaatggagtgcatatggagctggagttccattggtgttgaatgaaggtgatagtgtagttcagtactatgtgccctatttgtcaggtattcagttgtatggtgactcgacaaaggcttcaacaaaaacaaggtatatatttgcatggtttctccttccccatttagcctaaacttagtttttgttactcatggcctagtcaaattgccggccacatcaatcgatgctctcaataccccactgaagatgcttgttttgtattctctggttttccgtgtactccttgatccttatttcagttccacaattattattgcgtatatttaggctttgtaagtaatgattatttgtttttacatgaaatgctgatacacaatagttgtggcactaatgcaaaattgtgtctaatactctcggtcagattatcaggactcagctagggaaaagctttggatgatatattatttgtgtgtctttcactttccgcatgttgttactgaagcatgttttgcgagaaaaaattaaacgcaatagtgatagagaaatagttctatttgcatcttttcaataaaaaagtggatggcaaaaagttcattccgctttgcaatgtcataaagttaccgaatgagcttttaggtggatttgtgggtcattgctataaatcaaattggtacccgaatttgagatgtggctctttaaccaagtacttcagtctccttttttagatggcctcttaacattgttgcattcggaaacaacttctctacccccttggggtagggataaggtctgcgtacactctaccctccctagaccccacttgtgggattttactgggttgttgttgttcttaacattgttgcacatttctttggacctgtaaacttctgcttctataagagctgtctgatcagctacagctgctcctaaaccttattgttactatctaacttagtgtttgtggatagatgcaaaacctattgcacttcactaattgaggagtccaaagaacacgttcaactctcaccttagtgtatatagatggaaataaaacctttaccatctcattgacatgtgaaacagtgaaggaacttctgcattcctgattttagtttctttcaagtcaattattgttttttttagaaagaattttttgtcccactcccattatagtccttcgttgtcatacaaatagcgccctgagtaaatttatctagaagtatgcctgcataacgatttaggcctgaatgagtataaaattgttgattttacttggtgaatgctttttcataggttttgatcccttctcaacctaatattcaccttttaaaattaacttattcttaccatacttgtagttccttcttgaacaatatacaacttcaacatgcttttgtttcttttttgataagaagcaaacttcttcttttgaagctgctgttgatgcagtcgtatgcaccatctttggtactttttattaataaaactttacattatgaaaaaaattcttattctactctcttcatttcaatttgtgtgtcttagtttgattgcgcacggagattaagaaaagtaaagaagactttaaatcttgtggtcttaaactaaagatgtgtgtaatgtaccaaaaatgccatgtggattctgggagttaaagaattattaaacatagaaaaagacgttctttttgaaacaaactaaaaaggaaagcaagacatataaattgaaacggagggagtaattttttttcctggataaccatggtgtccgggctagcttgcccgcaccttgattaattccacggggtacctgctacctcccaccaccatagatactgggcaattcgatctatcaaggcttcgataaatgagaagaaatcacctagtgttttagcctctgctggaatttgaacgtgagacctcatgattttcaacgccacacccttgggtgctcttcttttaaaataaactagcactaagtaaatgctgggtaattacattgaataaacttttgtacttacccaacattttgattcgcactcggttgctttccttccctttatctcatcttatttcatgcaatttgcttgctctatgcctctatcccatctaagttttatatgttaaccaattcatgttactattagtgaaatgtgaattgatttgcctagtgcctaatgaaatgacggatctcacttcgacacagctattatttattgtcagtttttctttttaatggttgttgtttttcttttattttgataaatcaaggtcttttgttaaaaatcatggcaagatggtgtaactatacaaaagaaattccattctcaaaaaaaagaaaagaaatttcatgtgttaggcctcttgaaaatctgtccaactttctatacaataaggaatttcctttttacaccaaaagtatgagtagaataatacggagctttttccccagtttttattttttattttaataatagggaatttttgactagaattttgtttagaaatgggagtttaggatgtggatatgtccctagttgctcattcaagcagttgaatgaagaactgatatcgttattccgaagtaccataaagtgaatattagaaacgtgttcagtaaaagaaatatttgcattatcagagtctatgaaatggatgtcctagtcattcaggcaagtagctcattgctattccatctatttttattggtcagcactgaaccgggcttctcagttgtactttgccatgttctctctctctccctccctccctccttaaacctgcctttccctattcacgatgtgttttttctgtaggagcagaatatttcgtcaaaggctatggaattttcaatttgccattctttctgctgcatgcatctttttgatgtatcattgataccattctgctctttcctgagacattgttgatttcaatgcactccttagattgtacatgggtggacaggtcggttatagcttgaaattgtgtattatgagtgtgtttatgttgtaaatgtgcatcattgtgtctctttactaccccctcaccccctcctcttttctgaaagggtctgaatcttgaattgaagttgtttaaattttgtatgagtaactttaaaaaactatgatattgacttgcattttcttacacagtaggcgaccaggtgaggaaagtgatagtgactatttccgggattctagtagtgatggaagcagtgactctgaacatgagagacgttgcttgaattatacaagggagcagcggatgtatcatagccaaacaagtgaaagctcccttagtattgaccggttatcattaagagacaggaatgctaccttccaagaaggattttcaagcgatgagggtgaatctgggtcttctcagggtgccttgttgtttgagtatcttgagcatggtcagccttacggttgtgaacctttggcagataaggttggtgtttcctccctcttcctcaaagtagttttcgtttgtctttcatctatggggagtaatgtataagttacccttcttgcttctttattatatctcatattttcttttatgtttcttgttctgtagatatctgatcttgctcaacgttttccggaattaaaaacaatgagaagttgtgatctccttccttccagttggatctctgtagcctggtattttgtttttctttgcgcatatcatatgagtgtaattttttgtatcttgaattgtaatgctgatgtctctcttgcaggtatccaatttaccggatacctactggaccaactttaaaagctttggacgcttgttttctgacctttcattctcttcatacacccatgacaggtatctgacagtttctaccctagattgcttttgtttttcggttggatattacaactactgcttcttacccatccatatcactgtgtgtattcttttcataatgtaatgtggtgttttttgacgtttgtctatgtattttcatctgtattagtatgtgacaatcatatatttccttaatgttcttgataaaaaaagaaaagaaattattcactgtcaaacaaagagtgattgaccgtatcaacttcttaaattcatcgcaatcaatatattttactcatttgatccgctaataaggattcaagaagcttccaaaatagctacataaaagaaaaacacaatccatgtgaaagttgtgtagcgaagtctcctcttcctttttttaaatttatttcggtctcgctgaaatgaagtggattggagattttcgtttgtttcaaaatattttattaggaataagaatataatattcttttaagcactgtgttgtgtatcttgaagtatatagtctgaaaggaatagaatggcaagaaattggaagttcccattggtaatttggtgtcattaaaagtgaggttagaacaagacttagtttgtgtgatgcagtgtttggtttgaatgacttacgaaacctaaaattgatttcctccaaattttcaactttctcttcggttgaatttcacatgtcgtccgggaaagaaccggggcgttaatatgttaaagaaagttagtcgtgaaaaagatgtccaccttaattcattaactcttttctagcatgttagaaagtcaatctcttcttgaatttaggttttgttattcttgagcatcctggtgaacaaattagtcatgactgtaaaaatgtttacattggcattaaaaataaaatctgctctccgtttatggtcatggaagggagaagagttgggagggtgagagcacaggaacttaaaatatctcttgtttgtttcctttcttttccctcccatgtttcacttcttttattccttcgcctgcctacttcttcctcctactttacgatgtgtggacaaatactatggacctcgaaatggtaacataacatgaggtgtgtgtaggcatattccctaaaaatggtaggtcactaataatttaggctgaaattgttctatctggtgaagtatgtattaaattttagagtatgttacatagcttattgtgggttgcttttggcatttaactaaatttgttatgttgcgagtgagttgttgaaattgcacatggactttgtgcaggaagtcaaagtgctcatcctgcggcggtaacatgtccgagtgatacggatccggtccctaagattccattgcctgcttttggccttgcttcatacaagtttaaagcctcactttggaccccaaatggaggatctggaagacagttaatgagctcgcttttgcagtctgctgaaaactggctaacgctgcttcaggtcaatcacccagatttcagtttcttctgccaaaggtga

SEQ ID NO: 43 - DNA, 3439 bp, synthetic construct
  source 1..3439: mol_type = other DNA
atgggttcatgtgtttcagtgcataaagactctgaatcagccatgaaaattcgtcttgtttttggttcttccaaaactgacaagcttgtcactccttcacctctcaacaaagatgataccaaagtctctgatcttcaactcaaatctcaaactcctgctgttactgcttttcgtgactttggtactaccctattactctctctttattcatttttttattttacaacagaaactctgtgataactctgttatattgtttggcaatgtggaaaaaaggggaaagttgctgtctttttgaattgaaggttaacatatgtttctggttttgaatgttttttgagtaattagtttgtgttgagttgtctcacattggttgtgggatgtgaggcctaaggttcatttctttatatggtaccggtgacagagctagacctgtacgatttcttggtttatcctatgttgggctctatattatgttgtccatgcgcaatctcaccccatgacttagcttttagagttgagttaaagtccgttcatgccgtattagagttaagttatacccaaagtccgtttctttaccgcgtgtcgccatgtcatgctgtattagattcttgcctctggtaataggttttgtggtgttcctaatttttggtatttttcaatttctatgagacacacgtgttatttgattactgactattgattatctgggcattttggaatgtaaaggaaagttgaagaggtgttctgtttgatctgatggacattatttttgtattttttgtgatattagttgaatatgtgattgagataaaattgttgttataggaacagttaaggttggtggcctaaaaagaaacactttattatattttacagagtagctttctattccctttgtggacttagaaatattgaccaagaagctattttcagaatcttgaataatgccaaaaattcttcagttgatggggccatctgcagaacttgttatcatgggcagggcaattttttaatttgtggagaaaattattaagttaggaacttaaagtatcaccagaaaagaaaaaggaaatacttagcgtaggagtataccttaaacaaaggagatattgtagtacttctaatattatatacaaagtggtactttttagcattcctcagctcattacatgaattgtaaagaatgagtctttgcagccttgtaattagtcattggagcattctcttttttgctgggctgctacttttattaaacaaatatttcttatataaaaatgtgtgtgattgctgccactattattttatttttttggtaattaaagaattttatttaccaattgtaatatgtacagtgagatttcttcctctactattaaaagccattttcactagaaaagggtagtctggtgcacaaggcatcccgcgttcacgcaaggtctgggaaagggccggttccttaagaggtgtgatgtagacagcctaacctaatacaagcattagtggctgcttccacgcccgaacccgtgacttataggtcacacggagacaaccttaccattgttccaaggctccccttcaaaagccatcttcactaacaccggccaatatcacattataccattatgcacttgacaaggacatcaatcttgctgaatattgcctcttgcttctgttgttcctcttcgtcacttaaagggaggataatatcattaacatttgatgattagatggtcctatggaaatcttagttcatgtcttgattgtcgtattcatcattatctccatgtctaagagaatatttgagttgttgtattaaaatattctttgagatagttaaaactgaggttgacatttgttataaaaccactgcccttgtttgacatatcatgttgtgggtctacactgccatcacttctactatccgtctataataatcagcaaatagtcgacaattttctcctttttttttcctaaggataaaagtaattttaccagtattctcggagctttggttcaatggttaaggtagtacattgtgtgttgtgtggtaggcacacatcacgagttgaagcttgctatgaatcaagtttagtgtttaatgaagagaattaaattaactaaataaattttacaagtaaaataaacacactgtacagcttttagtttacctcacttttaaggaaatatgttaagttctctgtctttcttgaatcttttccttgtttctagcagaagataaatgttttacaattagtttgatatggctaatggcctcagccttctcatagttcagttttattcatgtggattaacaaaggttagttgaggatactaacatcatatccaccgttatttccgctgcatcatcttgtgttcttgaatcatctgcctaataatagcactctttcttgaaagtcatttgtggtttgccaatgagtgttcccaaccaatctcatttctctttatctgtgttagatggatttattttggatatgattatctggccttctggttgaataacgataaggacatggtgtatcctccttttcagttttcttctttagtttctcaaagtaccactctgattcactgcatgaaacagaggaaaccgtgctaagtgtttaaaattttcatgcatagtgcgtgcgtttatatttacggtggtctaatttaattgcatcttttatctgagtcagagacatttctttcttgtaggtagcaaggaggagaccttctttgattcccagccttggttggattcagattgtgaagatgacttcttaagcgtgaatggaggtaagatcttcaatcgatggtccgtgttattccatcttctcccctttccctttccttgtcttcataaatgagctacaaaatggcactttatttattgtatccagctaacagatgtgcgttcttcagtccaaaaccagtgtttagaattgtccatcagccatttcttagttagtcgtgtatatcatcttgtatttttataatctctctgaacttgactccagattttacgccatctcgtggaaatacacctgtccatcccttggctggaaatttaacagggaacagaacccctgcttattttcagcaatcatccccaacagataaaaagaagagattgtctgaactttttgaagaaagcttgagaagcgatctggaccaaaatgggcaaaatgctgaagaaaaacaaaatggtacaaacactaaaaaagaaactgcgtcaactggtactcaacttccaccaagatccacacctgggacaccttatgcatccgtgtgtagtagcgaaagaacacccagtggtctgttaaaatctgacgtcaaaacgtcaaagtcagcacaatgttgtcttccaaggttgctctcaagtcgtagctttaacggaaggaggagtagaatgagtcctgcgcgtaatgttggctga

SEQ ID NO: 44 - DNA, 7325 bp, synthetic construct
  source 1..7325: mol_type = other DNA
atgaaggatgatgaatccataccagtttcaacacctacaacacattcatcatcaaatacaatttcaacatattcgaagaattccacatctgatgccacaatattcggtcgtggtcggtacaagttctgggcattagccgctattcttctgctagcattttggtcaatgtttactggtactgttactctccgatggtccgccggcaacctcaacggtctctccgattacttcaatgtgcctctctccgataatctcgacgtcatcgtatggttactatgccctatactttttctttttgtaaataagttcatccattattgttcaattttgactatgatttttgcacgatcatctcacttaataggtaaatacttgtaaatcttaaaaattaatcggtaatctagtaaatgaaggtaattaatctgctatgacatgttaaaattcactgatagtgtacaaactctttacattgtcagtgtacgtaacttaaatgtagaaaatatttatacaatcagatcacttaataggtaatacaagtaaatattttgataagcgttaattgatagtgaaaatggttacttactaacctgctattataggttaaaattcactgatagtataaaaaatagtatgtatgtaacttaaatgtttttaccattttgatacgagaagaattgtaaaattttaatttgtaaatgaggaacaatctagttcgatttagctaggagaattagttaaattgactattcataagctaacactggcaaataatttgggtgcagggggggtacattataatagtgtttgatttgattctttgagaaaatgatttaatcttatttggtaggaaatggaagagagggagaagttggtgaagcatatgtgggatgtgtatactaatactcctcggatcagattgcctaagttttggcaggaggcattcgaggcggcctatgaagagttaaccagtgaccttgctgatattcgagaggctgctatctctgagatctctaagatgtctctccgatttgttcatatggagccaccacctctgcgttcattggttagctctctctctctctctctctctctctctctctctctctctctgtttcaatctagttatttgctgtattctacatgtgcctgttgatacctcaatttaaccttctgtttaggccaactgggaattgttgtctttcttagttttcgagaaccctccagtttgctatcattataaagtattggaaagaagcgaacccaaatggagggtgtggatgtagaggattcatgtaactgacccaactaatttaggattgagtcatagtagtagcagttgttgtgcttgggaattgatcatactgcagattggtgtttttcatggagaagttgcaaatcatagttcaggaattagttgggtgcctcttgttcattgaaagttgtttcctttatgttatggcttctgagcatgctacttcgagtagttagtaacagtccttaaaacctgagctgctgttatatgaggatataataagtttcagcaagaatcgttttgtggaagctaacgtcgaatatacacaacctgcaagcagcctttcatgaattagatcttgtaggattttgggtgcttaggtgatagatattcggtgcaaatgcaagtagccagtataatttagtaaatattttcaaggcatagaaatagttcagataagctgcatctgcactattgactttgtcttgtggggttgttgctggaacttgatcctttgtgcaaattcactttacacatcaagaaaatggttggaatattactattaatttgaacattgctctaaatttgcctcacaaatttgaccttatttgtcttgagtgtattgacttgttatgttaattgtctatttactcgagattccttcttgtgagttagaaaagtggatttcccattggaaagaacttcctctttccaaaattgtataatttggtgacttttcatgtcgattaaaagaataatgttttaatgagttactattatattattagggttaatatgttgtttagaagaaccgaagctggtataaagattgcatatagattctaagtaacagatacagtgtcatctacgttaatttggctttcaagatagattttgccacatagagaaagaggtaattcaggacagcccaaaggtgaaagtcaatcaaagtgaggaggtagtaatattcttcttcttgcatgtcaatgacaatgtaggattgaagtgcataagaacagtatacatctcgatagtatcactttttctgaaattaatcattttgaaggaataggatggaaaggaaaggaataaaaaagaaaggtattgtacctttaccgatccacatgctatcttactttgtttccttctcactcatattttcgtgatcatatctagttgacccttttgtaactcgttcagctccagttaccttctaaacttccaaaatagagcgctgctacactttctcatcgaaaaatttattatcatcctacatatatttatccttatgcagatatcgggtgtgtctatgttggtgcaatccgtgatggtggtggtcataatcccaccacttctaaattttactaaaagtcttgataggcgtgtaaacatgattattttgcacaaatttgagagaaagagaatggaccccattccctttgttggcgcaagactacaactataattgtgcctcaatttcaaacaagttgaagtcggctatatgaatcctcactgaccatatcgaccatttttgatgagtcttgttggcacatgactcgcaaaaatctcttcctccctcatcatttgacaaaacattttcctccatcctcaccatttgtcatcatgcacaccatatgttagaaaataagaatccaacattgataccttgattgttgctgctatcacaaatcaccagtaagtggcatgaccaattccaaccaactttcgtagtatactggcatccacataagcacttaccatctaaacattggagcagcacatacacaatacagaggcaccagctagatcaactactttcaagttctggaaggctgagtggtcaatatttagaaataactacgtggatctcgggtttgactctcattatgatattcattgtgagtccatctgctctagccaggcggacaagattacccgcactgggttgtagcagactgtccaatggatttagttaaggtgcgcgcccaagatttggcctggacagcactatatttaacaagtatcaactgtttagtccttttccagctctacttgagaaatttgttcctctgttgcatgtttgcctctgtatccaaattctattaggggataggagagttatgaggacagtgaaaatcaggacgtaggaaatgttataactgtgataatggactaaatacaaatcctttgctctccttccatttcctatggggataagattgtgaagctgattgttttctttcttaacttatttcacgtttgtttacaatttccatcaaattacctgcttcttctcataatatatagacctgaagcattctctgattaaaacaatgaatagatatgtccttctcaaaaaaaaaaaaaaacaatgaacaggtatgaagcatttttactacttttacgtgacgccctgaattaatgtatagtgggagaatccgggctaatcaagtaatatgccttaaacaagctttcagatcttcaaatcatcatctctatcacttcattgcaattatttgggttgtcatttagaaaaagattattctgagctctgttctgaactgctgataaaagaagtaataggcaccagtgaaaaagaaaaataggaattttcttttatgttgttctgttatctcaaaatggtagaaacatgaatacagaatgtgtctggaattcgatatctatcttcattatctcttgctggattagtgttcgtctgtatccatgaatgttaggtgatttttcccctattgaagtttgtgtttggcatgcaaacttcttttaaattttttttgcgttttgaaattttgcttggccggttaattctcaaagaaaaaaaaattgagaagcaaaagaaagtaattttttgaacacatttttagttgtttaaaattattttaaaaacgattctccaaaacttttccagaaatgcgtttcagtgccactgaagtattgttaaattaaagtccctgaggattaacacatggagtgcaacctaatttggacttccctttttgtaatttttcttcgtttagttgagatttttgaaatccttcatcatctacaaactttattagttcaaatgcttagttcttaaacagtaattcaaagttttattagttcaaatgcttagttcttaaacagtaattcaaagtttgtcgagtgaaatgcagtcaatctattgttgcagccattcgtctgtctgttctgcttcttcctatctatgcattgttcaggaattttttttccgtaactaatttttcttcatttttctaaaatccttggatgataaaactaaattttcctcttttagtcagcagtaaacttttatgctctgttatggaatctggatacctcgtgattttgttttacttaatggactgtcagcattagcagctatcatttattaaaccgaaaattatcctctagctgctcctgtgcaaatgtgtgaggtcttagctgtatatttcgtcacttgtaggggttaatgaccactcttgattatgatcattgaatagctcagtcttttctgtcttttcatttttatctttaaattgagaatgcatttttttaatcctaaatctgccttgcctttgcatccacaatactcgactctacttttcggaggtgattgttattcagtgtaggacttagtcgtctttaagttaatatgcagtccgggcatcaagagaaggaaacaacttacccacactcagtgtttgcaccaagcaaataaatgcaagacatgtttcttggatataaatttttatcacttaaccatgcttaagtagtagatattttcagtttgatttgaatctcttaaggttcaattgcttggttttgtgtaaacactgagtgctgataagtttcattccatctgctttcgtgagtttcagctaatttctaactgttgctagagttgattataaggatgttgtcatcaagtggtggcatgatacttattgcttctatttttcagtcttccttacattagagagcctgcgaattcattcgttcttggcatgaaaatcacaaaggagttagataagtacaatatcagccattatactttggctgaatttttcctggagttgcaaaactgcactttagcaagttaactattcaagttctgaaagcaagcaaatagtccattcagaggagtttttatgaatttttaagggaaaattgtatcattgccacacttgtgatcttttaaggatgatagaggatgatactactttcagagagaaacaaaggaattcagtaatgtaaagaagcatgtaactaaaagagaacagatctccacaactccattcccattctccttctattcctctctagtgaacttttaccatccctaactcttgttatccaccttaacatatttaagtgcaggatagttacaggagaactcccttgtgagtgcttttgtcagatgtagtaagacttatggaaaaggttacatgatacttttgtcgatctaattgttactttatgctaaatgatccgtggttgatcttcatgagggttactgtggatttgttgcgatgtgcctatttaggtttgacagatgaaccaactgctgctagctattacttagaaatatttttgttattttttttaggttaagataatgaattgatttttggtcagctgctcttagttctctccattgtcccttcaacctgctcttccacccttgttatcctgcatttgctccttgaacattttcaatcattgtaattgacatgcatgtgtgatcttttcataaatcatttcaggcagtaagagaattaagtcaaaaacaagctgaggacaaacagaggactgcaaaaggaagtaagttatgactggaagaatatcaaggatccaattttgcgtggttgccacttatttatacaaaatatacaagatgaatgaaaacatacaacaacctgctctctggtatttgactaaagggagcatttgagaacacattacattcaggttcctcgcacttttgcaacagggataaacataatcaattttttgttacttctacctcttttttcccccccatgtacccatctccccattgaaagctcatacgagtggcggaattcaacaatgtcatgatttaattatttcatgcaaagaaattggaagttacttcctaggcttttacattcgaagacccttactaattcgtttgttttccacttgaaatacccattacacaaacttgcactggaatagaaagattatatttagttcttgtggtgaaactttgtccacatttgcatttcaagtgttttgtgtagcacccagccttatcttaggagttttcttcctgtataattatgtgtttcttcagatccagtgttggaaactgttgatgccctagctatttttttattttaaatttatgcgggaatggtgtccgggccagcttgtgcgcatctttttctctttgctttttttacccttctgaattcactgatcatgcaaaaggttccattacatgcctaattactggctttataatcctcctttcttcttgactacctggaccaccttgaaatttgacatgagatacacatgaacaatcaaaccaagaaaaattagatgtaaagccaaagttactgaattattttcttgttaaaattttatgacttcaaccaagatctctggtgactaaaatctataagtatcatgtcattcagaatagtcattttcggaataatacaggtacactctttcacactgaagcaccactcacctgtgttgctggggcttactatagaaaataaagttgaatttttgtcacctttttttatcatcctgtaaccttgatgtgatagatcatgttaaaatgttttttttcctggtaaataaagaattttatggacacagttttcacattattatttgatgcagcaaagactcaaagtaagcagattgctgcccaagtagaatcatattttgtcttaaactttcaaaacaagcatattgacctcatttatttgcatcagtttggctaagtgcagtccttatggaggaaaataaaagagaagaaaaaaggggacctgattag

SEQ ID NO: 45 - DNA, 1221 bp, synthetic construct
  source 1..1221: mol_type = other DNA
atggccgatagtgaatcgaggcaattttcacctgataaggagccatgttttgcagaagaactacttgactggcagaggagggaactgccccgcaggttcttgtctggtcaggttgtctgggatgacgacgctttcatccccgaaggcgcttcggagaagcacagggttatatttcgcagaatttataggaaatacttccttcagatttttgcttcggatgttcgtcttctttcactactcttttattctaatcttgcattatatgtatacacacacatcaattagagctggtcaaatttctaattagagcttacaattgttatcactttcaacaattccaagtttttcccagttttacttttagttggctaaggctgttcttaacattaggagtatatttttagaaaattagttgtttagcctgcttattgtctgagcattcttaacaactaatttgtaatatcccaagctctgcccagtctgagctccttaattttgtttagagccttaagtgtggcagacaagattatggggcttacttggttacgtgttaaaataaatttaggaattcatggctgttttggtggtacttttagccatagtgttcaagtgatttgattagcccctgttctctttattttatttagcaaatagaccaaattctaaaacagcatgttagctccacaaattaaacttctttacttgattgaatatcagcttctttgactttcggcttcaattatgaccccttcttgttatcgcttgagtatgaattcatgactgattggcttatttggcagggtttcgacattgacatctatccgggtaaagccaaggcagctatgcttataccatacctggattttgagaaagaaattgacttgctgatggaactggctaaccatgctattcaggattacaacagtaaagaaaccaatgtaggcctttattcatcttatcatctttgctctggttgaacaatctccctcaccccaagtgtccttacagtattttctacgtgccttgcaggtttacaaatacgaggtcttgtatgttgaaaaagtgaactttattttggccgaatgccgcgaattttttatgactgttaaagttaaaaatctcactctgcgttcacctaaagaaacttttcaaatccatgcatataaaggaccagatggggagaatattttctgcctttgccggagaaaattgctggtttga

SEQ ID NO: 46 - DNA, 909 bp, synthetic construct
  source 1..909: mol_type = other DNA
atgagagactttgcttcttgtttcaatgaatatgcagtacaagtttctgatacttcttgttctagttactcaaactctgcttgtatccctccttctctgattccttcaattcaaaacactgtcacttgtttgtataaagtcactctctccaataaaaaacaggtcttgatcacagtttcatggtccaaaacaaatagtataacacaaggcctaagtgtacactttggtgatgatccttcaaatgtcttcaaactcaacacgaattcgcgtctttttaggaagaaaaggggaagcaaatcactggatttggatcatttaaaggttgaaattttctgggatttatatgcagccaggtatttatcaggtcctgaaccaattgatggctattatttactagtcaaagttgattcacaacttggccttattattggtgacatggctgaagaagcttcattaagaaagttaaaaaatggaactccaatggccaagttctcattggtttcaagaaaagaatacttttcagggaataccctttattcaacaaaggctcaattttgtgacaatggcacaagtcatgacatattaattcgttgtagcggcgaaaatgaaggtctaaagcatcctgttttatcagtttatattgataaaaagatggtgattagagttaaaaggctgcaatggaatttcaggggaaatcagagcatatttgtggatggattgttggttgatctaatgtgggatgttcatgattggttctttaatccaacatctggttgtgcactttttatgtttaggacaagaagtggaatggatagcagactgtggttagatgataaagacaaattgctgcacaaagatccagataaagttgagttttcattgttgatttatgcctctaagaccacataa

SEQ ID NO: 47 - DNA, 504 bp, synthetic construct
  source 1..504: mol_type = other DNA
atggcggcttcaaagagctatttcgctggagcaaactaccgattcctgtcgagcgagcgagacgttccgatgatgagtcctgaatctgcgttcgaattcgatgaatcggacctgtggaactcagcggtttcacagtcgccggagttccgaaagtccgttcagagttcaaaaatctcgagaaagcagtgcgatacgaagagccgccgaagcggttccgtagcatcggcggcgtcattgccggtgaacgtgccggattggtcgaagatactgaaggatgaatatagagagtatggaaggagagatagcgatgatgatgatttcaacgatgaggacggcgatgatttggagaatcggattccgcctcacgagtttttggcgaagcagttagagaggacgagaattgcatcgttctccgtgcacgaaggagttggccgtactctcaaaggtagagatctgagtagagtcagaaatgctatttgggagaaaactggattccaggattaa

SEQ ID NO: 48 - DNA, 2534 bp, synthetic construct
  source 1..2534: mol_type = other DNA
atggaaagacggccaaagcagagtggtgaaagaaagacaatggagaatgaagagcttgtcaaatacatgtcaagtttgccaagttacctggaaaagggggaaaatctgcaggagaaagctttcagtgttggcgtcctagattggcgttttcttgaaaaatggcgacacgaacatgtaaaagaaccttgtagaactagtggttgttcaccatccaccagcaataccttgtcattttcctcaatggagggatcttcatccaattctagcagagccagaagctgctctcctgctcgtcggaggattcatcgtcctacatcacaatcttattatgcatcaccacctaaaggtagtcgtctttgttagattttctcccactgattaagggaatgggttgttttctcctttccttaataagtttggacaatcctcacctcgtgagctagcttttggggttgagttatgcccaaggtccaatttcttaacagtcttgatgtcaaatcctttgcaggaagttatgtacagttgcaaaaccaaaaacctggcgtaattaaatccccggagattgagcaacttgtctgcaggatatatcagtcttttgatgagtatccacaaagaaatatgcaagaactcggcaatgctctacacccgcgtaggactcctcatgttaggagatcacttgatgtagagactaagacgagaacatcacgttccaagggaaagatgaagatccaagatagagaatgtttatcgaaaggagattttgatgattttgattgcatagagaaacataaatcagatgtccttcaagtaccagaacctggccaagaaactaacagctgcacaactttctgcccacctgattcagtagtgaaagaccagagcgcggtgaaatcaagcagacggagcttttcttgtggatttatttctgcgttctactatggacagtcctcttcagatatctctagttccagcacccttcctcatgatgctgatgagtcgaaggtgggacaagcgagtccgatagatgcaaaggatagttgcctttcatctaagacaattcagccttcaacatattcaggaaacaaattgtccagcccacctggaaccaattccaaacaagagaagaaatcaactgtgatgcttaagaatccaactacacttaattccgcagaatctacaaacatcagaaattcctctccaactcgtcaatttagcatggcaatgggaaggattggccagatttcaggtataaaagatatgatcactggatcacaaggtgtcaaatggcctgcagaacaatctagtccaaataaaactcaaagttcgtcctcaattgatacaggctgtgataaatcagacaccactggccgagccaggactagtcctttgagaaggttgctagatccactgctgaagccaaagacaggcaactctgatcacgttaaatcttcaacaagacgtggtgaatctcctacgaaacgctcattgaaagtcaaattggatttgaagagctgtaaatcgattgatatcgacgatccacgttctaatggcacatttgtaccatcaagattgcaagctctcctccaagtagctgtgaaaaatggtcttcctctattcacatttgcagttgacaatgaagttgacattttagcagccacaatgaagaaacttaatccgaatttgaaggattacagttgctggatttataatttcttcaccgttcgtgaaacaaagaaaaagagtggaaattggttaaatcaagtagaaaaagacagaagtcatggaattatgcctaatatcgttggaaagatgaaggtttctgatgttccattctccgaattgaataggcagaaacttgactcacagtttaggattacggaatttgttttgtttgctacagatcagaaagcatctgaccttcacccaaacgatgagcttgcagctattgttgtcaaattacccaatagaacctccatttgtcctaatggaagtggccatcaggatagaaactgcagtaatacttcaacttcaggtttaacaaatccttttgaagatcttaacatgacagttatccttcctggtggtgcacatagcgtaccaagtaaaggcgagccttcttcactgattaaccgatggaggtcaggtggatcgtgtgattgtggtggctgggatttgggctgcaaattaagacttcttaggaatcacactaatcatcaaaggatatcaagttgttccaaacccaaacttaatgctgcaagatttgaacttttatcccaggttatgatctacataaatacagattatcaaaattttacagtaattttcgaatgtctaatacaccattgtgaaatctttttgttcttgtttggcagggggaagcacgggacagtaagcctgtcttcagcttgtccgcattcaaggatggcatattttcagttgaattcagttcttcactgaaggttctacaagcattctccatttgtatagcagttttaaatggtagaaatcaagaaagcttccaaatgcaaacttga

SEQ ID NO: 49 - DNA, 1048 bp, synthetic construct
  source 1..1048: mol_type = other DNA
atgctgcaacatttttgtcttggtcaattttctgcgatgtttcttcttctgttatgttcaatttttgcagtattatcaactgcagaatcatcaatatacgaagttcttgaatcccatggattgccaaggggtttacttccaaagggtgtgaagaatttcacattagacaattcggggaaatttgtggtccatttggatcaagcttgcaatgccaaattcgagaatgaatttcactatgataggaatgtatcgggtacaataagttatggacagatccatgcactttcaggaattgaggctcaagatttgtttctatggtttccagtgaaggatattcgggttgatatacccagttctggattgatttacttcaacgttggcgttgtatctaagcaattctctttgtcttcatttgagactcctagggattgtactgttgttcaactcatggatcttcaggatgacatgcacatagctgacgctgtttctaaggtcttttctctttctggattggttcttttctatgttgaccattttctgctcgagtttcttttgcttattttgtttggaaaaattgaagacagtaatttggctacagatgtaagtccctgtaactcaggaacttgggccaaggtttgaacttgtctctgaaacatttattccccaaatggattgttgaatctaagcttggttattttactcaagtttatagttctttgcattatttggtagctgcctcatttgttgtccaaattgcagtactagtatttgaacaaattgctggcagattattgcaagcactggccttttcttgttcaatttggatgttgtatataaatattttgaagataatgaaatgttcacataatgttctttaagtagtatatatgatatctgcttctgcattttgatgtttattttgtgaaagtgtccactgtttgtttactcctattaattattggaatggccatttgttgagcagagtcaatcagaggagcttcgatacaagtttgatcagggcaatatcgggaggaaggctatattgtag

SEQ ID NO: 50 - DNA, 1198 bp, synthetic construct
  source 1..1198: mol_type = other DNA
atggcttctacagtaccggcgaagtcgaataatccactttacaacttcgatttttcccacttgaaatggaagaagaatcatcattcgaacaatcatcaacgtcgccgttccaacaagctgtcgtcggactcttcctctccgtcacggcacgactcgccgctaaggcactctcagtctcagtccccgatgcgtgaatccctcgcggcggcgcgtcagtctccagtaagtgaatctgctgagacggcgcgtatatcgccgatgcacaattcggctgcggcggcgcgtcagtctccgatgcgtgaatcggcacgtcagtctccgatgcgtgatcccgttccgtcggttcaacgatcaaagcataaggtaccagagattaatgttgtcagtagcaaagaaagcagatcgaaaatcttaattaaaatccctcgcaaaaacaaatccgaagaaattcaaatcaacgaggatcaaaatcaaaaggaggcagatgagtctcacgacgaagctgcagcagctgaagaaacagcacaaaaaacgtggaatctgagacctcggaagccgatacacaaatcactgaatataaacggaggagtcccatttcgatccagtggatctgcgatgcaggagatcaaatctcaatcaccccatcacatgatgaacgtgaacaagccggaaaataatgaaacccacgctgcttctgctcagaagaaggtaaaaaggcaaaggttttccatagcgttatccagagaagagattgatgaagatctatatgctatgactggattaaaagctgctagaagacctaagaagagagttaaagttgttcagaaacaacttgatgtaagtatttatcacttttctttttctttctattttgctcttatgatttctagttatctgatgatatgaatttcttttgcagacactcttccctggtttatggcttgcttcaataactcctgattcatataaagtttgtgaaaatcttccaaaggtagaatttactgtttgatggatttaagtttacactatcaatgcaatgtaacctgttataacacgttacttgccatttatgtaggttacaattagattttaagcgacctgattgtgcataaaacttaactcttgataagattagattttcatttctcccatagttttctgcgtgtatgtgacagtgtgattttccttgcagggttag

SEQ ID NO: 51 - DNA, 1113 bp, synthetic construct
  source 1..1113: mol_type = other DNA
atgatgaacaaccagtcctcaatctgttccagcttaaatgaagataaagttcacgatacaccagatagcttccatcagttacacccgaagaaagattcagattctctggatggaagtgagttagagagtggagatacaatttcaacagtcgaacatctgaaatcagcattaaaagctgagaggaaggctttacattctctgtacacggagttggaagaagagagaagtgcttctgctgtggctgcaagtcagacaatggctatgatcaataggcttcaagaagagaaagcagcaatgcagatggaagctttgcaataccaaagaatgatggaagaacaatcggagtacgatcaagaagccttgcagctaatgaatgagcttatggtgaagagagagagggaaaagcaagagttagagaaagaattggaagcatataggaaaagattattggaatatgaggcaacgaggatgttgaagagaagcaaggacagtagcacattctcctctgaggatagtgatggactctctattggtttgaatcaagaagaagcaaaggaagatgatagcggcatgaatcatagcactcctgttggtgcagttataaatttggaggaagagagaatggcgattcttgagcagctaaaggttttggaggaaaggcttgtcagcttggacttggatgatgaagatgcaaaacattttgaggatgttaggctgatggaagattcatatcaagataatatagaggaagattctcatgccaatggtttcttgaaggaaatgaatgggaaacatcatcatgccaagggaaagagacttcttcctctatttgacaaaatgagtgatgaaaatggagatggtacactaaatgccaatggggttcataactttgatttagagaacaagaagctagctgtggaacaagagttggatcatctccatcaaaggctacaagcccttgaggcagatagggagttcctaaacaactgtgtaagctcgctgaagaaaggcgataaaggcatggatcttcttcaggaaatcctactacatcttcgtcatctaaggaacgttgagcttcgtgcaacaagcttcagtgatgctgccatactatag

SEQ ID NO: 52 - DNA, 1113 bp, synthetic construct; identical to SEQ ID NO: 51
  source 1..1113: mol_type = other DNA

SEQ ID NO: 53 - DNA, 2035 bp, synthetic construct
  source 1..2035: mol_type = other DNA
atgctgtggcggaattcgacaaaattgcagctgtgttcttcatccgctaggagtcgtttgctactgatttgtcgcctcgaacatcaattcatcgtcacacacaggtttggtacttctaagatcaaccttgcttcattttctgaaaattcattcatcgcaacattcagttttgattccttgagaataagcactgaatttttactcgattgtctgagttgattgagtctgaattcgttagtcgactcgaagttgttattagtttgaaatattgtatgaagttctggtttatgaattcagttgagaattttgattgagtttcaaagtgttaactgaaagatctcattgatagcttggtcgaggttctgtatttgctcttgttgttggtagttgcgcatgagtttctgaattctattggagcaaagcttatgaaaattgaaaaaaaattgataaacaattgagcagatttcaaatagctacagcttagttcttcaatccagattgttttactgagcttcagttgccaagttttctaatttcaagctccttgaaacttaaagaggagtgaacagattttctttaagcatcagctcgacactgcccagttttcttagccaaagtgggattgattgttgctgttgctagctctagaattgggttggaatttgaatctaagtgttgaattgagattggttacaattcttgtgcacttttggacagttttctgcactttttgagccaagtttagcttcagtttactgcagctttatagcctgtttggccaagcttctttcgaggcaaaagcgcttttttttgggtcaaaagtgtttttttttctaaaattaaggtgtttggccaagcttttgaaaggaaaaaaaagtgattttgaggtgaagcagaagcagttttgtagaagcagaaaaaagtagtttctctccaaaagcacttttgagaaaaatacacttacaagcaggtttttaaagcttggccaaacactaattgctgctcagaagtacttttcaaattaattagccaaacacaaattgcttctcaccaaaagtactattgagaaaagcacttttgagaaaaaacacttctcaaaataaactgatttttgcagctgggccaaacaggctattagactttctactgtttgatttgtttttcagaagttcttccctactgaacttctacagtttggtaaactgcttagctgctgtgaaattggttgacgttagctgatttgagtttgatcaactatttgaaatctgctaatttgaggttggatgttttggagtttagtttggacttgagtttggcagaggaagctcaacagtttgagaattcatttgtgaggactaaacattaaatgttgttgcttacaaaaatcttgtgatagcacctgcagttctgccagattttgttaatttgtccaagattaattctgcagcagattttcagtttctccattgtttagttgctaaaaatctgagttgctacccagttctgcactagtagtactctgcaaattatgtgttgtttaagctgattgtctgcactggttaggtttctgacatggctgctaaagtttaaatccttggactgttttagggtgttattgactgagaaaaaaaccagaagaattttcaggcattccagctgcagtttgaactaaatctgcagtatttgaacttgtgtttgaagcagaaaaccagttgatctttggggattgaaaatctgtgaattgaagaggcctatagctgctgttaagttgaattgcaaactgcaggttgtagttccctgctgctacagtttggctgcaattgtgaacatcttcagtagcttgcagtcactgatgaactttgcatttgagctacaacttggagtctttttacagaattgtgaagaagattgctcaggagatcaccaacagtctgcaaccttgtcagttattgtgaggctgtccagaattgcagatttgaccagtccttacctatgtggccattctttgcaaatccgaccaattttacaaaactag

SEQ ID NO: 54 - DNA, 1068 bp, synthetic construct
  source 1..1068: mol_type = other DNA
atgtttgatgactctgtatctactgtttattcttcaatatggacatccatgaatagctggttcactcccactgtcctttttgttcttctcaatctcatgattggtaccattgcttttacttcttccttagctaaccaaaagccaaatcaccctcccaaacaagaacaacaacaagaagaacaagaatactcacaaaatcacccacaacaaccctctaaactcattagatctccttctattcttcaacgcatcaaatctttaaatttctacaacaatagatctcaagaacctgttaaagattacaactttgacgatacccataatcaagaaaccacctttgaaccacaaacacattacattttcgaaccagctccagaacatactaccgtagtaccagaatctacccagtatattttcaatcaatcccatcaagaaaatgctcaaaaaacacaaacccagtacgatttccagcaaacccatctagaaaatctcccagatacacaaacccagtacctttttcaacacacccccgaccaagaactcaaaaatatggatcaagatttccactttcagcaaacccatcaagaaaatattcaagaaaaatcggcccgttacattattcaacacatccatgaacaaaaacttgataatacagatacccatttcgattttcagcaaacccatgaagaaaatgaggtagaagaagaggaagaagaaggtgagctaaagagcttggatgaagtatacaatcagttaaaggaacgtcacgtgagcaggagcagatcagatacaaatccatcggccggtgaagctccgataaagttgccgacgaagatgaagaaatcagcaagcatgaaatcgccatttgcgcattttgaagaggaggctattgttgaagctcgccggccggccactacaagggagagaaatgtcaagacaagcgacggagacattaatgaagtagatgctaaagcagatgatttcatcaacaagttcaagcaacagttgaagttacagaggttggattcaattctcaggtacaaggagatgattggtagagggagctaa

SEQ ID NO: 55 - DNA, 1158 bp, synthetic construct
  source 1..1158: mol_type = other DNA
atggaaattgttgccaagtcaaaaaaacaccaccattattattctcctttcccttcttgtttccgccgccctttagaggtagctgccgccgcctccgctcgtcctcttccgccgccgccgccgccgccgcagccaccggttgctgctaatcccaacctcgccacctccctctaccaaacccacctcggcctctttgctctcacttggtctcgcaatctcttcggcagatcttttcacatccatttcctcctcaacgattctgatggagttggagctgactacaataataatacaatctcttctcctcatttatcttccactagtaccccatctttccacctcaacatcaaaccctttattttctggaaaaaacatgggtcaaaaaagctcgacggagataacaaggttgttcatatcttttgggatctttctaaatctaaatttggatctggccctgaacctatatctggattttacgtagctgttattgttaacgaagaaatggttcttctggtcggcgatttaaacaaagaagcttacgctaaaacccgagctcgaaagccagaaaagaagcagaatctaaacccgaatccaaacccaaacctggtgctgagaagggaacacgtatgtggaaacaaactctacaaaacaaaagcaaacttcggtgggaaagagaaagaaatctcaatcgattgcaggttaggagaagatccaaggctgtatttcagcgtagataacaaaagggttttgcaaatcaaacacttgaaatggaaattcagaggaaatgaaaggattgaagttgacggagttccagttttggtttcatgggatgtgtacaactggttatttgacgatgacgaagatgggtatgctctgttcatgttcaaattcgaaaaatcaagttacgaatatgtagctgctgatgattacagcttaaataatggagttcaattgtggtctcaacaatcttgtggattcgggttcgagacgaagatgatgaaaaagggtgtattacgaagctcgagaagttcatcttcttcatctttatcttcagcatcttcaacttgtagctctgtcatggaatgggctagtactgaagaaaatgaattgaaaggtccttcgggattttctttgctggtttatgcttggaaaagctga

SEQ ID NO: 56 - DNA, 715 bp, synthetic construct
  source 1..715: mol_type = other DNA
atgtggcagtttaacaagatatggtgccaaaatcaaagaattcaaattacctattggggtaagtatccaacattgtgttgaaagagttctcatacttagacaaaatcttggatccaattggtcttctatatactatgacaattatgaattatcaggttatcaacttatttcacctgttttaggcctattagcatacaatgctggtgatgatatggtaacttctacaaccccttttgagcttataattcaatctgataaaattccaatcacaattgattttagcaatacaacaagactagtaaatgatactagtacatctgggataataccattatgtgctagctttgagcgcgacggaaaggtgacactaacaaaacaagtgtcacaaaatgtttgtgctgctaaaaaacaaggacattttggtttagtaattgagtcacctctaatgccattgaaaaataaaagggcaagtaattggaaattagctattgggagcgcgattggagctgcgttgggcgcgttccttttaggtttgctactgattgcaatatttgtgaaggtgaagaagaaagcaagaatggaggaattggttagaagagcatatgaagaagaagctttacaagtttctatggttggacatgtgagagctccaactgcagctggtactaggactgttccaactattgaacatgactataatcctcataattcttga

SEQ ID NO: 57 - DNA, 2732 bp, synthetic construct
  source 1..2732: mol_type = other DNA
atggatcagatattgaacaaagtgggttcctactggattggtaagagagctaacaaggagctcaattccgtcggcgatgacattaacgtatgccccttcttttccttttgcttcaatcttaattatttcagattttgctatgatctaatttggaaattcttaattatgtctgtggaaccagtaaattttataggtttgattgcataatttaccttaatgactaggatgtttatcccttaatagtctgaatttgctaaatgaaggggagtcttggagcaatggtaaaaacctgtaggtcacgggttcaagtcgtgcaattagtcactgatagtaggctgcctacatcacatccttgagtgcggcccttccccggaccctacgcgattgcgggatgctttgtgcaccggcctacccctttagtctgaatttgctaaataagctccttctagctagatgtggatctatgattttaagcccctcagtgcgttggttcaatacaaaggtagtacagatgtgcggtaggtgcacattacgagttcgaacataataggtaaaccaagtgtgtggttaaatagtgaaggatagaggagcagtctcaatatttaccaagttccgaagttggaaatgactgatttttcagttatttttaatgaaaaagaaagtgatagagaaatccgctgtttccaaaaaagaaagcagcacctaagggtgtgaggtagtgaaatataacagagtgcatatgatgattccattttgccaattcctttgcagtttggttatcaacaggtgccactatatgatattttggtccgtgtcatagctaatcaacgtttaaggttaaatacgactttcatagcatgttagaagctaatttcaagggggggagggggatactttcctttggcatatcttgagatcattcacagagtgatgacttcttaatttgggttctatctatttctaattaatgatccaccattttctttttataccatggtatttcaactgttgtttattgggaaatggctctgtgactgtaactgggaatgcaaacacttcttcatggaaaatggattgataaccaagaagtgcggctagatttaattttgttggataagactcccgcagatgcagagcggaaaagctataatggaggcatagtactactaggttttgtcactgacgtgctaaaattcagaactttttgttagatcagtctcttctccctcatgctgttattcttcaagttttattgattttttttataactgagatatagttgagggctagtggcacacggtttagaacttggaggacaatgggctcgcctctctactcttctccacttaaataccaggcttttatatgcagtagggtttgagcctcttcaaaatttttgtatttcctgtgtggttcctaaacaatgtcatagtagacaaattctagcttttcttattgctagtctaatgctcatgcagacttttttgcgtggttggccttcgattttgatgaattgcttctcttgttgcatttattgcttatttgcatttccaagatcaatataagttattgtatacagtggaaataaaggaacctgatgtgcgtcttctccacctttattccttaaaacatgagcataaactggtcagctgagcatcccaatgaatgtaagagtgaagctgtttactgttaatggaagaaacaaatggaaaacctttatcctcaagatgttggtaatagccaatagaaacttttatgttctaatatgattataagtattataatgttatgatagttcttcattaaaaaaaaggtaaccgaagccacttagtgttcctgcttctctgaatataatgactgtaattgatggggatatttaaaaaaaatttccacttcagaaaaatcttgataatgtcacttttctagcatagcaactgaaaccattttagaattactgttaatattactaagtttgctaaatttggaattacttggagaactcaggataagtgctgttggttcccattagtcgtttctctgcattggatttttctctatctggtttttcagtttttatggttcatactgatcttttccctatcaataatcagtcattgcaaagcagtattgaaggaggaacaaaatggctggtgaacaagcttaaaggtttgtgttctctttgcataactcaccatgctcttccatttttgcccccccttttgtatgttcaaaatttgctatctgatccactagcgtccctcgttcatcttcttgaggattttctacctatatcaacaagcttaaaaagaacttggtacaagtcataacctttatcttccctggttaatcttggttttgtaggaaaaatgcaaaagccattgccagatcttttgaaggagtatgatgttccagtaggtattttccctcgggatgccaccaattacgagtttaatgaagagacaaggaagctcactgtctatataccctctgtatgtgaagttggttacaaggattcatctgtattacgcttctctacagaagttactggatttttagaaaaaggaaagctagctgacattgaaggaatgaaaacgaaagtgatgatgtgggtaaaagttactgccatctcatctgaaaaatccaaggttcatttcacagctgggttgaagaaaacccggagtagggaggcttatgaggttttgagagatggagtagctattgaaaaattctaa

SEQ ID NO: 58 - DNA, 1377 bp, synthetic construct
  source 1..1377: mol_type = other DNA
atgggtgccattagtttcttggacaccatattagtccccttgagtcttttcatcacaattggctatcatttctatctttggcaccacttgaagcataaaccttctcgcaccaccattggtatgaatatactcaagaagagatcttggctgcgagagctcaatcaagcaagtcttccttttcattctttagtttttaatttcaattaattatttatgaagagaaccttagagcaacgataaagttattttcatgtgacttataggttacatgttcgagccatgaaatcagagtgcttgcatcatggtaagttgtctatgttacaccccttaggggttgtttggtacgcaggatagtgagataaatttataccatcaaccaaacacagtataaatttaatctcaaatttaatcctgaatattcccttatcctatcaaacttgggattattttatcgcacctctcaggtgggataaagggaggtggaataaattagtccagagattacaatccagggactataatccaggataatttagtccgcatagaaaatgactccttaacatacggccgtttcctgatgcgctttctgaaaataacttaaataggtatttgagccagctcgaaagatctctgcttgaacacgcacaaggtggttcgtgcatcgggcagtcctttatttactaagagtctaatgaacaaaggcggatctaaatgttgctatagggttcaattctgtttcaactctaactatgtatacatatacaaaaagaaaattaatcttactcattcttaacccgctgactctagatcttgaataagcctccgttgatgatttattaagtttttttttttgtttgtgtgtggcagggaaacgagaagaaaggcacgttagctgtacaaagcttaagaaatgcactaatggaaacaatactcacggcaacaatcacaatgatcataactttagcattggcagctctaacaaacaacacttacaacgtgagaaacctctttactagtgctttctttggatcacaaacagggaagataatcgtgttgaaatatggctcagcaactattttcttgttggcaagtttcttgtgtagttctttggcacttagttgtttgatcgatgctaatttcttggtcaatgcattgggggaattttccataaatccaatgtatacagagacagtatttgaaagaggatttacattggcttttgtaggggatagagtgctttgcatggcttttcctcttttgttatggatgtttggccctgtgcctgtggttgtgtcttctttggctttggtttggggattacatgagcgtgattttgctggaaatttgtcaagaactatgaataagagttgtatttga

SEQ ID NO: 59 - DNA, 474 bp, synthetic construct
  source 1..474: mol_type = other DNA
atgtcacatctcactgcagctccgccatcagaaccaccgtcggcggcggtgaattcattgtacaaacagaaatcgtggtcgccggacacgtttcgcgatgaggcgtggcagcggcggaagggtacccatggaagccgcttaaagcagcggagcaagagcgttaccgatgaagacctcgatgagctcaaggcttgtattgagttagggtttggatttgactcgcctgaaatagatcaacgattgtctgatactttcccggcttatggccttttttacgctgttaataaacaatatgccgacaccctttcgaagacttcttctttatcgtcggtcatctcctattgcgagtcacccgctcctcccggcagtccccacaccattgtcgatcaaggagataatcctcagacagtgaagacaaggttgcggcagtgggcacaagtggtcgcttggtcagttcgtcaatcttcacattaa

SEQ ID NO: 60 - DNA, 2073 bp, synthetic construct
  source 1..2073: mol_type = other DNA
atggagaaaaagcagctggatttcaatgcaccacttttatcagtgaggaaaatttcatcatctttatcccctcatgagagagcaaataaaaagattatagaaagggcgccaccgaatcgacaacaatcacttcctgtcaaaaaatcagattgggaattgagtgaggtgactaaaccagtagctgttccatttatgtgggaacagatccctggaagacgaaaaggtgacaatgaagcccgagctaaccttcgagtggagtggtcaagtagtcctaggctgcccccgggaagattgccagagactattcggttctattcgggtgaaaggcctcgtactcaaaacatttacaagtctccagctgaaggacttccttggattgatcatgcagctttacttgatagcctagcggaaagtatatatacaagaggagacagagcaagtgaggatgatgccaattctgatgctcctgaaacattgtcacctaccgaatccttatccttggactgcagtgtcagtggtttgagcggacatcaaagttcagattcaaaaccatctggaaccttttccattgactcccaaactagagactttatgatgagtcgattcttacctgcagcaaaagctgttgttttggagacgcctcagtatgttcaaaaaaagcaagttccagtcagtactgaacaacccaagccagtacctgtggaacgaaagccaatagttaagcagatggagtctaaacctgtctcatattatagcggctatccagacgatgtaggaagtgaaattgaagatgatgtgtctgagaatcagcacaaaagaccaagtaaagggtggaaatttttccctcggatttgtgtgaagaattctttatgcctattaaatccactgccaggactaaaagtgaagacacatgttcccacaccctcagctcaagcagtaaagagagtttcaggaatgaaaccaaagacgccacagagccccacatcttatgctcatgaagtcaagagattagctagaaaagcttatagtggacctcttgagaagaatacttgtgatactataaataagcaaagatttcactctggagtactgtctcgcgagctgtacaaagctgaaaatagaagtttttccggccaactgcctaaccctagtgattcatgtaagctagttggaatctctcctggaagacgttcaagaagtggggctatatctccctgtcgaaatgtagcacccccatctccattcaatgaaggtactaggtttcttggtgtgccaaaggaaatggagagtctttgggctagccgctttgattcattccggaaaggttgctacactgtcaaggacaaagtaccacagcagattggcacaggaaggttttctgattcaccaagtgaagtagttgagaaaacattgtacatagattctgtagataatgtgcaaatttcagcccgtaattctgcttcttcaaagcccaaggaattggtgaactcctctggtaagaatatgaagccactggttaaaagcagtaaagtactagaaaatatggatgccaccgctcgtactcaaggtgccaaagaccggaatgtttcagagaaagagagcaaacaggtttttgagaaggagtcccttgatttggtggaagcttctcccatttccatatcaacacgcaaaggccctgctgatcatgagtctttgaaactaaagcaaaatcttgatacactatctggagccttagagtcttcaaaagtgcatccctatggaaatttaggtacagaaaatgaagataaccaaaatgcaaatggcccaaaagattctaaccttacctccttggaatctcctataccaccacccttacctaaatctccctctgaatcttggctttggcgcacgctgccttccatccctttgagaactccgttctcaagtttgagctccaagaagcagaataagaagtcccatgctgatggtagtaagtgggagactattgtgaaaacttctaacttgcataaggatcatgtccgttactccgaggaactatatactcttggttcttgtcagcagagcaaagcttga

SEQ ID NO: 61 - DNA, 1260 bp, synthetic construct
  source 1..1260: mol_type = other DNA
atggaagtcatatctaccaacacaaatggctcgaccatcttcaagaatggtgccattcccatgaatggccaccagagtggcacttccaaacacctcaacggctaccagaacggcacttccaaacaccaaaacggccaccataatggcacttccgaacatcggaacggccaccagaatgggatttccgaacaccaaaacggccaccagaatgggacttccgaacatcggaacggccaccagaatgggatttccgaacaccaaaacggccaccagaatgggacttccgaacaccaaaacggccaccagaatgggacttccgaacaacagaacgggacaatcagccatgacaatggcaacgagctactgggaaactccaactctattaagcttggttggttttcagagtttagcgcattatggccaggtgaagcattctcccttaaggttgagaagttactatttcaggggaagtctgactaccaagatgtcatgctctttgagtcagcaacatatgggaaggttttgactttggatggagcaattcaacacacagagaatggtggatttccatacactgaaatgattgttcatcttccacttggttccatcccaaacccaaaaaaggttttgatcatcggcggaggaattggttttacattattcgaaatgcttcgttatcctacaatcgaaaaaattgacattgttgaaatcgatgacgtggtagttgatgtatctagaaaatctttcccttatctcgcagctaattttaatgatcctcgtgtaaccctcgttctcggagatggggctgcatttgtaaaggctgcacaagcaggatattatgatgctattatagtggactcttctgatcccattggtccagcaaaagatttgtttgagaggccattctttgaggcagtagccaaagccctaaggccaggaggagttgtatgcacacaggccgaaagcatttggcttcatatgcatattattaagcaaatcattgctaactgtcgtcaagtctttaagggctctgtcaactacgcttggactactgttccaacatatcccactggtgtaattgggtatatgctctgctctactgaagggccagaagttgacttcaagaatccaataaatccaattgacaaagagacaactcaagtcaagtccaaattagcacctctcaagttttacaattctgatattcacaaagcagcattcattttgccatctttcgccagaagtatgatcgagtcttaa

SEQ ID NO: 62 - DNA, 1062 bp, synthetic construct
  source 1..1062: mol_type = other DNA
atggaagtcatatctaccaacacaaatggctctaccatcttcaagagtggtgccattcccatgaatggccaccataatggcacttccaaacaccaaaacggccacaagaatgggacttccgaacaacagaacgggacaatcagccttgataatggcaacgagctactgggaaactccaattgtattaagcctggttggttttcagagtttagcgcattatggccaggtgaagcattctcacttaaggttgagaagttactgttccaggggaagtctgactaccaagatgtcatgctctttgagtcagcaacttatgggaaggttctgactttggatggagcaattcaacacacagagaatggtggatttccatacactgaaatgattgttcatcttccacttggttccatcccaaacccaaaaaaggttttgatcatcggcggaggaattggttttacattattcgaaatgcttcgttatcctacaatcgaaaaaattgacattgttgagatcgatgacgtggtagttgatgtatctagaaaatttttcccttatctcgctgctaattttaacgatcctcgtgtaaccctagtccttggagatggggctgcatttgtaaaggctgcacaagcagaatattatgatgctattatagtggactcttctgatcccattggtccagcaaaagatttgtttgagaggccattctttgaggcagtagctaaagccctaaggccaggaggagttgtatgcacacaggctgaaagcatttggcttcatatgcatattattaagcaaatcattgctaactgtcgtcaagtctttaagggctctgtcaactatgcttggactactgttccaacatatccaaccggtgtgattggttatatgctctgctctactgaaggaccagaaattgacttcaagaatccagtaaatccaattgacaaagagacagctcaagtcaagtccaaattagcacctctcaagttctacaactctgatattcacaaagcagcattcattttgccatctttcgccagaagtatgatcgagtcttaa

SEQ ID NO: 63 - DNA, 1146 bp, synthetic construct
  source 1..1146: mol_type = other DNA
atggaagtcatatctaccaacacaaatggctctactatcttcaagaatggtgccattcccatgaacggttaccagaatggcacttccaaacaccaaaacggccaccagaatggcacttccgaacatcggaacggccaccagaatgggatttccgaacaccaaaacggccaccagaatggcacttccgagcatcagaacggccatcagaatgggacaatcagccatgacaacggcaacgagctacagctactgggaagctccaactctattaagcctggttggttttcagagtttagcgcattatggccaggtgaagcattctcacttaaggttgagaagttactattccaggggaagtctgattaccaagatgtcatgctctttgagtcagcaacatatgggaaggttctgactttggatggagcaattcaacacacagagaatggtggatttccatacactgaaatgattgttcatcttccacttggttccatcccaaaccctaaaaaggttttgatcatcggcggaggaattggttttacattattcgaaatgcttcgttatcctacaatcgaaaaaattgacattgttgagatcgatgacgtggtagttgatgtatctagaaaatttttcccttatcttgctgctaattttagcgatcctcgtgtaaccctagtccttggagatggggctgcatttgtaaaggccgcacaagcaggatattatgatgctattatagtggactcttctgatcccattggtccagcaaaagacttgtttgagaggccattctttgaggcagtagccaaagccctaaggccaggaggagttgtatgcacacaggctgaaagcatttggcttcatatgcatattattaagcaaatcattgctaactgtcgtcaagtctttaagggctctgtcaactatgcttggactactgttccaacatatccaaccggtgtgattggttatatgctctgttctactgaaggaccagaagttgacttcaagaatccagtaaatccaattgacaaagagacaactcaagtcaagtccaaattagcacctctcaagttctacaactctgatattcacaaagcagcattcattttgccatctttcgccagaagtatgatcgagtcttaa

SEQ ID NO: 64 - DNA, 510 bp, synthetic construct
  source 1..510: mol_type = other DNA
atgccaaatcaaaagcaaggaagtgaacagctacaaacactgatgcaatcaggtcaaatttcggggtctttgagttttaatggggctttaactaaagaagatgaagagatgtctaggtctgctctttctactttcaaagctaaagaagaagaaattgagaaaaagaaaatggaagtcaaagaaagagttcaagctcagttgggtcgggtcgaagaagaaactcggcgtttagccattattcgtgaggagctagaagcattagctgatccaaagaagaaagatgttcaaatcgttcggaagaagattgatgctgtcaacaaagagttaaaacccctgggacagacctgccagaagaaggaaaaagagtacaaagaagctcttgaggcgttcaatgaaaagcacaaggaaaaagtacagctaataacaagattgatggagttggtgggtgaaagcgagaaactgcgactgaagaagttggagctgagcaagagcatagaaacaatacgctga

SEQ ID NO: 65 - DNA, 1308 bp, synthetic construct
  source 1..1308: mol_type = other DNA
atggatccgtgtccttttgtgagaatagttatcggaaacttagccttgaagtttccggcggaacataaactctcctcaagtacggtttttgattgtaaattcaagcttagaggtttctccacacaagtttcaaccatctcagcttttgttcaagatccaattttggacaacagaactcactcttgtttcaacttgaacaaaacagaacttgacaaactggtcgaaaaatcaactgccaaaggaaaaacttgcagcttaaagattgaaatttttgcacggaagaaggggattggttgtgggttattgaatggtgggaagctattggggagtgtaatagtggatttaaaggggttggaaagtgatggtcggagaggagttgtgattcagaatgaatgggttttggttggtggatctagtactgcagcacaattgcatttgaatgtgaaagctgaacctgatccaagatttgttttccagtttgatggtgaacctgagtgtagtcctcaagtttttcaagtcaatggcaatgttaaacaacctgtttttacttgcaagttcagtttcaggaataatggtgacaggaatttgagatcaagatcttcactatcaaaaccaagtacatcaacaagctgctttaattcttgcacatctgataaagaatcatcgccgctaaaagagcgaaagggatggtcaatcacaattcatgatctctctggctcaccaattgccgcagcatctatggttacaccatttgtcccatcacagggttcaagtaatgtcagcagatcaaaccccggggcgtggctcatccttcgcccaggcaacggtacatggaagccgtggggccgcttagaagcatggcaagagcgcggtggtgacctcggttatcgctttgaaatcatccctgatggtgctactgatgccataactttgtccaattcaactatcagcaccaagaatggtgggaaatttggtgtagacatcaccaatggtgctaccccattgactagtccaaacagcagctttgacttatgctcgggttctggatccgggtcggatttcgggtcagcacccggatccgggtcgtgggcacaactcttgtaccgtggtttcgtgatgtcgtccacggtggagggtgagggaaaatgcagcaagccagaggtggttgtcggggtgcagcacgtgaactgcgtggaggatgctgcagctttcgttgcattggcagctgccatggatcttagtatggatgcttgtcagtcttttactaagaagctccggaaagagttgagacagtcggatcaagaatga

SEQ ID NO: 66 - DNA, 426 bp, synthetic construct
  source 1..426: mol_type = other DNA
atgtcaggcgaggatgttgcagttgctgttgccgagactcctgctccagcacttggagagcccatggacatcatgactgctttgcagctggtgctgaggaagtctaaggctcatggaggcctagctcggggactacatgaaggtgcaaaggtgattgagaagcatgcagcacagctttgtgtgttagcagaggactgcgaccagccagattatgtcaaattggtcaaagcactctgtgctgatcacaatgtcagcttgatcacagttccgaatgcaaaaactcttggcgaatgggctggtttgtgtaagattgactctgaagggaaagcaaggaaagtggttggctgcggctgtgttgttgtgaaggattatggggaagagactgagggtctgcatattgtccaggagtacgtaaagtcccattaa

SEQ ID NO: 67 - DNA, 432 bp, synthetic construct
  source 1..432: mol_type = other DNA
atgtcaggagaggatgctgctgttcctgttgttgctgccgagactcctgctccagcacttggggagcccatggacatcatgaccgctctgcagctggtgctcaggaagtctaaagctcatggaggacttgctcgaggactccatgaaggtgctaaggtgattgagaagcatgctgcgcagctttgtgtgctagcagaggactgcgaccagccagactatgtcaagctggtcaaagctctttgtgctgatcacaatgtcagtttgattacagttcccaatgcaaaaactcttggcgaatgggctggtttatgcaaaattgattctgaagggaaagcaaggaaagttgttggttgtggctgtgttgtcgtgaaggattatggggaagagactgagggtctccatatcgtccaggagtacgtgaagtctcattaa

SEQ ID NO: 68 - DNA, 1299 bp, synthetic construct
  source 1..1299: mol_type = other DNA
atggatcctcaggctttcattaggctgtcaatagggtcgctggggttgagactgtctggaacaactactttgaacagtacaaaaacagggataagtgcagtctcttctccctgtgtgtgcgagatccgtcttcgaggtttccctgtgcagacatcatctattccctatatttcctcacctgaagctacaccggatattcacaacgttgcatccagcttttatcttgaagaatctgatttgaaagctttactggcacctgccccctgtttttatgcggctcatgcatgtctagagatagttgtttacacaggacgcaaaggaggccactgtggtgttggtattaagaggcagcaagttgggacatttaagttggaagtaggtcccgaatggggtgaaggaaaaccagccattctgtttaatggctggataggaattggcaagaacaagctggagactggaaaacctggagcggagcttcatttgagagtgaagctggaccctgatccaagatatgttttccagtttgaagataaaacgaaactaagccctcaaatagtgcagcttcagggaaccatcaagcaacctattttcagttgcgagtttagtcaggacagggtatctccggtagatccattaaataatttttggtcaagttcatttgacggttctgaactagaggtagagaaaagagagaggaagggatggaaggtgaagatacacgatctctctgggtcggctgttgcagcagccttcataacaactccgtttgtgccatcaacaggttgtgattgggtggccaaatccaacccaggagcttggttgattgttcgtcctgatatttgcaggcccgaaagttggcagccatggggaaagcttgaagcatggcgtgaacgtgggatcagagattccatttgctgtcgcttccatcttctatcagaggggcaggaatgtggtggtgatcttctcatgtctgaaatcttgatcagtgcagagaagggtggtgagttctacattgacacggacagacaggtacaagcagcagcgagtccactgcccagtccaagaagcagcggagactttgcagcactaagtcctgttgcaggcggttttgtcatgagctgtcgagtgcaaggggaagggaaatgcagcaaacccctcgtgcaacttgccatgcgacacatcacctgtgtggaagatgcagccattttcatggcgcttgctgctgcagttgatcttagcattgaggcatgcaggccttttcgcagaaagctacggagaagtactcgccattcctggtga

SEQ ID NO: 69 - DNA, 1284 bp, synthetic construct
  source 1..1284: mol_type = other DNA
atggctaaggtgtgcttttacaatgtgaagatgaatatgcaagtgatgttttgcttgtttttggtgtcactatgtcgtgatgatgcagctgctgaagctaaacaggggatgtctcttgctgcccaaaagttcgaggttcaaaagcacttgaatcgcttaaacaaacctgcaatcaaatccataaagagcccagatggtgatattattgattgtgttcatatgtctcaccaaccagcttttgatcatcctttgctcaagaatcacactgtacagatgatgccaaattatcacccagaagggctatttcgtgatggaaaattgtctacagcaaaaaccaagaatgaaggttcaaaatcaattactcaactgtggcatttgaatggaaaatgtccagaaggaacaattccaattagaagaaccaaaaaagatgatattttaagagcaagttccatgaaaagctatggtaggaagaaaaaacattctactattcctaaaccagagtctggtagacctggccttgttagccaaactggtcatcagcatgcaatagcctatgttgaaggagacaaatattatggtgcaaaagcaaccataaatgtttgggaacctaaaattcagcaacccaatgagtttagcttgtctcagctttggattcttggaggttcttttgattcagatcttaatagcattgaagctggatggcaggttagcccagatttgtatggagataacaacacaagactttttacctactggactagtgatgcttatcaagccacaggctgctacaatctgttatgttcaggatttattcaaatcaataatgaaatagcaatgggggccaccatttcccctctttccagctatcatggttcccaatatgatataagcattcttgtctggaaggatccaaaagagggaaactggtggatgcaatttgggaatgactatgtattgggatattggccaggctttttattttcatatttaacagacagtgcttcaatgattgaatggggtggggaagtggtgaattcagaatcagatggacttcacaccacaactcaaatggggagtggccattttccagatgaaggttttgggaaatcaagctatttcaggaatatacaagtagttgatggttcaaataatttgagagctcctcaagatcttgggatttatactgaggataacaattgttatgatgttcaactaggaaaaaataatgactgggggaactacttttactatggtggacctggcagaaatcctaattgtccatga

SEQ ID NO: 70 - DNA, 939 bp, synthetic construct
  source 1..939: mol_type = other DNA
atgtcaatagacaaaagctcaatgcaatcaaaccttgattgcttcttggaatgcaccacaccgttagtcccttcccagtttctaccccagagtgagattaggaatctgaacaggttatggcatccatgggaaagggagaaggttgaatattttactttggctgatctttggaattgttacgacgaattgagtgcttatggggctggagttcctattaaaatggatacaggcgaaactttagttcaatattacgtgccttatctttcagctattcaaatctttaccagcaggtcttctgttaattttctgagggaagagactgagtctgtttgcgagacgagggattcgttcagtgattcgttcagcgacgagagtgaaagtgaaaagctatcaagatgggatggttgttcttctgaggaaggagatagtctttggcaaatgaatgatagatggggctatctttattttcagtactttgagagatctactccttatggaagagtccctttaatggacaagattagtggtttagctgaaagataccccggattaatgtcattgagaagtgtagatctttcaccagctagttggatgtcagttgcttggtatccgatatatcacattcctatgggaagaaccattaaagacttgtcagcatgctttctcacattccacaccctttcttcttctttccaagatatggaccttgaagatgacacggagaatggtaaacggaaacgaaaggaaggagaaagcatcccacttccacctttcggtttgggcacttacaagatgcagggtgatgtgtggctttcagacaggaaaggaagggaccaagagaggctggcatcactttttagcgtggcggattcttggctaaagcagttgggtgtccagcaccatgacttcaactatttcatgggtattcgtcgtggctga

SEQ ID NO: 71 - DNA, 951 bp, synthetic construct; identical to SEQ ID NO: 13
  source 1..951: mol_type = other DNA

SEQ ID NO: 72 - DNA, 1281 bp, synthetic construct
  source 1..1281: mol_type = other DNA
atgaggacattgtgctccaacttggatagagaagatgggctggaaacagtgctggaagtccccatcccagaagaaatgtttgcttccaacaagcacagggcacggcagagcaagaattctggggtcaaatctcacatggacaagtctgctgcttccgtttttggtagccgaaatgctgagattcaactcttgcttggcgttgttggagctccgttgatccctcatcctatccgttgcgactactctctcaacacaaaaatcaacgatcatcccatagaagcttcagtggcgaaatatatagtacaacagtacatagcagcagcagggggagaacatgctctaaattccattgatagtatgtatgcaatggggaaaataaagatggtggcgtctgagtttattgctggagatggaatgggattgaacaacggtaatatgatgaagattaagagtgtgaaaaatgggcctggtgaaatgggaggatttgtgttgtggcaaaagagacctgacctgtggagcatagagctggtggtttcagggtgtaaaattagtgctggaagtgatggtaaagtagcttggaggcaaactccatggcataattcccatgcatctcgtggtcctgctaggcctcttcgtcgttctttacagggtcttgatcccaaatccgttgcgaatttattctcgaattccatttgtattggagagaagacggtgaatatagaagactgttttgtactaaagcttgaaacagagccctcaactctgaaagcaagaagcagcagcaacgttgaagtaatgaggcacactgtttggggttacttcagccaacgaacagggctcttactccagcttgaagacactcatcttcttagaatcaaagccccaggaaatgatgtcttttgggaaacaacaatggactcattaatactcgattatcgaactctcgacggtgttaatattgcacatggtggaagaacatctgtttctttatttaggtttggtaagaactcagaaggactcactaggacaagaatggaagaggtttggaccattgaagaaatcgattttaacataaagggactatctgcggattgtttcttaccacctagtgacttgaaaaaggaagatgaagtgagtcatgatgtgaacaagaagtcaaggttggggttcaaggctcgtactaataatgctaaactaagaactgctaggaaaggtgcgtctaaaatcatgtctattgacgaagaagatcttgaagattacgaagcagatgaagaatcttga

SEQ ID NO: 73 - DNA, 378 bp, synthetic construct
  source 1..378: mol_type = other DNA
atgattattggctctagcttctcagaaaaattgatgggaaagaagaaaattttgaagaagacaaaggagttatcagtagcaatagcagaatcatcagcaatgagtggagactctcaacaacaacaacaacagattactcctagaaaaagaggaagaccaaggaaaattattgtaaaagatgaagaagaggctactggagaaataaagaaactcaaaataaatgaaggtgaagaattagaaaataaagaagcagcagaaggagaaaagaaagaagcagaagaacaatctgaagaggaaaaaaatttacagccacaaaagcagcaacctgtagcaaagagcagagctagaagaaaaagcaagccaagaaaaagctgctaa

SEQ ID NO: 74 - DNA, 1578 bp, synthetic construct
  source 1..1578: mol_type = other DNA
atgatggcacagcatattgagattagcccactgcttgatcaaactcctccgaatgaagctacgaaagatgagtcgccagctcgcccgcggttgctaaagccttcacccactgccgctgcttccttcttcgcctttgccgtcgccagtcatcaagtgatacaagaaggcgggaaagcagattatataatcgagataaggacagcagatcaaacaaatgaatcagcaaatcaagtctttgatgaaggatcatctaccatatttaaagtaaatgtggggctatctgaatcaaatccagatgcttataccccaaaattgatctccattggtccttaccataaccaaaatccgagacttcgctcgatggaaaaatataaactgcgttaccgacaacggtttctgcagaggaaagcgtggagagatgtggagtattactttagtgaaatggagaaactaaaggatgaagcactaaagtgttacgatgatataagggaccttgacagtaatattgttagcaaatttttagatttgctgttacttgatggctgcttcgtggttgagtatatacgagagttttacgaaggggtgccagaaggagaagacaagattatcaacgcggcttggatggaatctctggtagatcgtgacttgttgttactagaaaaccaacttcctttctttatcctcgccaaacttcatgagatgactaaggatcctacagacgccccattcatacagatggtgaaatacaactttggtagtagtttaccaaaagtgactcctaaattcataaatgctactgatgatgatgccgaagaaatcaaacatttacttcaagtagtacacatgtgttgttgcccttcagagatgaatactggcctaactaggaatagcagaaagaaaaaaggaagtagcaagaaaagttgtaactggaatcctttgcgaataggaaagtccaaaaagaagtttaaaactaaggatggtgacttatggcacgatcgcatgcgaagtgcaacagaacttgatgaagctggaattcgcttctctaatgttgggaaaatttatagaaagttgaataaaaacaacaaggaggatgctataagtttatttgatatcaaattcaataagggattgcttgaaatcccttgttttgaagtcattaatagtacggagactatcctgagaaatctcattgcctacgaacaacactcctctgatgtgcatccaaaatatttcacggattatataatttttatggaccatcttattaactcaggcaaagatgtgaatttgcttcggttgaacggaattatcagaaataggataggagatgacgaagaagtggctattatgttcaacaaactaggagaaggtgtcatcccttcaactgatttcttttacaaagtggaatgcagaaaagtgattgagcattgtgaaaaaccatggaatgaaaacatggcaagtttgaggcacaattatcttaatagcccttgggcgaaaatttcaactgcggcagccatcattctccttctgctcacagttgcacaaacagttctagctttcatcagtactttaaagtag

SEQ ID NO: 75 - DNA, 909 bp, synthetic construct; identical to SEQ ID NO: 17
  source 1..909: mol_type = other DNA

SEQ ID NO: 76 - DNA, 1128 bp, synthetic construct
  source 1..1128: mol_type = other DNA
atggaagtcatatctaccaacacaaatggctctaccatcttcaagaatggtaccattcccatgaacggccaccaaaatggctcttccgaacacctcaacggctaccagaatggcatttccaaacaccaaaacgggcaccagaatggcacttccgaacatcggaacggccaccagaatgggacatccgaacaacagaacgggacaatcagccatgacaatggcaacgagctactgggaagctccaactctattaagcctggttggttttcagagtttagcgcattatggccaggtgaagcattctcacttaaggtcgagaagttactattccaggggaaatctgattaccaagatgtcatgctctttgagtcagcaacttatgggaaggttctgactttggatggagcaattcaacatacagagaatggtggatttccatacactgaaatgattgttcatctaccacttggttccatcccaaacccaaaaaaggttttgatcatcggcggaggaattggttttacattattcgaaatgcttcgttatccttcaatcgaaaaaattgacattgttgagatcgatgacgtggtagttgatgtatccagaaaatttttcccttatctggcagctaattttaacgatcctcgtgtaaccctagttctcggagatggagctgcatttgtaaaggctgcacaagcgggatattatgatgctattatagtggactcttctgatcccattggtccagcaaaagatttgtttgagaggccattctttgaggcagtagccaaagcccttaggccaggaggagttgtatgcacacaggctgaaagcatttggcttcatatgcatattattaagcaaatcattgctaactgtcgtcaagtctttaagggttctgtcaactatgcttggacaaccgttccaacatatcccaccggtgtgattggttatatgctctgctctactgaagggccagaagttaacttcaagaatccagtaaatccaattgacaaagagacaactcaagtcaagtccaaattaggacctctcaagttctacaactctgatattcacaaagcagcattcattttgccatctttcgcccgaagtatgatcgagtcttaa

SEQ ID NO: 77 - DNA, 1128 bp, synthetic construct
  source 1..1128: mol_type = other DNA
atggaagtcatatctaccaacacaaatggctctaccatcttcaagaatggtgccattcccatgaacggccaccaaaatggcacttctgaacacctcaacggctaccagaatggcacttccaaacaccaaaacgggcaccagaatggcactttcgaacatcggaacggccaccagaatgggacatccgaacaacagaacgggacaatcagccatgacaatggcaacgagctactgggaagctccgactctattaagcctggctggttttcagagtttagcgcattatggccaggtgaagcattctcacttaaggttgagaagttactattccaggggaagtctgattaccaagatgtcatgctctttgagtcagcaacttatgggaaggttctgactttggatggagcaattcaacatacagagaatggtggatttccatacactgaaatgattgttcatctaccacttggttccatcccaaacccaaaaaaggttttgatcatcggcggaggaattggttttacattattcgaaatgcttcgttatccttcaatcgaaaaaattgacattgttgagatcgatgacgtggtagttgatgtatccagaaaatttttcccttatctggcagctaattttaacgatcctcgtgtaaccctagttctcggagatggagctgcatttgtaaaggctgcacaagcgggatattatgatgctattatagtggactcttctgatcccattggtccagcaaaagatttgtttgagaggccattctttgaggcagtagccaaagcccttaggccaggaggagttgtatgcacacaggctgaaagcatttggcttcatatgcatattattaagcaaatcattgctaactgtcgtcaagtctttaagggttctgtcaactatgcttggacaaccgttccaacatatcccaccggtgtgatcggttatatgctctgctctactgaagggccagaagttgacttcaagaatccagtaaatccaattgacaaagagacaactcaagtcaagtccaaattaggacctctcaagttctacaactctgatattcacaaagcagcattcattttaccatctttcgccagaagtatgatcgagtcttaa

SEQ ID NO: 78 - DNA, 1443 bp, synthetic construct; identical to SEQ ID NO: 20
  source 1..1443: mol_type = other DNA

SEQ ID NO: 79 - DNA, 324 bp, synthetic construct; identical to SEQ ID NO: 21
  source 1..324: mol_type = other DNA

SEQ ID NO: 80 - DNA, 804 bp, synthetic construct
  source 1..804: mol_type = other DNA
atggcgatgggaccagaaagatcgaagccgcctctacacaatttcaccttaccgtgtgggctcaagtgggggaaccaaaagttcttaaggtgcgctaaggttgaatccgacggacaaatctccgccattcatcgcaggtcatttgggtctgagttgatcggacggcgccgatcaaatgatcgaaaattcaggccaccggaaaagcaagatgccggtgaaggaatcggagctgtgagagagaaactcatgtttgatctccaaacagaagctgataagataaaagacgcaatttttagagaaggtttggaggagcaacaactgtctccggcgccggcgaaaactgcgactgcggctgtatcatatgccggtgaattgtccaggccgtggaacttgaggactcggcgtgcggcttgtaaggagcctaatggattcgttgccggcgccggtgccgccggaagtggtggtggtggatcgaaaggagggttgaagattgatgcttatagaactaatgctccgtcgccgttaaggacggaaaacaaatctccgacacttcgaagtgattttgccggtggagcggccgccggagcttctgccagcggcgagaagagacagagagtgaagttttcggtacccctttcgcgaggagagatcgaggaggatttcatggcgatggtgggacatagaccccctcgtagacccaagaaacgagctaaatttgttcaaaagaatttggatacgttatttccggggttgtggttgacggaaattacacctgacttatacaaagttcctgaagatcagtag

SEQ ID NO: 81 - DNA, 1956 bp, synthetic construct
  source 1..1956: mol_type = other DNA
atggaaattatcagtgagagaagaagaacccgatctcaagcggctgctattctagtggagctaaaatccatcaattttcaatcagaaaaaagacccaaagtacatgaaaaagatggtgtgaaaaattttgggaaaaggaagcgtcgaaagggtagtaatgtcaggagaaaaaagaagggaaaatatgtacaagttgactctgatgatgatgaagactatgtgtttgataaaagtcccaaaaggagaaagctggattatggacacaaggggaagagttccagctcggatttgaagatttgggatttttatgttcaaccgagaaagcatttcacagcaagaattgggacacacactaatgttgatgttgttaaattgttgaatgcgaagttggatgataggcagattcagatgttcagagaaacatgcttcgggcattttcttgatttgcccgatgtattggttcagcctcaattgatacactcgctattgctgagggaggtggttcacgagagagaggatgagttgtggatttcagtgaatgatgtcaggctgcgttttgggcttgttgagtttgggattatcaccgggttaaagtgtacgggtgatgctgatatgtgccttgatttcgacggaacgaatcggttgtttgatacgtacttttctgaacttaccagggtacctaagcaatccttaattgactgcttccttaataagagatggaaatccgatgaggatgcagtcaacattgctgtcctatatttcatacacacattcttgctctccactctgaatcgtaattatatcacgagggatgactttgggctcgttgagagcggtgcatatcaaacgtacccttggggaaaagttgtctttaaagccacactgaagtctattaagggtaggttgcttgggaagccctcgatgtacaggcttcgtggattaccattggcattccaatgttggttttacgagtgctgcccttatgttaataagaagattgctttccgggttgatgacaaagtgccccgcatacttagttggaaagttacaaagcagccaaacctcaagaagttgtcgaatgggattttcatgcagaggcgggatcagtttatgctgaggaatatttctccaacagaatttgagcaaacaacactgaatttgcctgaatcatttgaaaatgaaagggataatgaggtggcatctggagatggcgctgaagttcatcttagtgatgatgactttagcggttcgccgcctcaaacaagtaggaagcagcctaaaaccaaacctgatcatcccttgaacaatgatgactggagcatcgagttaaaaagactgagtgatggacagtcggagttgaagagtgagattcagatgctcaacaaggaggttgcatcgctcaaggactgcatggtagcatcctttgcagatgcttttaaagccatcaagtctctgtcgaagaaacaaagagaaaagattgcttccgagcttgatggagaaaatggccaccatgacagacatggcgaaagcgattctagtgactttagtaatagtgaagacaatggttgtgaaggtcaagagcatggcaaggactctatgggcgacaaggacaatagtgagaaagggaatgaagtagctttgggcgatgctgagggccgggagacagttagagatcctgtggaggagggtgaaaggatggacgtatctgcgtctccgattcttccagatacatgtgaggtttcagaccacattacaccggaacaaagtcatccgacaccaattctagagtcaccatatgtgaaccaaggcgacactggtgttgagggttcttccgagaagtccattttaaaaggagagcaccctttagagagcaaaattgttgataatgttgattatagtttgttgtctgagtttgataagtgggttgacgaagggatgaagaaagaaagcaagtaa

SEQ ID NO: 82 - DNA, 897 bp, synthetic construct
  source 1..897: mol_type = other DNA
atgagtagtcacgaacgccgaggcccggtggccgtcgtcggcagcggcggttacagtcacgagagcgaacctgacttggctgctatggttagtgattttttggaaagtagtagtgtcggtgcggagtctcggtatagcagtgataatgactctggtttctccgatctcgctcttctcgctgataggatttcgttacacaagcactcagtggaccagtatgaaagtgatctaacaatggtggttcattcgctgattctttcgttgggcgagtcctgccatcttagcaagcctgagacttgcaatgcgagctgcatcagatctaatctagtgaagctcctacagtcttgcggttatgatgcagctttgtgttcaaccaagtggcagggttgtggaaagattcctggaggtgaacatgagtatattgaggtgatctcacgtggaaatgatggatgctctgaaaggtacatcattgacatcgacttcaggagccactttgagattgcaagagctgtcaaatcctacaatgtggtcttgagttgtcttccaccagtgtatgttggcacagtaagaaagcttaagcaatatcttcagactatggtagaagcagcgagatgttcgctcaaacagaactcaatgcctcttcctccgtggcgatcccttgcttatctggaagccaagtgggaatcatccagtcaaagagtagccaactttcaggtccaaagcagcattggcccctctaattcctctcatcagcattgtactgagctgttgtggaggataaaatccagtattgggtctgaaatcaacgccaaaagtttcttggttcctaaaagctgtagaaagaagcagggactaaagattgataaattgaggcattcttcaccagtgacaccgtga

SEQ ID NO: 83 - DNA, 840 bp, synthetic construct; identical to SEQ ID NO: 25
  source 1..840: mol_type = other DNA

SEQ ID NO: 84 - DNA, 2073 bp, synthetic construct
  source 1..2073: mol_type = other DNA
atggagaaaaagcagctggatttcaatgcaccacttttatcagtgaggaaaatttcatcatctttatcccctcatgagagagcaaataaaaagattatagaaagggcgccaccgaatcgacaacaatcacttcctgtcaaaaaatcagattgggaattgagtgaagtgactaaaccagtagctgttccatttatgtgggaacaaatccctggtagacgaaaaggtaacaatgatgcccgagctaaccttcgagtggagtgctcaagtagtcctaggctgccccccggaagattgccagagactattcggttctattcgggtgaaaggcctcgtgctcaaaacatttacaagtctccagctgaaggacttccttggattgatcatgcagctttactggatagcctagtggaaagtatatatacaagaggagacagagaaagtgaggatgatgcttattctgatgctcctgaaaccttgtcacctaccgaatccttatccttggactgcagtgtcagtggtctgagcggacatcaaagttcggattcaaaaccatctggaaccttttccattgactcccaaactagagactttatgatgagccgattcttacctgcagcaaaagctgttgttttggagacgcctcagtatgttcaaaaaaagcaagttccagtcagtactgaacaaccgaagccagtacccgtggaacgaaagccaacagttaagcggatggagtctaaacctgtctcatattatagcggctatccagacgatgtaggaagtgaaattgaagatgatgtgtctgagaatcagcacaaaaggccaagtaaagggtggaaatttttccctcggatttgtgtgaagaattctttatgcctattaaatccactgccaggactgaaagtgaaaacacacgttcccacaccctcatctcaagcagtaaagagagtttcaggattgaaaccaaagacgccccagagtcccacatcttatgctcatgaagtcaaaagattagctagaaaagcttatagtggtcctcttgagaagaattcttgtgatactataaataagcaaagatttcactctggagtactgtctcgcgagctttacaaagctgataatagaagtttttccggccaactacctaaccctagtgattcatgtaagctagttggaatctctcctggaagacgttcaagaagtggggctatatctccctatcgaaatgtagcacccccatctccattcaatgaaggtacaaggtttcttggtgtgccaaaggaaatggagagtctttgggctagtcgctttgattcattccggaaaggttgctacactgtcaaggacaaagtaccacagcagattggcacaggaaggttttctgattcaccaagtgaagtagttgagaaaacattgtacatagattctgtagataatgtgcaaatttcagcccgtaattctgcttcttcaaaacccaagggattagtgaactcctctagtaagaatatgaagacactggttaaaagcagtgtagtactagaaaatatggatgccacggctcgtactcaaggtgccaagaactggaatgtttcagagaaagagagcaaacagattgctgaaaaggagtcccttgatttggtggaagcttctcccgtttccatatcaactctcaaaggccctgctgatcaggagtctttgaaactaaagcaaaatcttgatacactatctggagccttagagtcttcaaaagtgcatccctatggaaatttaggtacagaaaatgaagataacaaaaatgcaaatgacccaaaagattctaaccttacctccgtggaatctcctataccaccacccttgcctaaatctccctctgaatcttggctttggcgcacgttgccttccattcctttgagaaccccattctcaagtttgagctccaagaagcagaacaagaagtcccacactgatggtaccaagtgggagactattgtgaaaacttctaacttgcataaggatcatgtccgttactccgaggaactatatactcttggttcttgtcagcagagcaaagcttga

SEQ ID NO: 85 - DNA, 1176 bp, synthetic construct
  source 1..1176: mol_type = other DNA
atggaaatagctcggagctcaagaagatgcaaatgggttacagcatttcttgttctactagcttgctcagctgctggacaagttgaagatggtccgttagttaacggtgattttgagacacctccgtcaggcggtttctcttccggcgacggattctctgatggccctctcgtactccctagctggaaaacaaacggcaccgtcgagctagtggaatcagggcaaaaacagggtgggatgatcctcatcgtaccacaaggtagacacgcagttcggctcggaaacgacgccgagataagtcaagagctcaaagtcgagaaaggctccatttattcagtcacgttcagcgcggctcgcacctgcgcccagctagagtcactgaacgtgtcggttcctccggcgtcacagaccatcgatcttcagactctgtataatgtccagggctgggattcctacgcgtgggcctttcaagccgaggaagatgacgtgcgtgccgttttcacaaatcctggcatggaagatgaccctacttgtgggcccattattgatgatattgctatcaagaagcttttcactccagataagtccaaagataatgcagtacttaatggcgactttgaagaaggtccatggatgttcaggaatgcttccctcggtgttctgcttccaaccaacctcgacgaggaaacatcatcgctacctggttggatagttgaatcaaatcgagcggttcggtatattgataattatcactttacagttccagaagggaaaagagctatagaattgctttcaggaaaagaaggcattatatctcagatggttgaaaccaagcccaacaagccatacagattgacatttttgttgggccatgcaggggactcatgcaagcagccactggctgttatggcgttcgccggtgatcaggcccaaaacatccattacactcccaattccaattcttcatttcagattgctaatctgaatttcacggctaaggcggacaggacacgtgttgcgttctatagtatctattacaacacaaggagtgatgatatgagctctctttgtggacctgttgtggatgatgtgagggttgaagtatctgggtctagcacagtcaaggttttggggttcgggtttatgttgtggctgttagtcttagtattggtttaa

SEQ ID NO: 86 - DNA, 1128 bp, synthetic construct
  source 1..1128: mol_type = other DNA
atgagaacattgtgtccaaatttggacaaggcagatgggttggagacagttttggaggtccccataccagaggagatgtttaacaatatgggtagcaatggtgccttacaatggcggaatatgcgcaatcttatgagggctcaaagtgcagacagatattcttcttcttctcatcatgccgctgtatcaagtaatgatcaattcatgttcttgtttaaaatcgttggctctgctcttgtcccttttcaagttcaattggaccatattgctgataacatgcctgttaggaatggttcaatggaggctgctactgctaaatacatagtacaacaatatttggcagcatgtggagggcaggcggcgttaaattcgctaaatagcatgtgtgcagtagggcaggtgaagatggctacatgggacatccatcaaagtggtggtgacgccaactcgaaacgccattgtgaggttggagcctttgtgctttggcagaagaaccctgacctgtgggttttagaattggttgtttcaggttgcaagataagtgcaggaagtaatgggaaggttgcttggagtcagtcttcctctacttctagtgcttcaaaaggtcctccaagaccccttagaaggtttttccagggcttggaccctagatcaacggccaatttgttcttgaatgctatttgcgttggagaaaagactattaaggatgaagaatgttttattttaaaactagaatcaagcatagacatgctcaaggcacaaagtacggcaaatacagaagttgtccatcacacaatattgggatacttcagccaaaggacagggcttctgattcaatttgaagacacaaaattagtaagattgaagtcaccaaagggggatagcaatgtcttttgggaaacaagtatggaatctatgcttgaagattatagatatattgaaggtatcaacattgctcacagtgggaagactgcagcaacaatttacagatatggaaagaacatagactatagagcaaagattgaggagacttggatgattgaagagattgatttcaatatatctgatttgccaatggactgcttcttgcctcctgctgaatttgaagagcaaggaatgggttgctga

SEQ ID NO: 87 - DNA, 558 bp, synthetic construct
  source 1..558: mol_type = other DNA
atgctgcaacatctttatcttggtcaattttctatgatgtttcttcttctattatgttctatttttgcagtattaacgactgcagaatcatctatatacgaagtgcttgaatcccatggattgccaatgggtttacttccaaaaggtgtgaagaatttcacattagacaattcggggaaatttgtagtccatttggatcaagcttgcaatgctaaattcgagaatgagtttcactatgataggaatgtatcgggtacaataagttacggacagatccatgcactttcaggaattgaggctcaagatttgtttctatggtttccagtgaaggatattcgggttgatatacccagttctggtttgatttacttcaacgttggcgttgtatctaagcaattctctttgtcttcatttgagactcctagggattgtactgctgttcaactcacggatcttcaggatgacatgcacatagctgacgctatttctaagagtcaatcaggggagcttcggtacaagtttgatcagggcaatatcgggaggaaggctatattgtag

SEQ ID NO: 88 - DNA, 918 bp, synthetic construct; identical to SEQ ID NO: 30
  source 1..918: mol_type = other DNA

SEQ ID NO: 89 - DNA, 1086 bp, synthetic construct; identical to SEQ ID NO: 31
  source 1..1086: mol_type = other DNA

SEQ ID NO: 90 - DNA, 1212 bp, synthetic construct
  source 1..1212: mol_type = other DNA
atgccttttccgatgaagattcaacctgttgatttcaataccgtggaagaaccgagtcgatatgactcgtttaagccggttcaaaagtcacggttcaagcggctattcgagaggcaattttcaggacttttgagatcatcggcgccgctggagaagctggtggccggcgaagagttgatttgtaataagaaggatgttgcgtccgacgagttcgagccgagctccgtttgtttggctaaaatggtacagactttcattgaagaaggtgaggccgataaacatcggtgcaatcggaaccgaaaatgcaattgctttaactgtaatggtactgacagctcagaggaggaaactgattcactcagttgcttcggtgaatcgaatcatacttgttgcaacgatgcttgtgaaatcctcaagagtttggtgccgtgtccttccgttattgaaaggaacgttttagctgatattacaaagatcattgagaaaaacaagatgggaaagcgaaaggacaatttcatccgaaagatagttgttgatggcctcttagctgcgggatatgatgcttcaatctgcgagtctcgctgggacaaaactccttccactcctgccggaacgtacgagtatgttgatgtggtgattgaaggagaaagactactaattgacattgatttcagatcagagtttgaaatagcaaggtcgaccaggtcctacaaatttctcctccaattgctgcccaacacttttgtaggcaaagcggatcgccttcagaagattgttcatttgctaacagaggctgcaaagcagagcttgaagaagaaaggaatgccttttcctccatggcgtaaagctgaatacgtgaaagccaaatggctgtcaccttacacccgtatcaaaccaactctaatgggggcaccaaccgtgtcaaactctgctcccgaaactggcactgcttgcaaaacctatcaacaagcggtaaaggaggagtcttcagaggattcttgcggggagctgaatctgatttttggagagagcagccaactcttggagaataacgccaagagcgtgattacctatcctctatctgcttgtgatgacgaagaaaaggatgtgatggtgcagcaaggcaaaccacctgacactaaacacaaggattcaagcaatggtgccaggaagataactggtttaacttctctgattgaagaccacacatga

SEQ ID NO: 91 - DNA, 495 bp, synthetic construct; identical to SEQ ID NO: 33
  source 1..495: mol_type = other DNA

SEQ ID NO: 92 - DNA, 327 bp, synthetic construct
  source 1..327: mol_type = other DNA
atggaggagctccgcccgagtttccggtgctccggcgaccgaagactagagatcgtaagcggaaaagggttcagcagcaaccagggaagtcgggctcgctcaccggatctaccagtagtaacgagcaagggaacttggccgagccaggtggcagcagcaacttcatcgtcagctaagccctggggattcaatgatccagagatgaagagacgaaagagaatagcaaagtacaaagtgtatactattgaaggaaagttcaagacttccattaggaatggactgaggtggttcaagaataaatgctctgaaatcatccatggctattaa

SEQ ID NO: 93 - DNA, 828 bp, synthetic construct
  source 1..828: mol_type = other DNA
atggggttttcggagaacaatagaggattagttctagcaatggtgtccagcttgttcattggaacgagttttatattgaagaagaaaggtctccggcgtgctgctgctgctggcactcgtgcaggagttggagattatacttatttgcttgaacccctttggtgggcaggcatgattacaatgattgttggggaggcttccaattttgtggcttatatttatgctccggcggttcttgtgacccctcttggcgcgttgagtataattataagtgctcttttggcacactttatgttgagggaacgattacaacggttgggtgtggtaggatgcatattgtgcattgtaggatctgtggtaatttttatacatgcacctcaggagcatatgccaacttctgtacaagaaatctggattttggcaattcaaccagcatttatgatttatatagcagcaacagtatccacagtagtagctttgatgttgcattttgggcctcgttatgggcagacgaacttactggtctatttggtaatatgttccctaatgggtgcacttacggttgtcagcataaaggctattggaattgcaataaaacttactttggaaggaattagtcagattgcctatccgcagacttggttttttctagctgttgcagtaatctgtgtaatcacacagttgaattatctaaacaaggcactggatacattcaatgctgcaattgtttccccaatatattatgtaatgttcaccactttgaccatcattgcaagcgcaataatgttcaaggactgggcgggacaagatgctagcagcatagtatag

SEQ ID NO: 94 - DNA, 699 bp, synthetic construct
  source 1..699: mol_type = other DNA
atgggtacagatacagaggtagcagaaatgagttcaacactaaagcttcctctgttttcagtagagtcatcacaagaggatttagggatgctaactcctcttcacactttagcttcaatacctttcaaatgggaagaagaacctggaaaacctcgtccatgcactgatcttatccctcttccaaactccatatgtttagaaccacctcctaggctttacaatatggaattttccaagacatcttcttctcctacaactgtatttgatggaccttatattaccaacaagcctaggttttcttcgtttaggttgttgaggagggatcgtcgtcatcgtcgacaagggtcttttgatagtactactagctctgaaacaggacaacttagtactacacttgttcttggtaacaagaaatcggaaagcaaaagctggtggcgtcggccgactactcatcatgttaaacataatggctgtgagattaatagtggttttgtgtttccatcttctatagattattcaacagattgtgttgaagaatgcagcagttccagtataaaaatggaaacatttagaagaagtggaagcttctcaggtcactcgcaagctaagactcacatctgggcagctatatacgagggtttcaagcaagtaataccatggaagaacagaaagtctaaaaaggaagcgctaatcggctaa

SEQ ID NO: 95 - DNA, 435 bp, synthetic construct
  source 1..435: mol_type = other DNA
atgtcaggagaggatgctgctgttcctgttgtcgctgctgccgagactcctgctccagcacttggggagcccatggacatcatgaccgcactacagctggtgctaaagaagtctaaagctcatggaggacttgctcgaggactccatgaaggtgctaaggtgattgagaagcatgctgcacagctttgtgtgctagctgaggactgtgaccagccagattacgtcaaactggtgaaagcactttgtgctgatcacaatgtcagtttaattacagttcccaatgcaaaaactcttggcgaatgggctggtttatgcaaaattgattctgaagggaaagcaaggaaggttgttggttgtggctgtgttgtcgtgaaggattatggtgaagagactgagggtctccatatcgtccaagagtacgtgaagtctcattaa

SEQ ID NO: 96 - DNA, 1623 bp, synthetic construct
  source 1..1623: mol_type = other DNA
atggggaacagaagttcaaatcttgataatgacaacaatatttcacgtccaattgatactccatttaagcttccttctccgttgcccacttggccctcaggtcaagcctttgctactggagttattgatcttggaggtttagaagtgtctcaaacatcatcattggctaaagtttgggctactcaagaaagtggaccagatgatcttggagctacattctttgaaccatcaaatttaccaaatggcttctttatgcttggatcctatagccaacctaacaatttacccctcttcggatcggttcttgttggaaaagattcaaaaggagacgcgctaaagattccaattgactatacacttgtatggagtagtgagaacttgaatatcaagcaggatagtgttggctatatttggctgccaattcctcctgaaggctataaagccgtaggccacgttgtaacaacgtcgcctcaaaagccttctcttgacaaaattcgttgtgttcgttctgatttaaccgatgtgtctgaaagtgatgattggatttggggcaataatggattgaatgtgtattcttcaagaccaagagacagaggaatcaatgctttaggagtctctactggtgcttttgtggcttcaaataatggaactgcagattcattagcttgtttgaaaaatgtcaatgctaatttatctgctatgccaaatttgaaccaagttaaagcactatttcaagcctactctcctttgtactacttccatcctgatgaacaatattatccttcttctgtcacttggttttttcagaatggagcattattatataccaaaggtcaagaatctgcaccagttgctattgagtcaaatggttcaaatcttcctcaaggtggttcaaatgatggtgctttttggttggatttgccaactgatgattccgcgaaagatcaagtcaaaaaaggagatttgcaggctgctacagcctacttacacattaaaccaatgtttggcgcgacgtatactgatattgctctgtggctattttacccctttaatggccctgctagagcaaaaattgaattcatgactattccattagggaaaattggacaacatgtcggcgattgggaacatgttacgttaaggattagcaacttcaatggggagttacaaggtgtgtacttttctcaacacagtggaggaaattgggtaagtgcttctcagcttgaattccaaaatggtaacaaacccgtggcctattcatcgttgcacggtcacgcttcttatgcccaaccaggaaaaaatctgcaggggaataacaacataggcttaagaaatgacacagcaaaagggcaaatgatggacactggagcaaactattcaatagttgctgctgaatacttaacaattgttgagccagcatggttgaactatgctagagaatggggtctgaaaatcagttatgatattgcaaatgaactccaaaaagtggagagatttttgccaggaaaactgaagaatgctgttgagaaacttgtgaaaagtctaccaaatgaggtgttgggtgaggaaggaccaactggacccaagtttaaggacatgtggaatggggatgaaaggggttaa

SEQ ID NO: 97 - DNA, 492 bp, synthetic construct; identical to SEQ ID NO: 39
  source 1..492: mol_type = other DNA

SEQ ID NO: 98 - DNA, 468 bp, synthetic construct
  source 1..468: mol_type = other DNA
atgttgcttggaaagaggccaaggccaccaatcaaaagaacaacaagcatgaaagaattcaccttggatctcaacataaacgatccttctgtagccgttataaattatcagccctttgatcctctcaacccctacaacttcactggtcccgtcccaatgccctccaacggcctagatcaacgcttaatcttgactcgcagaagatcagctgatttcgttggaactgcccatttccttagagcttgctccctctgcaaacgccagttaatccctggtcgtgacatctacatgtacagaggagatagtgctttctgcagcctagactgtagagaaaagcagatgaagcaagatgaaaagaaagagaagtatccgaaggttgcaactaggaaaaaagttactaactcagccgccatggccgccgtggcaagcggaacaggatccgacgctccacctcaggccagaccgtaa

SEQ ID NO: 99 - DNA, 1443 bp, synthetic construct; identical to SEQ ID NO: 41
  source 1..1443: mol_type = other DNA

SEQ ID NO: 100 - DNA, 1233 bp, synthetic construct
  source 1..1233: mol_type = other DNA
atgatggggagtggattacagtttaatcggagttttaacggtgaagatcggttttatagtgcggggaagtttcgccggaatgttaatcggagtttttctaccggcagtgaggataatctccggcgagctaagagtgatgtcgccgttattcattccccgacgaagccgaaaatgaaagttgccgccgatgagtcgcctgatgagccggtatctgccgtgccgccgacggatgttctgcccccgccgctttgtaatcttcagcggtttttgaaatctgtttctccttcagtacctgctcaatatctatctaagacaactatgaggggttggaggacgtgtgatgtggagtttcagccatactttgttcttggtgacttatgggagtcttttagagaatggagtgcatatggagctggagttccattggtgttgaatgaaggtgatagtgtagttcagtactatgtgccctatttgtcaggtattcagttgtatggtgactcgacaaaggcttcaacaaaaacaagtaggcgaccaggtgaggaaagtgatagtgactatttccgggattctagtagtgatggaagcagtgactctgaacatgagagacgttgcttgaattatacaagggagcagcggatgtatcatagccaaacaagtgaaagctcccttagtattgaccggttatcattaagagacaggaatgctaccttccaagaaggattttcaagcgatgagggtgaatctgggtcttctcagggtgccttgttgtttgagtatcttgagcatggtcagccttacggttgtgaacctttggcagataagatatctgatcttgctcaacgttttccggaattaaaaacaatgagaagttgtgatctccttccttccagttggatctctgtagcctggtatccaatttaccggatacctactggaccaactttaaaagctttggacgcttgttttctgacctttcattctcttcatacacccatgacaggaagtcaaagtgctcatcctgcggcggtaacatgtccgagtgatacggatccggtccctaagattccattgcctgcttttggccttgcttcatacaagtttaaagcctcactttggaccccaaatggaggatctggaagacagttaatgagctcgcttttgcagtctgctgaaaactggctaacgctgcttcaggtcaatcacccagatttcagtttcttctgccaaaggtga

SEQ ID NO: 101 - DNA, 681 bp, synthetic construct
  source 1..681: mol_type = other DNA
atgggttcatgtgtttcagtgcataaagactctgaatcagccatgaaaattcgtcttgtttttggttcttccaaaactgacaagcttgtcactccttcacctctcaacaaagatgataccaaagtctctgatcttcaactcaaatctcaaactcctgctgttactgcttttcgtgactttggtagcaaggaggagaccttctttgattcccagccttggttggattcagattgtgaagatgacttcttaagcgtgaatggagattttacgccatctcgtggaaatacacctgtccatcccttggctggaaatttaacagggaacagaacccctgcttattttcagcaatcatccccaacagataaaaagaagagattgtctgaactttttgaagaaagcttgagaagcgatctggaccaaaatgggcaaaatgctgaagaaaaacaaaatggtacaaacactaaaaaagaaactgcgtcaactggtactcaacttccaccaagatccacacctgggacaccttatgcatccgtgtgtagtagcgaaagaacacccagtggtctgttaaaatctgacgtcaaaacgtcaaagtcagcacaatgttgtcttccaaggttgctctcaagtcgtagctttaacggaaggaggagtagaatgagtcctgcgcgtaatgttggctga

SEQ ID NO: 102 - DNA, 600 bp, synthetic construct
  source 1..600: mol_type = other DNA
atgaaggatgatgaatccataccagtttcaacacctacaacacattcatcatcaaatacaatttcaacatattcgaagaattccacatctgatgccacaatattcggtcgtggtcggtacaagttctgggcattagccgctattcttctgctagcattttggtcaatgtttactggtactgttactctccgatggtccgccggcaacctcaacggtctctccgattacttcaatgtgcctctctccgataatctcgacgtcatcgaaatggaagagagggagaagttggtgaagcatatgtgggatgtgtatactaatactcctcggatcagattgcctaagttttggcaggaggcattcgaggcggcctatgaagagttaaccagtgaccttgctgatattcgagaggctgctatctctgagatctctaagatgtctctccgatttgttcatatggagccaccacctctgcgttcattggcagtaagagaattaagtcaaaaacaagctgaggacaaacagaggactgcaaaaggaatttggctaagtgcagtccttatggaggaaaataaaagagaagaaaaaaggggacctgattag

SEQ ID NO: 103 - DNA, 549 bp, synthetic construct
  source 1..549: mol_type = other DNA
atggccgatagtgaatcgaggcaattttcacctgataaggagccatgttttgcagaagaactacttgactggcagaggagggaactgccccgcaggttcttgtctggtcaggttgtctgggatgacgacgctttcatccccgaaggcgcttcggagaagcacagggttatatttcgcagaatttataggaaatacttccttcagatttttgcttcggatggtttcgacattgacatctatccgggtaaagccaaggcagctatgcttataccatacctggattttgagaaagaaattgacttgctgatggaactggctaaccatgctattcaggattacaacagtaaagaaaccaatgtttacaaatacgaggtcttgtatgttgaaaaagtgaactttattttggccgaatgccgcgaattttttatgactgttaaagttaaaaatctcactctgcgttcacctaaagaaacttttcaaatccatgcatataaaggaccagatggggagaatattttctgcctttgccggagaaaattgctggtttga

SEQ ID NO: 104 - DNA, 909 bp, synthetic construct; identical to SEQ ID NO: 46
  source 1..909: mol_type = other DNA

SEQ ID NO: 105 - DNA, 504 bp, synthetic construct; identical to SEQ ID NO: 47
  source 1..504: mol_type = other DNA

SEQ ID NO: 106 - DNA, 2277 bp, synthetic construct
  source 1..2277: mol_type = other DNA
atggaaagacggccaaagcagagtggtgaaagaaagacaatggagaatgaagagcttgtcaaatacatgtcaagtttgccaagttacctggaaaagggggaaaatctgcaggagaaagctttcagtgttggcgtcctagattggcgttttcttgaaaaatggcgacacgaacatgtaaaagaaccttgtagaactagtggttgttcaccatccaccagcaataccttgtcattttcctcaatggagggatcttcatccaattctagcagagccagaagctgctctcctgctcgtcggaggattcatcgtcctacatcacaatcttattatgcatcaccacctaaaggaagttatgtacagttgcaaaaccaaaaacctggcgtaattaaatccccggagattgagcaacttgtctgcaggatatatcagtcttttgatgagtatccacaaagaaatatgcaagaactcggcaatgctctacacccgcgtaggactcctcatgttaggagatcacttgatgtagagactaagacgagaacatcacgttccaagggaaagatgaagatccaagatagagaatgtttatcgaaaggagattttgatgattttgattgcatagagaaacataaatcagatgtccttcaagtaccagaacctggccaagaaactaacagctgcacaactttctgcccacctgattcagtagtgaaagaccagagcgcggtgaaatcaagcagacggagcttttcttgtggatttatttctgcgttctactatggacagtcctcttcagatatctctagttccagcacccttcctcatgatgctgatgagtcgaaggtgggacaagcgagtccgatagatgcaaaggatagttgcctttcatctaagacaattcagccttcaacatattcaggaaacaaattgtccagcccacctggaaccaattccaaacaagagaagaaatcaactgtgatgcttaagaatccaactacacttaattccgcagaatctacaaacatcagaaattcctctccaactcgtcaatttagcatggcaatgggaaggattggccagatttcaggtataaaagatatgatcactggatcacaaggtgtcaaatggcctgcagaacaatctagtccaaataaaactcaaagttcgtcctcaattgatacaggctgtgataaatcagacaccactggccgagccaggactagtcctttgagaaggttgctagatccactgctgaagccaaagacaggcaactctgatcacgttaaatcttcaacaagacgtggtgaatctcctacgaaacgctcattgaaagtcaaattggatttgaagagctgtaaatcgattgatatcgacgatccacgttctaatggcacatttgtaccatcaagattgcaagctctcctccaagtagctgtgaaaaatggtcttcctctattcacatttgcagttgacaatgaagttgacattttagcagccacaatgaagaaacttaatccgaatttgaaggattacagttgctggatttataatttcttcaccgttcgtgaaacaaagaaaaagagtggaaattggttaaatcaagtagaaaaagacagaagtcatggaattatgcctaatatcgttggaaagatgaaggtttctgatgttccattctccgaattgaataggcagaaacttgactcacagtttaggattacggaatttgttttgtttgctacagatcagaaagcatctgaccttcacccaaacgatgagcttgcagctattgttgtcaaattacccaatagaacctccatttgtcctaatggaagtggccatcaggatagaaactgcagtaatacttcaacttcaggtttaacaaatccttttgaagatcttaacatgacagttatccttcctggtggtgcacatagcgtaccaagtaaaggcgagccttcttcactgattaaccgatggaggtcaggtggatcgtgtgattgtggtggctgggatttgggctgcaaattaagacttcttaggaatcacactaatcatcaaaggatatcaagttgttccaaacccaaacttaatgctgcaagatttgaacttttatcccagggggaagcacgggacagtaagcctgtcttcagcttgtccgcattcaaggatggcatattttcagttgaattcagttcttcactgaaggttctacaagcattctccatttgtatagcagttttaaatggtagaaatcaagaaagcttccaaatgcaaacttga

SEQ ID NO: 107 - DNA, 558 bp, synthetic construct
  source 1..558: mol_type = other DNA
atgctgcaacatttttgtcttggtcaattttctgcgatgtttcttcttctgttatgttcaatttttgcagtattatcaactgcagaatcatcaatatacgaagttcttgaatcccatggattgccaaggggtttacttccaaagggtgtgaagaatttcacattagacaattcggggaaatttgtggtccatttggatcaagcttgcaatgccaaattcgagaatgaatttcactatgataggaatgtatcgggtacaataagttatggacagatccatgcactttcaggaattgaggctcaagatttgtttctatggtttccagtgaaggatattcgggttgatatacccagttctggattgatttacttcaacgttggcgttgtatctaagcaattctctttgtcttcatttgagactcctagggattgtactgttgttcaactcatggatcttcaggatgacatgcacatagctgacgctgtttctaagagtcaatcagaggagcttcgatacaagtttgatcagggcaatatcgggaggaaggctatattgtag

SEQ ID NO: 108 - DNA, 915 bp, synthetic construct
  source 1..915: mol_type = other DNA
atggcttctacagtaccggcgaagtcgaataatccactttacaacttcgatttttcccacttgaaatggaagaagaatcatcattcgaacaatcatcaacgtcgccgttccaacaagctgtcgtcggactcttcctctccgtcacggcacgactcgccgctaaggcactctcagtctcagtccccgatgcgtgaatccctcgcggcggcgcgtcagtctccagtaagtgaatctgctgagacggcgcgtatatcgccgatgcacaattcggctgcggcggcgcgtcagtctccgatgcgtgaatcggcacgtcagtctccgatgcgtgatcccgttccgtcggttcaacgatcaaagcataaggtaccagagattaatgttgtcagtagcaaagaaagcagatcgaaaatcttaattaaaatccctcgcaaaaacaaatccgaagaaattcaaatcaacgaggatcaaaatcaaaaggaggcagatgagtctcacgacgaagctgcagcagctgaagaaacagcacaaaaaacgtggaatctgagacctcggaagccgatacacaaatcactgaatataaacggaggagtcccatttcgatccagtggatctgcgatgcaggagatcaaatctcaatcaccccatcacatgatgaacgtgaacaagccggaaaataatgaaacccacgctgcttctgctcagaagaaggtaaaaaggcaaaggttttccatagcgttatccagagaagagattgatgaagatctatatgctatgactggattaaaagctgctagaagacctaagaagagagttaaagttgttcagaaacaacttgatacactcttccctggtttatggcttgcttcaataactcctgattcatataaagtttgtgaaaatcttccaaagggttag

SEQ ID NO: 109 - DNA, 1113 bp, synthetic construct; identical to SEQ ID NO: 51
  source 1..1113: mol_type = other DNA

SEQ ID NO: 110 - DNA, 1113 bp, synthetic construct; identical to SEQ ID NO: 51
  source 1..1113: mol_type = other DNA

SEQ ID NO: 111 - DNA, 435 bp, synthetic construct
  source 1..435: mol_type = other DNA
atgctgtggcggaattcgacaaaattgcagctgtgttcttcatccgctaggagtcgtttgctactgatttgtcgcctcgaacatcaattcatcgtcacacacagaaaaccagttgatctttggggattgaaaatctgtgaattgaagaggcctatagctgctgttaagttgaattgcaaactgcaggttgtagttccctgctgctacagtttggctgcaattgtgaacatcttcagtagcttgcagtcactgatgaactttgcatttgagctacaacttggagtctttttacagaattgtgaagaagattgctcaggagatcaccaacagtctgcaaccttgtcagttattgtgaggctgtccagaattgcagatttgaccagtccttacctatgtggccattctttgcaaatccgaccaattttacaaaactag

SEQ ID NO: 112 - DNA, 1068 bp, synthetic construct; identical to SEQ ID NO: 54
  source 1..1068: mol_type = other DNA

SEQ ID NO: 113 - DNA, 1158 bp, synthetic construct; identical to SEQ ID NO: 55
  source 1..1158: mol_type = other DNA

SEQ ID NO: 114 - DNA, 591 bp, synthetic construct
  source 1..591: mol_type = other DNA
atgtggcagtttaacaagatatggtgccaaaatcaaagaattcaaattacctattggggcctattagcatacaatgctggtgatgatatggtaacttctacaaccccttttgagcttataattcaatctgataaaattccaatcacaattgattttagcaatacaacaagactagtaaatgatactagtacatctgggataataccattatgtgctagctttgagcgcgacggaaaggtgacactaacaaaacaagtgtcacaaaatgtttgtgctgctaaaaaacaaggacattttggtttagtaattgagtcacctctaatgccattgaaaaataaaagggcaagtaattggaaattagctattgggagcgcgattggagctgcgttgggcgcgttccttttaggtttgctactgattgcaatatttgtgaaggtgaagaagaaagcaagaatggaggaattggttagaagagcatatgaagaagaagctttacaagtttctatggttggacatgtgagagctccaactgcagctggtactaggactgttccaactattgaacatgactataatcctcataattcttga

SEQ ID NO: 115 - DNA, 513 bp, synthetic construct
  source 1..513: mol_type = other DNA
atggatcagatattgaacaaagtgggttcctactggattggtaagagagctaacaaggagctcaattccgtcggcgatgacattaactcattgcaaagcagtattgaaggaggaacaaaatggctggtgaacaagcttaaaggaaaaatgcaaaagccattgccagatcttttgaaggagtatgatgttccagtaggtattttccctcgggatgccaccaattacgagtttaatgaagagacaaggaagctcactgtctatataccctctgtatgtgaagttggttacaaggattcatctgtattacgcttctctacagaagttactggatttttagaaaaaggaaagctagctgacattgaaggaatgaaaacgaaagtgatgatgtgggtaaaagttactgccatctcatctgaaaaatccaaggttcatttcacagctgggttgaagaaaacccggagtagggaggcttatgaggttttgagagatggagtagctattgaaaaattctaa

SEQ ID NO: 116 - DNA, 675 bp, synthetic construct
  source 1..675: mol_type = other DNA
atgggtgccattagtttcttggacaccatattagtccccttgagtcttttcatcacaattggctatcatttctatctttggcaccacttgaagcataaaccttctcgcaccaccattggtatgaatatactcaagaagagatcttggctgcgagagctcaatcaaggaaacgagaagaaaggcacgttagctgtacaaagcttaagaaatgcactaatggaaacaatactcacggcaacaatcacaatgatcataactttagcattggcagctctaacaaacaacacttacaacgtgagaaacctctttactagtgctttctttggatcacaaacagggaagataatcgtgttgaaatatggctcagcaactattttcttgttggcaagtttcttgtgtagttctttggcacttagttgtttgatcgatgctaatttcttggtcaatgcattgggggaattttccataaatccaatgtatacagagacagtatttgaaagaggatttacattggcttttgtaggggatagagtgctttgcatggcttttcctcttttgttatggatgtttggccctgtgcctgtggttgtgtcttctttggctttggtttggggattacatgagcgtgattttgctggaaatttgtcaagaactatgaataagagttgtatttga

SEQ ID NO: 117 - protein, 157 aa, synthetic construct
MSHLTAAPPSEPPSAAVNSLYKQKSWSPDTFRDEAWQRRKGTHGSRLKQRSKSVTDEDLDELKACIELGFGFDSPEIDQRLSDTFPAYGLFYAVNKQYADTLSKTSSLSSVISYCESPAPPGSPHTIVDQGDNPQTVKTRLRQWAQVVAWSVRQSSH

SEQ ID NO: 118 - protein, 690 aa, synthetic construct
MEKKQLDFNAPLLSVRKISSSLSPHERANKKIIERAPPNRQQSLPVKKSDWELSEVTKPVAVPFMWEQIPGRRKGDNEARANLRVEWSSSPRLPPGRLPETIRFYSGERPRTQNIYKSPAEGLPWIDHAALLDSLAESIYTRGDRASEDDANSDAPETLSPTESLSLDCSVSGLSGHQSSDSKPSGTFSIDSQTRDFMMSRFLPAAKAVVLETPQYVQKKQVPVSTEQPKPVPVERKPIVKQMESKPVSYYSGYPDDVGSEIEDDVSENQHKRPSKGWKFFPRICVKNSLCLLNPLPGLKVKTHVPTPSAQAVKRVSGMKPKTPQSPTSYAHEVKRLARKAYSGPLEKNTCDTINKQRFHSGVLSRELYKAENRSFSGQLPNPSDSCKLVGISPGRRSRSGAISPCRNVAPPSPFNEGTRFLGVPKEMESLWASRFDSFRKGCYTVKDKVPQQIGTGRFSDSPSEVVEKTLYIDSVDNVQISARNSASSKPKELVNSSGKNMKPLVKSSKVLENMDATARTQGAKDRNVSEKESKQVFEKESLDLVEASPISISTRKGPADHESLKLKQNLDTLSGALESSKVHPYGNLGTENEDNQNANGPKDSNLTSLESPIPPPLPKSPSESWLWRTLPSIPLRTPFSSLSSKKQNKKSHADGSKWETIVKTSNLHKDHVRYSEELYTLGSCQQSKA

SEQ ID NO: 119 - protein, 419 aa, synthetic construct
MEVISTNTNGSTIFKNGAIPMNGHQSGTSKHLNGYQNGTSKHQNGHHNGTSEHRNGHQNGISEHQNGHQNGTSEHRNGHQNGISEHQNGHQNGTSEHQNGHQNGTSEQQNGTISHDNGNELLGNSNSIKLGWFSEFSALWPGEAFSLKVEKLLFQGKSDYQDVMLFESATYGKVLTLDGAIQHTENGGFPYTEMIVHLPLGSIPNPKKVLIIGGGIGFTLFEMLRYPTIEKIDIVEIDDVVVDVSRKSFPYLAANFNDPRVTLVLGDGAAFVKAAQAGYYDAIIVDSSDPIGPAKDLFERPFFEAVAKALRPGGVVCTQAESIWLHMHIIKQIIANCRQVFKGSVNYAWTTVPTYPTGVIGYMLCSTEGPEVDFKNPINPIDKETTQVKSKLAPLKFYNSDIHKAAFILPSFARSMIES

SEQ ID NO: 120 - protein, 381 aa, synthetic construct
MEVISTNTNGSTIFKNGAIPMNGYQNGTSKHQNGHQNGTSEHRNGHQNGISEHQNGHQNGTSEHQNGHQNGTISHDNGNELQLLGSSNSIKPGWFSEFSALWPGEAFSLKVEKLLFQGKSDYQDVMLFESATYGKVLTLDGAIQHTENGGFPYTEMIVHLPLGSIPNPKKVLIIGGGIGFTLFEMLRYPTIEKIDIVEIDDVVVDVSRKFFPYLAANFSDPRVTLVLGDGAAFVKAAQAGYYDAIIVDSSDPIGPAKDLFERPFFEAVAKALRPGGVVCTQAESIWLHMHIIKQIIANCRQVFKGSVNYAWTTVPTYPTGVIGYMLCSTEGPEVDFKNPVNPIDKETTQVKSKLAPLKFYNSDIHKAAFILPSFARSMIES

SEQ ID NO: 121 - protein, 381 aa, synthetic construct; identical to SEQ ID NO: 120

SEQ ID NO: 122 - protein, 169 aa, synthetic construct
MPNQKQGSEQLQTLMQSGQISGSLSFNGALTKEDEEMSRSALSTFKAKEEEIEKKKMEVKERVQAQLGRVEEETRRLAIIREELEALADPKKKDVQIVRKKIDAVNKELKPLGQTCQKKEKEYKEALEAFNEKHKEKVQLITRLMELVGESEKLRLKKLELSKSIETIR

SEQ ID NO: 123 - protein, 435 aa, synthetic construct
MDPCPFVRIVIGNLALKFPAEHKLSSSTVFDCKFKLRGFSTQVSTISAFVQDPILDNRTHSCFNLNKTELDKLVEKSTAKGKTCSLKIEIFARKKGIGCGLLNGGKLLGSVIVDLKGLESDGRRGVVIQNEWVLVGGSSTAAQLHLNVKAEPDPRFVFQFDGEPECSPQVFQVNGNVKQPVFTCKFSFRNNGDRNLRSRSSLSKPSTSTSCFNSCTSDKESSPLKERKGWSITIHDLSGSPIAAASMVTPFVPSQGSSNVSRSNPGAWLILRPGNGTWKPWGRLEAWQERGGDLGYRFEIIPDGATDAITLSNSTISTKNGGKFGVDITNGATPLTSPNSSFDLCSGSGSGSDFGSAPGSGSWAQLLYRGFVMSSTVEGEGKCSKPEVVVGVQHVNCVEDAAAFVALAAAMDLSMDACQSFTKKLRKELRQSDQE

SEQ ID NO: 124 - protein, 141 aa, synthetic construct
MSGEDVAVAVAETPAPALGEPMDIMTALQLVLRKSKAHGGLARGLHEGAKVIEKHAAQLCVLAEDCDQPDYVKLVKALCADHNVSLITVPNAKTLGEWAGLCKIDSEGKARKVVGCGCVVVKDYGEETEGLHIVQEYVKSH

SEQ ID NO: 125 - protein, 143 aa, synthetic construct
MSGEDAAVPVVAAETPAPALGEPMDIMTALQLVLRKSKAHGGLARGLHEGAKVIEKHAAQLCVLAEDCDQPDYVKLVKALCADHNVSLITVPNAKTLGEWAGLCKIDSEGKARKVVGCGCVVVKDYGEETEGLHIVQEYVKSH

SEQ ID NO: 126 - protein, 432 aa, synthetic construct
MDPQAFIRLSIGSLGLRLSGTTTLNSTKTGISAVSSPCVCEIRLRGFPVQTSSIPYISSPEATPDIHNVASSFYLEESDLKALLAPAPCFYAAHACLEIVVYTGRKGGHCGVGIKRQQVGTFKLEVGPEWGEGKPAILFNGWIGIGKNKLETGKPGAELHLRVKLDPDPRYVFQFEDKTKLSPQIVQLQGTIKQPIFSCEFSQDRVSPVDPLNNFWSSSFDGSELEVEKRERKGWKVKIHDLSGSAVAAAFITTPFVPSTGCDWVAKSNPGAWLIVRPDICRPESWQPWGKLEAWRERGIRDSICCRFHLLSEGQECGGDLLMSEILISAEKGGEFYIDTDRQVQAAASPLPSPRSSGDFAALSPVAGGFVMSCRVQGEGKCSKPLVQLAMRHITCVEDAAIFMALAAAVDLSIEACRPFRRKLRRSTRHSW

SEQ ID NO: 127 - protein, 427 aa, synthetic construct
MAKVCFYNVKMNMQVMFCLFLVSLCRDDAAAEAKQGMSLAAQKFEVQKHLNRLNKPAIKSIKSPDGDIIDCVHMSHQPAFDHPLLKNHTVQMMPNYHPEGLFRDGKLSTAKTKNEGSKSITQLWHLNGKCPEGTIPIRRTKKDDILRASSMKSYGRKKKHSTIPKPESGRPGLVSQTGHQHAIAYVEGDKYYGAKATINVWEPKIQQPNEFSLSQLWILGGSFDSDLNSIEAGWQVSPDLYGDNNTRLFTYWTSDAYQATGCYNLLCSGFIQINNEIAMGATISPLSSYHGSQYDISILVWKDPKEGNWWMQFGNDYVLGYWPGFLFSYLTDSASMIEWGGEVVNSESDGLHTTTQMGSGHFPDEGFGKSSYFRNIQVVDGSNNLRAPQDLGIYTEDNNCYDVQLGKNNDWGNYFYYGGPGRNPNCP

SEQ ID NO: 128 - protein, 312 aa, synthetic construct
MSIDKSSMQSNLDCFLECTTPLVPSQFLPQSEIRNLNRLWHPWEREKVEYFTLADLWNCYDELSAYGAGVPIKMDTGETLVQYYVPYLSAIQIFTSRSSVNFLREETESVCETRDSFSDSFSDESESEKLSRWDGCSSEEGDSLWQMNDRWGYLYFQYFERSTPYGRVPLMDKISGLAERYPGLMSLRSVDLSPASWMSVAWYPIYHIPMGRTIKDLSACFLTFHTLSSSFQDMDLEDDTENGKRKRKEGESIPLPPFGLGTYKMQGDVWLSDRKGRDQERLASLFSVADSWLKQLGVQHHDFNYFMGIRRG

SEQ ID NO: 129 - protein, 316 aa, synthetic construct
MTKTSKVQRVPGLLSCRSKVCKKIQKEKRPKSSEKKEWEGATCSVCMEHPHNAVLLLCSSYDKGCRPYMCATSCRLSNCLEQYKKAYAKVTSIEGSEPELLSIDDPNCLSGAGSCAGKSVVELLCPLCRGQVKGWTVVEPARKYLNAKKRTCMHENCSFVGTYKKLRKHVRREHPSARPLEVDPSHAEKWKKLEHERELNDVFSTIRSAMPGAIVMGDYVIEGNFGGFHRNFGLDDHLGDTLFRSESNGIPWNDIVHSDDVFDDGYHSFDEDDFFVNHSGSGAASNVFNRISRLRSRLLLGRSRRRQRHRASSRIR

SEQ ID NO: 130 - protein, 426 aa, synthetic construct
MRTLCSNLDREDGLETVLEVPIPEEMFASNKHRARQSKNSGVKSHMDKSAASVFGSRNAEIQLLLGVVGAPLIPHPIRCDYSLNTKINDHPIEASVAKYIVQQYIAAAGGEHALNSIDSMYAMGKIKMVASEFIAGDGMGLNNGNMMKIKSVKNGPGEMGGFVLWQKRPDLWSIELVVSGCKISAGSDGKVAWRQTPWHNSHASRGPARPLRRSLQGLDPKSVANLFSNSICIGEKTVNIEDCFVLKLETEPSTLKARSSSNVEVMRHTVWGYFSQRTGLLLQLEDTHLLRIKAPGNDVFWETTMDSLILDYRTLDGVNIAHGGRTSVSLFRFGKNSEGLTRTRMEEVWTIEEIDFNIKGLSADCFLPPSDLKKEDEVSHDVNKKSRLGFKARTNNAKLRTARKGASKIMSIDEEDLEDYEADEES

SEQ ID NO: 131 - protein, 125 aa, synthetic construct
MIIGSSFSEKLMGKKKILKKTKELSVAIAESSAMSGDSQQQQQQITPRKRGRPRKIIVKDEEEATGEIKKLKINEGEELENKEAAEGEKKEAEEQSEEEKNLQPQKQQPVAKSRARRKSKPRKSC

SEQ ID NO: 132 - protein, 525 aa, synthetic construct
MMAQHIEISPLLDQTPPNEATKDESPARPRLLKPSPTAAASFFAFAVASHQVIQEGGKADYIIEIRTADQTNESANQVFDEGSSTIFKVNVGLSESNPDAYTPKLISIGPYHNQNPRLRSMEKYKLRYRQRFLQRKAWRDVEYYFSEMEKLKDEALKCYDDIRDLDSNIVSKFLDLLLLDGCFVVEYIREFYEGVPEGEDKIINAAWMESLVDRDLLLLENQLPFFILAKLHEMTKDPTDAPFIQMVKYNFGSSLPKVTPKFINATDDDAEEIKHLLQVVHMCCCPSEMNTGLTRNSRKKKGSSKKSCNWNPLRIGKSKKKFKTKDGDLWHDRMRSATELDEAGIRFSNVGKIYRKLNKNNKEDAISLFDIKFNKGLLEIPCFEVINSTETILRNLIAYEQHSSDVHPKYFTDYIIFMDHLINSGKDVNLLRLNGIIRNRIGDDEEVAIMFNKLGEGVIPSTDFFYKVECRKVIEHCEKPWNENMASLRHNYLNSPWAKISTAAAIILLLLTVAQTVLAFISTLK

SEQ ID NO: 133 - protein, 302 aa, synthetic construct
MRDFPSCFGENGVQVADASCSAVGGVTKTPQNLVTCVYQCKLLGKSCLITIVWTKSLMGQCLSVEIDDMSHQCLCKVDVKPSLFSKRKGSRSLEVNSCKIDLHWDFSLGKFGSGPEPIEGYYLALVCKGQMVLVIGDLRKEAFKKTNATPSFSNAMFISKREHIFGKRVFGTKAQFCYTGPIHDITIECDSNGIDDPCLLVRIDSKTVMQVKHLRWKFRGNYTVLIDGLPVEVFWDVHNWLFSSNFGNAVFMFQTCLSAEKLWTTQTLSDLSVMPWSYTESLSNSKSSGLGFSLVLYVWKNE

SEQ ID NO: 134 - protein, 375 aa, synthetic construct
MEVISTNTNGSTIFKNGTIPMNGHQNGSSEHLNGYQNGISKHQNGHQNGTSEHRNGHQNGTSEQQNGTISHDNGNELLGSSNSIKPGWFSEFSALWPGEAFSLKVEKLLFQGKSDYQDVMLFESATYGKVLTLDGAIQHTENGGFPYTEMIVHLPLGSIPNPKKVLIIGGGIGFTLFEMLRYPSIEKIDIVEIDDVVVDVSRKFFPYLAANFNDPRVTLVLGDGAAFVKAAQAGYYDAIIVDSSDPIGPAKDLFERPFFEAVAKALRPGGVVCTQAESIWLHMHIIKQIIANCRQVFKGSVNYAWTTVPTYPTGVIGYMLCSTEGPEVNFKNPVNPIDKETTQVKSKLGPLKFYNSDIHKAAFILPSFARSMIES

SEQ ID NO: 135 - protein, 375 aa, synthetic construct
MEVISTNTNGSTIFKNGAIPMNGHQNGTSEHLNGYQNGTSKHQNGHQNGTFEHRNGHQNGTSEQQNGTISHDNGNELLGSSDSIKPGWFSEFSALWPGEAFSLKVEKLLFQGKSDYQDVMLFESATYGKVLTLDGAIQHTENGGFPYTEMIVHLPLGSIPNPKKVLIIGGGIGFTLFEMLRYPSIEKIDIVEIDDVVVDVSRKFFPYLAANFNDPRVTLVLGDGAAFVKAAQAGYYDAIIVDSSDPIGPAKDLFERPFFEAVAKALRPGGVVCTQAESIWLHMHIIKQIIANCRQVFKGSVNYAWTTVPTYPTGVIGYMLCSTEGPEVDFKNPVNPIDKETTQVKSKLGPLKFYNSDIHKAAFILPSFARSMIES

SEQ ID NO: 136 - protein, 480 aa, synthetic construct
MANKVSNFSDLIQRVTASCLLHPLSSGRIDGNISDARYSDESEDDKYSTEDEEEKEDPPYVETREKFQNVEVTKEEIRTEKVTEMEMLLGEVFDAVSAVKTAYVSLQEAHCPWDQDKMRVADVAVISELRRLGVLRERFRRNVGGGIRKGDWRVGAATLREVVAPYEATVEELRQEVKAKEIEIDNLREKLKTATSLSSSGGKKSKSKRKVSGSSQAPVTMSPAPDVFETTVSLVKEASKSFTSLLLSLMRSAHWDIAAAVRSIEAASSNTNTPTADSIVGANHAKYALESYVNRKMFQGFDHETFYMDGSLSSLIHPDQHRRDCFTQYRDMKAMDPMELLGILPTCSFGNFCFKKYLAIVHPKMEESLFGDLEQRRQVLAGNHPRSQFYGEFLGLAKAVWLLHLLTFSLDPPPSHFEASKGSEFHPQYMESVVKYSISMGIGGRMGMGLVVGFPVSPGFKLGNGSVVKARVYLVPKNGF

SEQ ID NO: 137 - protein, 107 aa, synthetic construct
MDRLINSDKDVKLFRLKGIIRNRIGDDNEAASIFNRLGEGVIPSTQFYYKEACKKVVEHCEKPWSERKASFWHNYFNGPWVGLSTAAAVILLSLTVMQTVLTFISTL

SEQ ID NO: 138 - protein, 267 aa, synthetic construct
MAMGPERSKPPLHNFTLPCGLKWGNQKFLRCAKVESDGQISAIHRRSFGSELIGRRRSNDRKFRPPEKQDAGEGIGAVREKLMFDLQTEADKIKDAIFREGLEEQQLSPAPAKTATAAVSYAGELSRPWNLRTRRAACKEPNGFVAGAGAAGSGGGGSKGGLKIDAYRTNAPSPLRTENKSPTLRSDFAGGAAAGASASGEKRQRVKFSVPLSRGEIEEDFMAMVGHRPPRRPKKRAKFVQKNLDTLFPGLWLTEITPDLYKVPEDQ

SEQ ID NO: 139 - protein, 651 aa, synthetic construct
MEIISERRRTRSQAAAILVELKSINFQSEKRPKVHEKDGVKNFGKRKRRKGSNVRRKKKGKYVQVDSDDDEDYVFDKSPKRRKLDYGHKGKSSSSDLKIWDFYVQPRKHFTARIGTHTNVDVVKLLNAKLDDRQIQMFRETCFGHFLDLPDVLVQPQLIHSLLLREVVHEREDELWISVNDVRLRFGLVEFGIITGLKCTGDADMCLDFDGTNRLFDTYFSELTRVPKQSLIDCFLNKRWKSDEDAVNIAVLYFIHTFLLSTLNRNYITRDDFGLVESGAYQTYPWGKVVFKATLKSIKGRLLGKPSMYRLRGLPLAFQCWFYECCPYVNKKIAFRVDDKVPRILSWKVTKQPNLKKLSNGIFMQRRDQFMLRNISPTEFEQTTLNLPESFENERDNEVASGDGAEVHLSDDDFSGSPPQTSRKQPKTKPDHPLNNDDWSIELKRLSDGQSELKSEIQMLNKEVASLKDCMVASFADAFKAIKSLSKKQREKIASELDGENGHHDRHGESDSSDFSNSEDNGCEGQEHGKDSMGDKDNSEKGNEVALGDAEGRETVRDPVEEGERMDVSASPILPDTCEVSDHITPEQSHPTPILESPYVNQGDTGVEGSSEKSILKGEHPLESKIVDNVDYSLLSEFDKWVDEGMKKESK

SEQ ID NO: 140 - protein, 298 aa, synthetic construct
MSSHERRGPVAVVGSGGYSHESEPDLAAMVSDFLESSSVGAESRYSSDNDSGFSDLALLADRISLHKHSVDQYESDLTMVVHSLILSLGESCHLSKPETCNASCIRSNLVKLLQSCGYDAALCSTKWQGCGKIPGGEHEYIEVISRGNDGCSERYIIDIDFRSHFEIARAVKSYNVVLSCLPPVYVGTVRKLKQYLQTMVEAARCSLKQNSMPLPPWRSLAYLEAKWESSSQRVANFQVQSSIGPSNSSHQHCTELLWRIKSSIGSEINAKSFLVPKSCRKKQGLKIDKLRHSSPVTP

SEQ ID NO: 141 - protein, 279 aa, synthetic construct
MQEEEFFCPSFSSYSSNRAAEIAAKISDEIERNSKVAEQAEVEANAGEDDFEFSLVCENPEASVGEFFYDRQIQPVFPVFNRDLLLNVVSYDVDHKGGVDGESSENANSSIQVSLKDLFLEDREPPSSSSSEVDELESVPPGTYCVWKPQITEPSPSRCKKSNSTGSAFKRWSIRDLMRRSNSDGKDSFVFLTQEKGTKNETSKTKDSVEASKVTGKLKAKGSSNVGEKASSSAAVYLRNQAAAKEMDKNKRKSYLPYRQDLVGIFANVNSLGRTFPPF

SEQ ID NO: 142 - protein, 690 aa, synthetic construct
MEKKQLDFNAPLLSVRKISSSLSPHERANKKIIERAPPNRQQSLPVKKSDWELSEVTKPVAVPFMWEQIPGRRKGNNDARANLRVECSSSPRLPPGRLPETIRFYSGERPRAQNIYKSPAEGLPWIDHAALLDSLVESIYTRGDRESEDDAYSDAPETLSPTESLSLDCSVSGLSGHQSSDSKPSGTFSIDSQTRDFMMSRFLPAAKAVVLETPQYVQKKQVPVSTEQPKPVPVERKPTVKRMESKPVSYYSGYPDDVGSEIEDDVSENQHKRPSKGWKFFPRICVKNSLCLLNPLPGLKVKTHVPTPSSQAVKRVSGLKPKTPQSPTSYAHEVKRLARKAYSGPLEKNSCDTINKQRFHSGVLSRELYKADNRSFSGQLPNPSDSCKLVGISPGRRSRSGAISPYRNVAPPSPFNEGTRFLGVPKEMESLWASRFDSFRKGCYTVKDKVPQQIGTGRFSDSPSEVVEKTLYIDSVDNVQISARNSASSKPKGLVNSSSKNMKTLVKSSVVLENMDATARTQGAKNWNVSEKESKQIAEKESLDLVEASPVSISTLKGPADQESLKLKQNLDTLSGALESSKVHPYGNLGTENEDNKNANDPKDSNLTSVESPIPPPLPKSPSESWLWRTLPSIPLRTPFSSLSSKKQNKKSHTDGTKWETIVKTSNLHKDHVRYSEELYTLGSCQQSKA

SEQ ID NO: 143 - protein, 391 aa, synthetic construct
MEIARSSRRCKWVTAFLVLLACSAAGQVEDGPLVNGDFETPPSGGFSSGDGFSDGPLVLPSWKTNGTVELVESGQKQGGMILIVPQGRHAVRLGNDAEISQELKVEKGSIYSVTFSAARTCAQLESLNVSVPPASQTIDLQTLYNVQGWDSYAWAFQAEEDDVRAVFTNPGMEDDPTCGPIIDDIAIKKLFTPDKSKDNAVLNGDFEEGPWMFRNASLGVLLPTNLDEETSSLPGWIVESNRAVRYIDNYHFTVPEGKRAIELLSGKEGIISQMVETKPNKPYRLTFLLGHAGDSCKQPLAVMAFAGDQAQNIHYTPNSNSSFQIANLNFTAKADRTRVAFYSIYYNTRSDDMSSLCGPVVDDVRVEVSGSSTVKVLGFGFMLWLLVLVLV

SEQ ID NO: 144 - protein, 375 aa, synthetic construct
MRTLCPNLDKADGLETVLEVPIPEEMFNNMGSNGALQWRNMRNLMRAQSADRYSSSSHHAAVSSNDQFMFLFKIVGSALVPFQVQLDHIADNMPVRNGSMEAATAKYIVQQYLAACGGQAALNSLNSMCAVGQVKMATWDIHQSGGDANSKRHCEVGAFVLWQKNPDLWVLELVVSGCKISAGSNGKVAWSQSSSTSSASKGPPRPLRRFFQGLDPRSTANLFLNAICVGEKTIKDEECFILKLESSIDMLKAQSTANTEVVHHTILGYFSQRTGLLIQFEDTKLVRLKSPKGDSNVFWETSMESMLEDYRYIEGINIAHSGKTAATIYRYGKNIDYRAKIEETWMIEEIDFNISDLPMDCFLPPAEFEEQGMGC

SEQ ID NO: 145 - protein, 185 aa, synthetic construct
MLQHLYLGQFSMMFLLLLCSIFAVLTTAESSIYEVLESHGLPMGLLPKGVKNFTLDNSGKFVVHLDQACNAKFENEFHYDRNVSGTISYGQIHALSGIEAQDLFLWFPVKDIRVDIPSSGLIYFNVGVVSKQFSLSSFETPRDCTAVQLTDLQDDMHIADAISKSQSGELRYKFDQGNIGRKAIL

SEQ ID NO: 146 - protein, 305 aa, synthetic construct
MQAYGRMKRVTDPLDDKVKARIIGRDQQELGYLSSGSEHSAHADDDASCSFSTLIFGLPDDVAAENMSSENDSDSDDGDVSMYESTDVIEDLPKPVFYSDLDLFRNSLATKITKALEMFSFSKSNKPILRRNVMTYLRDFGYNAAICKTKWESSGGLKAGNYEFLDVIRSDSGNRITRYFIDLDFAAEFEIARPTNHYERLSQSLPRVFVGKSEELKKMLKVMSDAGRRSLKSKDLSIPPWRKHRFMQNKWLGAYKRTTNVLPSANSSALLSPSKQTNVAKCRSVGFSAVAVNGRLLFPAATRTR

SEQ ID NO: 147 - protein, 361 aa, synthetic construct
MASRKRSISNDVDMHVLYKELDEASCPVCMDHPHNAVLLICSSHDKGCRSYICDTSYRHSNCLDRFKKLRAENRDSPPIMTQGNLDIAVENPDEQLRNLSDRSVVHGNSNRDNHMEMHEGTVQTSGAATVWGSSQETASADGSSDSKLKLKCPMCRGDVLGWKVVEEARKYLNLKNRSCSRESCSFVGNYRELRRHARRVHPTARPADIDPSRQRAWRRLESQREYDDIVSAVRSAMPGAVVLGDYVIESGDRLSGERERGAGGNSRWLSTFFLFQMIGSMDPISEARGGRSRALSRHRRSTGPLSRRRYLWGENLLGLQDDDEDEDERDLNILSDMSGDIPTNPRRRRRLMRSRSDEDQQ

SEQ ID NO: 148 - protein, 403 aa, synthetic construct
MPFPMKIQPVDFNTVEEPSRYDSFKPVQKSRFKRLFERQFSGLLRSSAPLEKLVAGEELICNKKDVASDEFEPSSVCLAKMVQTFIEEGEADKHRCNRNRKCNCFNCNGTDSSEEETDSLSCFGESNHTCCNDACEILKSLVPCPSVIERNVLADITKIIEKNKMGKRKDNFIRKIVVDGLLAAGYDASICESRWDKTPSTPAGTYEYVDVVIEGERLLIDIDFRSEFEIARSTRSYKFLLQLLPNTFVGKADRLQKIVHLLTEAAKQSLKKKGMPFPPWRKAEYVKAKWLSPYTRIKPTLMGAPTVSNSAPETGTACKTYQQAVKEESSEDSCGELNLIFGESSQLLENNAKSVITYPLSACDDEEKDVMVQQGKPPDTKHKDSSNGARKITGLTSLIEDHT

SEQ ID NO: 149 - protein, 164 aa, synthetic construct
MPMSLTSILCFFFLFFSATTASRNEKPSAYEELQRYDFPMGILPKGVKDYKLNTKTGEFSAYLNSTCSFRLENSYQLNYKPVIKGVISKGRLTKLSGVSVKVVLLWLNIVEVRRKGENLEFSVGLTSANFPIENFEECPQCGCGLDCVSKEERKIRQKVFVSSS

SEQ ID NO: 150 - protein, 108 aa, synthetic construct
MEELRPSFRCSGDRRLEIVSGKGFSSNQGSRARSPDLPVVTSKGTWPSQVAAATSSSAKPWGFNDPEMKRRKRIAKYKVYTIEGKFKTSIRNGLRWFKNKCSEIIHGY

SEQ ID NO: 151 - protein, 275 aa, synthetic construct
MGFSENNRGLVLAMVSSLFIGTSFILKKKGLRRAAAAGTRAGVGDYTYLLEPLWWAGMITMIVGEASNFVAYIYAPAVLVTPLGALSIIISALLAHFMLRERLQRLGVVGCILCIVGSVVIFIHAPQEHMPTSVQEIWILAIQPAFMIYIAATVSTVVALMLHFGPRYGQTNLLVYLVICSLMGALTVVSIKAIGIAIKLTLEGISQIAYPQTWFFLAVAVICVITQLNYLNKALDTFNAAIVSPIYYVMFTTLTIIASAIMFKDWAGQDASSIV

SEQ ID NO: 152 - protein, 232 aa, synthetic construct
MGTDTEVAEMSSTLKLPLFSVESSQEDLGMLTPLHTLASIPFKWEEEPGKPRPCTDLIPLPNSICLEPPPRLYNMEFSKTSSSPTTVFDGPYITNKPRFSSFRLLRRDRRHRRQGSFDSTTSSETGQLSTTLVLGNKKSESKSWWRRPTTHHVKHNGCEINSGFVFPSSIDYSTDCVEECSSSSIKMETFRRSGSFSGHSQAKTHIWAAIYEGFKQVIPWKNRKSKKEALIG

SEQ ID NO: 153 - protein, 144 aa, synthetic construct
MSGEDAAVPVVAAAETPAPALGEPMDIMTALQLVLKKSKAHGGLARGLHEGAKVIEKHAAQLCVLAEDCDQPDYVKLVKALCADHNVSLITVPNAKTLGEWAGLCKIDSEGKARKVVGCGCVVVKDYGEETEGLHIVQEYVKSH

SEQ ID NO: 154 - protein, 540 aa, synthetic construct
MGNRSSNLDNDNNISRPIDTPFKLPSPLPTWPSGQAFATGVIDLGGLEVSQTSSLAKVWATQESGPDDLGATFFEPSNLPNGFFMLGSYSQPNNLPLFGSVLVGKDSKGDALKIPIDYTLVWSSENLNIKQDSVGYIWLPIPPEGYKAVGHVVTTSPQKPSLDKIRCVRSDLTDVSESDDWIWGNNGLNVYSSRPRDRGINALGVSTGAFVASNNGTADSLACLKNVNANLSAMPNLNQVKALFQAYSPLYYFHPDEQYYPSSVTWFFQNGALLYTKGQESAPVAIESNGSNLPQGGSNDGAFWLDLPTDDSAKDQVKKGDLQAATAYLHIKPMFGATYTDIALWLFYPFNGPARAKIEFMTIPLGKIGQHVGDWEHVTLRISNFNGELQGVYFSQHSGGNWVSASQLEFQNGNKPVAYSSLHGHASYAQPGKNLQGNNNIGLRNDTAKGQMMDTGANYSIVAAEYLTIVEPAWLNYAREWGLKISYDIANELQKVERFLPGKLKNAVEKLVKSLPNEVLGEEGPTGPKFKDMWNGDERG

SEQ ID NO: 155 - protein, 163 aa, synthetic construct
MAFSQKSQLLFCSLLFILISLSLSFNPSRPNKDDDVHDLLPSYNLPIGLLPNNVKSYTLSPKDNSFTVQLTHPCYVQFQDQLVYYQKEIKGKMSYGEVSDVSGIQAKKLFVWVPVTGINVDEESHMIEFHVGFLSEKLPAKDFETIPTCKNNKGCQDSLLSSI

SEQ ID NO: 156 - protein, 155 aa, synthetic construct
MLLGKRPRPPIKRTTSMKEFTLDLNINDPSVAVINYQPFDPLNPYNFTGPVPMPSNGLDQRLILTRRRSADFVGTAHFLRACSLCKRQLIPGRDIYMYRGDSAFCSLDCREKQMKQDEKKEKYPKVATRKKVTNSAAMAAVASGTGSDAPPQARP

SEQ ID NO: 157 - protein, 480 aa, synthetic construct
MANKVSNFSDLIQRVTASCLLHPLSSGRIDSNISDAHYSDESEDDKYSTEDEEEKGDLPYVETQEKFQNVEVTKEEIKTEKVTEMEMLLGEVFDVVSAVKTAYVSLQEAHCPWDQDKMRVADVAVISELRRLGVLRERFRRNIGGGIRKGDWRVGAATLREVVAPYEATMEELRQEVKAKEIEIDNLREKLKMATSLSSSGGKKSKSKRKISCSSQAPVAMSPAPDVFETTVSLVKEASKSFTSLLLSLMRSAHWDIAAAVRSIEAASSNTNTPTADSIVGSNHAKYALESYVNHKMFQGFDHETFYLDGSLSSLLHPDQHRRDCFTQYRDMKAMDPMELLGILPTCSFGNFCFKKYLAIVHPKMEESLFGDLEQRRQVLAGNHPRSQFYGEFLGLAKAVWLLHLLTFSLDPPPSHFEASKGSEFHPQYMESVVKYSISMGIGGRMGMGLVVGFPVSPGFKLGNGSVVKARVYLVPKNGF

SEQ ID NO: 158 - protein, 410 aa, synthetic construct
MMGSGLQFNRSFNGEDRFYSAGKFRRNVNRSFSTGSEDNLRRAKSDVAVIHSPTKPKMKVAADESPDEPVSAVPPTDVLPPPLCNLQRFLKSVSPSVPAQYLSKTTMRGWRTCDVEFQPYFVLGDLWESFREWSAYGAGVPLVLNEGDSVVQYYVPYLSGIQLYGDSTKASTKTSRRPGEESDSDYFRDSSSDGSSDSEHERRCLNYTREQRMYHSQTSESSLSIDRLSLRDRNATFQEGFSSDEGESGSSQGALLFEYLEHGQPYGCEPLADKISDLAQRFPELKTMRSCDLLPSSWISVAWYPIYRIPTGPTLKALDACFLTFHSLHTPMTGSQSAHPAAVTCPSDTDPVPKIPLPAFGLASYKFKASLWTPNGGSGRQLMSSLLQSAENWLTLLQVNHPDFSFFCQR

SEQ ID NO: 159 - protein, 226 aa, synthetic construct
MGSCVSVHKDSESAMKIRLVFGSSKTDKLVTPSPLNKDDTKVSDLQLKSQTPAVTAFRDFGSKEETFFDSQPWLDSDCEDDFLSVNGDFTPSRGNTPVHPLAGNLTGNRTPAYFQQSSPTDKKKRLSELFEESLRSDLDQNGQNAEEKQNGTNTKKETASTGTQLPPRSTPGTPYASVCSSERTPSGLLKSDVKTSKSAQCCLPRLLSSRSFNGRRSRMSPARNVG

SEQ ID NO: 160 - protein, 199 aa, synthetic construct
MKDDESIPVSTPTTHSSSNTISTYSKNSTSDATIFGRGRYKFWALAAILLLAFWSMFTGTVTLRWSAGNLNGLSDYFNVPLSDNLDVIEMEEREKLVKHMWDVYTNTPRIRLPKFWQEAFEAAYEELTSDLADIREAAISEISKMSLRFVHMEPPPLRSLAVRELSQKQAEDKQRTAKGIWLSAVLMEENKREEKRGPD

SEQ ID NO: 161 - protein, 182 aa, synthetic construct
MADSESRQFSPDKEPCFAEELLDWQRRELPRRFLSGQVVWDDDAFIPEGASEKHRVIFRRIYRKYFLQIFASDGFDIDIYPGKAKAAMLIPYLDFEKEIDLLMELANHAIQDYNSKETNVYKYEVLYVEKVNFILAECREFFMTVKVKNLTLRSPKETFQIHAYKGPDGENIFCLCRRKLLV

SEQ ID NO: 162 - protein, 302 aa, synthetic construct
MRDFASCFNEYAVQVSDTSCSSYSNSACIPPSLIPSIQNTVTCLYKVTLSNKKQVLITVSWSKTNSITQGLSVHFGDDPSNVFKLNTNSRLFRKKRGSKSLDLDHLKVEIFWDLYAARYLSGPEPIDGYYLLVKVDSQLGLIIGDMAEEASLRKLKNGTPMAKFSLVSRKEYFSGNTLYSTKAQFCDNGTSHDILIRCSGENEGLKHPVLSVYIDKKMVIRVKRLQWNFRGNQSIFVDGLLVDLMWDVHDWFFNPTSGCALFMFRTRSGMDSRLWLDDKDKLLHKDPDKVEFSLLIYASKTT

SEQ ID NO: 163 - protein, 167 aa, synthetic construct
MAASKSYFAGANYRFLSSERDVPMMSPESAFEFDESDLWNSAVSQSPEFRKSVQSSKISRKQCDTKSRRSGSVASAASLPVNVPDWSKILKDEYREYGRRDSDDDDFNDEDGDDLENRIPPHEFLAKQLERTRIASFSVHEGVGRTLKGRDLSRVRNAIWEKTGFQD

SEQ ID NO: 164 - protein, 758 aa, synthetic construct
MERRPKQSGERKTMENEELVKYMSSLPSYLEKGENLQEKAFSVGVLDWRFLEKWRHEHVKEPCRTSGCSPSTSNTLSFSSMEGSSSNSSRARSCSPARRRIHRPTSQSYYASPPKGSYVQLQNQKPGVIKSPEIEQLVCRIYQSFDEYPQRNMQELGNALHPRRTPHVRRSLDVETKTRTSRSKGKMKIQDRECLSKGDFDDFDCIEKHKSDVLQVPEPGQETNSCTTFCPPDSVVKDQSAVKSSRRSFSCGFISAFYYGQSSSDISSSSTLPHDADESKVGQASPIDAKDSCLSSKTIQPSTYSGNKLSSPPGTNSKQEKKSTVMLKNPTTLNSAESTNIRNSSPTRQFSMAMGRIGQISGIKDMITGSQGVKWPAEQSSPNKTQSSSSIDTGCDKSDTTGRARTSPLRRLLDPLLKPKTGNSDHVKSSTRRGESPTKRSLKVKLDLKSCKSIDIDDPRSNGTFVPSRLQALLQVAVKNGLPLFTFAVDNEVDILAATMKKLNPNLKDYSCWIYNFFTVRETKKKSGNWLNQVEKDRSHGIMPNIVGKMKVSDVPFSELNRQKLDSQFRITEFVLFATDQKASDLHPNDELAAIVVKLPNRTSICPNGSGHQDRNCSNTSTSGLTNPFEDLNMTVILPGGAHSVPSKGEPSSLINRWRSGGSCDCGGWDLGCKLRLLRNHTNHQRISSCSKPKLNAARFELLSQGEARDSKPVFSLSAFKDGIFSVEFSSSLKVLQAFSICIAVLNGRNQESFQMQT

SEQ ID NO: 165 - protein, 185 aa, synthetic construct
MLQHFCLGQFSAMFLLLLCSIFAVLSTAESSIYEVLESHGLPRGLLPKGVKNFTLDNSGKFVVHLDQACNAKFENEFHYDRNVSGTISYGQIHALSGIEAQDLFLWFPVKDIRVDIPSSGLIYFNVGVVSKQFSLSSFETPRDCTVVQLMDLQDDMHIADAVSKSQSEELRYKFDQGNIGRKAIL

SEQ ID NO: 166 - protein, 304 aa, synthetic construct
MASTVPAKSNNPLYNFDFSHLKWKKNHHSNNHQRRRSNKLSSDSSSPSRHDSPLRHSQSQSPMRESLAAARQSPVSESAETARISPMHNSAAAARQSPMRESARQSPMRDPVPSVQRSKHKVPEINVVSSKESRSKILIKIPRKNKSEEIQINEDQNQKEADESHDEAAAAEETAQKTWNLRPRKPIHKSLNINGGVPFRSSGSAMQEIKSQSPHHMMNVNKPENNETHAASAQKKVKRQRFSIALSREEIDEDLYAMTGLKAARRPKKRVKVVQKQLDTLFPGLWLASITPDSYKVCENLPKG

SEQ ID NO: 167 - protein, 370 aa, synthetic construct
MMNNQSSICSSLNEDKVHDTPDSFHQLHPKKDSDSLDGSELESGDTISTVEHLKSALKAERKALHSLYTELEEERSASAVAASQTMAMINRLQEEKAAMQMEALQYQRMMEEQSEYDQEALQLMNELMVKREREKQELEKELEAYRKRLLEYEATRMLKRSKDSSTFSSEDSDGLSIGLNQEEAKEDDSGMNHSTPVGAVINLEEERMAILEQLKVLEERLVSLDLDDEDAKHFEDVRLMEDSYQDNIEEDSHANGFLKEMNGKHHHAKGKRLLPLFDKMSDENGDGTLNANGVHNFDLENKKLAVEQELDHLHQRLQALEADREFLNNCVSSLKKGDKGMDLLQEILLHLRHLRNVELRATSFSDAAIL

SEQ ID NO: 168 - protein, 370 aa, synthetic construct; identical to SEQ ID NO: 167

SEQ ID NO: 169 - protein, 144 aa, synthetic construct
MLWRNSTKLQLCSSSARSRLLLICRLEHQFIVTHRKPVDLWGLKICELKRPIAAVKLNCKLQVVVPCCYSLAAIVNIFSSLQSLMNFAFELQLGVFLQNCEEDCSGDHQQSATLSVIVRLSRIADLTSPYLCGHSLQIRPILQN

SEQ ID NO: 170 - protein, 355 aa, synthetic construct
MFDDSVSTVYSSIWTSMNSWFTPTVLFVLLNLMIGTIAFTSSLANQKPNHPPKQEQQQEEQEYSQNHPQQPSKLIRSPSILQRIKSLNFYNNRSQEPVKDYNFDDTHNQETTFEPQTHYIFEPAPEHTTVVPESTQYIFNQSHQENAQKTQTQYDFQQTHLENLPDTQTQYLFQHTPDQELKNMDQDFHFQQTHQENIQEKSARYIIQHIHEQKLDNTDTHFDFQQTHEENEVEEEEEEGELKSLDEVYNQLKERHVSRSRSDTNPSAGEAPIKLPTKMKKSASMKSPFAHFEEEAIVEARRPATTRERNVKTSDGDINEVDAKADDFINKFKQQLKLQRLDSILRYKEMIGRGS

SEQ ID NO: 171 - protein, 385 aa, synthetic construct
MEIVAKSKKHHHYYSPFPSCFRRPLEVAAAASARPLPPPPPPPQPPVAANPNLATSLYQTHLGLFALTWSRNLFGRSFHIHFLLNDSDGVGADYNNNTISSPHLSSTSTPSFHLNIKPFIFWKKHGSKKLDGDNKVVHIFWDLSKSKFGSGPEPISGFYVAVIVNEEMVLLVGDLNKEAYAKTRARKPEKKQNLNPNPNPNLVLRREHVCGNKLYKTKANFGGKEKEISIDCRLGEDPRLYFSVDNKRVLQIKHLKWKFRGNERIEVDGVPVLVSWDVYNWLFDDDEDGYALFMFKFEKSSYEYVAADDYSLNNGVQLWSQQSCGFGFETKMMKKGVLRSSRSSSSSSLSSASSTCSSVMEWASTEENELKGPSGFSLLVYAWKS

SEQ ID NO: 172 - protein, 196 aa, synthetic construct
MWQFNKIWCQNQRIQITYWGLLAYNAGDDMVTSTTPFELIIQSDKIPITIDFSNTTRLVNDTSTSGIIPLCASFERDGKVTLTKQVSQNVCAAKKQGHFGLVIESPLMPLKNKRASNWKLAIGSAIGAALGAFLLGLLLIAIFVKVKKKARMEELVRRAYEEEALQVSMVGHVRAPTAAGTRTVPTIEHDYNPHNS

SEQ ID NO: 173 - protein, 170 aa, synthetic construct
MDQILNKVGSYWIGKRANKELNSVGDDINSLQSSIEGGTKWLVNKLKGKMQKPLPDLLKEYDVPVGIFPRDATNYEFNEETRKLTVYIPSVCEVGYKDSSVLRFSTEVTGFLEKGKLADIEGMKTKVMMWVKVTAISSEKSKVHFTAGLKKTRSREAYEVLRDGVAIEKF

SEQ ID NO: 174 - protein, 224 aa, synthetic construct
MGAISFLDTILVPLSLFITIGYHFYLWHHLKHKPSRTTIGMNILKKRSWLRELNQGNEKKGTLAVQSLRNALMETILTATITMIITLALAALTNNTYNVRNLFTSAFFGSQTGKIIVLKYGSATIFLLASFLCSSLALSCLIDANFLVNALGEFSINPMYTETVFERGFTLAFVGDRVLCMAFPLLLWMFGPVPVVVSSLALVWGLHERDFAGNLSRTMNKSCI

SEQ ID NO: 175 - DNA, 3155 bp, synthetic construct
  source 1..3155: mol_type = other DNA
aagcttccagaaggtaattatccaagatgtagcatcaagaatccaatgtttacgggaaaaactatggaagtattatgtgagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtggatcctgcaggctagcgtgcactctagactcgacgaactgacgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactatgataaattatcgcgcgcggtgtcatctatgttactagatcgggaattcctcgagcaactatttttatgtatgcaagagtcagcatatgtataattgattcagaatcgttttgacgagttcggatgtagtagtagccattatttaatgtacatactaatcgtgaatagtgaatatgatgaaacattgtatcttattgtataaatatccataaacacatcatgaaagacactttctttcacggtctgaattaattatgatacaattctaatagaaaacgaattaaattacgttgaattgtatgaaatctaattgaacaagccaaccacgacgacgactaacgttgcctggattgactcggtttaagttaaccactaaaaaaacggagctgtcatgtaacacgcggatcgagcaggtcacagtcatgaagccatcaaagcaaaagaactaatccaagggctgagatgattaattagtttaaaaattagttaacacgagggaaaaggctgtctgacagccaggtcacgttatctttacctgtggtcgaaatgattcgtgtctgtcgattttaattatttttttgaaaggccgaaaataaagttgtaagagataaacccgcctatataaattcatatattttcctctccgctttgaattgtctcgttgtcctcctcactttcatcagccgttttgaatctccggcgacttgacagagaagaacaaggaagaagactaagagagaaagtaagagataatccaggagattcattctccgttttgaatcttcctcaatctcatcttcttccgctctttctttccaaggtaataggaactttctggatctactttatttgctggatctcgatcttgttttctcaatttccttgagatctggaattcgtttaatttggatctgtgaacctccactaaatcttttggttttactagaatcgatctaagttgaccgatcagttagctcgattatagctaccagaatttggcttgaccttgatggagagatccatgttcatgttacctgggaaatgatttgtatatgtgaattgaaatctgaactgttgaagttagattgaatctgaacactgtcaatgttagattgaatctgaacactgtttaaggttagatgaagtttgtgtatagattcttcgaaactttaggatttgtagtgtcgtacgttgaacagaaagctatttctgattcaatcagggtttatttgactgtattgaactctttttgtgtgtttgcagctcataaaaggtaccaaacaatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttttgagcgggactctggcgatcgccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggactagt

SEQ ID NO: 176 - DNA, 465 bp, synthetic construct
  source 1..465: mol_type = other DNA
atggctccagctaaagctgatccgtccaaaaaatctgaccccaaggcacaggcagctaaggttgccaaggctgtcaagtcaggatcaaccttgaagaaaaagtcacaaaagataaggacaaaagttacattccaccgacctaagactttgaagaaggatagaaaccccaagtaccctcgtattagtgcacctggaaggaacaaacttgatcagtatgggattctaaagtatcccctcaccacagagtctgcaatgaagaagattgaggacaacaacacccttgttttcattgtggacatcaaggctgataaaaagaagattaaggatgccgtgaagaagatgtatgacattcagacaaagaaagtcaataccttaattaggcctgatgggacgaagaaagcatatgtgaggttgactcctgactatgatgctttggacgttgccaacaaaattggaatcatctaa

SEQ ID NO: 177 - DNA, 22 bp, synthetic construct
  source 1..22: mol_type = other DNA
aaagaagattaaggatgccgtg

SEQ ID NO: 178 - DNA, 22 bp, synthetic construct
  source 1..22: mol_type = other DNA
gcatcatagtcaggagtcaacc